(19) United States
(12) Patent Application Publication
(10) Pub. No.: US 2026/0217858 A1
(43) Pub. Date: Jul. 30, 2026

(54) COMPOSITIONS AND METHODS RELATED TO TUMOR ACTIVATED ANTIBODIES TARGETING PSMA AND EFFECTOR CELL ANTIGENS

(71) Applicant: Janux Therapeutics, Inc., San Diego, CA (US)

(72) Inventors: David CAMPBELL, San Diego, CA (US); Thomas R. DiRaimondo, San Diego, CA (US)

(21) Appl. No.: 19/570,589

(22) Filed: Mar. 18, 2026

Related U.S. Application Data
(63) Continuation of application No. 19/320,207, filed on Sep. 5, 2025.
Application No. 19/320,207 is a continuation of application No. 18/055,932, filed on Nov. 16, 2022, now Pat. No. 12,617,865.
Application No. 18/055,932 is a continuation of application No. 17/544,539, filed on Dec. 7, 2021, now Pat. No. 11,555,078.
(60) Provisional application No. 63/123,329, filed on Dec. 9, 2020.
(60) Provisional application No. 63/187,699, filed on May 12, 2021.

(51) Int. Cl. C07K 16/30; C07K 14/765; C07K 16/28

(52) U.S. Cl. CPC C07K 16/3069; C07K 14/765; C07K 16/2809; C07K 2317/565; C07K 2317/569; C07K 2317/622; C07K 2317/92; C07K 2319/30; C07K 2319/31

(57) ABSTRACT

Provided herein are multispecific antibodies that selectively bind to PSMA and effector cell antigens such as CD3, pharmaceutical compositions thereof, as well as nucleic acids, and methods for making and discovering the same.

### CROSS-REFERENCE

The present application is a continuation of U.S. patent application Ser. No. 19/320,207, filed Sep. 5, 2025, which is a continuation of U.S. patent application Ser. No. 18/055,932, filed Nov. 16, 2022, which is a continuation of U.S. patent application Ser. No. 17/544,539, filed Dec. 7, 2021, now U.S. Pat. No. 11,555,078, which claims the benefit of U.S. Provisional Application No. 63/187,699, filed May 12, 2021, and U.S. Provisional Application No. 63/123,329, filed Dec. 9, 2020, each of which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 11, 2022, is named 52426-730_303SL.xml and is 1,248,610 bytes in size.

### SUMMARY

Disclosed herein, in certain embodiments, are isolated polypeptides or polypeptide complexes according to Formula I:

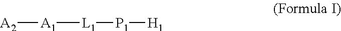

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA). In some embodiments, the first antigen recognizing molecule comprises an antibody or antibody fragment. In some embodiments, first antigen recognizing molecule comprises an antibody or antibody fragment that is human or humanized. In some embodiments, L₁ is bound to a N-terminus of the first antigen recognizing molecule. In some embodiments, A₂ is bound to a C-terminus of the first antigen recognizing molecule. In some embodiments, L₁ is bound to a C-terminus of the first antigen recognizing molecule. In some embodiments, A₂ is bound to a N-terminus of the first antigen recognizing molecule. In some embodiments, the antibody or antibody fragment comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, the antibody or antibody fragment comprises a single chain variable fragment (scFv), a heavy chain variable domain (VH domain), a light chain variable domain (VL domain), or a variable domain (VHH) of a camelid derived single domain antibody. In some embodiments, A₁ comprises an anti-CD3e single chain variable fragment. In some embodiments, A₁ comprises an anti-CD3e single chain variable fragment that has a K_{D} binding of 1 μM or less to CD3 on CD3 expressing cells. In some embodiments, the effector cell antigen comprises CD3. In some embodiments, A₁ comprises a variable light chain and variable heavy chain each of which is capable of specifically binding to human CD3. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the polypeptide or polypeptide complex of Formula I binds to an effector cell when L₁ is cleaved by the tumor specific protease. In some embodiments, the polypeptide or polypeptide complex of Formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3e. In some embodiments, the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv comprise: HC-CDR1: SEQ ID NO: 1, HC-CDR2: SEQ ID NO: 2, and HC-CDR3: SEQ ID NO: 3; and the scFv comprises CDRs: LC-CDR1, LC-CDR2, and LC-CDR3, wherein the LC-CDR1, the LC-CDR2, and the LC-CDR3 of the scFv comprises: LC-CDR1: SEQ ID NO: 4, LC-CDR2: SEQ ID NO:5, and LC-CDR3: SEQ ID NO: 6. In some embodiments, the effector cell antigen comprises CD3, and the scFv comprises an amino acid sequence according to SEQ ID NO: 7. In some embodiments, second antigen recognizing molecule comprises an antibody or antibody fragment. In some embodiments, the antibody or antibody fragment thereof comprises a single chain variable fragment, a single domain antibody, or a Fab. In some embodiments, the antibody or antibody fragment thereof comprises a single chain variable fragment (scFv), a heavy chain variable domain (VH domain), a light chain variable domain (VL domain), or a variable domain (VHH) of a camelid derived single domain antibody. In some embodiments, the antibody or antibody fragment thereof is humanized or human. In some embodiments, A₂ is the Fab. In some embodiments, the Fab comprises (a) a Fab light chain polypeptide and (b) a Fab heavy chain polypeptide. In some embodiments, the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 8, HC-CDR2: SEQ ID NO: 9, and HC-CDR3: SEQ ID NO: 10; and the Fab comprises CDRs: LC-CDR1, LC-CDR2, and LC-CDR3, wherein the LC-CDR1, the LC-CDR2, and the LC-CDR3 of the Fab comprise LC-CDR1: SEQ ID NO: 11, LC-CDR2: SEQ ID NO:12, and LC-CDR3: SEQ ID NO: 13. In some embodiments, the Fab light chain polypeptide comprises an amino acid sequence according to SEQ ID NO: 14. In some embodiments, the Fab heavy chain polypeptide comprises an amino acid sequence according to SEQ ID NO: 15. In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁. In some embodiments, A₂ further comprises P₂ and L₂, wherein P₂ comprises a peptide that binds to A₂; and L₂ comprises a linking moiety that connects A₂ to P₂ and is a substrate for a tumor specific protease. In some embodiments, the polypeptide or polypeptide complex is according to Formula Ia:

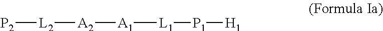

In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂. In some embodiments, P₁ impairs binding of A₁ to the effector cell antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ has less than 70% sequence homology to the effector cell antigen. In some embodiments, P₂ impairs binding of A₂ to PSMA. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A₂ at or near an antigen binding site. In some embodiments, P₂ has less than 70% sequence homology to PSMA. In some embodiments, P₁ or P₂ comprises a peptide sequence of at least 10 amino acids in length. In some embodiments, P₁ or P₂ comprises a peptide sequence of at least 10 amino acids in length and no more than 20 amino acids in length. In some embodiments, P₁ or P₂ comprises a peptide sequence of at least 16 amino acids in length. In some embodiments, P₁ or P₂ comprises a peptide sequence of no more than 40 amino acids in length. In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide In some embodiments, P₁ comprises at least two cysteine amino acid residues. In some embodiments, P₁ comprises an amino acid sequence according to any one of SEQ ID NOs: 16-19 or 78. In some embodiments, L₁ is bound to a N-terminus of A₁. In some embodiments, L₁ is bound to a C-terminus of A₁. In some embodiments, L₂ is bound to a N-terminus of A₂. In some embodiments, L₂ is bound to a C-terminus of A₂. In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments, L₁ or L₂ has a formula comprising (G₂S)ₙ, wherein n is an integer from 1 to 3 (SEQ ID NO: 118). In some embodiments, L₁ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1. In some embodiments, P₁ becomes unbound from A₁ when L₁ is cleaved by the tumor specific protease thereby exposing A₁ to the effector cell antigen. In some embodiments, P₂ becomes unbound from A₂ when L₂ is cleaved by the tumor specific protease thereby exposing A₂ to PSMA. In some embodiments, the tumor specific protease is selected from the group consisting of a matrix metalloprotease (MMP), serine protease, cysteine protease, threonine protease, and aspartic protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, L₁ or L₂ comprises a urokinase cleavable amino acid sequence, a matriptase cleavable amino acid sequence, a matrix metalloprotease cleavable amino acid sequence, or a legumain cleavable amino acid sequence. In some embodiments, L₁ or L₂ comprises an amino acid sequence according to SEQ ID NO: 23. In some embodiments, L₁ or L₂ comprises an amino acid sequence according to any one of SEQ ID NOs: 20-49. In some embodiments, L₁ or L₂ comprises an amino acid sequence of Linker 25 (ISSGLLSGRSDAG) (SEQ ID NO: 45), Linker 26 (AAGLLAPPGGLSGRSDAG) (SEQ ID NO: 46), Linker 27 (SPLGLSGRSDAG) (SEQ ID NO: 47), or Linker 28 (LSGRSDAGSPLGLAG) (SEQ ID NO: 48), or an amino acid sequence that has 1, 2, or 3 amino acid substitutions, additions, or deletions relative to the amino acid sequence of Linker 25, Linker 26, Linker 27, or Linker 28. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7 h-11-15. In some embodiments, the single domain antibody is Alb-1, Alb-8, or Alb-23. In some embodiments, the single domain antibody is 10E. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 58, HC-CDR2: SEQ ID NO: 59, and HC-CDR3: SEQ ID NO: 60. In some embodiments, the single domain antibody is SA21. In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification. In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1. In some embodiments, L₃ comprises an amino acid sequence according to SEQ ID NO: 22. In some embodiments, the polypeptide or polypeptide complex comprises an amino acid sequence with at least 95% sequence identity to SEQ ID NOs: 62-77. In some embodiments, the polypeptide or polypeptide complex comprises an amino acid sequence with at least 95% sequence identity to SEQ ID NO: 72. In some embodiments, the polypeptide or polypeptide complex comprises an amino acid sequence with at least 95% sequence identity to SEQ ID NO: 73. In some embodiments, the polypeptide or polypeptide complex comprises an amino acid sequence with at least 95% sequence identity to SEQ ID NO: 62 and SEQ ID NO: 63. In some embodiments, the polypeptide or polypeptide complex comprises an amino acid sequence with at least 95% sequence identity to SEQ ID NO: 64 and SEQ ID NO: 65. In some embodiments, the polypeptide or polypeptide complex comprises an amino acid sequence with at least 95% sequence identity to SEQ ID NO: 66 and SEQ ID NO: 67. In some embodiments, the polypeptide or polypeptide complex comprises an amino acid sequence with at least 95% sequence identity to SEQ ID NO: 68 and SEQ ID NO: 69. In some embodiments, the polypeptide or polypeptide complex comprises an amino acid sequence with at least 95% sequence identity to SEQ ID NO: 70 and SEQ ID NO: 71. In some embodiments, the polypeptide or polypeptide complex comprises an amino acid sequence with at least 95% sequence identity to SEQ ID NO: 72 and SEQ ID NO: 73. In some embodiments, the polypeptide or polypeptide complex comprises an amino acid sequence with at least 95% sequence identity to SEQ ID NO: 74 and SEQ ID NO: 75. In some embodiments, the polypeptide or polypeptide complex comprises an amino acid sequence with at least 95% sequence identity to SEQ ID NO: 76 and SEQ ID NO: 77.

Disclosed herein, in certain embodiments, are pharmaceutical compositions comprising: (a) the polypeptide or polypeptide complex described herein; and (b) a pharmaceutically acceptable excipient.

Disclosed herein, in certain embodiments, are isolated recombinant nucleic acid molecules encoding the polypeptide or polypeptide complex described herein.

Disclosed herein, in certain embodiments, are isolated polypeptides or polypeptide complexes according to Formula II:

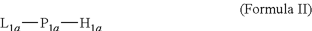

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to PSMA; P₁ₐ comprises a peptide that binds to the first antigen recognizing molecule when L₁ₐ is uncleaved; and H₁ₐ comprises a half-life extending molecule. In some embodiments, P₁ₐ when L₁ₐ is uncleaved impairs binding of the first antigen recognizing molecule to the effector cell antigen. In some embodiments, the first antigen recognizing molecule comprises an antibody or antibody fragment. In some embodiments, the effector cell antigen is an anti-CD3 effector cell antigen. In some embodiments, P₁ₐ has less than 70% sequence homology to the effector cell antigen. In some embodiments, P₁ₐ comprises a peptide sequence of at least 10 amino acids in length. In some embodiments, P₁ₐ comprises a peptide sequence of at least 10 amino acids in length and no more than 20 amino acids in length. In some embodiments, P₁ₐ comprises a peptide sequence of at least 16 amino acids in length. In some embodiments, P₁ₐ comprises a peptide sequence of no more than 40 amino acids in length. In some embodiments, P₁ₐ comprises at least two cysteine amino acid residues. In some embodiments, P₁ₐ comprises a cyclic peptide or a linear peptide. In some embodiments, P₁ₐ comprises a cyclic peptide. In some embodiments, P₁ₐ comprises a linear peptide. In some embodiments, P₁ₐ comprises an amino acid sequence selected from the group consisting of any one of SEQ ID NOs: 16-19. In some embodiments, H₁ₐ comprises a polymer. In some embodiments, the polymer is polyethylene glycol (PEG). In some embodiments, H₁ₐ comprises albumin. In some embodiments, H₁ₐ comprises an Fc domain. In some embodiments, the albumin is serum albumin. In some embodiments, the albumin is human serum albumin. In some embodiments, H₁ₐ comprises a polypeptide, a ligand, or a small molecule. In some embodiments, the polypeptide, the ligand or the small molecule binds a serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the antibody comprises a single domain antibody that binds to albumin. In some embodiments, the antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7 h-11-15. In some embodiments, the single domain antibody is Alb-1, Alb-8, or Alb-23. In some embodiments, the single domain antibody is 10E. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 58, HC-CDR2: SEQ ID NO: 59, and HC-CDR3: SEQ ID NO: 60. In some embodiments, the single domain antibody is SA21. In some embodiments, H₁ₐ comprises a linking moiety (L₁ₐ) that connects H₁ₐ to P₁ₐ. In some embodiments, L₁ₐ is a peptide sequence having at least 5 to no more than 50 amino acids. In some embodiments, L₁ₐ is a peptide sequence having at least 10 to no more than 30 amino acids. In some embodiments, L₁ₐ is a peptide sequence having at least 10 amino acids. In some embodiments, L₁ₐ is a peptide sequence having at least 18 amino acids. In some embodiments, L₁ₐ is a peptide sequence having at least 26 amino acids. In some embodiments, L₁ₐ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1. In some embodiments, L₁ₐ comprises an amino acid sequence according to SEQ ID NO: 23. Disclosed herein some embodiments are polypeptide complexes comprising a structural arrangement according to the configuration shown in FIG. 1C, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to the heavy chain variable domain of the scFv with a linking moiety that is a substrate for a tumor specific protease, and the peptide further comprises a half-life extending molecule; and a Fab or a Fab′ that binds to prostate-specific membrane antigen (PSMA), wherein the Fab or Fab′ comprises a Fab light chain polypeptide chain and a Fab heavy chain polypeptide chain, and wherein the Fab heavy chain polypeptide chain is linked to a C terminus of the light chain variable domain of the scFv. Disclosed herein in some embodiments are polypeptide complexes comprising a structural arrangement according to the configuration shown in FIG. 1D, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab or Fab′ that binds to prostate-specific membrane antigen (PSMA), wherein the Fab or Fab′ comprises a Fab light chain polypeptide and a Fab heavy chain polypeptide, wherein the Fab light chain polypeptide is linked to a C terminus of the light chain variable domain of the scFv.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1R illustrate polypeptide complexes of this disclosure in a normal orientation (FIG. 1A), flipped orientation (FIG. 1B), and in several structural arrangements (FIGS. 1C-1R).

FIG. 2A illustrates titration data for PSMA binding for several polypeptide complexes of this disclosure.

FIG. 2B illustrates titration data for PSMA binding for several polypeptide complexes of this disclosure.

FIG. 2C illustrates titration data for PSMA binding for several polypeptide complexes of this disclosure.

FIG. 3A illustrates titration data for CD3ε binding for several polypeptide complexes of this disclosure.

FIG. 3B illustrates titration data for CD3ε binding for several polypeptide complexes of this disclosure.

FIG. 3C illustrates titration data for CD3ε binding for several polypeptide complexes of this disclosure.

FIG. 4 illustrates cell viability data for 22Rv1 tumor cells treated with PC1 or PC2.

FIG. 5A illustrates cell viability data for 22Rv1 tumor cells treated with PC1, PC5 or MTSP1 treated PC5.

FIG. 5B illustrates cell viability data for 22Rv1 tumor cells treated with PC2, or PC4 or PC6 with and without MTSP1 treatment.

FIG. 6 illustrates cell viability data for LNCaP tumor cells treated with PC1 or PC2.

FIG. 7 illustrates cell viability data for LNCaP tumor cells treated with PC2, PC4 or MTSP1 treated PC4.

FIGS. 8A-8B illustrates polypeptide complex mediated 22Rv1 tumor cell killing in the presence of CD8+ T cells.

FIG. 9A illustrates polypeptide (PSMA TCEs) pharmacokinetics in cynomolgus monkeys after a single IV bolus injection.

FIG. 9B illustrates polypeptide (PSMA TRACTrs) pharmacokinetics in cynomolgus monkeys after a single IV bolus injection.

FIG. 10A illustrates cytokine release in cynomolgus monkeys after single IV bolus of PSMA TCE.

FIG. 10B illustrates cytokine release in cynomolgus monkeys after single IV bolus of PSMA polypeptide TRACTr complex.

FIG. 10C illustrates cytokine release in cynomolgus monkeys using PSMA TCE versus PSMA TRACTRs.

FIG. 11A illustrates serum liver enzymes in cynomolgus monkeys after single IV bolus of PSMA TCE.

FIG. 11B illustrates serum liver enzymes in cynomolgus monkeys after single IV bolus of PSMA polypeptide TRACTr complex.

FIGS. 12A-12F illustrate anti-CD3 scFv binding by alanine scanning peptides of anti-CD3 scFv Peptide-A and Peptide-B as measured by ELISA.

FIGS. 13A-13F illustrate inhibition of anti-CD3 scFv binding to CD3 by alanine scanning peptides of anti-CD3 scFv Peptide-A and Peptide-B as measured by ELISA.

FIGS. 14A-14B illustrate anti-CD3 scFv binding by optimized anti-CD3 scFv Peptide-B sequences as measured by ELISA.

FIGS. 15A-15B illustrate inhibition of anti-CD3 scFv binding to CD3 by optimized anti-CD3 scFv Peptide-B sequences as measured by ELISA.

FIG. 16 illustrates the core sequence motif of optimized anti-CD3 scFv Peptide-B sequences generated using WebLogo 3.7.4.

### DETAILED DESCRIPTION

Multispecific antibodies combine the benefits of different binding specificities derived from two or more antibodies into a single composition. Multispecific antibodies for redirecting T cells to cancers have shown promise in both pre-clinical and clinical studies. This approach relies on binding of one antigen interacting portion of the antibody to a tumor-associated antigen or marker, while a second antigen interacting portion can bind to an effector cell antigen on a T cell, such as CD3, which then triggers cytotoxic activity.

One such tumor-associated antigen is PSMA. Prostate-specific membrane antigen (PSMA), also known as glutamate carboxypeptidase II (GCPII), N-acetyl-L-aspartyl-L-glutamate peptidase I (NAALADase I), or NAAG peptidase is an enzyme that in humans is encoded by the FOLH1 (folate hydrolase 1) gene. PSMA is a zinc metalloenzyme that resides in membranes. Most of the enzyme resides in the extracellular space. Human PSMA is highly expressed in the prostate, roughly a hundred times greater than in most other tissues. In some prostate cancers, PSMA is the second-most upregulated gene product, with an 8- to 12-fold increase over levels in noncancerous prostate cells.

T cell engagers (TCEs) therapeutics have several benefits including they are not cell therapies and thus can be offered as off-the-shelf therapies as opposed to chimeric antigen receptor T cell (CAR T cell) therapies. While TCE therapeutics have displayed potent anti-tumor activity in hematological cancers, developing TCEs to treat solid tumors has faced challenges due to the limitations of prior TCE technologies, namely (i) overactivation of the immune system leading to cytokine release syndrome (CRS), (ii) on-target, healthy tissue toxicities and (iii) poor pharmacokinetics (PK) leading to short half-life. CRS arises from the systemic activation of T cells and can result in life-threatening elevations in inflammatory cytokines such as interleukin-6 (IL-6). Severe and acute CRS leading to dose limited toxicities and deaths have been observed upon the dosing of T cell engagers develop using other platforms to treat cancer patients in poor clinical studies. This toxicity restricts the maximum blood levels of T cell engagers that can be safely dosed. T cell engager effectiveness has also been limited because of on-target, healthy tissue toxicity. T cell engagers developed using a platform not designed for tumor-specification activation have resulted in clinicals holds and dose-limiting toxicities resulting from target expression in healthy tissues. T cell engagers have also been limited by short half-lives. T cell engagers quickly reach sub-therapeutic levels after being administered as they are quickly eliminated from the body due to their short exposure half-lives. For this reason, T cell engagers such as blinatumomab are typically administered by a low-dose, continuous infusion pump over a period of weeks to overcome the challenge of a short half-life and to maintain therapeutic levels of drug in the body. A continuous dosing regimen represents a significant burden for patients.

To overcome these challenges associated with the effectiveness of T cell engagers, described herein, are polypeptide or polypeptide complexes that comprise binding domains that selectively bind to an effector cell antigen and PSMA, in which one or more of the binding domains is selectively activated in the tumor microenvironment and the polypeptide or polypeptide complex comprises a half-life extending molecule. Such modifications reduce CRS and on-target healthy tissue toxicity risk, improves stability in the bloodstream and serum half-life prior to activation. The polypeptide or polypeptide complexes described herein have activity at low levels of target expression, and are easily manufactured.

In some embodiments, the polypeptides or polypeptide complexes described herein are used in a method of treating cancer. In some embodiments, the cancer has cells that express PSMA. In some embodiments, the polypeptides or polypeptide complexes described herein are used in a method of treating prostate cancer. In some embodiments, the prostate cancer is metastatic castrate resistant prostate cancer (mCRPC). Prostate cancer is the second most common cancer in men worldwide with over 3 million men living with prostate cancer in the United States alone. Early diagnoses and effective therapies mean that most prostate cancer patients have a prognosis with a mean five-year survival rate of approximately 98 percent. However, an estimated six percent of prostate cancer patients develop metastatic disease, which is associated with a five-year survival rate of approximately 30 percent. There were an estimated 33,000 deaths in the United States due to prostate cancer in 2020.

In some instances, the polypeptides or polypeptide complexes described herein are used to treat a solid tumor cancer. In some embodiments, the cancer is lung, breast (e.g. HER2+; ER/PR+; TNBC), cervical, ovarian, colorectal, pancreatic or gastric. In some embodiments, are methods of treating cancer comprising administering to a subject in need thereof an isolated polypeptide or polypeptide complex according to Formula I:

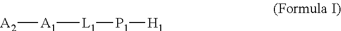

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to PSMA.

Disclosed herein, in some embodiments, are isolated polypeptides or polypeptide complexes according to Formula I:

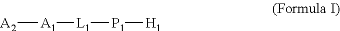

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA).

Disclosed herein, in some embodiments, are isolated polypeptides or polypeptide complexes according to Formula I:

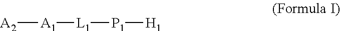

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to prostate-specific membrane antigen (PSMA).

Disclosed herein, in some embodiments, are isolated polypeptides or polypeptide complexes comprising Formula I:

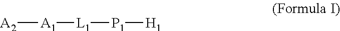

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA).

Disclosed herein, in some embodiments, are isolated polypeptides or polypeptide complexes comprising Formula I:

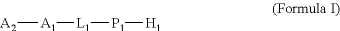

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to prostate-specific membrane antigen (PSMA).

Disclosed herein, in some embodiments, are isolated polypeptides or polypeptide complexes according to Formula I:

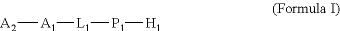

wherein: A₁ comprises a first antigen recognizing molecule that binds to prostate-specific membrane antigen (PSMA); P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to an effector cell antigen.

Disclosed herein, in some embodiments, are isolated polypeptides or polypeptide complexes according to Formula I:

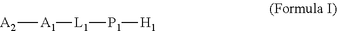

wherein: A₁ is a first antigen recognizing molecule that binds to prostate-specific membrane antigen (PSMA); P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to effector cell antigen.

Disclosed herein, in some embodiments, are isolated polypeptides or polypeptide complexes comprising Formula I:

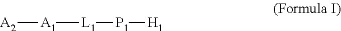

wherein: A₁ comprises a first antigen recognizing molecule that binds to prostate-specific membrane antigen (PSMA); P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to an effector cell antigen.

Disclosed herein, in some embodiments, are isolated polypeptides or polypeptide complexes comprising Formula I:

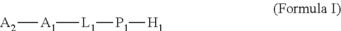

wherein: A₁ is a first antigen recognizing molecule that binds to prostate-specific membrane antigen (PSMA); P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to an effector cell antigen.

Disclosed herein, in some embodiments, are isolated polypeptides or polypeptide complexes according to Formula Ia:

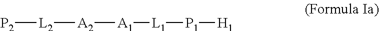

wherein A₂ further comprises P₂ and L₂, wherein P₂ comprises a peptide that binds to A₂; and L₂ comprises a linking moiety that connects A₂ to P₂ and is a substrate for a tumor specific protease.

Disclosed herein, in some embodiments, are isolated polypeptides or polypeptide complexes according to Formula Ia:

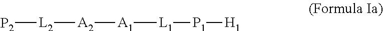

wherein A₂ further comprises P₂ and L₂, wherein P₂ is a peptide that binds to A₂; and L₂ is a linking moiety that connects A₂ to P₂ and is a substrate for a tumor specific protease.

Disclosed herein, in some embodiments, are isolated polypeptides or polypeptide complexes comprising Formula Ia:

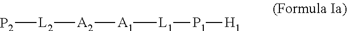

wherein A₂ further comprises P₂ and L₂, wherein P₂ comprises a peptide that binds to A₂; and L₂ comprises a linking moiety that connects A₂ to P₂ and is a substrate for a tumor specific protease.

Disclosed herein, in some embodiments, are isolated polypeptides or polypeptide complexes comprising Formula Ia:

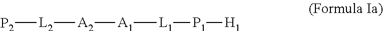

wherein A₂ further comprises P₂ and L₂, wherein P₂ is a peptide that binds to A₂; and L₂ is a linking moiety that connects A₂ to P₂ and is a substrate for a tumor specific protease.

Disclosed herein, in some embodiments, are isolated polypeptides or polypeptide complexes according to Formula II:

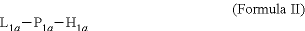

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ comprises a peptide that binds to the first antigen recognizing molecule when L₁ₐ is uncleaved; and H₁ₐ comprises a half-life extending molecule.

Disclosed herein, in some embodiments, are isolated polypeptides or polypeptide complexes comprising Formula II:

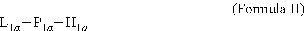

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ comprises a peptide that binds to the first antigen recognizing molecule when L₁ₐ is uncleaved; and H₁ₐ comprises a half-life extending molecule.

Disclosed herein, in some embodiments, are isolated polypeptides or polypeptide complexes according to Formula II:

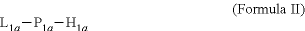

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ is a peptide that binds to the first antigen recognizing molecule when L₁ₐ is uncleaved; and H₁ₐ is a half-life extending molecule.

Disclosed herein, in some embodiments, are isolated polypeptides or polypeptide complexes comprising Formula II:

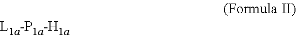

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ is a peptide that binds to the first antigen recognizing molecule when L₁ₐ is uncleaved; and H₁ₐ is a half-life extending molecule.

### First Antigen Recognizing Molecule (A₁)

Disclosed herein, in some embodiments, are isolated polypeptides or polypeptide complexes, wherein the first antigen recognizing molecule binds to an effector cell antigen and the second antigen recognizing molecule binds to PSMA. In some embodiments, the effector cell antigen comprises CD3. In some embodiments, A₁ comprises a first antigen recognizing molecule that binds to an effector cell antigen.

In some embodiments, A₁ comprises an antibody or antibody fragment. In some embodiments, A₁ comprises an antibody or antibody fragment that is human or humanized. In some embodiments, L₁ is bound to a N-terminus of the antibody or antibody fragment. In some embodiments, L₁ is bound to a N-terminus of the antibody or antibody fragment and A₂ is bound to the other N-terminus of the antibody or antibody fragment. In some embodiments, A₂ is bound to a C-terminus of the antibody or antibody fragment. In some embodiments, L₁ is bound to a C-terminus of the antibody or antibody fragment. In some embodiments, A₂ is bound to a N-terminus of the antibody or antibody fragment. In some embodiments, the antibody or antibody fragment comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, A₁ comprises a variable light chain and variable heavy chain each of which is capable of specifically binding to human CD3. In some embodiments, the effector cell antigen comprises CD3. In some embodiments, A₁ comprises an anti-CD3e single chain variable fragment. In some embodiments, A₁ comprises an anti-CD3e single chain variable fragment that has a K_{D} binding of 1 μM or less to CD3 on CD3 expressing cells. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

In some embodiments, A₁ comprises a first antigen recognizing molecule that binds PSMA. In some embodiments, A₁ comprises a variable light chain and variable heavy chain each of which is capable of specifically binding to human PSMA.

In some embodiments, the scFv that binds to CD3 comprises a scFv light chain variable domain and a scFv heavy chain variable domain. In some embodiments, the scFv heavy chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity). In some embodiments, the scFv light chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity).

In some embodiments, the scFv heavy chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity); and the scFv light chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity).

TABLE 1
anti-CD3 amino acid sequences
(CDRs as determined by IMGT numbering system)

| | | SEQ |
|---|---|---|
| | Amino Acid Sequence | ID |
| Construct Description | (N to C) | NO: |
| SP34.185 CD3: HC: CDR1 | GFTFNKYA | 1 |
| SP34.185 CD3: HC: CDR2 | IRSKYNNYAT | 2 |
| SP34.185 CD3: HC: CDR3 | VRHGNFGNSYISYWAY | 3 |
| SP34.185 CD3: LC: CDR1 | TGAVTSGNY | 4 |
| SP34.185 CD3: LC: CDR2 | GT | 5 |
| SP34.185 CD3: LC: CDR3 | VLWYSNRWV | 6 |
| SP34.185 scFv | EVQLVESGGGLVQPGGSLKLSCA | 7 |
| (VH - linker 1 - VL) | ASGFTFNKYAMNWVRQAPGKG | |
| | LEWVARIRSKYNNYATYYADSV | |
| | KDRFTISRDDSKNTAYLQMNNLK | |
| | TEDTAVYYCVRHGNFGNSYISY | |
| | WAYWGQGTLVTVSSGGGGSGGG | |
| | GSGGGGSQTVVTQEPSLTVSPGG | |
| | TVTLTCGSSTGAVTSGNYPNWV | |
| | QQKPGQAPRGLIGGTKFLAPGTP | |
| | ARFSGSLLGGKAALTLSGVQPED | |
| | EAEYYCVLWYSNRWVFGGGTKL | |
| | TVL | |

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3.

In some embodiments, the polypeptide or polypeptide complex of Formula I binds to an effector cell when L₁ is cleaved by the tumor specific protease. In some embodiments, the polypeptide or polypeptide complex of Formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε. In some embodiments, the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv comprise: HC-CDR1: SEQ ID NO: 1, HC-CDR2: SEQ ID NO: 2, and HC-CDR3: SEQ ID NO: 3; and the scFv comprises CDRs: LC-CDR1, LC-CDR2, and LC-CDR3, wherein the LC-CDR1, the LC-CDR2, and the LC-CDR3 of the scFv comprise: LC-CDR1: SEQ ID NO: 4, LC-CDR2: SEQ ID NO:5, and LC-CDR3: SEQ ID NO: 6. In some embodiments, the effector cell antigen comprises CD3, and the scFv comprises an amino acid sequence according to SEQ ID NO: 7.

In some embodiments, the effector cell antigen comprises CD3, and wherein A₁ comprises complementarity determining regions (CDRs): HC-CDR1, HC-CDR2, and HC-CDR3, wherein the HC-CDR1, the HC-CDR2, and the HC-CDR3 of A₁ comprises: HC-CDR1: SEQ ID NO: 1, HC-CDR2: SEQ ID NO: 2, and HC-CDR3: SEQ ID NO: 3; and A₁ comprises CDRs: LC-CDR1, LC-CDR2, and LC-CDR3, wherein the LC-CDR1, the LC-CDR2, and the LC-CDR3 of A₁ comprise: LC-CDR1: SEQ ID NO: 4, LC-CDR2: SEQ ID NO:5, and LC-CDR3: SEQ ID NO: 6. In some embodiments, the effector cell antigen comprises CD3, and A₁ comprises an amino acid sequence according to SEQ ID NO: 7.

In some embodiments, A₁ comprises an amino acid sequence according to SEQ ID NO: 7. In some embodiments, A₁ comprises an amino acid sequence that has at least 80% sequence identity to SEQ ID NO: 7. In some embodiments, A₁ comprises an amino acid sequence that has at least 85% sequence identity to SEQ ID NO: 7. In some embodiments, A₁ comprises an amino acid sequence that has at least 90% sequence identity to SEQ ID NO: 7. In some embodiments, A₁ comprises an amino acid sequence that has at least 95% sequence identity to SEQ ID NO: 7. In some embodiments, A₁ comprises an amino acid sequence that has at least 99% sequence identity to SEQ ID NO: 7.

In some embodiments, the polypeptide or polypeptide complex has weaker binding affinity for the tumor cell antigen as compared to the binding affinity for the tumor cell antigen of an isolated polypeptide or polypeptide complex that does not have P₁ or L₁. In some embodiments, the polypeptide or polypeptide complex has weaker binding affinity for the tumor cell antigen that is at least 5× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 8× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 10× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 15× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 20× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 25× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 30× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 35× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 40× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 45× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 50× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 55× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 60× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 65× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 70× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 75× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 80× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 85× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 90× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 95× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 100× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 120× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 1000× higher than the binding affinity for the tumor cell antigen of a form of the polypeptide or polypeptide complex that does not have P₁ or L₁.

In some embodiments, the polypeptide or polypeptide complex has weaker binding affinity for the tumor cell antigen as compared to the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 5× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 8× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 10× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 15× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 20× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 25× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 30× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 35× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 40× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 45× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 50× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 55× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 60× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 65× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 70× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 75× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 80× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 85× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 90× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 95× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 100× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 120× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 1000× higher than the binding affinity for the tumor cell antigen of the polypeptide or polypeptide complex in which L₁ has been cleaved by the tumor specific protease.

In some embodiments, the polypeptide or polypeptide complex has an increased EC₅₀ in an IFNγ release T-cell activation assay as compared to the EC₅₀ in an IFNγ release T-cell activation assay of an isolated polypeptide or polypeptide complex that does not have P₁ or L₁. In some embodiments, the polypeptide or polypeptide complex has an increased EC₅₀ in an IFNγ release T-cell activation assay that is at least 10× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 20× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 30× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 40× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 50× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 60× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 70× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 80× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 90× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 100× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 1000× higher than the EC₅₀ in an IFNγ release T-cell activation assay of a form of the polypeptide or polypeptide complex that does not have P₁ or L₁.

In some embodiments, the polypeptide or polypeptide complex has an increased EC₅₀ in an IFNγ release T-cell activation assay as compared to the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 10× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 20× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 30× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 40× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 50× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 60× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 70× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 80× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 90× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 100× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 1000× higher than the EC₅₀ in an IFNγ release T-cell activation assay of the polypeptide or polypeptide complex in which L₁ has been cleaved by the tumor specific protease.

In some embodiments, the polypeptide or polypeptide complex has an increased EC₅₀ in a T-cell cytolysis assay as compared to the EC₅₀ in a T-cell cytolysis assay of an isolated polypeptide or polypeptide complex that does not have P₁ or L₁. In some embodiments, the polypeptide or polypeptide complex has an increased EC₅₀ in a T-cell cytolysis assay that is at least 10× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 20× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 30× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 40× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 50× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 60× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 70× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 80× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 90× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 100× higher than the EC₅₀ in a T-cell cytolysis assay of an isolated polypeptide or polypeptide complex that does not have P₁ or L₁. In some embodiments, the polypeptide or polypeptide complex has an increased EC₅₀ in a T-cell cytolysis assay that is at least 1000× higher than the EC₅₀ in a T-cell cytolysis assay of an isolated polypeptide or polypeptide complex that does not have P₁ or L₁.

In some embodiments, the polypeptide or polypeptide complex has an increased EC₅₀ in a T-cell cytolysis assay as compared to the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 10× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 20× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 30× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 40× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 50× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 60× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 70× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 80× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 90× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 100× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 1,000× higher than the EC₅₀ in a T-cell cytolysis assay of the polypeptide or polypeptide complex in which L₁ has been cleaved by the tumor specific protease.

In some embodiments, the polypeptide or polypeptide complex P₂-L₂-A₂-A₁-L₁-P₁-H₁ (Formula Ia) has weaker binding affinity for the tumor cell antigen as compared to the binding affinity for the tumor cell antigen of an isolated polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 10× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 50× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 75× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 100× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 120× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 200× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 300× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 400× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 500× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 600× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 700× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 800× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 900× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 1000× higher than the binding affinity for the tumor cell antigen of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has weaker binding affinity for the tumor cell antigen that is at least 10,000× higher than the binding affinity for the tumor cell antigen of a form of the polypeptide or polypeptide complex of Formula Ia that does not have P₁, L₁, P₂, or L₂.

In some embodiments, the polypeptide or polypeptide complex P₂-L₂-A₂-A₁-L₁-P₁-H₁ (Formula Ia) has weaker binding affinity for the tumor cell antigen as compared to the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 10× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 50× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 75× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 100× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 120× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 200× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 300× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 400× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 500× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 600× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 700× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 800× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 900× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 1000× higher than the binding affinity for the tumor cell antigen of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has weaker binding affinity for the tumor cell antigen that is at least 10,000× higher than the binding affinity for the tumor cell antigen of the polypeptide or polypeptide complex of Formula Ia in which L₁ and L₂ have been cleaved by the tumor specific proteases.

In some embodiments, the polypeptide or polypeptide complex P₂-L₂-A₂-A₁-L₁-P₁-H₁ (Formula Ia) has an increased EC₅₀ in an IFNγ release T-cell activation assay as compared to the EC₅₀ in an IFNγ release T-cell activation assay of an isolated polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 10× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 50× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 75× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 100× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 200× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 300× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 400× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 500× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 600× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 700× higher than the EC₅₀ in an IFNγ release T-cell activation assay of a form of the polypeptide or polypeptide complex of Formula Ia that does not P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 800× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 900× higher than the EC₅₀ in an IFNγ release T-cell activation assay of a form of the polypeptide or polypeptide complex of Formula Ia that does not have P₁, L₁, P₂, or L₂.

In some embodiments, the polypeptide or polypeptide complex P₂-L₂-A₂-A₁-L₁-P₁-H₁ (Formula Ia) has an increased EC₅₀ in an IFNγ release T-cell activation assay that is at least 1000× higher than the EC₅₀ in an IFNγ release T-cell activation assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in an IFNγ release T-cell activation assay that is at least 10,000× higher than the EC₅₀ in an IFNγ release T-cell activation assay of a form of the polypeptide or polypeptide complex of Formula Ia that does not have P₁, L₁, P₂, or L₂.

In some embodiments, the polypeptide or polypeptide complex P₂-L₂-A₂-A₁-L₁-P₁-H₁ (Formula Ia) has an increased EC₅₀ in a T-cell cytolysis assay as compared to the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 10× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 50× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 75× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 100× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 200× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 300× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 400× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 500× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 600× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 700× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 800× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 900× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 1000× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 10,000× higher than the EC₅₀ in a T-cell cytolysis assay of the polypeptide or polypeptide complex of Formula Ia in which L₁ and L₂ have been cleaved by the tumor specific proteases.

In some embodiments, the polypeptide or polypeptide complex P₂-L₂-A₂-A₁-L₁-P₁-H₁ (Formula Ia) has an increased EC₅₀ in a T-cell cytolysis assay as compared to the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 10× higher than the EC₅₀ in a T-cell cytolysis assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 50× higher than the EC₅₀ in a T-cell cytolysis assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 75× higher than the EC₅₀ in a T-cell cytolysis assay of a form of the polypeptide or polypeptide complex of Formula Ia that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 100× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 200× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 300× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 400× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 500× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 600× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 700× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 800× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 900× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 1000× higher than the EC₅₀ in a T-cell cytolysis assay of an isolated polypeptide or polypeptide complex that does not have P₁, L₁, P₂, or L₂. In some embodiments, the polypeptide or polypeptide complex P₂-L₂-A₂-A₁-L₁-P₁-H₁ (Formula Ia) has an increased EC₅₀ in a T-cell cytolysis assay that is at least 10,000× higher than the EC₅₀ in a T-cell cytolysis assay of an isolated polypeptide or polypeptide complex that does not have P₁, L₁, P₂, or L₂.

In some embodiments, the polypeptide or polypeptide complex P₂-L₂-A₂-A₁-L₁-P₁-H₁ (Formula Ia) has an increased EC₅₀ in a T-cell cytolysis assay as compared to the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 10× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 50× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 75× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 100× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 200× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 300× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 400× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 500× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 600× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 700× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 800× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 900× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 1000× higher than the EC₅₀ in a T-cell cytolysis assay of the polypeptide or polypeptide complex of Formula Ia in which L₁ and L₂ have been cleaved by the tumor specific proteases. In some embodiments, the polypeptide or polypeptide complex P₂-L₂-A₂-A₁-L₁-P₁-H₁ (Formula Ia) has an increased EC₅₀ in a T-cell cytolysis assay that is at least 10,000× higher than the EC₅₀ in a T-cell cytolysis assay of the polypeptide or polypeptide complex of Formula Ia in which L₁ and L₂ have been cleaved by the tumor specific proteases.

### Second Antigen Recognizing Molecule (A₂)

In some embodiments, A₂ comprises an antibody or antibody fragment. In some embodiments, the antibody or antibody fragment thereof comprises a single chain variable fragment, a single domain antibody, a Fab, or a Fab′. In some embodiments, the antibody or antibody fragment thereof comprises a single chain variable fragment (scFv), a heavy chain variable domain (VH domain), a light chain variable domain (VL domain), or a variable domain (VHH) of a camelid derived single domain antibody. In some embodiments, the antibody or antibody fragment thereof is humanized or human. In some embodiments, A₂ is the Fab or Fab′. In some embodiments, the Fab or Fab′ comprises (a) a Fab light chain polypeptide and (b) a Fab heavy chain polypeptide. In some embodiments, the antibody or antibody fragment thereof comprises a PSMA binding domain.

In some embodiments, the antigen binding fragment (Fab) or Fab′ that binds to PSMA comprises a Fab light chain polypeptide chain and a Fab heavy chain polypeptide. In some embodiments, the Fab light chain polypeptide comprises a Fab light chain variable domain. In some embodiments, the Fab heavy chain polypeptide comprises a Fab heavy chain variable domain. In some embodiments, the Fab heavy chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity). In some embodiments, the Fab light chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity).

In some embodiments, the Fab heavy chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity); and the Fab light chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity).

TABLE 2
anti-PSMA amino acid sequences
(CDRs as determined by IMGT numbering system)

| Construct | Amino Acid Sequence | SEQ ID |
|---|---|---|
| Description | (N to C) | NO: |
| PSMA: HC: CDR1 | GFAFSRYG | 8 |
| PSMA: HC: CDR2 | IWYDGSNK | 9 |
| PSMA: HC: CDR3 | ARGGDFLYYYYYGMDV | 10 |
| PSMA: LC: CDR1 | QGISNY | 11 |
| PSMA: LC: CDR2 | EA | 12 |
| PSMA: LC: CDR3 | QNYNSAPFT | 13 |
| 006 PSMA Fab LC | DIQMTQSPSSLSASVGDRVTITCR | 14 |
| | ASQGISNYLAWYQQKTGKVPKF | |
| | LIYEASTLQSGVPSRFSGGGSGTD | |
| | FTLTISSLQPEDVATYYCQNYNSA | |
| | PFTFGPGTKVDIKRTVAAPSVFIFP | |
| | PSDEQLKSGTASVVCLLNNFYPRE | |
| | AKVQWKVDNALQSGNSQESVTE | |
| | QDSKDSTYSLSSTLTLSKADYEKH | |
| | KVYACEVTHQGLSSPVTKSFNRG | |
| | EC | |
| 006 PSMA Fab HC | QVQLVESGGGVVQPGRSLRLSCA | 15 |
| | ASGFAFSRYGMHWVRQAPGKGL | |
| | EWVAVIWYDGSNKYYADSVKG | |
| | RFTISRDNSKNTQYLQMNSLRAE | |
| | DTAVYYCARGGDFLYYYYYGM | |
| | DVWGQGTTVTVSSASTKGPSVFP | |
| | LAPSSKSTSGGTAALGCLVKDYFP | |
| | EPVTVSWNSGALTSGVHTFPAVL | |
| | QSSGLYSLSSVVTVPSSSLGTQTYI | |
| | CNVNHKPSNTKVDKKVEPKSC | |

In some embodiments, the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 8, HC-CDR2: SEQ ID NO: 9, and HC-CDR3: SEQ ID NO: 10; and the Fab comprises CDRs: LC-CDR1, LC-CDR2, and LC-CDR3, wherein the LC-CDR1, the LC-CDR2, and the LC-CDR3 of the Fab comprise LC-CDR1: SEQ ID NO: 11, LC-CDR2: SEQ ID NO: 12, and LC-CDR3: SEQ ID NO: 13. In some embodiments, the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 8, HC-CDR2: SEQ ID NO: 9, and HC-CDR3: SEQ ID NO: 10 and wherein the CDRs comprise from 0-2 amino acid modifications in at least one of the HC-CDR1, HC-CDR2, or HC-CDR3; and the Fab comprises CDRs: LC-CDR1, LC-CDR2, and LC-CDR3, wherein the LC-CDR1, the LC-CDR2, and the LC-CDR3 of the Fab comprise LC-CDR1: SEQ ID NO: 11, LC-CDR2: SEQ ID NO:12, and LC-CDR3: SEQ ID NO: 13 and wherein the CDRs comprise from 0-2 amino acid modifications in at least one of the LC-CDR1, LC-CDR2, or LC-CDR3.

In some embodiments, A₂ comprises complementarity determining regions (CDRs): HC-CDR1, HC-CDR2, and HC-CDR3, wherein the HC-CDR1, the HC-CDR2, and the HC-CDR3 of A₂ comprise: HC-CDR1: SEQ ID NO: 8, HC-CDR2: SEQ ID NO: 9, and HC-CDR3: SEQ ID NO: 10; and A₂ comprises CDRs: LC-CDR1, LC-CDR2, and LC-CDR3, wherein the LC-CDR1, the LC-CDR2, and the LC-CDR3 of A₂ comprise LC-CDR1: SEQ ID NO: 11, LC-CDR2: SEQ ID NO:12, and LC-CDR3: SEQ ID NO: 13. In some embodiments, A₂ comprises complementarity determining regions (CDRs): HC-CDR1, HC-CDR2, and HC-CDR3, wherein the HC-CDR1, the HC-CDR2, and the HC-CDR3 of A₂ comprise: HC-CDR1: SEQ ID NO: 8, HC-CDR2: SEQ ID NO: 9, and HC-CDR3: SEQ ID NO: 10 and wherein the CDRs comprise from 0-2 amino acid modifications in at least one of the HC-CDR1, HC-CDR2, or HC-CDR3; and A2 comprises CDRs: LC-CDR1, LC-CDR2, and LC-CDR3, wherein the LC-CDR1, the LC-CDR2, and the LC-CDR3 of A₂ comprise LC-CDR1: SEQ ID NO: 11, LC-CDR2: SEQ ID NO:12, and LC-CDR3: SEQ ID NO: 13 and wherein the CDRs comprise from 0-2 amino acid modifications in at least one of the LC-CDR1, LC-CDR2, or LC-CDR3.

In some embodiments, the Fab light chain polypeptide comprises an amino acid sequence according to SEQ ID NO: 14. In some embodiments, the Fab light chain polypeptide comprises an amino acid sequence that has at least 80% sequence identity according to SEQ ID NO: 14. In some embodiments, the Fab light chain polypeptide comprises an amino acid sequence that has at least 85% sequence identity according to SEQ ID NO: 14. In some embodiments, the Fab light chain polypeptide comprises an amino acid sequence that has at least 90% sequence identity according to SEQ ID NO: 14. In some embodiments, the Fab light chain polypeptide comprises an amino acid sequence that has at least 95% sequence identity according to SEQ ID NO: 14. In some embodiments, the Fab light chain polypeptide comprises an amino acid sequence that has at least 99% sequence identity according to SEQ ID NO: 14.

In some embodiments, the Fab heavy chain polypeptide comprises an amino acid sequence according to SEQ ID NO: 15. In some embodiments, the Fab heavy chain polypeptide comprises an amino acid sequence that has at least 80% sequence identity according to SEQ ID NO: 15. In some embodiments, the Fab heavy chain polypeptide comprises an amino acid sequence that has at least 85% sequence identity according to SEQ ID NO: 15. In some embodiments, the Fab heavy chain polypeptide comprises an amino acid sequence that has at least 90% sequence identity according to SEQ ID NO: 15. In some embodiments, the Fab heavy chain polypeptide comprises an amino acid sequence that has at least 95% sequence identity according to SEQ ID NO: 15. In some embodiments, the Fab heavy chain polypeptide comprises an amino acid sequence that has at least 99% sequence identity according to SEQ ID NO: 15.

In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁.

In some embodiments, A₂ further comprises P₂ and L₂, wherein P₂ comprises a peptide that binds to A₂; and L₂ comprises a linking moiety that connects A₂ to P₂ and is a substrate for a tumor specific protease. In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

Peptide (P₁ and P₂ and P₁ₐ)

In some embodiments, P₁, P₂, or P₁ₐ comprises a sequence as disclosed in Table 3 or a sequence substantially identical thereto (e.g., a sequence that has 0, 1, or 2 amino acid modifications).

TABLE 3
P₁ and P₂ and P₁ₐ Sequences

| Construct | Amino Acid Sequence | SEQ ID |
|---|---|---|
| Description | (N to C) | NO: |
| SP34.185 scFv mask | GGGSQCLGPEWEVCPY | 16 |
| SP34.185 scFv mask | GGVYCGPEFDESVGCM | 17 |
| SP34.185 scFv mask | GSQCLGPEWEVCPY | 18 |
| Peptide-A | | |
| SP34.185 scFv mask | VYCGPEFDESVGCM | 19 |
| Peptide-B | | |
| SP34.194 scFv mask | YLWGCEWNCAGITT | 78 |
| Peptide-AM | | |

In some embodiments, P₁ impairs binding of A₁ to a first target antigen. In some embodiments, P₁ impairs binding of A₁ to the effector cell antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ is bound to A₁ at or near an antigen binding site. In some embodiments, P₁ becomes unbound from A₁ when L₁ is cleaved by the tumor specific protease thereby exposing A₁ to the effector cell antigen. In some embodiments, the protease comprises a matrix metalloprotease (MMP) or a serine protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, P₁ has less than 70% sequence identity to the effector cell antigen. In some embodiments, P₁ has less than 75% sequence identity to the effector cell antigen. In some embodiments, P₁ has less than 80% sequence identity to the effector cell antigen. In some embodiments, P₁ has less than 85% sequence identity to the effector cell antigen. In some embodiments, P₁ has less than 90% sequence identity to the effector cell antigen. In some embodiments, P₁ has less than 95% sequence identity to the effector cell antigen. In some embodiments, P₁ has less than 98% sequence identity to the effector cell antigen. In some embodiments, P₁ has less than 99% sequence identity to the effector cell antigen. In some embodiments, P₁ comprises a de novo amino acid sequence that shares less than 10% sequence identity to the effector cell antigen. In some embodiments, P₁ comprises an amino acid sequence according to any one of SEQ ID NOs: 16-19. In some embodiments, P₁ comprises the amino acid sequence of SEQ ID NO: 16. In some embodiments, P₁ comprises the amino acid sequence of SEQ ID NO: 17. In some embodiments, P₁ comprises the amino acid sequence of SEQ ID NO: 18. In some embodiments, P₁ comprises the amino acid sequence of SEQ ID NO: 19. In some embodiments, P₁ comprises the amino acid sequence of SEQ ID NO: 78.

In some embodiments, P₁ comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; Z₁₄ is selected from D, Y, N, F, I, P, V, A, T, H, L and S. In some embodiments, Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; Z₁₄ is selected from D, Y, N, F, I, and P. In some embodiments, Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N.

In some embodiments, P₁ comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

In some embodiments, P₁ comprises the amino acid sequences according to any one of SEQ ID NOs: 79-105. In some embodiments, P₁ comprises an amino acid sequences according to any of the sequences of Table 20. In some embodiments, P₁ comprises the amino acid sequences according to any one of SEQ ID NOs: 106-117.

In some embodiments, P₁ comprises the amino acid sequence according to SEQ ID NO: 18 or a peptide sequence that has 1, 2, or 3, amino acid substitutions, additions, or deletions relative to the amino acid sequence of SEQ ID NO: 18.

In some embodiments, P₁ comprises the amino acid sequence according to SEQ ID NO: 19 or a peptide sequence that has 1, 2, or 3, amino acid substitutions, additions, or deletions relative to the amino acid sequence of SEQ ID NO: 19.

In some embodiments, P₁ comprises the amino acid sequence according to SEQ ID NO: 116 or a peptide sequence that has 1, 2, or 3, amino acid substitutions, additions, or deletions relative to the amino acid sequence of SEQ ID NO: 116.

In some embodiments, P₁ comprises the amino acid sequence according to SEQ ID NO: 18.

In some embodiments, P₁ comprises the amino acid sequence according to SEQ ID NO: 19.

In some embodiments, P₁ comprises the amino acid sequence according to SEQ ID NO: 116.

In some embodiments, P₂ impairs binding of A₂ to the second target antigen. In some embodiments, wherein P₂ impairs binding of A₂ to PSMA. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A2 at or near an antigen binding site. In some embodiments, P₂ becomes unbound from A₂ when L₂ is cleaved by the tumor specific protease thereby exposing A₂ to the PSMA. In some embodiments, the protease comprises a matrix metalloprotease (MMP) or a serine protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, P₂ has less than 70% sequence identity to the PSMA. In some embodiments, P₂ has less than 75% sequence identity to the PSMA. In some embodiments, P₂ has less than 80% sequence identity to the PSMA. In some embodiments, P₂ has less than 85% sequence identity to the PSMA. In some embodiments, P₂ has less than 90% sequence identity to the PSMA. In some embodiments, P₂ has less than 95% sequence identity to the PSMA. In some embodiments, P₂ has less than 98% sequence identity to the PSMA. In some embodiments, P₂ has less than 99% sequence identity to the PSMA. In some embodiments, P₂ comprises a de novo amino acid sequence that shares less than 10% sequence identity to the PSMA.

In some embodiments, P₁ₐ when L₁ₐ is uncleaved impairs binding of the antigen recognizing molecule to the target antigen. In some embodiments, the antigen recognizing molecule comprises an antibody or antibody fragment. In some embodiments, the target antigen is an anti-CD3 effector cell antigen. In some embodiments, the target antigen is prostate-specific membrane antigen (PSMA). In some embodiments, P₁ₐ has less than 70% sequence identity to the target antigen. In some embodiments, P₁ₐ has less than 75% sequence identity to the target antigen. In some embodiments, P₁ₐ has less than 80% sequence identity to the target antigen. In some embodiments, P₁ₐ has less than 85% sequence identity to the target antigen. In some embodiments, P₁ₐ has less than 90% sequence identity to the target antigen. In some embodiments, P₁ₐ has less than 95% sequence identity to the target antigen. In some embodiments, P₁ₐ has less than 98% sequence identity to the target antigen. In some embodiments, P₁ₐ has less than 99% sequence identity to the target antigen. In some embodiments, P₁ₐ comprises a de novo amino acid sequence that shares less than 10% sequence identity to the second target antigen.

In some embodiments, P₁ₐ comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S. In some embodiments, Z₁ is selected from D, Y, F, I, and N; Z₂ is selected from D, Y, L, F, I, and N; Z₄ is selected from G and W; Z₆ is selected from E and D; Z, is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P. In some embodiments, Z₁ is selected from D, Y, and F; Z₂ is selected from D, Y, L, and F; Z₄ is selected from G and W; Z₆ is selected from E and D; Z, is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N.

In some embodiments, P₁ₐ comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

In some embodiments, P₁ₐ comprises the amino acid sequences according to any one of SEQ ID NOs: 79-105.

In some embodiments, P₁ₐ comprises an amino acid sequences according to any of the sequences of Table 20.

In some embodiments, P₁ₐ comprises the amino acid sequences according to any one of SEQ ID NOs: 106-117.

In some embodiments, P₁ₐ comprises the amino acid sequence according to SEQ ID NO: 18 or a peptide sequence that has 1, 2, or 3, amino acid substitutions, additions, or deletions relative to the amino acid sequence of SEQ ID NO: 18.

In some embodiments, P₁ₐ comprises the amino acid sequence according to SEQ ID NO: 19 or a peptide sequence that has 1, 2, or 3, amino acid substitutions, additions, or deletions relative to the amino acid sequence of SEQ ID NO: 19.

In some embodiments, P₁ₐ comprises the amino acid sequence according to SEQ ID NO: 116 or a peptide sequence that has 1, 2, or 3, amino acid substitutions, additions, or deletions relative to the amino acid sequence of SEQ ID NO: 116.

In some embodiments, P₁ₐ comprises the amino acid sequence according to SEQ ID NO: 18.

In some embodiments, P₁ₐ comprises the amino acid sequence according to SEQ ID NO: 19.

In some embodiments, P₁ₐ comprises the amino acid sequence according to SEQ ID NO: 116.

In some embodiments, P₁, P₂, or P₁ₐ comprises a peptide sequence of at least 5 amino acids in length. In some embodiments, P₁, P₂, or P₁ₐ comprises a peptide sequence of at least 6 amino acids in length. In some embodiments, P₁, P₂, or P₁ₐ comprises a peptide sequence of at least 10 amino acids in length. In some embodiments, P₁, P₂, or P₁ₐ comprises a peptide sequence of at least 10 amino acids in length and no more than 20 amino acids in length. In some embodiments, P₁, P₂, or P₁ₐ comprises a peptide sequence of at least 16 amino acids in length. In some embodiments, P₁, P₂, or P₁ₐ comprises a peptide sequence of no more than 40 amino acids in length. In some embodiments, P₁, P₂, or P₁ₐ comprises at least two cysteine amino acid residues. In some embodiments, P₁, P₂, or P₁ₐ comprises a cyclic peptide or a linear peptide. In some embodiments, P₁, P₂, or P₁ₐ comprises a cyclic peptide. In some embodiments, P₁, P₂, or P₁ₐ comprises a linear peptide.

In some embodiments, P₁, P₂, or P₁ₐ or P₁, P₂, and P₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments P₁, P₂, or P₁ₐ or P₁, P₂, and P₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁, P₂, or Pia or P₁, P₂, and P₁ₐ including the peptide backbone, the amino acid side chains, and the terminus.

In some embodiments, P₁, P₂, or P₁ₐ does not comprise albumin or an albumin fragment. In some embodiments, P₁, P₂, or P₁ₐ does not comprise an albumin binding domain.

Linking Moiety (L₁, L₂, L₃, and L₁ₐ)

In some embodiments, L₁, L₂, L₃, or L₁ₐ is a peptide sequence having at least 5 to no more than 50 amino acids. In some embodiments L₁, L₂, L₃, or L₁ₐ is a peptide sequence having at least 10 to no more than 30 amino acids. In some embodiments, L₁, L₂, L₃, or L₁ₐ is a peptide sequence having at least 10 amino acids. In some embodiments, L₁, L₂, L₃, or L₁ₐ is a peptide sequence having at least 18 amino acids. In some embodiments, L₁, L₂, L₃, or L₁ₐ is a peptide sequence having at least 26 amino acids. In some embodiments, L₁, L₂, L₃, or L₁ₐ has a formula comprising (G₂S)ₙ, wherein n is an integer from 1 to 3 (SEQ ID NO: 118). In some embodiments, L₁, L₂, L₃, or L₁ₐ has a formula comprising (G₂S)ₙ, wherein n is an integer of at least 1. In some embodiments, L₁, L₂, L₃, or L₁ₐ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1. In some embodiments, the tumor specific protease is selected from the group consisting of metalloprotease, serine protease, cysteine protease, threonine protease, and aspartic protease. In some embodiments L₁, L₂, L₃, or L₁ₐ comprises a urokinase cleavable amino acid sequence, a matriptase cleavable amino acid sequence, a legumain cleavable amino acid sequence, or a matrix metalloprotease cleavable amino acid sequence. In some embodiments, the matrix metalloprotease comprises MMP2, MMP7, MMP9, MMPS13, or MM14. In some embodiments, the serine protease comprises matriptase (MTSP1), urokinase, or hepsin.

In some embodiments, L₁, L₂, L₃, or L₁ₐ comprises a sequence as disclosed in Table 4 or a sequence substantially identical thereto (e.g., a sequence that has 0, 1, or 2 amino acid modifications).

In some embodiments, L₁, comprises the sequence of Linker-25 (SEQ ID NO: 45). In some embodiments, L₁, comprises the sequence of Linker-26 (SEQ ID NO: 46). In some embodiments, L₁, comprises the sequence of Linker-27 (SEQ ID NO: 47). In some embodiments, L₁, comprises the sequence of Linker-28 (SEQ ID NO: 48).

In some embodiments, L₂, comprises the sequence of Linker-25 (SEQ ID NO: 45). In some embodiments, L₂, comprises the sequence of Linker-26 (SEQ ID NO: 46). In some embodiments, L₂, comprises the sequence of Linker-27 (SEQ ID NO: 47). In some embodiments, L₂, comprises the sequence of Linker-28 (SEQ ID NO: 48).

TABLE 4
L₁, L₂, L₃, and L₁ₐ Sequences

| Construct | Amino Acid Sequence | SEQ ID |
|---|---|---|
| Description | (N to C) | NO: |
| Linker 1 | GGGGSGGGGSGGGGS | 20 |
| Linker 2 | GGGGS | 21 |
| Linker 3 | GGGGSGGGS | 22 |
| Cleavable | GGGGSGGGLSGRSDAGSPLGLAG | 23 |
| linker | SGGGS | |
| Linker 4 | GGGGSLSGRSDNHGSSGT | 24 |
| Linker 5 | GGGGSSGGSGGSGLSGRSDNHGS | 25 |
| | SGT | |
| Linker 6 | ASGRSDNH | 26 |
| Linker 7 | LAGRSDNH | 27 |
| Linker 8 | ISSGLASGRSDNH | 28 |
| Linker 9 | ISSGLLAGRSDNH | 29 |
| Linker 10 | LSGRSDNH | 30 |
| Linker 11 | ISSGLLSGRSDNP | 31 |
| Linker 12 | ISSGLLSGRSDNH | 32 |
| Linker 13 | LSGRSDNHSPLGLAGS | 33 |
| Linker 14 | SPLGLAGSLSGRSDNH | 34 |
| Linker 15 | SPLGLSGRSDNH | 35 |
| Linker 16 | LAGRSDNHSPLGLAGS | 36 |
| Linker 17 | LSGRSDNHVPLSLKMG | 37 |
| Linker 18 | LSGRSDNHVPLSLSMG | 38 |
| Linker 19 | GSSGGSGGSGGSGISSGLLSGRSD | 39 |
| | NHGSSGT | |
| Linker 20 | GSSGGSGGSGGISSGLLSGRSDNH | 40 |
| | GGGS | |
| Linker 21 | ASGRSDNH | 41 |
| Linker 22 | LAGRSDNH | 42 |
| Linker 23 | ISSGLASGRSDNH | 43 |
| Linker 24 | LSGRSDAG | 44 |
| Linker 25 | ISSGLLSGRSDAG | 45 |
| Linker 26 | AAGLLAPPGGLSGRSDAG | 46 |
| Linker 27 | SPLGLSGRSDAG | 47 |
| Linker 28 | LSGRSDAGSPLGLAG | 48 |
| Non-cleavable | GGGGSGGGSGGGGSGGASSGAG | 49 |
| linker | GSGGGS | |

In some embodiments, L₁ is bound to a N-terminus of A₁. In some embodiments, L₁ is bound to a C-terminus of A₁. In some embodiments, L₂ is bound to a N-terminus of A₂. In some embodiments, L₂ is bound to a C-terminus of A₂. In some embodiments, P₁ becomes unbound from A₁ when L₁ is cleaved by the tumor specific protease thereby exposing A₁ to the effector cell antigen. In some embodiments, P₂ becomes unbound from A₂ when L₂ is cleaved by the tumor specific protease thereby exposing A₂ to PSMA.

In some embodiments, L₁, L₂, L₃, or L₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments, L₁, L₂, L₃, or L₁ₐ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₁, L₂, L₃, or L₁ₐ including the peptide backbone, or the amino acid side chains.

In some embodiments, the cleavable linker is cleavable by a protease. In some embodiments, the protease is present in higher levels in a disease-state microenvironment relative to levels in healthy tissue or a microenvironment that is not the disease-state microenvironment. In some embodiments, the protease comprises a tumor specific protease. In some embodiments, the protease comprises a matrix metalloprotease (MMP) or a serine protease. In some embodiments, the matrix metalloprotease comprises MMP2, MMP7, MMP9, MMP13, or MMP14. In some embodiments, the matrix metalloprotease is selected from the group consisting of MMP2, MMP7, MMP9, MMP13, and MMP14. In some embodiments, the matrix metalloprotease comprises MMP2. In some embodiments, the matrix metalloprotease comprises MMP7. In some embodiments, the matrix metalloprotease comprises MMP9. In some embodiments, the matrix metalloprotease comprises MMP13. In some embodiments, the matrix metalloprotease comprises MMP14. In some embodiments, the serine protease comprises matriptase (MTSP1), urokinase, or hepsin. In some embodiments, the serine protease is selected from the group consisting of matriptase (MTSP1), urokinase, and hepsin. In some embodiments, the serine protease comprises matriptase (MTSP1). In some embodiments, the serine protease comprises urokinase. In some embodiments, the serine protease comprises hepsin. In some embodiments, the cleavable linker is cleaved by a variety of proteases. In some embodiments, the cleavable linker is cleaved by at least 1, 2, 3, 4, 5, 6, 7, 8, 9, 10, 15, 20, or more than 20 different proteases.

Half-Life Extending Molecule (H₁ and H₁ₐ)

In some embodiments, H₁ does not block A₁ binding to the effector cell antigen. In some embodiments, H₁ comprises a linking moiety (L₃) that connects H₁ to P₁. In some embodiments, H₁ₐ does not block the first antigen recognizing molecule binding to the effector cell antigen. In some embodiments, H₁ₐ comprises a linking moiety (L₃) that connects H₁ₐ to P₁ₐ. In some embodiments, the half-life extending molecule (H₁ or H₁ₐ) does not have binding affinity to antigen recognizing molecule. In some embodiments, the half-life extending molecule (H₁ or H₁ₐ) does not have binding affinity to the effector cell antigen. In some embodiments, the half-life extending molecule (H₁ or H₁ₐ) does not shield antigen recognizing molecule from the effector cell antigen. In some embodiments, the half-life extending molecule (H₁ or H₁ₐ) is not directly linked to antigen recognizing molecule.

In some embodiments, H₁ or H₁ₐ comprises a sequence as disclosed in Table 5 or a sequence substantially identical thereto (e.g., a sequence that has at least 90%, 95%, %%, 97%, 98%, or 99% sequence identity).

TABLE 5
H₁ and H₁ₐ Sequences

| | Amino Acid Sequence | |
|---|---|---|
| Construct Description | (N to C) | SEQ ID NO: |
| Anti-Albumin: CDR-H1 | GSTFYTAV | 54 |
| Anti-Albumin: CDR-H2 | IRWTALTT | 55 |
| Anti-Albumin: CDR-H3 | AARGTLGLFTTADSYDY | 56 |
| Anti-albumin | EVQLVESGGGLVQPGGSLRLSCAASGSTF | 57 |
| | YTAVMGWVRQAPGKGLEWVAAIRWTA | |
| | LTTSYADSVKGRFTISRDGAKTTLYLQM | |
| | NSLRPEDTAVYYCAARGTLGLFTTADSY | |
| | DYWGQGTLVTVSS | |
| 10G Anti-Albumin: CDR-H1 | GFTFSKFG | 58 |
| 10G Anti-Albumin: CDR-H2 | ISGSGRDT | 59 |
| 10G Anti-Albumin: CDR-H3 | TIGGSLSV | 60 |
| 10G Anti-albumin | EVQLVESGGGLVQPGNSLRLSCAASGFT | 61 |
| | FSKFGMSWVRQAPGKGLEWVSSISGSGR | |
| | DTLYADSVKGRFTISRDNAKTTLYLQMN | |
| | SLRPEDTAVYYCTIGGSLSVSSQGTLVTV | |
| | SS | |

In some embodiments, H₁ or H₁ₐ comprise an amino acid sequence that has repetitive sequence motifs. In some embodiments, H₁ or H₁ₐ comprises an amino acid sequence that has highly ordered secondary structure. “Highly ordered secondary structure,” as used in this context, means that at least about 50%, or about 70%, or about 80%, or about 90%, of amino acid residues of H₁ or H₁ₐ contribute to secondary structure, as measured or determined by means, including, but not limited to, spectrophotometry (e.g. by circular dichroism spectroscopy in the “far-UV” spectral region (190-250 nm), and computer programs or algorithms, such as the Chou-Fasman algorithm and the Gamier-Osguthorpe-Robson (“GOR”) algorithm.

In some embodiments, H₁ or H₁ₐ comprises a polymer. In some embodiments, the polymer is polyethylene glycol (PEG). In some embodiments, H₁ or H₁ₐ comprises albumin. In some embodiments, H₁ or H₁ₐ comprises an Fc domain. In some embodiments, the albumin is serum albumin. In some embodiments, the albumin is human serum albumin. In some embodiments, H₁ or H₁ₐ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the single domain antibody comprises a single domain antibody that binds to albumin. In some embodiments, the single domain antibody is a human or humanized antibody. In some embodiments, the single domain antibody is selected from the group consisting of 645gH1gL1, 645dsgH5gL4, 23-13-A01-sc02, A10m3 or a fragment thereof, DOM7r-31, DOM7 h-11-15, Alb-1, Alb-8, Alb-23, 10G, 10E and SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 58, HC-CDR2: SEQ ID NO: 59, and HC-CDR3: SEQ ID NO: 60. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 58, HC-CDR2: SEQ ID NO: 59, and HC-CDR3: SEQ ID NO: 60; and wherein the CDRs comprise from 0-2 amino acid modifications in at least one of the HC-CDR1, HC-CDR2, or HC-CDR3.

In some embodiments, H₁ comprises an amino acid sequence according to SEQ ID NO: 57. In some embodiments, H₁ comprises an amino acid sequence that has at least 80% sequence identity to SEQ ID NO: 57. In some embodiments, H₁ comprises an amino acid sequence that has at least 85% sequence identity to SEQ ID NO: 57. In some embodiments, H₁ comprises an amino acid sequence that has at least 90% sequence identity to SEQ ID NO: 57. In some embodiments, H₁ comprises an amino acid sequence that has at least 95% sequence identity to SEQ ID NO: 57. In some embodiments, H₁ comprises an amino acid sequence that has at least 99% sequence identity to SEQ ID NO: 57.

In some embodiments, H₁ₐ comprises an amino acid sequence according to SEQ ID NO: 57. In some embodiments, H₁ₐ comprises an amino acid sequence that has at least 80% sequence identity to SEQ ID NO: 57. In some embodiments, H₁ₐ comprises an amino acid sequence that has at least 85% sequence identity to SEQ ID NO: 57. In some embodiments, H₁ₐ comprises an amino acid sequence that has at least 90% sequence identity to SEQ ID NO: 57. In some embodiments, H₁ₐ comprises an amino acid sequence that has at least 95% sequence identity to SEQ ID NO: 57. In some embodiments, H₁ₐ comprises an amino acid sequence that has at least 99% sequence identity to SEQ ID NO: 57.

In some embodiments, H₁ comprises an amino acid sequence according to SEQ ID NO: 61. In some embodiments, H₁ comprises an amino acid sequence that has at least 80% sequence identity to SEQ ID NO: 61. In some embodiments, H₁ comprises an amino acid sequence that has at least 85% sequence identity to SEQ ID NO: 61. In some embodiments, H₁ comprises an amino acid sequence that has at least 90% sequence identity to SEQ ID NO: 61. In some embodiments, H₁ comprises an amino acid sequence that has at least 95% sequence identity to SEQ ID NO: 61. In some embodiments, H₁ comprises an amino acid sequence that has at least 99% sequence identity to SEQ ID NO: 61.

In some embodiments, H₁ₐ comprises an amino acid sequence according to SEQ ID NO: 61. In some embodiments, H₁ₐ comprises an amino acid sequence that has at least 80% sequence identity to SEQ ID NO: 61. In some embodiments, H₁ₐ comprises an amino acid sequence that has at least 85% sequence identity to SEQ ID NO: 61. In some embodiments, H₁ₐ comprises an amino acid sequence that has at least 90% sequence identity to SEQ ID NO: 61. In some embodiments, H₁ₐ comprises an amino acid sequence that has at least 95% sequence identity to SEQ ID NO: 61. In some embodiments, H₁ comprises an amino acid sequence that has at least 99% sequence identity to SEQ ID NO: 61.

In some embodiments, H₁ or H₁ₐ or H₁ and H₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments H₁ or H₁ₐ or H₁ and H₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to H₁ or H₁ₐ or H₁ and H₁ₐ including the peptide backbone, the amino acid side chains, and the terminus.

In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1. In some embodiments, L₃ comprises an amino acid sequence according to SEQ ID NO: 22.

In some embodiments, H₁ₐ comprises a linking moiety (L₁ₐ) that connects H₁ₐ to P₁ₐ. In some embodiments, L₁ₐ is a peptide sequence having at least 5 to no more than 50 amino acids. In some embodiments, L₁ₐ is a peptide sequence having at least 10 to no more than 30 amino acids. In some embodiments, L₁ₐ is a peptide sequence having at least 10 amino acids. In some embodiments, L₁ₐ is a peptide sequence having at least 18 amino acids. In some embodiments, L₁ₐ is a peptide sequence having at least 26 amino acids. In some embodiments, L₁ₐ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1. In some embodiments, L₁ₐ comprises an amino acid sequence according to SEQ ID NO: 22.

Antibodies that Bind to PSMA and CD3

In some embodiments, the polypeptide or polypeptide complex comprises an amino acid sequence disclosed in Table 6 or a sequence substantially identical thereto (e.g., a sequence that has at least 90%, 95%, 96%, 97%, 98%, or 99% sequence identity). In some embodiments, the polypeptide or polypeptide complex comprises an amino acid sequence with at least 95% sequence identity to any one of SEQ ID NOs: 62-77. In some embodiments, the polypeptide or polypeptide complex comprises an amino acid sequence with at least 95% sequence identity to SEQ ID NO: 72. In some embodiments, the polypeptide or polypeptide complex comprises an amino acid sequence with at least 95% sequence identity to SEQ ID NO: 73.

TABLE 6
Polypeptide complex sequences

| Construct | Amino Acid Sequence | SEQ ID |
|---|---|---|
| Description | (N to C) | NO: |
| PC1: LC: | DIQMTQSPSSLSASVGDRVTITCRASQGISNYLAWYQ | 62 |
| 006 PSMA Fab LC | QKTGKVPKFLIYEASTLQSGVPSRFSGGGSGTDFTLTIS | |
| | SLQPEDVATYYCQNYNSAPFTFGPGTKVDIKRTVAAP | |
| | SVFIFPPSDEQLKSGTASVVCLLNNFYPREAKVQWKV | |
| | DNALQSGNSQESVTEQDSKDSTYSLSSTLTLSKADYE | |
| | KHKVYACEVTHQGLSSPVTKSFNRGEC | |
| PC1: HC: | EVQLVESGGGLVQPGGSLKLSCAASGFTFNKYAMN | 63 |
| SP34.185 scFv | WVRQAPGKGLEWVARIRSKYNNYATYYADSVKDRF | |
| Linker 2 | TISRDDSKNTAYLQMNNLKTEDTAVYYCVRHGNFGN | |
| 006 PSMA Fab HC | SYISYWAYWGQGTLVTVSSGGGGSGGGGSGGGGSQT | |
| | VVTQEPSLTVSPGGTVTLTCGSSTGAVTSGNYPNWV | |
| | QQKPGQAPRGLIGGTKFLAPGTPARFSGSLLGGKAAL | |
| | TLSGVQPEDEAEYYCVLWYSNRWVFGGGTKLTVLG | |
| | GGGSQVQLVESGGGVVQPGRSLRLSCAASGFAFSRY | |
| | GMHWVRQAPGKGLEWVAVIWYDGSNKYYADSVKG | |
| | RFTISRDNSKNTQYLQMNSLRAEDTAVYYCARGGDF | |
| | LYYYYYGMDVWGQGTTVTVSSASTKGPSVFPLAPSS | |
| | KSTSGGTAALGCLVKDYFPEPVTVSWNSGALTSGVHT | |
| | FPAVLQSSGLYSLSSVVTVPSSSLGTQTYICNVNHKPS | |
| | NTKVDKKVEPKSC | |
| PC: 2: LC | EVQLVESGGGLVQPGGSLKLSCAASGFTFNKYAMN | 64 |
| SP34.185 scFv | WVRQAPGKGLEWVARIRSKYNNYATYYADSVKDRF | |
| Linker 2 | TISRDDSKNTAYLQMNNLKTEDTAVYYCVRHGNFGN | |
| 006 PSMA Fab LC | SYISYWAYWGQGTLVTVSSGGGGSGGGGSGGGGSQT | |
| | VVTQEPSLTVSPGGTVTLTCGSSTGAVTSGNYPNWV | |
| | QQKPGQAPRGLIGGTKFLAPGTPARFSGSLLGGKAAL | |
| | TLSGVQPEDEAEYYCVLWYSNRWVFGGGTKLTVLG | |
| | GGGSDIQMTQSPSSLSASVGDRVTITCRASQGISNYLA | |
| | WYQQKTGKVPKFLIYEASTLQSGVPSRFSGGGSGTDF | |
| | TLTISSLQPEDVATYYCQNYNSAPFTFGPGTKVDIKRT | |
| | VAAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNRGEC | |
| PC2: HC | QVQLVESGGGVVQPGRSLRLSCAASGFAFSRYGMH | 65 |
| 006 PSMA Fab HC | WVRQAPGKGLEWVAVIWYDGSNKYYADSVKGRFTI | |
| | SRDNSKNTQYLQMNSLRAEDTAVYYCARGGDFLYY | |
| | YYYGMDVWGQGTTVTVSSASTKGPSVFPLAPSSKSTS | |
| | GGTAALGCLVKDYFPEPVTVSWNSGALTSGVHTFPA | |
| | VLQSSGLYSLSSVVTVPSSSLGTQTYICNVNHKPSNTK | |
| | VDKKVEPKSC | |
| PC3: LC | DIQMTQSPSSLSASVGDRVTITCRASQGISNYLAWYQ | 66 |
| 006 PSMA Fab LC | QKTGKVPKFLIYEASTLQSGVPSRFSGGGSGTDFTLTIS | |
| | SLQPEDVATYYCQNYNSAPFTFGPGTKVDIKRTVAAP | |
| | SVFIFPPSDEQLKSGTASVVCLLNNFYPREAKVQWKV | |
| | DNALQSGNSQESVTEQDSKDSTYSLSSTLTLSKADYE | |
| | KHKVYACEVTHQGLSSPVTKSFNRGEC | |
| PC3: HC | EVQLVESGGGLVQPGGSLRLSCAASGSTFYTAVMGW | 67 |
| Anti-albumin (SEQ | VRQAPGKGLEWVAAIRWTALTTSYADSVKGRFTISR | |
| ID NO: 57) + Linker | DGAKTTLYLQMNSLRPEDTAVYYCAARGTLGLFTT | |
| 3 + SP34.185 scFv | ADSYDYWGQGTLVTVSSGGGGSGGGSGGGSQCLGPE | |
| mask (SEQ ID NO: | WEVCPYGGGGSGGGLSGRSDAGSPLGLAGSGGGSEV | |
| 16) + cleavable | QLVESGGGLVQPGGSLKLSCAASGFTFNKYAMNWV | |
| linker + SP34.185 | RQAPGKGLEWVARIRSKYNNYATYYADSVKDRFTIS | |
| scFv (VH-linker 1- | RDDSKNTAYLQMNNLKTEDTAVYYCVRHGNFGNSY | |
| VL) + Linker 2 + | ISYWAYWGQGTLVTVSSGGGGSGGGGSGGGGSQTV | |
| 006 PSMA Fab HC | VTQEPSLTVSPGGTVTLTCGSSTGAVTSGNYPNWVQ | |
| | QKPGQAPRGLIGGTKFLAPGTPARFSGSLLGGKAALT | |
| | LSGVQPEDEAEYYCVLWYSNRWVFGGGTKLTVLGG | |
| | GGSQVQLVESGGGVVQPGRSLRLSCAASGFAFSRYG | |
| | MHWVRQAPGKGLEWVAVIWYDGSNKYYADSVKGR | |
| | FTISRDNSKNTQYLQMNSLRAEDTAVYYCARGGDFL | |
| | YYYYYGMDVWGQGTTVTVSSASTKGPSVFPLAPSSK | |
| | STSGGTAALGCLVKDYFPEPVTVSWNSGALTSGVHTF | |
| | PAVLQSSGLYSLSSVVTVPSSSLGTQTYICNVNHKPSN | |
| | TKVDKKVEPKSC | |
| PC4: LC | EVQLVESGGGLVQPGGSLRLSCAASGSTFYTAVMGW | 68 |
| Anti-albumin (SEQ | VRQAPGKGLEWVAAIRWTALTTSYADSVKGRFTISR | |
| ID NO: 57) + Linker | DGAKTTLYLQMNSLRPEDTAVYYCAARGTLGLFTT | |
| 3 + SP34.185 scFv | ADSYDYWGQGTLVTVSSGGGGSGGGSGGGSQCLGPE | |
| mask (SEQ ID NO: | WEVCPYGGGGSGGGLSGRSDAGSPLGLAGSGGGSEV | |
| 16) + cleavable | QLVESGGGLVQPGGSLKLSCAASGFTFNKYAMNWV | |
| linker + SP34.185 | RQAPGKGLEWVARIRSKYNNYATYYADSVKDRFTIS | |
| scFv (VH-linker 1- | RDDSKNTAYLQMNNLKTEDTAVYYCVRHGNFGNSY | |
| VL) + Linker 2 + | ISYWAYWGQGTLVTVSSGGGGSGGGGSGGGGSQTV | |
| 006 PSMA Fab LC | VTQEPSLTVSPGGTVTLTCGSSTGAVTSGNYPNWVQ | |
| | QKPGQAPRGLIGGTKFLAPGTPARFSGSLLGGKAALT | |
| | LSGVQPEDEAEYYCVLWYSNRWVFGGGTKLTVLGG | |
| | GGSDIQMTQSPSSLSASVGDRVTITCRASQGISNYLAW | |
| | YQQKTGKVPKFLIYEASTLQSGVPSRFSGGGSGTDFTL | |
| | TISSLQPEDVATYYCQNYNSAPFTFGPGTKVDIKRTV | |
| | AAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNRGEC | |
| PC4: HC | QVQLVESGGGVVQPGRSLRLSCAASGFAFSRYGMH | 69 |
| 006 PSMA Fab HC | WVRQAPGKGLEWVAVIWYDGSNKYYADSVKGRFTI | |
| | SRDNSKNTQYLQMNSLRAEDTAVYYCARGGDFLYY | |
| | YYYGMDVWGQGTTVTVSSASTKGPSVFPLAPSSKSTS | |
| | GGTAALGCLVKDYFPEPVTVSWNSGALTSGVHTFPA | |
| | VLQSSGLYSLSSVVTVPSSSLGTQTYICNVNHKPSNTK | |
| | VDKKVEPKSC | |
| PC5: LC | DIQMTQSPSSLSASVGDRVTITCRASQGISNYLAWYQ | 70 |
| 006 PSMA Fab LC | QKTGKVPKFLIYEASTLQSGVPSRFSGGGSGTDFTLTIS | |
| | SLQPEDVATYYCQNYNSAPFTFGPGTKVDIKRTVAAP | |
| | SVFIFPPSDEQLKSGTASVVCLLNNFYPREAKVQWKV | |
| | DNALQSGNSQESVTEQDSKDSTYSLSSTLTLSKADYE | |
| | KHKVYACEVTHQGLSSPVTKSFNRGEC | |
| PC5: HC | EVQLVESGGGLVQPGGSLRLSCAASGSTFYTAVMGW | 71 |
| Anti-albumin (SEQ | VRQAPGKGLEWVAAIRWTALTTSYADSVKGRFTISR | |
| ID NO: 57) + Linker | DGAKTTLYLQMNSLRPEDTAVYYCAARGTLGLFTT | |
| 3 + SP34.185 scFv | ADSYDYWGQGTLVTVSSGGGGSGGGSGGVYCGPEFD | |
| mask (SEQ ID NO: | ESVGCMGGGGSGGGLSGRSDAGSPLGLAGSGGGSEV | |
| 17) + cleavable | QLVESGGGLVQPGGSLKLSCAASGFTFNKYAMNWV | |
| linker + SP34.185 | RQAPGKGLEWVARIRSKYNNYATYYADSVKDRFTIS | |
| scFv (VH-linker 1- | RDDSKNTAYLQMNNLKTEDTAVYYCVRHGNFGNSY | |
| VL) + Linker 2 + | ISYWAYWGQGTLVTVSSGGGGSGGGGSGGGGSQTV | |
| 006 PSMA Fab HC | VTQEPSLTVSPGGTVTLTCGSSTGAVTSGNYPNWVQ | |
| | QKPGQAPRGLIGGTKFLAPGTPARFSGSLLGGKAALT | |
| | LSGVQPEDEAEYYCVLWYSNRWVFGGGTKLTVLGG | |
| | GGSQVQLVESGGGVVQPGRSLRLSCAASGFAFSRYG | |
| | MHWVRQAPGKGLEWVAVIWYDGSNKYYADSVKGR | |
| | FTISRDNSKNTQYLQMNSLRAEDTAVYYCARGGDFL | |
| | YYYYYGMDVWGQGTTVTVSSASTKGPSVFPLAPSSK | |
| | STSGGTAALGCLVKDYFPEPVTVSWNSGALTSGVHTF | |
| | PAVLQSSGLYSLSSVVTVPSSSLGTQTYICNVNHKPSN | |
| | TKVDKKVEPKSC | |
| PC6: LC | EVQLVESGGGLVQPGGSLRLSCAASGSTFYTAVMGW | 72 |
| Anti-albumin (SEQ | VRQAPGKGLEWVAAIRWTALTTSYADSVKGRFTISR | |
| ID NO: 57) + Linker | DGAKTTLYLQMNSLRPEDTAVYYCAARGTLGLFTT | |
| 3 + SP34.185 scFv | ADSYDYWGQGTLVTVSSGGGGSGGGSGGVYCGPEFD | |
| mask (SEQ ID NO: | ESVGCMGGGGSGGGLSGRSDAGSPLGLAGSGGGSEV | |
| 17) + cleavable | QLVESGGGLVQPGGSLKLSCAASGFTFNKYAMNWV | |
| linker + SP34.185 | RQAPGKGLEWVARIRSKYNNYATYYADSVKDRFTIS | |
| scFv (VH-linker 1- | RDDSKNTAYLQMNNLKTEDTAVYYCVRHGNFGNSY | |
| VL) + Linker 2 + | ISYWAYWGQGTLVTVSSGGGGSGGGGSGGGGSQTV | |
| 006 PSMA Fab LC | VTQEPSLTVSPGGTVTLTCGSSTGAVTSGNYPNWVQ | |
| | QKPGQAPRGLIGGTKFLAPGTPARFSGSLLGGKAALT | |
| | LSGVQPEDEAEYYCVLWYSNRWVFGGGTKLTVLGG | |
| | GGSDIQMTQSPSSLSASVGDRVTITCRASQGISNYLAW | |
| | YQQKTGKVPKFLIYEASTLQSGVPSRFSGGGSGTDFTL | |
| | TISSLQPEDVATYYCQNYNSAPFTFGPGTKVDIKRTV | |
| | AAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNRGEC | |
| PC6: HC | QVQLVESGGGVVQPGRSLRLSCAASGFAFSRYGMH | 73 |
| 006 PSMA Fab HC | WVRQAPGKGLEWVAVIWYDGSNKYYADSVKGRFTI | |
| | SRDNSKNTQYLQMNSLRAEDTAVYYCARGGDFLYY | |
| | YYYGMDVWGQGTTVTVSSASTKGPSVFPLAPSSKSTS | |
| | GGTAALGCLVKDYFPEPVTVSWNSGALTSGVHTFPA | |
| | VLQSSGLYSLSSVVTVPSSSLGTQTYICNVNHKPSNTK | |
| | VDKKVEPKSC | |
| PC7: LC | EVQLVESGGGLVQPGNSLRLSCAASGFTFSKFGMSW | 74 |
| | VRQAPGKGLEWVSSISGSGRDTLYADSVKGRFTISRD | |
| | NAKTTLYLQMNSLRPEDTAVYYCTIGGSLSVSSQGTL | |
| | VTVSSGGGGSGGGSGGVYCGPEFDESVGCMGGGGSG | |
| | GGLSGRSDAGSPLGLAGSGGGSEVQLVESGGGLVQPG | |
| | GSLKLSCAASGFTFNKYAMNWVRQAPGKGLEWVAR | |
| | IRSKYNNYATYYADSVKDRFTISRDDSKNTAYLQMN | |
| | NLKTEDTAVYYCVRHGNFGNSYISYWAYWGQGTLV | |
| | TVSSGGGGSGGGGSGGGGSQTVVTQEPSLTVSPGGTV | |
| | TLTCGSSTGAVTSGNYPNWVQQKPGQAPRGLIGGTK | |
| | FLAPGTPARFSGSLLGGKAALTLSGVQPEDEAEYYCV | |
| | LWYSNRWVFGGGTKLTVLGGGGSDIQMTQSPSSLSA | |
| | SVGDRVTITCRASQGISNYLAWYQQKTGKVPKFLIYE | |
| | ASTLQSGVPSRFSGGGSGTDFTLTISSLQPEDVATYYC | |
| | QNYNSAPFTFGPGTKVDIKRTVAAPSVFIFPPSDEQLK | |
| | SGTASVVCLLNNFYPREAKVQWKVDNALQSGNSQES | |
| | VTEQDSKDSTYSLSSTLTLSKADYEKHKVYACEVTHQ | |
| | GLSSPVTKSFNRGEC | |
| PC7: HC | QVQLVESGGGVVQPGRSLRLSCAASGFAFSRYGMH | 75 |
| | WVRQAPGKGLEWVAVIWYDGSNKYYADSVKGRFTI | |
| | SRDNSKNTQYLQMNSLRAEDTAVYYCARGGDFLYY | |
| | YYYGMDVWGQGTTVTVSSASTKGPSVFPLAPSSKSTS | |
| | GGTAALGCLVKDYFPEPVTVSWNSGALTSGVHTFPA | |
| | VLQSSGLYSLSSVVTVPSSSLGTQTYICNVNHKPSNTK | |
| | VDKKVEPKSC | |
| PC8: LC | EVQLVESGGGLVQPGNSLRLSCAASGFTFSKFGMSW | 76 |
| | VRQAPGKGLEWVSSISGSGRDTLYADSVKGRFTISRD | |
| | NAKTTLYLQMNSLRPEDTAVYYCTIGGSLSVSSQGTL | |
| | VTVSSGGGGSGGGSGGVYCGPEFDESVGCMGGGGSG | |
| | GGSGGGGSGGASSGAGGSGGGSEVQLVESGGGLVQP | |
| | GGSLKLSCAASGFTFNKYAMNWVRQAPGKGLEWVA | |
| | RIRSKYNNYATYYADSVKDRFTISRDDSKNTAYLQM | |
| | NNLKTEDTAVYYCVRHGNFGNSYISYWAYWGQGTL | |
| | VTVSSGGGGSGGGGSGGGGSQTVVTQEPSLTVSPGGT | |
| | VTLTCGSSTGAVTSGNYPNWVQQKPGQAPRGLIGGT | |
| | KFLAPGTPARFSGSLLGGKAALTLSGVQPEDEAEYYC | |
| | VLWYSNRWVFGGGTKLTVLGGGGSDIQMTQSPSSLS | |
| | ASVGDRVTITCRASQGISNYLAWYQQKTGKVPKFLIY | |
| | EASTLQSGVPSRFSGGGSGTDFTLTISSLQPEDVATYY | |
| | CQNYNSAPFTFGPGTKVDIKRTVAAPSVFIFPPSDEQL | |
| | KSGTASVVCLLNNFYPREAKVQWKVDNALQSGNSQE | |
| | SVTEQDSKDSTYSLSSTLTLSKADYEKHKVYACEVTH | |
| | QGLSSPVTKSFNRGEC | |
| PC8: HC | QVQLVESGGGVVQPGRSLRLSCAASGFAFSRYGMH | 77 |
| | WVRQAPGKGLEWVAVIWYDGSNKYYADSVKGRFTI | |
| | SRDNSKNTQYLQMNSLRAEDTAVYYCARGGDFLYY | |
| | YYYGMDVWGQGTTVTVSSASTKGPSVFPLAPSSKSTS | |
| | GGTAALGCLVKDYFPEPVTVSWNSGALTSGVHTFPA | |
| | VLQSSGLYSLSSVVTVPSSSLGTQTYICNVNHKPSNTK | |
| | VDKKVEPKSC | |

In some embodiments, the polypeptide or polypeptide complex comprises amino acid sequences according to SEQ ID NO: 62 and SEQ ID NO: 63. In some embodiments, the polypeptide or polypeptide complex comprises amino acid sequences with at least 90% sequence identity to SEQ ID NO: 62 and SEQ ID NO: 63. In some embodiments, the polypeptide or polypeptide complex comprises amino acid sequences with at least 95% sequence identity to SEQ ID NO: 62 and SEQ ID NO: 63. In some embodiments, the polypeptide or polypeptide complex comprises amino acid sequences with at least 99% sequence identity to SEQ ID NO: 62 and SEQ ID NO: 63.

In some embodiments, the polypeptide or polypeptide complex comprises amino acid sequences according to SEQ ID NO: 64 and SEQ ID NO: 65. In some embodiments, the polypeptide or polypeptide complex comprises amino acid sequences with at least 90% sequence identity to SEQ ID NO: 64 and SEQ ID NO: 65. In some embodiments, the polypeptide or polypeptide complex comprises amino acid sequences with at least 95% sequence identity to SEQ ID NO: 64 and SEQ ID NO: 65. In some embodiments, the polypeptide or polypeptide complex comprises amino acid sequences with at least 99% sequence identity to SEQ ID NO: 64 and SEQ ID NO: 65.

In some embodiments, the polypeptide or polypeptide complex comprises amino acid sequences according to SEQ ID NO: 66 and SEQ ID NO: 67. In some embodiments, the polypeptide or polypeptide complex comprises amino acid sequences with at least 90% sequence identity to SEQ ID NO: 66 and SEQ ID NO: 67. In some embodiments, the polypeptide or polypeptide complex comprises amino acid sequences with at least 95% sequence identity to SEQ ID NO: 66 and SEQ ID NO: 67. In some embodiments, the polypeptide or polypeptide complex comprises amino acid sequences with at least 99% sequence identity to SEQ ID NO: 66 and SEQ ID NO: 67.

In some embodiments, the polypeptide or polypeptide complex comprises amino acid sequences according to SEQ ID NO: 68 and SEQ ID NO: 69. In some embodiments, the polypeptide or polypeptide complex comprises amino acid sequences with at least 90% sequence identity to SEQ ID NO: 68 and SEQ ID NO: 69. In some embodiments, the polypeptide or polypeptide complex comprises amino acid sequences with at least 95% sequence identity to SEQ ID NO: 68 and SEQ ID NO: 69. In some embodiments, the polypeptide or polypeptide complex comprises amino acid sequences with at least 99% sequence identity to SEQ ID NO: 68 and SEQ ID NO: 69.

In some embodiments, the polypeptide or polypeptide complex comprises amino acid sequences according to SEQ ID NO: 70 and SEQ ID NO: 71. In some embodiments, the polypeptide or polypeptide complex comprises amino acid sequences with at least 90% sequence identity to SEQ ID NO: 70 and SEQ ID NO: 71. In some embodiments, the polypeptide or polypeptide complex comprises amino acid sequences with at least 95% sequence identity to SEQ ID NO: 70 and SEQ ID NO: 71. In some embodiments, the polypeptide or polypeptide complex comprises amino acid sequences with at least 99% sequence identity to SEQ ID NO: 70 and SEQ ID NO: 71.

In some embodiments, the polypeptide or polypeptide complex comprises amino acid sequences according to SEQ ID NO: 72 and SEQ ID NO: 73. In some embodiments, the polypeptide or polypeptide complex comprises amino acid sequences with at least 90% sequence identity to SEQ ID NO: 72 and SEQ ID NO: 73. In some embodiments, the polypeptide or polypeptide complex comprises amino acid sequences with at least 95% sequence identity to SEQ ID NO: 72 and SEQ ID NO: 73. In some embodiments, the polypeptide or polypeptide complex comprises amino acid sequences with at least 99% sequence identity to SEQ ID NO: 72 and SEQ ID NO: 73.

In some embodiments, the polypeptide or polypeptide complex comprises amino acid sequences according to SEQ ID NO: 74 and SEQ ID NO: 75. In some embodiments, the polypeptide or polypeptide complex comprises amino acid sequences with at least 90% sequence identity to SEQ ID NO: 74 and SEQ ID NO: 75. In some embodiments, the polypeptide or polypeptide complex comprises amino acid sequences with at least 95% sequence identity to SEQ ID NO: 74 and SEQ ID NO: 75. In some embodiments, the polypeptide or polypeptide complex comprises amino acid sequences with at least 99% sequence identity to SEQ ID NO: 74 and SEQ ID NO: 75.

In some embodiments, the polypeptide or polypeptide complex comprises amino acid sequences according to SEQ ID NO: 76 and SEQ ID NO: 77. In some embodiments, the polypeptide or polypeptide complex comprises amino acid sequences with at least 90% sequence identity to SEQ ID NO: 76 and SEQ ID NO: 77. In some embodiments, the polypeptide or polypeptide complex comprises amino acid sequences with at least 95% sequence identity to SEQ ID NO: 76 and SEQ ID NO: 77. In some embodiments, the polypeptide or polypeptide complex comprises amino acid sequences with at least 99% sequence identity to SEQ ID NO: 76 and SEQ ID NO: 77.

Polypeptides or polypeptide complexes, in some embodiments, comprise a sequence set forth in Table 6. In some embodiments, the sequence comprises at least or about 70%, 80%, 85%, 90%, 91%, 92%, 93%, 94%, 95%, %%, 97%, 98%, 99%, or 100% sequence identity to any one of SEQ ID NOs: 62-77. In some instances, the sequence comprises at least or about 95% homology to any one of SEQ ID NOs: 62-77.

In some instances, the sequence comprises at least or about 97% homology to any one of SEQ ID NOs: 62-77. In some instances, the sequence comprises at least or about 99% homology to any one of SEQ ID NOs: 62-77. In some instances, the sequence comprises at least or about 100% homology to any one of SEQ ID NOs: 62-77. In some instances, the sequence comprises at least a portion having at least or about 10, 20, 30, 40, 50, 60, 70, 80, 90, 100, 110, 120, 130, 140, 150, 160, 170, 180, 190, 200, 210, or more than 210 amino acids of any one of SEQ ID NOs: 62, 65, 66, 69, 70, 73, 75, or 77. In some instances, the sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or more than 450 amino acids of any one of SEQ ID NOs: 63 or 64. In some instances, the sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, or more than 640 amino acids of any one of SEQ ID NOs: 67, 68, 71, 72, 74, or 76.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

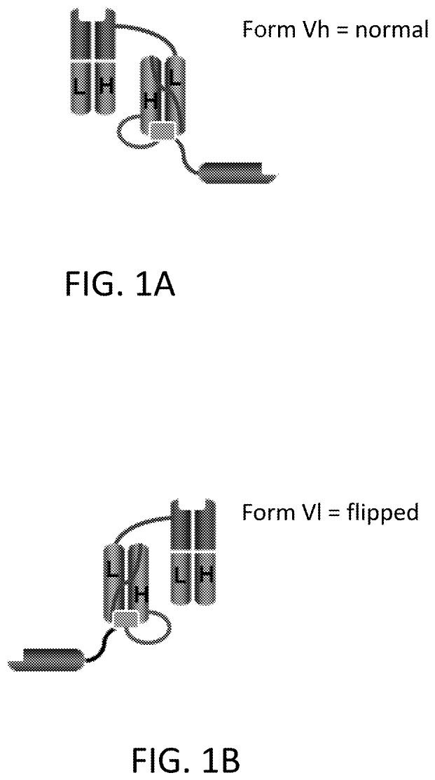

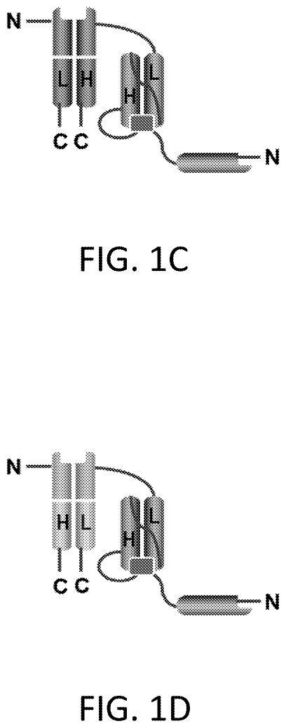

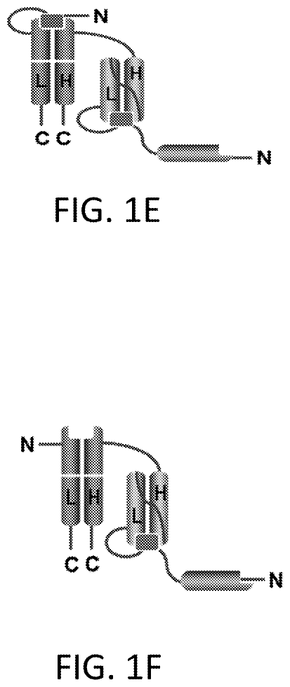

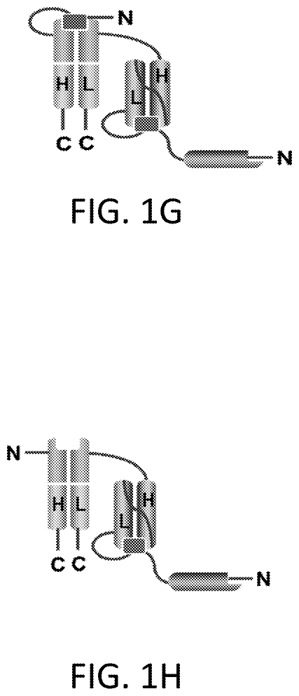

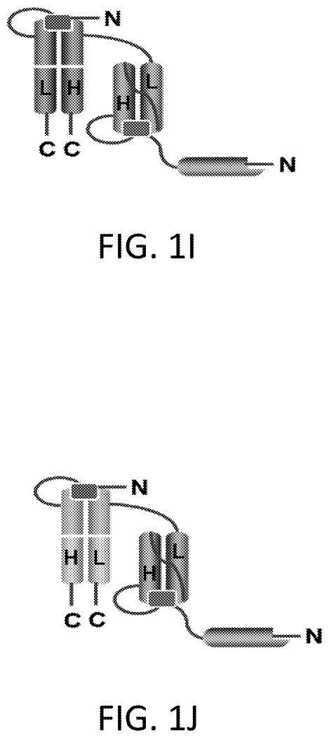

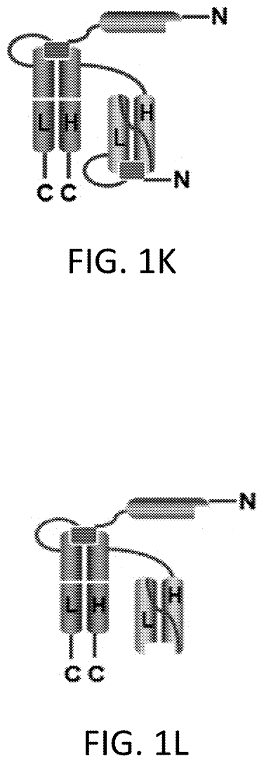

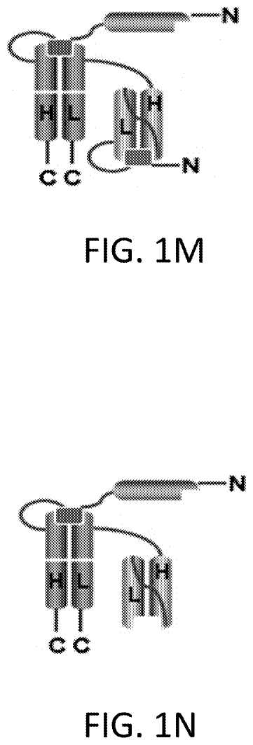

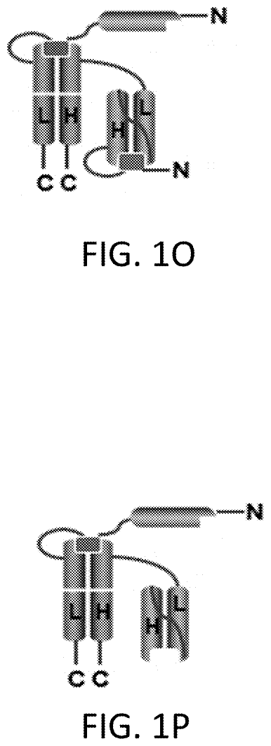

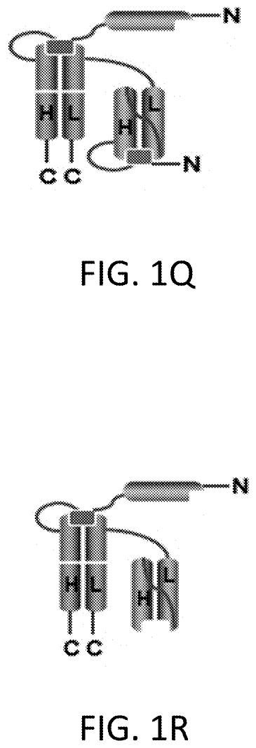

Disclosed herein, in some embodiments, are isolated polypeptides or polypeptide complexes comprising a structural arrangement according to the configuration shown in FIG. 1C, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to the heavy chain variable domain of the scFv with a linking moiety that is a substrate for a tumor specific protease, and the peptide further comprises a half-life extending molecule; and a Fab or a Fab′ that binds to prostate-specific membrane antigen (PSMA), wherein the Fab or Fab′ comprises a Fab light chain polypeptide chain and a Fab heavy chain polypeptide chain, and wherein the Fab heavy chain polypeptide chain is linked to a C terminus of the light chain variable domain of the scFv.

Disclosed herein, in some embodiments, are isolated polypeptides or polypeptide complexes comprising a structural arrangement according to the configuration shown in FIG. 1D, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab or Fab′ that binds to prostate-specific membrane antigen (PSMA), wherein the Fab or Fab′ comprises a Fab light chain polypeptide and a Fab heavy chain polypeptide, wherein the Fab light chain polypeptide is linked to a C terminus of the light chain variable domain of the scFv.

Disclosed herein, in some embodiments, are isolated polypeptides or polypeptide complexes comprising a structural arrangement according to the configuration shown in FIG. 1E, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety (L₁) that is a substrate for a tumor specific protease, and P₁ is further linked to a half-life extending molecule; and a Fab that binds to prostate-specific membrane antigen (PSMA), wherein the Fab comprises a Fab light chain polypeptide and a Fab heavy chain polypeptide, wherein the Fab heavy chain polypeptide is linked to a C terminus of the heavy chain variable domain of the scFv, and wherein the Fab is linked to P₂ and L₂, wherein P₂ comprises a peptide that impairs binding of the Fab to PSMA; and L₂ comprises a linking moiety that connects the Fab light chain polypeptide to P₂ and is a substrate for a tumor specific protease.

Disclosed herein, in some embodiments, are isolated polypeptides or polypeptide complexes comprising a structural arrangement according to the configuration shown in FIG. 1F, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the light chain variable domain of the scFv with a linking moiety that is a substrate for a tumor specific protease, and the peptide is further linked to a half-life extending molecule; and a Fab that binds to prostate-specific membrane antigen (PSMA), wherein the Fab comprises a Fab light chain polypeptide chain and a Fab heavy chain polypeptide chain, and wherein the Fab heavy chain polypeptide chain is linked to a C terminus of the heavy chain variable domain of the scFv.

Disclosed herein, in some embodiments, are isolated polypeptides or polypeptide complexes comprising a structural arrangement according to the configuration shown in FIG. 1G, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety that is a substrate for a tumor specific protease, and P₁ is further linked to a half-life extending molecule; and a Fab that binds to prostate-specific membrane antigen (PSMA), wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to PSMA; and L₂ comprises a linking moiety that connects the Fab heavy chain polypeptide to P₂ and is a substrate for a tumor specific protease.

Disclosed herein, in some embodiments, are isolated polypeptides or polypeptide complexes comprising a structural arrangement according to the configuration shown in FIG. 1H, wherein the polypeptide or polypeptide complex comprises a single chain variable fragment (scFv) comprising a light chain variable domain and a heavy chain variable domain, wherein the scFv is further linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a Fab that binds to prostate-specific membrane antigen (PSMA), wherein the Fab comprises a Fab light chain polypeptide and a Fab heavy chain polypeptide, wherein the Fab light chain polypeptide is linked to a C terminus of the heavy chain variable domain of the scFv.

Disclosed herein, in some embodiments, are isolated polypeptides or polypeptide complexes comprising a structural arrangement according to the configuration shown in FIG. 1I, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to prostate-specific membrane antigen (PSMA), wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding to PSMA; and L₂ comprises a linking moiety that connects the Fab light chain polypeptide to P₂ and is a substrate for a tumor specific protease.

Disclosed herein, in some embodiments, are isolated polypeptides or polypeptide complexes comprising a structural arrangement according to the configuration shown in FIG. 1J, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to prostate-specific membrane antigen (PSMA), wherein the Fab comprises a Fab light chain polypeptide and a Fab heavy chain polypeptide, wherein the Fab light chain polypeptide is linked to a C terminus of the light chain variable domain of the scFv, and wherein the Fab is linked to P₂ and L₂, wherein P₂ comprises a peptide that impairs binding to PSMA; and L₂ comprises a linking moiety that connects the Fab heavy chain polypeptide to P₂ and is a substrate for a tumor specific protease.

Disclosed herein, in some embodiments, are isolated polypeptides or polypeptide complexes comprising a structural arrangement according to the configuration shown in FIG. 1K, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a peptide (P₁) that impairs binding of the Fab to PSMA and P₁ is linked to a N terminus of the Fab light chain polypeptide with a linking moiety (L₁) that is a substrate for a tumor specific protease, and the P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are isolated polypeptides or polypeptide complexes comprising a structural arrangement according to the configuration shown in FIG. 1L, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a peptide that impairs binding of the Fab to PSMA and the peptide is linked to a N terminus of the Fab light chain polypeptide with a linking moiety that is a substrate for a tumor specific protease, and the peptide is further linked to half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide.

Disclosed herein, in some embodiments, are isolated polypeptides or polypeptide complexes comprising a structural arrangement according to the configuration shown in FIG. 1M, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a peptide (P₁) that impairs binding of the Fab to PSMA and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are isolated polypeptides or polypeptide complexes comprising a structural arrangement according to the configuration shown in FIG. 1N, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a peptide that impairs binding of the Fab to PSMA and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide.

Disclosed herein, in some embodiments, are isolated polypeptides or polypeptide complexes comprising a structural arrangement according to the configuration shown in FIG. 1O, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a peptide (P₁) that impairs binding of the Fab to PSMA and P₁ is linked to a N terminus of the Fab light chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are isolated polypeptides or polypeptide complexes comprising a structural arrangement according to the configuration shown in FIG. 1P, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a peptide that impairs binding of the Fab to PSMA and the peptide is linked to a N terminus of the Fab light chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide.

Disclosed herein, in some embodiments, are isolated polypeptides or polypeptide complexes comprising a structural arrangement according to the configuration shown in FIG. 1Q, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a (P₁) that impairs binding of the Fab to PSMA and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are isolated polypeptides or polypeptide complexes comprising a structural arrangement according to the configuration shown in FIG. 1R, wherein the polypeptide or polypeptide complex comprises a Fab that binds to prostate-specific membrane antigen (PSMA), the Fab comprising a Fab light chain polypeptide and a Fab heavy chain polypeptide, wherein the Fab is linked to a peptide that impairs binding of the Fab to PSMA and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide.

### Polynucleotides Encoding Polypeptides or Polypeptide Complexes

Disclosed herein, in some embodiments, are isolated recombinant nucleic acid molecules encoding polypeptides or polypeptide complexes as disclosed herein. In some embodiments, the polypeptides or polypeptide complexes comprise an antibody or an antibody fragment. In some embodiments, the polypeptides or polypeptide complexes comprise a Fab and a single chain variable fragment (scFv).

Disclosed herein, in some embodiments, are isolated recombinant nucleic acid molecules encoding polypeptides or polypeptide complexes according to Formula I:

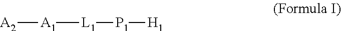

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA).

Disclosed herein, in some embodiments, are isolated recombinant nucleic acid molecules encoding polypeptides or polypeptide complexes according to Formula I:

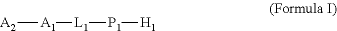

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to prostate-specific membrane antigen (PSMA).

Disclosed herein, in some embodiments, are isolated recombinant nucleic acid molecules encoding polypeptides or polypeptide complexes comprising Formula I:

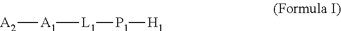

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA).

Disclosed herein, in some embodiments, are isolated recombinant nucleic acid molecules encoding polypeptides or polypeptide complexes comprising Formula I:

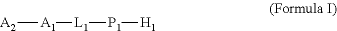

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to prostate-specific membrane antigen (PSMA).

Disclosed herein, in some embodiments, are isolated recombinant nucleic acid molecules encoding polypeptides or polypeptide complexes according to Formula Ia:

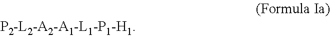

Disclosed herein, in some embodiments, are isolated recombinant nucleic acid molecules encoding polypeptides or polypeptide complexes according to Formula II:

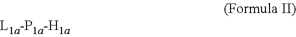

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ comprises a peptide that binds to the first antigen recognizing molecule when L₁ₐ is uncleaved; and H₁ₐ comprises a half-life extending molecule.

Disclosed herein, in some embodiments, are isolated recombinant nucleic acid molecules encoding polypeptides or polypeptide complexes comprising Formula II:

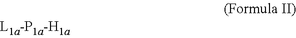

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ comprises a peptide that binds to the first antigen recognizing molecule when L₁ₐ is uncleaved; and H₁ₐ comprises a half-life extending molecule.

Disclosed herein, in some embodiments, are isolated recombinant nucleic acid molecules encoding polypeptides or polypeptide complexes according to Formula II:

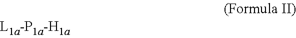

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ is a peptide that binds to the first antigen recognizing molecule when L₁ₐ is uncleaved; and H₁ₐ is a half-life extending molecule.

Disclosed herein, in some embodiments, are isolated recombinant nucleic acid molecules encoding polypeptides or polypeptide complexes comprising Formula II:

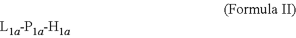

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ is a peptide that binds to the first antigen recognizing molecule when L₁ₐ is uncleaved; and H₁ₐ is a half-life extending molecule.

Disclosed herein, in some embodiments, are isolated nucleic acid molecules encoding polypeptides or polypeptide complexes comprising a structural arrangement according to the configuration shown in FIG. 1C, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to the heavy chain variable domain of the scFv with a linking moiety that is a substrate for a tumor specific protease, and the peptide further comprises a half-life extending molecule; and a Fab or a Fab′ that binds to prostate-specific membrane antigen (PSMA), wherein the Fab or Fab′ comprises a Fab light chain polypeptide chain and a Fab heavy chain polypeptide chain, and wherein the Fab heavy chain polypeptide chain is linked to a C terminus of the light chain variable domain of the scFv.

Disclosed herein, in some embodiments, are isolated nucleic acid molecules encoding polypeptides or polypeptide complexes comprising a structural arrangement according to the configuration shown in FIG. 1D, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab or Fab′ that binds to prostate-specific membrane antigen (PSMA), wherein the Fab or Fab′ comprises a Fab light chain polypeptide and a Fab heavy chain polypeptide, wherein the Fab light chain polypeptide is linked to a C terminus of the light chain variable domain of the scFv.

### Pharmaceutical Compositions

Disclosed herein, in some embodiments, are pharmaceutical compositions comprising: (a) the polypeptides or polypeptide complexes as disclosed herein; and (b) a pharmaceutically acceptable excipient.

In some embodiments, the pharmaceutical composition comprises (a) polypeptides or polypeptide complexes according to Formula I:

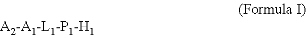

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA); and (b) a pharmaceutically acceptable excipient.

In some embodiments, the pharmaceutical composition comprises (a) polypeptides or polypeptide complexes according to Formula I:

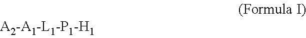

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to prostate-specific membrane antigen (PSMA); and (b) a pharmaceutically acceptable excipient.

In some embodiments, the pharmaceutical composition comprises (a) polypeptides or polypeptide complexes comprising Formula I:

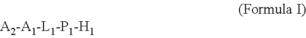

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA); and (b) a pharmaceutically acceptable excipient.

In some embodiments, the pharmaceutical composition comprises (a) polypeptides or polypeptide complexes comprising Formula I:

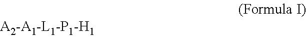

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to prostate-specific membrane antigen (PSMA); and (b) a pharmaceutically acceptable excipient.

In some embodiments, the pharmaceutical composition comprises (a) polypeptides or polypeptide complexes according to Formula Ia:

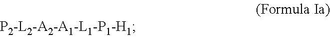

and (b) a pharmaceutically acceptable excipient.

In some embodiments, the pharmaceutical composition comprises (a) polypeptides or polypeptide complexes according to Formula II:

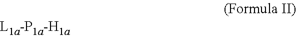

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ comprises a peptide that binds to the first antigen recognizing molecule when L₁ₐ is uncleaved; and H₁ₐ comprises a half-life extending molecule; and (b) a pharmaceutically acceptable excipient.

In some embodiments, the pharmaceutical composition comprises (a) polypeptides or polypeptide complexes comprising Formula II:

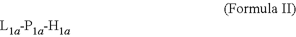

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ comprises a peptide that binds to the first antigen recognizing molecule when L₁ₐ is uncleaved; and H₁ₐ comprises a half-life extending molecule; and (b) a pharmaceutically acceptable excipient.

In some embodiments, the pharmaceutical composition comprises (a) polypeptides or polypeptide complexes according to Formula II:

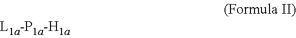

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ is a peptide that binds to the first antigen recognizing molecule when L₁ₐ is uncleaved; and His is a half-life extending molecule; and (b) a pharmaceutically acceptable excipient.

In some embodiments, the pharmaceutical composition comprises (a) polypeptides or polypeptide complexes comprising Formula II:

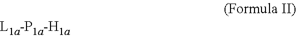

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to prostate-specific membrane antigen (PSMA); P₁ₐ is a peptide that binds to the first antigen recognizing molecule when L₁ is uncleaved; and His is a half-life extending molecule; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 1C, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to the heavy chain variable domain of the scFv with a linking moiety that is a substrate for a tumor specific protease, and the peptide further comprises a half-life extending molecule; and a Fab or a Fab′ that binds to prostate-specific membrane antigen (PSMA), wherein the Fab or Fab′ comprises a Fab light chain polypeptide chain and a Fab heavy chain polypeptide chain, and wherein the Fab heavy chain polypeptide chain is linked to a C terminus of the light chain variable domain of the scFv; and (b) a pharmaceutically acceptable excipient. Disclosed herein, in some embodiments, the pharmaceutical composition comprises (a) polypeptides or polypeptide complexes comprising a structural arrangement according to the configuration shown in FIG. 1D.

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab or Fab′ that binds to prostate-specific membrane antigen (PSMA), wherein the Fab or Fab′ comprises a Fab light chain polypeptide and a Fab heavy chain polypeptide, wherein the Fab light chain polypeptide is linked to a C terminus of the light chain variable domain of the scFv; and (b) a pharmaceutically acceptable excipient.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Treatment

In some embodiments, are methods of treating cancer in a subject need in need thereof comprising administering to the subject an isolated polypeptide or polypeptide complex as described herein. In some embodiments, the cancer has cells that express PSMA. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating prostate cancer in a subject need in need thereof comprising administering to the subject an isolated polypeptide or polypeptide complex as described herein. In some embodiments, are methods of treating metastatic castrate-resistant prostate cancer (mCRPC) in a subject need in need thereof comprising administering to the subject an isolated polypeptide or polypeptide complex as described herein.

Described herein, in some embodiments, are isolated polypeptides or polypeptide complexes, wherein the polypeptides or polypeptide complexes comprise a long half-life. In some instances, the half-life of the polypeptides or polypeptide complexes is at least or about 12 hours, 24 hours 36 hours, 48 hours, 60 hours, 72 hours, 84 hours, 96 hours, 100 hours, 108 hours, 119 hours, 120 hours, 140 hours, 160 hours, 180 hours, 200 hours, or more than 200 hours. In some instances, the half-life of the polypeptides or polypeptide complexes is in a range of about 12 hours to about 300 hours, about 20 hours to about 280 hours, about 40 hours to about 240 hours, about 60 hours to about 200 hours, or about 80 hours to about 140 hours.

Described herein, in some embodiments, are polypeptide or polypeptide complexes administered as once weekly. In some embodiments, the polypeptide or polypeptide complexes are administered once weekly by intravenous, intramuscular, intralesional, topical, subcutaneous, infusion, or oral. In some embodiments, the polypeptide or polypeptide complexes are administered once weekly by bolus injection. In some embodiments, the polypeptide or polypeptide complexes are administered once weekly by continuous infusion. In some embodiments, the polypeptide or polypeptide complex is administered to the subject once a week as a continuous infusion over a period of no more than 60 minutes. In some embodiments, the polypeptide or polypeptide complex is administered to the subject once a week as a continuous intravenous infusion over a period of no more than 30 minutes. In some embodiments, the polypeptide or polypeptide complex is administered to the subject once a week as a continuous intravenous infusion over a period of at least 10 minutes.

In some embodiments, the polypeptide or polypeptide complex is administered to the subject once a week and the polypeptide or polypeptide complex has a half-life of at least 30 hours. In some embodiments, the polypeptide or polypeptide complex is administered to the subject once a week and the polypeptide or polypeptide complex has a half-life of at least 50 hours. In some embodiments, the polypeptide or polypeptide complex is administered to the subject once a week and the polypeptide or polypeptide complex has a half-life of at least 60 hours. In some embodiments, the polypeptide or polypeptide complex is administered to the subject once a week and the polypeptide or polypeptide complex has a half-life of at least 70 hours. In some embodiments, the polypeptide or polypeptide complex is administered to the subject once a week and the polypeptide or polypeptide complex has a half-life of at least 80 hours. In some embodiments, the polypeptide or polypeptide complex is administered to the subject once a week and the polypeptide or polypeptide complex has a half-life of at least 90 hours. In some embodiments, the polypeptide or polypeptide complex is administered to the subject once a week and the polypeptide or polypeptide complex has a half-life of at least 100 hours. In some embodiments, the polypeptide or polypeptide complex is administered to the subject once a week and the polypeptide or polypeptide complex has a half-life of at least 110 hours. In some embodiments, the polypeptide or polypeptide complex is administered to the subject once a week and the polypeptide or polypeptide complex has a half-life of at least 115 hours. In some embodiments, the polypeptide or polypeptide complex is administered to the subject once a week and the polypeptide or polypeptide complex has a half-life of at least 119 hours.

Production of Antibodies that Bind to PSMA and CD3

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk−, hgprt− or aprt− cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in the culture of the host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vector derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cells include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVaF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD3 and a second antigen-binding site that specifically binds to PSMA as defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production. Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

Embodiment 1 comprises an isolated polypeptide or polypeptide complex according to Formula I: A₂-A₁-L₁-P₁-H₁ wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to prostate-specific membrane antigen (PSMA).

Embodiment 2 comprises an isolated polypeptide or polypeptide complex of embodiment 1, wherein the first antigen recognizing molecule comprises an antibody or antibody fragment.

Embodiment 3 comprises an isolated polypeptide or polypeptide complex of embodiment 1, wherein first antigen recognizing molecule comprises an antibody or antibody fragment that is human or humanized.

Embodiment 4 comprises an isolated polypeptide or polypeptide complex of any one of embodiments 1-3, wherein L₁ is bound to N-terminus of the first antigen recognizing molecule.

Embodiment 5 comprises an isolated polypeptide or polypeptide complex of any one of embodiments 1-3, wherein A₂ is bound to C-terminus of the first antigen recognizing molecule.

Embodiment 6 comprises an isolated polypeptide or polypeptide complex of any one of embodiments 1-3, wherein L₁ is bound to C-terminus of the first antigen recognizing molecule.

Embodiment 7 comprises an isolated polypeptide or polypeptide complex of any one of embodiments 1-3, wherein A₂ is bound to N-terminus of the first antigen recognizing molecule.

Embodiment 8 comprises an isolated polypeptide or polypeptide complex of any one of embodiments 2-7, wherein the antibody or antibody fragment comprises a single chain variable fragment, a single domain antibody, or a Fab fragment.

Embodiment 9 comprises an isolated polypeptide or polypeptide complex of embodiment 8, wherein A₁ is the single chain variable fragment (scFv).

Embodiment 10 comprises an isolated polypeptide or polypeptide complex of embodiment 9, wherein the scFv comprises a scFv heavy chain polypeptide and a scFv light chain polypeptide.

Embodiment 11 comprises an isolated polypeptide or polypeptide complex of embodiment 8, wherein A₁ is the single domain antibody,

Embodiment 12 comprises an isolated polypeptide or polypeptide complex of embodiment 8, wherein the antibody or antibody fragment comprises a single chain variable fragment (scFv), a heavy chain variable domain (VH domain), a light chain variable domain (VL domain), or a variable domain (VHH) of a camelid derived single domain antibody.

Embodiment 13 comprises an isolated polypeptide or polypeptide complex of any one of embodiments 1-12, wherein A₁ comprises an anti-CD3e single chain variable fragment.

Embodiment 14 comprises an isolated polypeptide or polypeptide complex of any one of embodiments 1-12, wherein A₁ comprises an anti-CD3e single chain variable fragment that has a K_{D} binding of 1 μM or less to CD3 on CD3 expressing cells.

Embodiment 15 comprises an isolated polypeptide or polypeptide complex of any one of embodiments 1-14, wherein the effector cell antigen comprises CD3.

Embodiment 16 comprises an isolated polypeptide or polypeptide complex of embodiment 1, wherein A₁ comprises a variable light chain and variable heavy chain each of which is capable of specifically binding to human CD3.

Embodiment 17 comprises an isolated polypeptide or polypeptide complex of embodiment 1, wherein A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 18 comprises an isolated polypeptide or polypeptide complex of embodiment 1, wherein the polypeptide or polypeptide complex of Formula I binds to an effector cell when L₁ is cleaved by the tumor specific protease.

Embodiment 19 comprises an isolated polypeptide or polypeptide complex of embodiment 1, wherein the polypeptide or polypeptide complex of Formula I binds to an effector cell when L₁ is cleaved by the tumor specific protease and A₁ binds to the effector cell.

Embodiment 20 comprises an isolated polypeptide or polypeptide complex of embodiment 19, wherein the effector cell is a T cell.

Embodiment 21 comprises an isolated polypeptide or polypeptide complex of embodiment 1, wherein A₁ binds to a polypeptide that is part of a TCR-CD3 complex on the effector cell.

Embodiment 22 comprises an isolated polypeptide or polypeptide complex of embodiment 21, wherein the polypeptide that is part of the TCR-CD3 complex is human CD3E.

Embodiment 23 comprises an isolated polypeptide or polypeptide complex of embodiment 1, wherein the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv comprise: HC-CDR1: SEQ ID NO: 1, HC-CDR2: SEQ ID NO: 2, and HC-CDR3: SEQ ID NO: 3; and the scFv comprises CDRs: LC-CDR1, LC-CDR2, and LC-CDR3, wherein the LC-CDR1, the LC-CDR2, and the LC-CDR3 of the scFv comprise: LC-CDR1: SEQ ID NO: 4, LC-CDR2: SEQ ID NO:5, and LC-CDR3: SEQ ID NO: 6.

Embodiment 24 comprises an isolated polypeptide or polypeptide complex of embodiment 1, wherein the effector cell antigen comprises CD3, and the scFv comprises an amino acid sequence according to SEQ ID NO: 7.

Embodiment 25 comprises an isolated polypeptide or polypeptide complex of any one of embodiments 1-24, wherein second antigen recognizing molecule comprises an antibody or antibody fragment.

Embodiment 26 comprises an isolated polypeptide or polypeptide complex of embodiment 25, wherein the antibody or antibody fragment thereof comprises a single chain variable fragment, a single domain antibody, or a Fab.

Embodiment 27 comprises an isolated polypeptide or polypeptide complex of embodiment 25, wherein the antibody or antibody fragment thereof comprises a single chain variable fragment (scFv), a heavy chain variable domain (VH domain), a light chain variable domain (VL domain), a variable domain (VHH) of a camelid derived single domain antibody.

Embodiment 28 comprises an isolated polypeptide or polypeptide complex of embodiment 25, wherein the antibody or antibody fragment thereof is humanized or human.

Embodiment 29 comprises an isolated polypeptide or polypeptide complex of embodiment 26, wherein A₂ is the Fab.

Embodiment 30 comprises an isolated polypeptide or polypeptide complex of embodiment 29, wherein the Fab comprises (a) a Fab light chain polypeptide and (b) a Fab heavy chain polypeptide.

Embodiment 31 comprises an isolated polypeptide or polypeptide complex of embodiment 29, wherein the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 8, HC-CDR2: SEQ ID NO: 9, and HC-CDR3: SEQ ID NO: 10; and the Fab comprises CDRs: LC-CDR1, LC-CDR2, and LC-CDR3, wherein the LC-CDR1, the LC-CDR2, and the LC-CDR3 of the Fab comprise: LC-CDR1: SEQ ID NO: 11, LC-CDR2: SEQ ID NO:12, and LC-CDR3: SEQ ID NO: 13.

Embodiment 32 comprises an isolated polypeptide or polypeptide complex of embodiment 30, wherein the Fab light chain polypeptide comprises an amino acid sequence according to SEQ ID NO: 14.

Embodiment 33 comprises an isolated polypeptide or polypeptide complex of embodiment 30, wherein Fab heavy chain polypeptide comprises an amino acid sequence according to SEQ ID NO: 15.

Embodiment 34 comprises an isolated polypeptide or polypeptide complex of embodiment 30, wherein the Fab light chain polypeptide of A₂ is bound to a C-terminus of the single chain variable fragment (scFv) of A₁.

Embodiment 35 comprises an isolated polypeptide or polypeptide complex of embodiment 30, wherein the Fab heavy chain polypeptide of A₂ is bound to a C-terminus of the single chain variable fragment (scFv) A₁.

Embodiment 36 comprises an isolated polypeptide or polypeptide complex of embodiment 30, wherein the Fab light chain polypeptide of A₂ is bound to a N-terminus of the single chain variable fragment (scFv) of A₁.

Embodiment 37 comprises an isolated polypeptide or polypeptide complex of embodiment 30, wherein the Fab heavy chain polypeptide of A₂ is bound to a N-terminus of the single chain variable fragment (scFv) A₁.

Embodiment 38 comprises a polypeptide or polypeptide complex of embodiment 30, wherein the Fab heavy chain polypeptide of A₂ is bound to the scFv heavy chain polypeptide of A₁.

Embodiment 39 comprises a polypeptide or polypeptide complex of embodiment 30, wherein the Fab light chain polypeptide of A₂ is bound to the scFv heavy chain polypeptide of A₁.

Embodiment 40 comprises a polypeptide or polypeptide complex of embodiment 30, wherein the Fab heavy chain polypeptide of A₂ is bound to the scFv light chain polypeptide of A₁.

Embodiment 41 comprises a polypeptide or polypeptide complex of embodiment 30, wherein the Fab light chain polypeptide of A₂ is bound to the scFv light chain polypeptide of A₁.

Embodiment 42 comprises a polypeptide or polypeptide complex of any one of embodiments 1-41, wherein A₂ further comprises P₂ and L₂, wherein P₂ comprises a peptide that binds to A₂; and L₂ comprises a linking moiety that connects A₂ to P₂ and is a substrate for a tumor specific protease.

Embodiment 43 comprises a polypeptide or polypeptide complex of embodiment 42, wherein the polypeptide or polypeptide complex is according to Formula Ia: P₂-L₂-A₂-A₁-L₁-P₁-H₁.

Embodiment 44 comprises a polypeptide or polypeptide complex of embodiment 43, wherein the Fab heavy chain polypeptide of A₂ is bound to the scFv heavy chain polypeptide of A₁ and L₂ is bound to the Fab light chain polypeptide of A₂.

Embodiment 45 comprises a polypeptide or polypeptide complex of embodiment 43, wherein the Fab light chain polypeptide of A₂ is bound to the scFv heavy chain polypeptide of A₁ and L₂ is bound to the Fab heavy chain polypeptide of A₂.

Embodiment 46 comprises a polypeptide or polypeptide complex of embodiment 43, wherein the Fab heavy chain polypeptide of A₂ is bound to the scFv light chain polypeptide of A₁ and L₂ is bound to the Fab light chain polypeptide of A₂.

Embodiment 47 comprises a polypeptide or polypeptide complex of embodiment 43, wherein the Fab light chain polypeptide of A₂ is bound to the scFv light chain polypeptide of A₁ and L₂ is bound to the Fab heavy chain polypeptide of A₂.

Embodiment 48 comprises a polypeptide or polypeptide complex of any one of embodiments 1-47, wherein P₁ impairs binding of A₁ to the effector cell antigen.

Embodiment 49 comprises a polypeptide or polypeptide complex of any one of embodiments 1-48, wherein P₁ is bound to A₁ through ionic interactions, electrostatic interactions, hydrophobic interactions, Pi-stacking interactions, or H-bonding interactions, or a combination thereof.

Embodiment 50 comprises a polypeptide or polypeptide complex of any one of embodiments 1-48, wherein P₁ has less than 70% sequence homology to the effector cell antigen.

Embodiment 51 comprises a polypeptide or polypeptide complex of any one of embodiments 1-50, wherein P₂ impairs binding of A₂ to PSMA.

Embodiment 52 comprises a polypeptide or polypeptide complex of any one of embodiments 1-50, wherein P₂ is bound to A₂ through ionic interactions, electrostatic interactions, hydrophobic interactions, Pi-stacking interactions, or H-bonding interactions, or a combination thereof.

Embodiment 53 comprises a polypeptide or polypeptide complex of any one of embodiments 1-50, wherein P₂ is bound to A2 at or near an antigen binding site.

Embodiment 54 comprises a polypeptide or polypeptide complex of any one of embodiments 1-50, wherein P₂ has less than 70% sequence homology to PSMA.

Embodiment 55 comprises a polypeptide or polypeptide complex of any one of embodiments 1-54, wherein P₁ or P₂ comprises a peptide sequence of at least 10 amino acids in length.

Embodiment 56 comprises a polypeptide or polypeptide complex of any one of embodiments 1-54, wherein P₁ or P₂ comprises a peptide sequence of at least 10 amino acids in length and no more than 20 amino acids in length.

Embodiment 57 comprises a polypeptide or polypeptide complex of any one of embodiments 1-54, wherein P₁ or P₂ comprises a peptide sequence of at least 16 amino acids in length.

Embodiment 58 comprises a polypeptide or polypeptide complex of any one of embodiments 1-54, wherein P₁ or P₂ comprises a peptide sequence of no more than 40 amino acids in length.

Embodiment 59 comprises an isolated polypeptide or polypeptide complex of any one of embodiments 1-54, wherein P₁ or P₂ comprises at least two cysteine amino acid residues.

Embodiment 60 comprises an isolated polypeptide or polypeptide complex of any one of embodiments 1-54, wherein P₁ or P₂ comprises a cyclic peptide or a linear peptide.

Embodiment 61 comprises an isolated polypeptide or polypeptide complex of any one of embodiments 1-54, wherein P₁ or P₂ comprises a cyclic peptide.

Embodiment 62 comprises an isolated polypeptide or polypeptide complex of any one of embodiments 1-54, wherein P₁ or P₂ comprises a linear peptide

Embodiment 63 comprises an isolated polypeptide or polypeptide complex of any one of embodiments 1-54, wherein P₁ comprises at least two cysteine amino acid residues.

Embodiment 64 comprises a polypeptide or polypeptide complex of any one of embodiments 1-54, wherein P₁ comprises an amino acid sequence according to any one of SEQ ID NOs: 16-19 or 78.

Embodiment 65 comprises an isolated polypeptide or polypeptide complex of any one of embodiments 1-64, wherein L₁ is bound to N-terminus of A₁.

Embodiment 66 comprises an isolated polypeptide or polypeptide complex of any one of embodiments 1-64, wherein L₁ is bound to C-terminus of A₁.

Embodiment 67 comprises an isolated polypeptide or polypeptide complex of any one of embodiments 1-66, wherein L₂ is bound to N-terminus of A₂.

Embodiment 68 comprises an isolated polypeptide or polypeptide complex of any one of embodiments 1-66, wherein L₂ is bound to C-terminus of A₂.

Embodiment 69 comprises an isolated polypeptide or polypeptide complex of any one of embodiments 1-68, wherein L₁ or L₂ is a peptide sequence having at least 5 to no more than 50 amino acids.

Embodiment 70 comprises an isolated polypeptide or polypeptide complex of any one of embodiments 1-68, wherein L₁ or L₂ is a peptide sequence having at least 10 to no more than 30 amino acids.

Embodiment 71 comprises an isolated polypeptide or polypeptide complex of any one of embodiments 1-68, wherein L₁ or L₂ is a peptide sequence having at least 10 amino acids.

Embodiment 72 comprises an isolated polypeptide or polypeptide complex of any one of embodiments 1-68, wherein L₁ or L₂ is a peptide sequence having at least 18 amino acids.

Embodiment 73 comprises an isolated polypeptide or polypeptide complex of any one of embodiments 1-68, wherein L₁ or L₂ is a peptide sequence having at least 26 amino acids.

Embodiment 74 comprises an isolated polypeptide or polypeptide complex of any one of embodiments 1-68, wherein L₁ or L₂ has a formula comprising (G₂S)ₙ, wherein n is an integer from 1 to 3 (SEQ ID NO: 118).

Embodiment 75 comprises an isolated polypeptide or polypeptide complex of any one of embodiments 1-68, wherein L₁ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1.

Embodiment 76 comprises an isolated polypeptide or polypeptide complex of any one of embodiments 1-68, wherein P₁ becomes unbound from A₁ when L₁ is cleaved by the tumor specific protease thereby exposing A₁ to the effector cell antigen.

Embodiment 77 comprises an isolated polypeptide or polypeptide complex of any one of embodiments 1-68, wherein P₂ becomes unbound from A₂ when L₂ is cleaved by the tumor specific protease thereby exposing A₂ to PSMA.

Embodiment 78 comprises an isolated polypeptide or polypeptide complex of any one of embodiments 1-68, wherein the tumor specific protease is selected from the group consisting of a matrix metalloprotease (MMP), serine protease, cysteine protease, threonine protease, and aspartic protease.

Embodiment 79 comprises an isolated polypeptide or polypeptide complex of embodiment 78, wherein the matrix metalloprotease comprises MMP2, MMP7, MMP9, MMP13, or MMP14.

Embodiment 80 comprises an isolated polypeptide or polypeptide complex of embodiment 78, wherein the serine protease comprises matriptase (MTSP1), urokinase, or hepsin.

Embodiment 81 comprises an isolated polypeptide or polypeptide complex of any one of embodiments 1-68, wherein L₁ or L₂ comprises a urokinase cleavable amino acid sequence, a matriptase cleavable amino acid sequence, matrix metalloprotease cleavable amino acid sequence, or a legumain cleavable amino acid sequence.

Embodiment 82 comprises an isolated polypeptide or polypeptide complex of any one of embodiments 1-68, wherein L₁ or L₂ comprises an amino acid sequence according to SEQ ID NO: 23.

Embodiment 83 comprises an isolated polypeptide or polypeptide complex of any one of embodiments 1-68, wherein L₁ or L₂ comprises an amino acid sequence according to any one of SEQ ID NOs: 20-49.

Embodiment 84 comprises an isolated polypeptide or polypeptide complex of any one of embodiments 1-68, wherein L₁ or L₂ comprises an amino acid sequence of Linker 25 (ISSGLLSGRSDAG) (SEQ ID NO: 45), Linker 26 (AAGLLAPPGGLSGRSDAG) (SEQ ID NO: 46), Linker 27 (SPLGLSGRSDAG) (SEQ ID NO: 47), or Linker 28 (LSGRSDAGSPLGLAG) (SEQ ID NO: 48), or an amino acid sequence that has 1, 2, or 3 amino acid substitutions, additions, or deletions relative to the amino acid sequence of Linker 25, Linker 26, Linker 27, or Linker 28.

Embodiment 85 comprises an isolated polypeptide or polypeptide complex of any one of embodiments 1-83, wherein H₁ comprises a polymer.

Embodiment 86 comprises an isolated polypeptide or polypeptide complex of any one of embodiments 1-83, wherein the polymer is polyethylene glycol (PEG).

Embodiment 87 comprises an isolated polypeptide or polypeptide complex of any one of embodiments 1-83, wherein H₁ comprises albumin.

Embodiment 88 comprises an isolated polypeptide or polypeptide complex of any one of embodiments 1-83, wherein H₁ comprises an Fc domain.

Embodiment 89 comprises an isolated polypeptide or polypeptide complex of embodiment 87, wherein the albumin is serum albumin.

Embodiment 90 comprises an isolated polypeptide or polypeptide complex of embodiment 87, wherein the albumin is human serum albumin.

Embodiment 91 comprises an isolated polypeptide or polypeptide complex of any one of embodiments 1-83, wherein H₁ comprises a polypeptide, a ligand, or a small molecule.

Embodiment 92 comprises an isolated polypeptide or polypeptide complex of embodiment 91, wherein the polypeptide, the ligand or the small molecule binds serum protein or a fragment thereof, a circulating immunoglobulin or a fragment thereof, or CD35/CR1.

Embodiment 93 comprises an isolated polypeptide or polypeptide complex of embodiment 88, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 94 comprises an isolated polypeptide or polypeptide complex of embodiment 88, wherein the circulating immunoglobulin molecule comprises IgG1, IgG2, IgG3, IgG4, slgA, IgM or IgD.

Embodiment 95 comprises an isolated polypeptide or polypeptide complex of embodiment 92, wherein the serum protein is albumin.

Embodiment 96 comprises an isolated polypeptide or polypeptide complex of embodiment 91, wherein the polypeptide is an antibody.

Embodiment 97 comprises an isolated polypeptide or polypeptide complex of embodiment 96, wherein the antibody comprises a single domain antibody, a single chain variable fragment, or a Fab.

Embodiment 98 comprises an isolated polypeptide or polypeptide complex of embodiment 97, wherein the single domain antibody comprises a single domain antibody that binds to albumin.

Embodiment 99 comprises an isolated polypeptide or polypeptide complex of embodiment 97, wherein the single domain antibody is a human or humanized antibody.

Embodiment 100 comprises an isolated polypeptide or polypeptide complex of embodiment 97, wherein the single domain antibody is 645gH1gL1.

Embodiment 101 comprises an isolated polypeptide or polypeptide complex of embodiment 97, wherein the single domain antibody is 645dsgH5gL4.

Embodiment 102 comprises an isolated polypeptide or polypeptide complex of embodiment 97, wherein the single domain antibody is 23-13-A01-sc02.

Embodiment 103 comprises an isolated polypeptide or polypeptide complex of embodiment 97, wherein the single domain antibody is A10m3 or a fragment thereof.

Embodiment 104 comprises an isolated polypeptide or polypeptide complex of embodiment 97, wherein the single domain antibody is DOM7r-31.

Embodiment 105 comprises an isolated polypeptide or polypeptide complex of embodiment 97, wherein the single domain antibody is DOM7 h-11-15.

Embodiment 106 comprises an isolated polypeptide or polypeptide complex of embodiment 97, wherein the single domain antibody is Alb-1, Alb-8, or Alb-23.

Embodiment 107 comprises an isolated polypeptide or polypeptide complex of embodiment 97, wherein the single domain antibody is 10E.

Embodiment 108 comprises an isolated polypeptide or polypeptide complex of embodiment 97, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56.

Embodiment 109 comprises an isolated polypeptide or polypeptide complex of embodiment 97, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 58, HC-CDR2: SEQ ID NO: 59, and HC-CDR3: SEQ ID NO: 60.

Embodiment 110 comprises an isolated polypeptide or polypeptide complex of embodiment 97, wherein the single domain antibody is SA21.

Embodiment 111 comprises an isolated polypeptide or polypeptide complex of any one of embodiments 1-110, wherein the polypeptide or polypeptide complex comprises a modified amino acid, a non-natural amino acid, a modified non-natural amino acid, or a combination thereof.

Embodiment 112 comprises an isolated polypeptide or polypeptide complex of embodiment 111, wherein the modified amino acid or modified non-natural amino acid comprises a post-translational modification.

Embodiment 113 comprises an isolated polypeptide or polypeptide complex of anyone of embodiments 1-112, wherein H₁ comprises a linking moiety (L₃) that connects H₁ to P₁.

Embodiment 114 comprises an isolated polypeptide or polypeptide complex of embodiment 113, wherein L₃ is a peptide sequence having at least 5 to no more than 50 amino acids.

Embodiment 115 comprises an isolated polypeptide or polypeptide complex of embodiment 113, wherein L₃ is a peptide sequence having at least 10 to no more than 30 amino acids.

Embodiment 116 comprises an isolated polypeptide or polypeptide complex of embodiment 113, wherein L₃ is a peptide sequence having at least 10 amino acids.

Embodiment 117 comprises an isolated polypeptide or polypeptide complex of embodiment 113, wherein L₃ is a peptide sequence having at least 18 amino acids.

Embodiment 118 comprises an isolated polypeptide or polypeptide complex of embodiment 113, wherein L₃ is a peptide sequence having at least 26 amino acids.

Embodiment 119 comprises an isolated polypeptide or polypeptide complex of embodiment 113, wherein L₃ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1.

Embodiment 120 comprises an isolated polypeptide or polypeptide complex of embodiment 113, wherein L₃ comprises an amino acid sequence according to SEQ ID NO: 22.

Embodiment 121 comprises an isolated polypeptide or polypeptide complex of embodiment 1, wherein the polypeptide or polypeptide complex comprises an amino acid sequence with at least 95% sequence identity to SEQ ID NOs: 62-77.

Embodiment 122 comprises an isolated polypeptide or polypeptide complex of embodiment 1, wherein the polypeptide or polypeptide complex comprises an amino acid sequence with at least 95% sequence identity to SEQ ID NO: 72.

Embodiment 123 comprises an isolated polypeptide or polypeptide complex of embodiment 1, wherein the polypeptide or polypeptide complex comprises an amino acid sequence with at least 95% sequence identity to SEQ ID NO: 73.

Embodiment 124 comprises an isolated polypeptide or polypeptide complex of embodiment 1, wherein the polypeptide or polypeptide complex comprises an amino acid sequence with at least 95% sequence identity to SEQ ID NO: 62 and SEQ ID NO: 63.

Embodiment 125 comprises an isolated polypeptide or polypeptide complex of embodiment 1, wherein the polypeptide or polypeptide complex comprises an amino acid sequence with at least 95% sequence identity to SEQ ID NO: 64 and SEQ ID NO: 65.

Embodiment 126 comprises an isolated polypeptide or polypeptide complex of embodiment 1, wherein the polypeptide or polypeptide complex comprises an amino acid sequence with at least 95% sequence identity to SEQ ID NO: 66 and SEQ ID NO: 67.

Embodiment 127 comprises an isolated polypeptide or polypeptide complex of embodiment 1, wherein the polypeptide or polypeptide complex comprises an amino acid sequence with at least 95% sequence identity to SEQ ID NO: 68 and SEQ ID NO: 69.

Embodiment 128 comprises an isolated polypeptide or polypeptide complex of embodiment 1, wherein the polypeptide or polypeptide complex comprises an amino acid sequence with at least 95% sequence identity to SEQ ID NO: 70 and SEQ ID NO: 71.

Embodiment 129 comprises an isolated polypeptide or polypeptide complex of embodiment 1, wherein the polypeptide or polypeptide complex comprises an amino acid sequence with at least 95% sequence identity to SEQ ID NO: 72 and SEQ ID NO: 73.

Embodiment 130 comprises an isolated polypeptide or polypeptide complex of embodiment 1, wherein the polypeptide or polypeptide complex comprises an amino acid sequence with at least 95% sequence identity to SEQ ID NO: 74 and SEQ ID NO: 75.

Embodiment 131 comprises an isolated polypeptide or polypeptide complex of embodiment 1, wherein the polypeptide or polypeptide complex comprises an amino acid sequence with at least 95% sequence identity to SEQ ID NO: 76 and SEQ ID NO: 77.

Embodiment 132 comprises a pharmaceutical composition comprising: (a) the polypeptide or polypeptide complex of any one of embodiments 1-131; and (b) a pharmaceutically acceptable excipient.

Embodiment 133 comprises an isolated recombinant nucleic acid molecule encoding the polypeptide or polypeptide complex of any one of embodiments 1-131.

Embodiment 134 comprises an isolated polypeptide or polypeptide complex according to Formula II: L₁ₐ-P₁ₐ-H₁ₐ wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to PSMA; P₁ₐ comprises a peptide that binds to the first antigen recognizing molecule when L₁ₐ is uncleaved; and H₁ₐ comprises a half-life extending molecule.

Embodiment 135 comprises an isolated polypeptide or polypeptide complex of embodiment 134, wherein P₁ₐ when L₁ₐ is uncleaved impairs binding of the first antigen recognizing molecule to the effector cell antigen.

Embodiment 136 comprises an isolated polypeptide or polypeptide complex of embodiment 134, wherein the first antigen recognizing molecule comprises an antibody or antibody fragment.

Embodiment 137 comprises an isolated polypeptide or polypeptide complex of embodiment 134, wherein the effector cell antigen is an anti-CD3 effector cell antigen.

Embodiment 138 comprises an isolated polypeptide or polypeptide complex of embodiment 134, wherein P₁ₐ has less than 70% sequence homology to the effector cell antigen.

Embodiment 139 comprises an isolated polypeptide or polypeptide complex of embodiment 134, wherein P₁ₐ comprises a peptide sequence of at least 10 amino acids in length.

Embodiment 140 comprises an isolated polypeptide or polypeptide complex of embodiment 134, wherein P₁ₐ comprises a peptide sequence of at least 10 amino acids in length and no more than 20 amino acids in length.

Embodiment 141 comprises an isolated polypeptide or polypeptide complex of embodiment 134, wherein P₁ₐ comprises a peptide sequence of at least 16 amino acids in length.

Embodiment 142 comprises an isolated polypeptide or polypeptide complex of embodiment 134, wherein P₁ₐ comprises a peptide sequence of no more than 40 amino acids in length.

Embodiment 143 comprises an isolated polypeptide or polypeptide complex of embodiment 134, wherein P₁ₐ comprises at least two cysteine amino acid residues.

Embodiment 144 comprises an isolated polypeptide or polypeptide complex of embodiment 134, wherein P₁ₐ comprises a cyclic peptide or a linear peptide.

Embodiment 145 comprises an isolated polypeptide or polypeptide complex of embodiment 134, wherein P₁ₐ comprises a cyclic peptide.

Embodiment 146 comprises an isolated polypeptide or polypeptide complex of embodiment 134, wherein P₁ₐ comprises a linear peptide.

Embodiment 147 comprises an isolated polypeptide or polypeptide complex of embodiment 134, wherein P₁ₐ comprises an amino acid sequence selected from the group consisting of any one of SEQ ID NOs: 16-19.

Embodiment 148 comprises an isolated polypeptide or polypeptide complex of any one of embodiments 132-145, wherein H₁ₐ comprises a polymer.

Embodiment 149 comprises an isolated polypeptide or polypeptide complex of embodiment 148, wherein the polymer is polyethylene glycol (PEG).

Embodiment 150 comprises an isolated polypeptide or polypeptide complex of any one of embodiments 134-147, wherein H₁ₐ comprises albumin.

Embodiment 151 comprises an isolated polypeptide or polypeptide complex of any one of embodiments 134-147, wherein H₁ₐ comprises an Fc domain.

Embodiment 152 comprises an isolated polypeptide or polypeptide complex of embodiment 150, wherein the albumin is serum albumin.

Embodiment 153 comprises an isolated polypeptide or polypeptide complex of embodiment 152, wherein the albumin is human serum albumin.

Embodiment 154 comprises an isolated polypeptide or polypeptide complex of any one of embodiments 134-147, wherein H₁ₐ comprises a polypeptide, a ligand, or a small molecule.

Embodiment 155 comprises an isolated polypeptide or polypeptide complex of embodiment 154, wherein the polypeptide, the ligand or the small molecule binds a serum protein or a fragment thereof, a circulating immunoglobulin or a fragment thereof, or CD35/CR1.

Embodiment 156 comprises an isolated polypeptide or polypeptide complex of embodiment 155, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 157 comprises an isolated polypeptide or polypeptide complex of embodiment 155, wherein the circulating immunoglobulin molecule comprises IgG1, IgG2, IgG3, IgG4, slgA, IgM or IgD.

Embodiment 158 comprises an isolated polypeptide or polypeptide complex of embodiment 153, wherein the serum protein is albumin.

Embodiment 159 comprises an isolated polypeptide or polypeptide complex of embodiment 154, wherein the polypeptide is an antibody.

Embodiment 160 comprises an isolated polypeptide or polypeptide complex of embodiment 159, wherein the antibody comprises a single domain antibody, a single chain variable fragment or a Fab.

Embodiment 161 comprises an isolated polypeptide or polypeptide complex of embodiment 159, wherein the antibody comprises a single domain antibody that binds to albumin.

Embodiment 162 comprises an isolated polypeptide or polypeptide complex of embodiment 159, wherein the antibody is a human or humanized antibody.

Embodiment 163 comprises an isolated polypeptide or polypeptide complex of embodiment 160, wherein the single domain antibody is 645gH1gL1.

Embodiment 164 comprises an isolated polypeptide or polypeptide complex of embodiment 160, wherein the single domain antibody is 645dsgH5gL4.

Embodiment 165 comprises an isolated polypeptide or polypeptide complex of embodiment 160, wherein the single domain antibody is 23-13-A01-sc02.

Embodiment 166 comprises an isolated polypeptide or polypeptide complex of embodiment 160, wherein the single domain antibody is A10m3 or a fragment thereof.

Embodiment 167 comprises an isolated polypeptide or polypeptide complex of embodiment 160, wherein the single domain antibody is DOM7r-31.

Embodiment 168 comprises an isolated polypeptide or polypeptide complex of embodiment 160, wherein the single domain antibody is DOM7 h-11-15.

Embodiment 169 comprises an isolated polypeptide or polypeptide complex of embodiment 160, wherein the single domain antibody is Alb-1, Alb-8, or Alb-23.

Embodiment 170 comprises an isolated polypeptide or polypeptide complex of embodiment 160, wherein the single domain antibody is 10E.

Embodiment 171 comprises an isolated polypeptide or polypeptide complex of embodiment 160, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56.

Embodiment 172 comprises an isolated polypeptide or polypeptide complex of embodiment 158, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 58, HC-CDR2: SEQ ID NO: 59, and HC-CDR3: SEQ ID NO: 60.

Embodiment 173 comprises an isolated polypeptide or polypeptide complex of embodiment 160, wherein the single domain antibody is SA21.

Embodiment 174 comprises an isolated polypeptide or polypeptide complex of any one of embodiments 134-173, wherein H₁ₐ comprises a linking moiety (L₁ₐ) that connects H₁ₐ to P₁ₐ.

Embodiment 175 comprises an isolated polypeptide or polypeptide complex of embodiment 174, wherein L₁ₐ is a peptide sequence having at least 5 to no more than 50 amino acids.

Embodiment 176 comprises an isolated polypeptide or polypeptide complex of embodiment 174, wherein L₁ₐ is a peptide sequence having at least 10 to no more than 30 amino acids.

Embodiment 177 comprises an isolated polypeptide or polypeptide complex of embodiment 174, wherein L₁ₐ is a peptide sequence having at least 10 amino acids.

Embodiment 178 comprises an isolated polypeptide or polypeptide complex of embodiment 174, wherein L₁ₐ is a peptide sequence having at least 18 amino acids.

Embodiment 179 comprises an isolated polypeptide or polypeptide complex of embodiment 174, wherein L₁ₐ is a peptide sequence having at least 26 amino acids.

Embodiment 180 comprises an isolated polypeptide or polypeptide complex of embodiment 174, wherein L₁ₐ has a formula selected from the group consisting of (G₂S)ₙ, (GS)ₙ, (GSGGS)ₙ (SEQ ID NO: 50), (GGGS)ₙ (SEQ ID NO: 51). (GGGGS)ₙ (SEQ ID NO: 52), and (GSSGGS)ₙ (SEQ ID NO: 53), wherein n is an integer of at least 1.

Embodiment 181 comprises an isolated polypeptide or polypeptide complex of embodiment 174, wherein L₁ₐ comprises an amino acid sequence according to SEQ ID NO: 23.

Embodiment 182 comprises an isolated polypeptide or polypeptide complex of any one of embodiments 134-181, wherein P₁ₐ comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S.

Embodiment 183 comprises an isolated polypeptide or polypeptide complex of embodiment 182, wherein Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P;

Embodiment 184 comprises an isolated polypeptide or polypeptide complex of any one of embodiments 182-183, wherein Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N.

Embodiment 185 comprises an isolated polypeptide or polypeptide complex of any one of embodiments 134-181, wherein P₁ₐ comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S.

Embodiment 186 comprises an isolated polypeptide or polypeptide complex of embodiment 185, wherein U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P.

Embodiment 187 comprises an isolated polypeptide or polypeptide complex of embodiment 186, wherein U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

Embodiment 188 comprises an isolated polypeptide or polypeptide complex of any one of embodiments 134-181 and 70-88, wherein P₁ₐ comprises the amino acid sequences according to SEQ ID NOs: 79-105.

Embodiment 189 comprises an isolated polypeptide or polypeptide complex of any one of embodiments 134-181 and 70-88, wherein P₁ₐ comprises an amino acid sequences according to any of the sequences of Table 20.

Embodiment 190 comprises an isolated polypeptide or polypeptide complex of any one of embodiments 134-181, or 189, wherein P₁ₐ comprises the amino acid sequences according to any one of SEQ ID NOs: 106-117.

Embodiment 191 comprises an isolated polypeptide or polypeptide complex of any one of embodiments 134-181, wherein P₁ₐ comprises the amino acid sequence according to SEQ ID NO: 18 or a peptide sequence that has 1, 2, or 3, amino acid substitutions, additions, or deletions relative to the amino acid sequence of SEQ ID NO: 18.

Embodiment 192 comprises an isolated polypeptide or polypeptide complex of any one of embodiments 134-187, wherein P₁ₐ comprises the amino acid sequence according to SEQ ID NO: 19 or a peptide sequence that has 1, 2, or 3, amino acid substitutions, additions, or deletions relative to the amino acid sequence of SEQ ID NO: 19.

Embodiment 193 comprises an isolated polypeptide or polypeptide complex of any one of embodiments 134-187, wherein P₁ₐ comprises the amino acid sequence according to SEQ ID NO: 116 or a peptide sequence that has 1, 2, or 3, amino acid substitutions, additions, or deletions relative to the amino acid sequence of SEQ ID NO: 116.

Embodiment 194 comprises an isolated polypeptide or polypeptide complex of embodiment 191, wherein P₁ₐ comprises the amino acid sequence according to SEQ ID NO: 18.

Embodiment 195 comprises an isolated polypeptide or polypeptide complex of embodiment 192, wherein P₁ₐ comprises the amino acid sequence according to SEQ ID NO: 19.

Embodiment 196 comprises an isolated polypeptide or polypeptide complex of embodiment 193, wherein P₁ₐ comprises the amino acid sequence according to SEQ ID NO: 116.

Embodiment 197 comprises a polypeptide complex comprising a structural arrangement according to the configuration shown in FIG. 1C, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to the heavy chain variable domain of the scFv with a linking moiety that is a substrate for a tumor specific protease, and the peptide further comprises a half-life extending molecule; and a Fab or a Fab′ that binds to prostate-specific membrane antigen (PSMA), wherein the Fab or Fab′ comprises a Fab light chain polypeptide chain and a Fab heavy chain polypeptide chain, and wherein the Fab heavy chain polypeptide chain is linked to a C terminus of the light chain variable domain of the scFv.

Embodiment 198 comprises a polypeptide complex comprising a structural arrangement according to the configuration shown in FIG. 1D, wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab or Fab′ that binds to prostate-specific membrane antigen (PSMA), wherein the Fab or Fab′ comprises a Fab light chain polypeptide and a Fab heavy chain polypeptide, wherein the Fab light chain polypeptide is linked to a C terminus of the light chain variable domain of the scFv.

Embodiment 199 comprises an isolated polypeptide or polypeptide complex of any one of embodiments 1-133, wherein P₁ comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, Land S.

Embodiment 200 comprises an isolated polypeptide or polypeptide complex of embodiment 199, wherein Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; and Z₁₄ is selected from D, Y, N, F, I, and P.

Embodiment 201 comprises an isolated polypeptide or polypeptide complex of anyone of embodiments 199-200, wherein Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁₁ is selected from I, Y, and F; Z₁₂ is selected from F, D, Y, and L; and Z₁₄ is selected from D, Y, and N.

Embodiment 202 comprises an isolated polypeptide or polypeptide complex of any one of embodiments 1-133, wherein P₁ comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S.

Embodiment 203 comprises an isolated polypeptide or polypeptide complex of embodiment 202, wherein U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P.

Embodiment 204 comprises an isolated polypeptide or polypeptide complex of embodiment 203, wherein U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

Embodiment 205 comprises an isolated polypeptide or polypeptide complex of any one of embodiments 1-133 and 200-204, wherein P₁ comprises the amino acid sequences according to any one of SEQ ID NOs: 79-105.

Embodiment 206 comprises an isolated polypeptide or polypeptide complex of any one of embodiments 1-133 and 200-204, wherein P₁ comprises an amino acid sequences according to any of the sequences of Table 20.

Embodiment 207 comprises an isolated polypeptide or polypeptide complex of any one of embodiments 1-133, or 206, wherein P₁ comprises the amino acid sequences according to SEQ ID NOs: 106-117.

Embodiment 208 comprises an isolated polypeptide or polypeptide complex of any one of embodiments 1-133, wherein P₁ comprises the amino acid sequence according to SEQ ID NO: 18 or a peptide sequence that has 1, 2, or 3, amino acid substitutions, additions, or deletions relative to the amino acid sequence of SEQ ID NO: 18.

Embodiment 209 comprises an isolated polypeptide or polypeptide complex of any one of embodiments 1-133, 200-204, wherein P₁ comprises the amino acid sequence according to SEQ ID NO: 19 or a peptide sequence that has 1, 2, or 3, amino acid substitutions, additions, or deletions relative to the amino acid sequence of SEQ ID NO: 19.

Embodiment 210 comprises an isolated polypeptide or polypeptide complex of anyone of embodiments 1-133, 200-204, wherein P₁ comprises the amino acid sequence according to SEQ ID NO: 116 or a peptide sequence that has 1, 2, or 3, amino acid substitutions, additions, or deletions relative to the amino acid sequence of SEQ ID NO: 116.

Embodiment 211 comprises an isolated polypeptide or polypeptide complex of embodiment 208, wherein P₁ comprises the amino acid sequence according to SEQ ID NO: 18.

Embodiment 212 comprises an isolated polypeptide or polypeptide complex of embodiment 209, wherein P₁ comprises the amino acid sequence according to SEQ ID NO: 19.

Embodiment 213 comprises an isolated polypeptide or polypeptide complex of embodiment 210, wherein P₁ comprises the amino acid sequence according to SEQ ID NO: 116.

Embodiment 214 comprises a pharmaceutical composition comprising: (a) the polypeptide or polypeptide complex of any of embodiments 1-213; and (b) a pharmaceutically acceptable excipient.

Embodiment 215 comprises an isolated recombinant nucleic acid molecule encoding the polypeptide or polypeptide complex of any of embodiments 1-213.

Embodiment 216 comprises a method of treating lung cancer comprising administering to a subject in need thereof an isolated polypeptide or polypeptide complex according to Embodiments 1-213.

Embodiment 217 comprises a method of treating breast cancer comprising administering to a subject in need thereof an isolated polypeptide or polypeptide complex according to Embodiments 1-213.

Embodiment 218 comprises a method of treating cervical cancer comprising administering to a subject in need thereof an isolated polypeptide or polypeptide complex according to Embodiments 1-213.

Embodiment 219 comprises a method of treating ovarian cancer comprising administering to a subject in need thereof an isolated polypeptide or polypeptide complex according to Embodiments 1-213.

Embodiment 220 comprises a method of treating pancreatic cancer comprising administering to a subject in need thereof an isolated polypeptide or polypeptide complex according to Embodiments 1-213.

Embodiment 221 comprises a method of treating colorectal cancer comprising administering to a subject in need thereof an isolated polypeptide or polypeptide complex according to Embodiments 1-213.

Embodiment 222 comprises a method of treating gastric cancer comprising administering to a subject in need thereof an isolated polypeptide or polypeptide complex according to Embodiments 1-213.

Embodiment 223 comprises a method of treating pancreatic cancer comprising administering to a subject in need thereof an isolated polypeptide or polypeptide complex according to Embodiments 1-213.

Embodiment 224 comprises a method of treating metastatic castrate-resistant prostate cancer comprising administering to a subject in need thereof an isolated polypeptide or polypeptide complex according to Embodiments 1-213.

### EXAMPLES

### Example 1: PSMA Polypeptide Complex Binding

The PSMA-CD3 polypeptide complexes of Table 7 were evaluated for PSMA and CD3e binding.

TABLE 7
Polypeptide complexes

| Poly- | | | | | | |
|---|---|---|---|---|---|---|
| peptide | | Fab | | CD3 | Cleavable | |
| complex | Form | Mask | CD3 | Mask | linker | sdA |
| PC1 | Vh | | | | | |
| PC3 | Vh | — | SEQ ID | SEQ ID | LSGRSD | SEQ |
| | | | NO. 7 | NO. 16 | AGSPLG | ID NO. |
| | | | | | LAG | 57 |
| | | | | | (SEQ ID | |
| | | | | | NO: 48) | |
| PC5 | Vh | — | SEQ ID | SEQ ID | LSGRSD | SEQ |
| | | | NO. 7 | NO. 17 | AGSPLG | ID NO. |
| | | | | | LAG | 57 |
| | | | | | (SEQ ID | |
| | | | | | NO: 48) | |
| PC2 | Vl | | | | | |
| PC4 | Vl | — | SEQ ID | SEQ ID | LSGRSD | SEQ |
| | | | NO. 7 | NO. 16 | AGSPLG | ID NO. |
| | | | | | LAG | 57 |
| | | | | | (SEQ ID | |
| | | | | | NO: 48) | |
| PC6 | Vl | — | SEQ ID | SEQ ID | LSGRSD | SEQ ID |
| | | | NO. 7 | NO. 17 | AGSPLG | NO. 57 |
| | | | | | LAG | |
| | | | | | (SEQ ID | |
| | | | | | NO: 48) | |

The polypeptide complex molecules of Table 7 were evaluated for their ability to bind PSMA as well as CD3 in a standard enzyme linked immunosorbent assay (ELISA) format. Polypeptide complex binding of PSMA or CD3 were measured before and after protease treatment. Briefly, biotinylated antigen was captured on neutravidin coated plates. Polypeptide complex molecules were treated with active matriptase (MTSP1) where indicated. Polypeptide complex molecules diluted in buffer were then added to the antigen coated plates. Bound polypeptide complex was detected using a standard horse radish peroxidase conjugate secondary antibody. The concentration of polypeptide complex required to achieve 50% maximal signal (EC50) was calculated in Graphpad Prism.

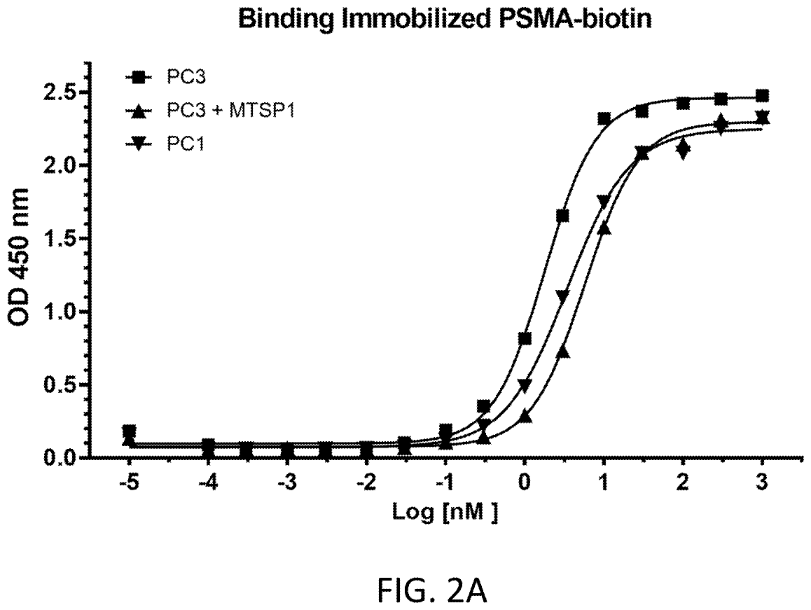

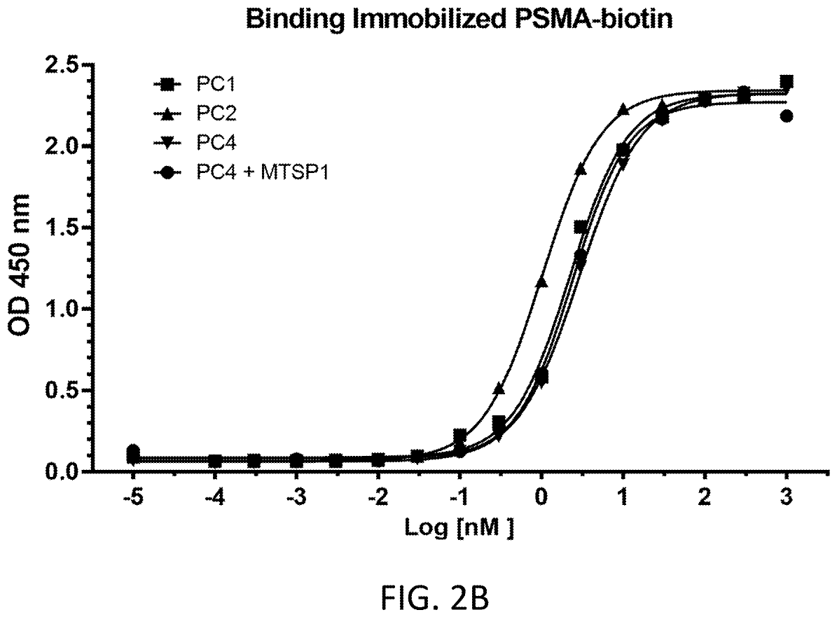

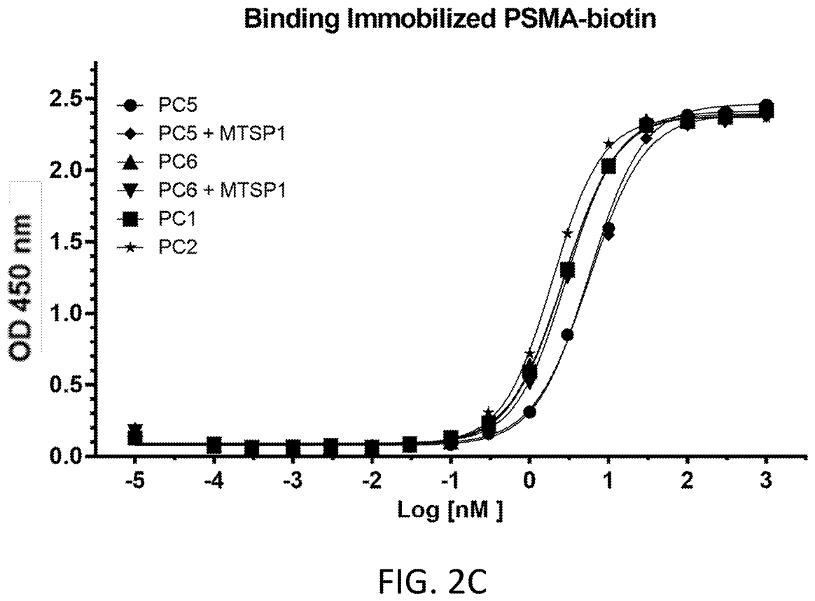

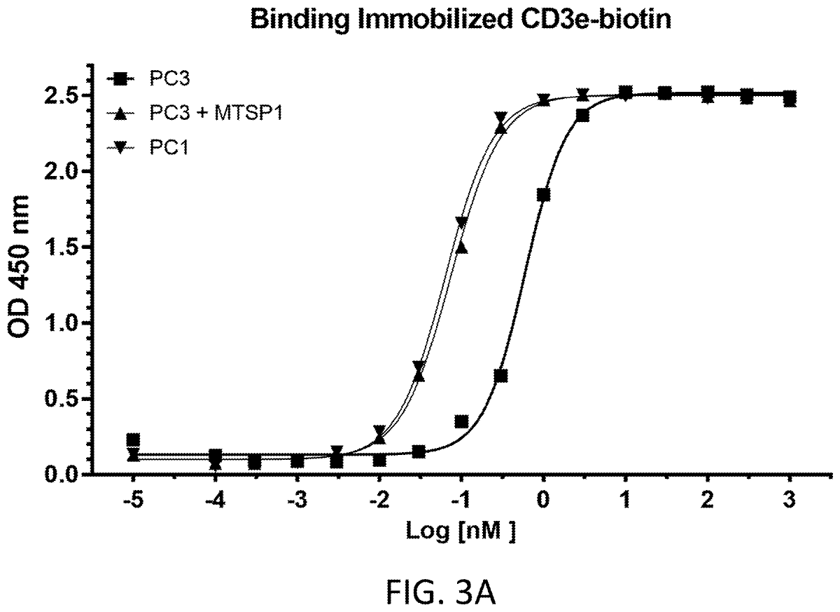

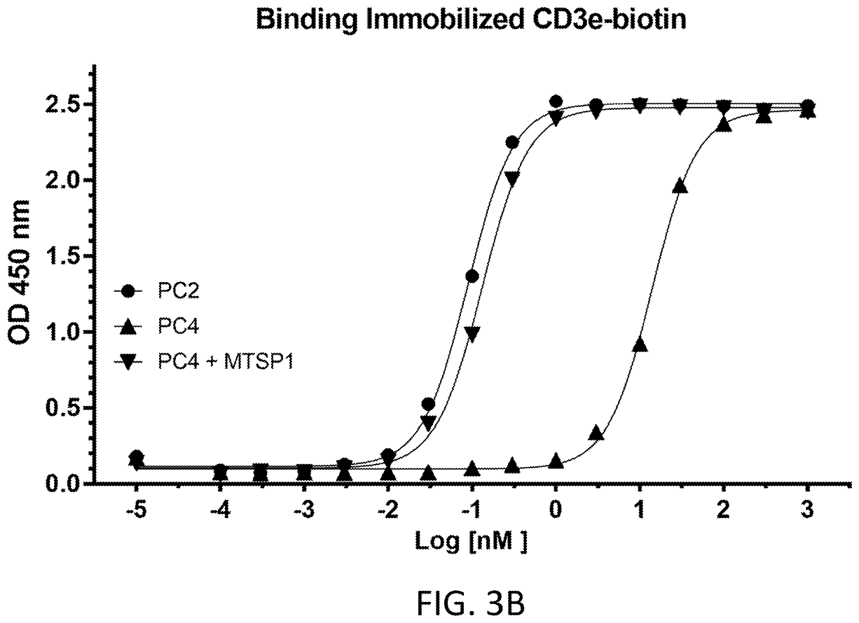

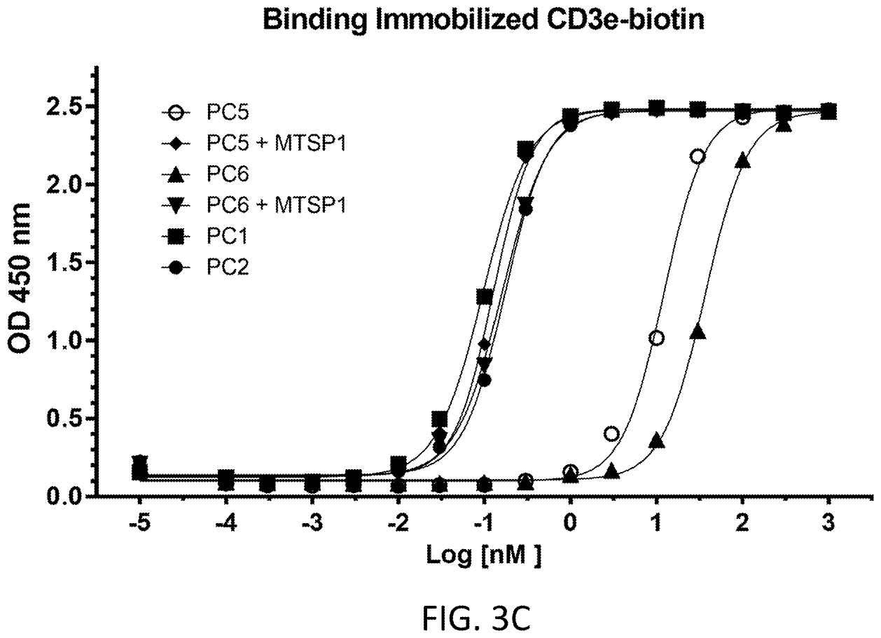

FIGS. 2A, 2B and 2C show representative PSMA binding ELISAs. This data is summarized in Table 8. FIGS. 3A, 3B and 3C show representative CD3 binding ELISAs. This data is summarized in Table 9. The masked polypeptide complex of PC3 has an EC5 about 8 fold higher than the protease treated PC3. The masked polypeptide complex of PC5 has an EC50 about 95 fold higher than the protease treated PC3. The masked polypeptide complexes of PC4 and PC6 had EC50s about 100 fold and about 230 fold higher than the respective protease treated polypeptide complexes.

TABLE 8
PSMA binding

| EC50 nM | Masked | Cleaved |
|---|---|---|
| PC1 | — | 2.68 |
| PC3 | 1.78 | 5.73 |
| PC5 | 5.67 | 5.65 |
| PC2 | — | 1.93 |
| PC4 | 2.88 | 2.40 |
| PC6 | 2.70 | 2.89 |

TABLE 9
CD3 binding

| EC50 nM | Masked | Cleaved | Fold shift |
|---|---|---|---|
| PC1 | — | 0.08 | |
| PC3 | 0.5923 | 0.07384 | 8x |
| PC5 | 12.05 | 0.1266 | 95.2x |
| PC2 | — | 0.10 | — |
| PC4 | 13.96 | 0.1313 | 106.3x |
| PC6 | 36.49 | 0.1593 | 229.1x |

### Example 2: Polypeptide Complex Mediated Tumor Cytotoxicity and T Cell Activation

Polypeptide complexes were evaluated in a functional in vitro tumor cell killing assay using the PSMA positive tumor cell lines 22Rv1 and LNCaP. Tumor cell killing was measured using a real time cell analyzer from Acea Biosciences that relies on sensor impedance measurements (cell index) that increased as tumor cells adhere, spread, and expand on the surface of the sensor. Likewise, as the tumor cells were killed the impedance decreased. 25,000 tumor cells were added per well and allowed to adhere overnight. The following day polypeptide complexes titrated in human serum supplemented medium along with 75,000 CD8+ T cells were added to the wells. Cell index measurements were taken every 10 minutes for an additional 96 hours. The cell index times number of hours (tumor cell growth kinetics) was then plotted versus concentration of polypeptide complex where the concentration required to reduce the tumor growth 50% (IC50) was calculated using Graphpad Prism.

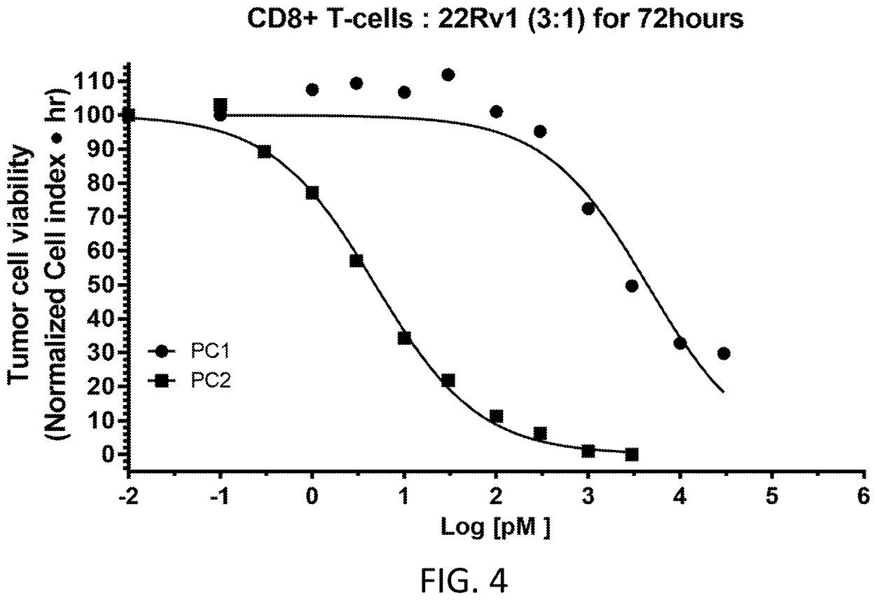

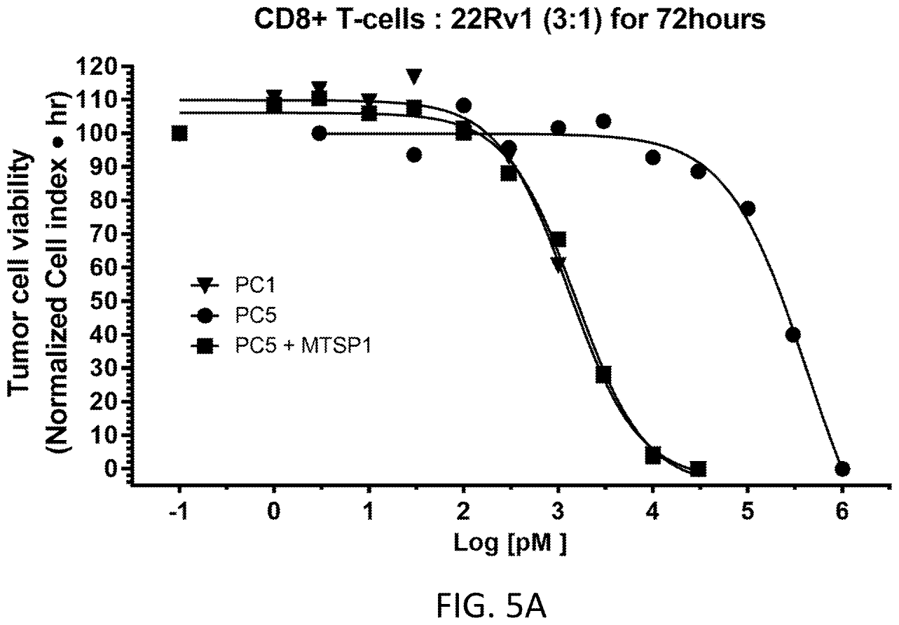

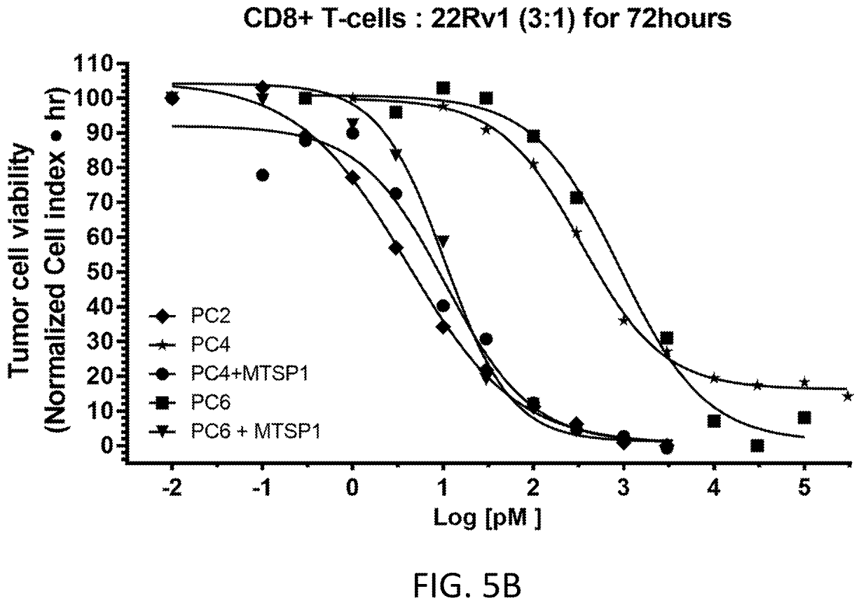

The 22Rv1 tumor cell line has a PSMA density of about 3000 copies per cell. FIG. 4 shows representative viability data for 22Rv1 treated with PC1 or PC2. This data is summarized in Table 10, and shows that PC2 is about 1000 times more potent than PC1. FIGS. 5A and 5B, and Tables 11 and 12, show viability data for 22Rv1 cells treated with masked or cleaved polypeptide complexes. The masked polypeptide complex of PC5 has an IC50 greater than 50 fold higher than the unmasked polypeptide complex of PC1, protease treatment reduced the IC50 to less than the IC50 of PC1. Similarly, the masked polypeptide complexes of PC4 and PC6 had IC50s about 150 and 200 fold higher than PC2 respectively, and protease treatment rescues both to about 1.7 and 2.5 fold higher than PC2.

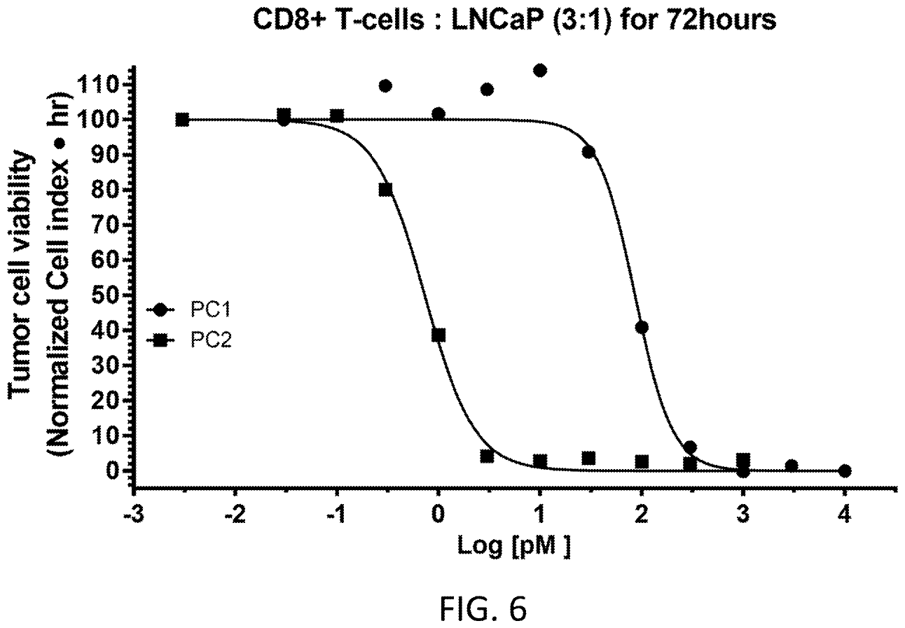

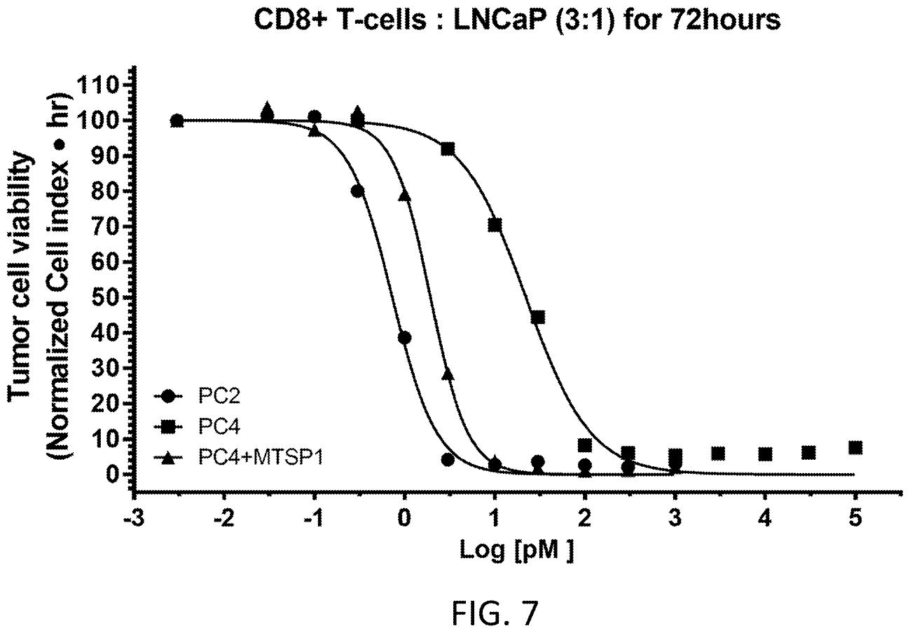

The LNCaP tumor cell line has a PSMA density of about 350,000 copies per cell. FIG. 6 shows representative viability data for LNCaP. This data is summarized in Table 13 and shows that PC2 is about 100 times more potent than PC1. FIG. 7, and Table 14 show viability data for LNCaP cells treated with masked or cleaved polypeptide complexes. The masked polypeptide complex of PC4 has an IC50 about 30 fold higher than the unmasked polypeptide complex of PC2, protease treatment rescues the IC50 to about 2.5 fold higher than the unmasked polypeptide complex.

TABLE 10
22Rv1 cell viability

| 22Rv1 IC50 pM | PC1.01 | PC2.01 |
|---|---|---|
| 72 hr | 4409 | 4.831 |

TABLE 11
22Rv1 cell viability

| 22Rv1 | | | PC5+ |
|---|---|---|---|
| 72 hr | PC1 | PC5 | MTSP1 |
| IC50 pM | 3,916 | 212810 | 1591 |
| Fold shift | 1x | 54.3x | 0.4x |

TABLE 12
22Rv1 cell viability

| 22Rv1 | | | PC4+ | | PC6+ |
|---|---|---|---|---|---|
| 72 hr | PC2 | PC4 | MTSP1 | PC6 | MTSP1 |
| IC50 pM | 4.831 | 757.3 | 8.169 | 984.9 | 12.03 |
| Fold shift | 1x | 156.8x | 1.7x | 203.9x | 2.5x |

TABLE 13
LNCaP cell viability

| LNCaP IC50 | | |
|---|---|---|
| pM | PC1 | PC2 |
| 72 hr | 85.97 | 0.73 |

TABLE 14
LNCaP cell viability

| LNCaP | | | |
|---|---|---|---|
| IC50 | | PC4+ | |
| pM | PC2 | MTSP1 | PC4 |
| 72 hr | 0.73 (1x) | 1.94 (2.65x) | 22.1 (30.2x) |

### Example 3: Polypeptide Complex Mediated Tumor Cell Killing

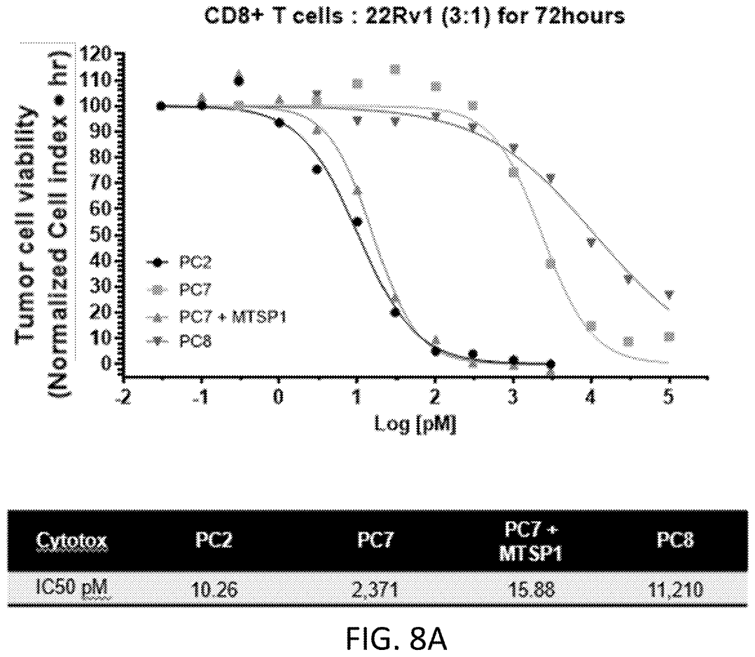

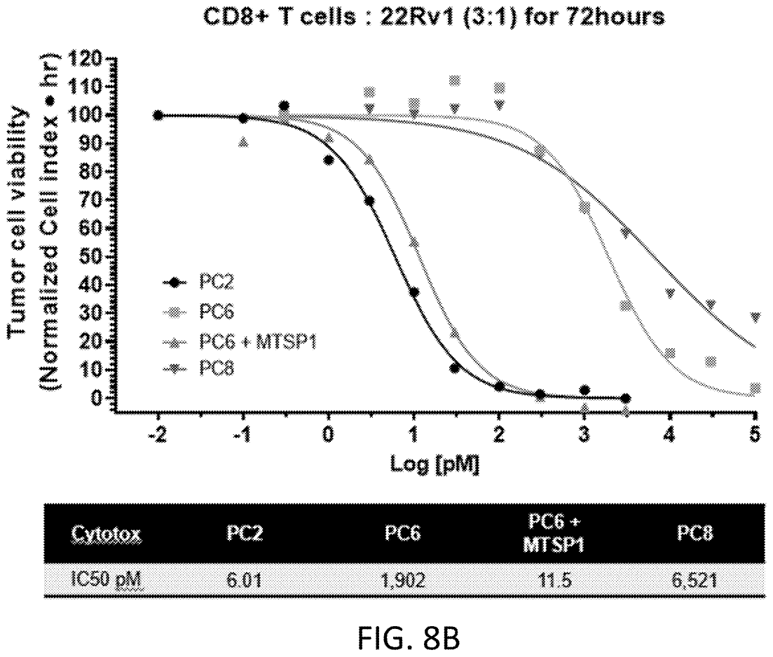

Polypeptide complexes were evaluated in a functional in vitro tumor cell killing assay using the PSMA positive tumor cell lines 22Rv1. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex where the concentration required to reduce the tumor growth 50% (IC50) was calculated using Graphpad Prism software. Data is seen in FIGS. 8A-8B.

### Example 4: Polypeptide Complex Pharmacokinetics in Cynomolgus Monkey

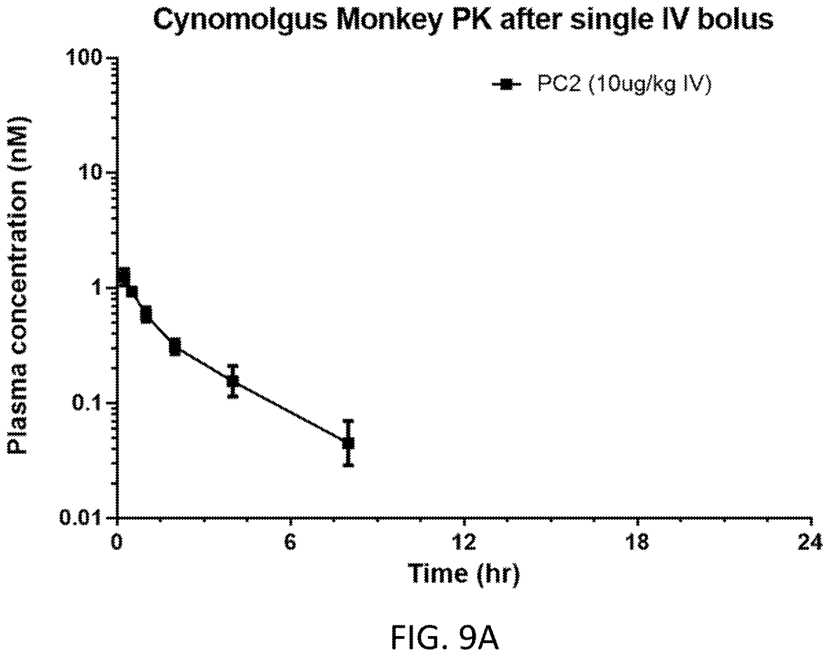

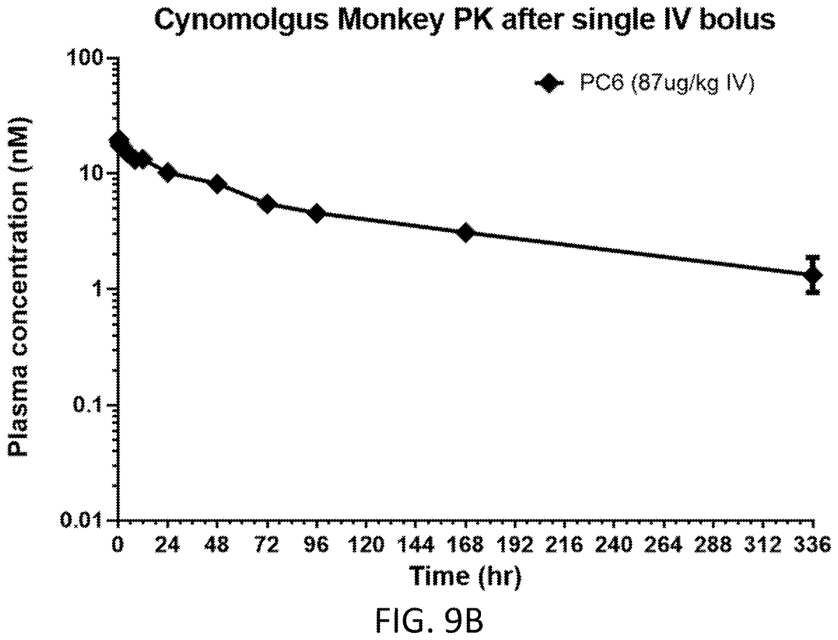

Pharmacokinetics and exploratory safety of polypeptide molecules were evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Concentration of polypeptide molecules in plasma was measured via standard ELISA techniques relative to a reference standard diluted in control cyno plasma. Plasma concentration curves were fit to a standard two phase exponential equation representing distribution and elimination phases. Fitting of pharmacokinetics enabled the calculation of Cmax, half-life, volume of distribution, clearance, and 7 day area under the curve (AUC) shown in Table 15 for PSMA TCE polypeptide complexes and Table 16 for PSMA TRACTr polypeptide complexes. Data is seen in FIGS. 9A-9B. Measured pharmacokinetics in port once weekly dosing in humans.

TABLE 15
PSMA TCE

| | PC2 10 ug/kg | Units |
|---|---|---|
| C_{MAX} | 1.69 | nM |
| t_{1/2} | 2.17 | hr |
| Vd | 0.23 | L |
| VSS | 0.67 | L |
| CL | 24.49 | mL/hr/kg |
| BW | 3.00 | kg |
| 7 day | 141 | nM · min |
| AUC | | |

TABLE 16
PSMA TRACTr

| | PC6 87 ug/kg | Units |
|---|---|---|
| C_{MAX} | 17.90 | nM |
| t_{1/2} | 118.99 | hr |
| Vd | 0.18 | L |
| VSS | 0.35 | L |
| CL | 0.34 | mL/hr/kg |
| BW | 3.00 | kg |
| 7 day | 63,731 | nM · min |
| AUC | | |

### Example 5: Polypeptide Complexes in Cynomolgus Cytokine Release

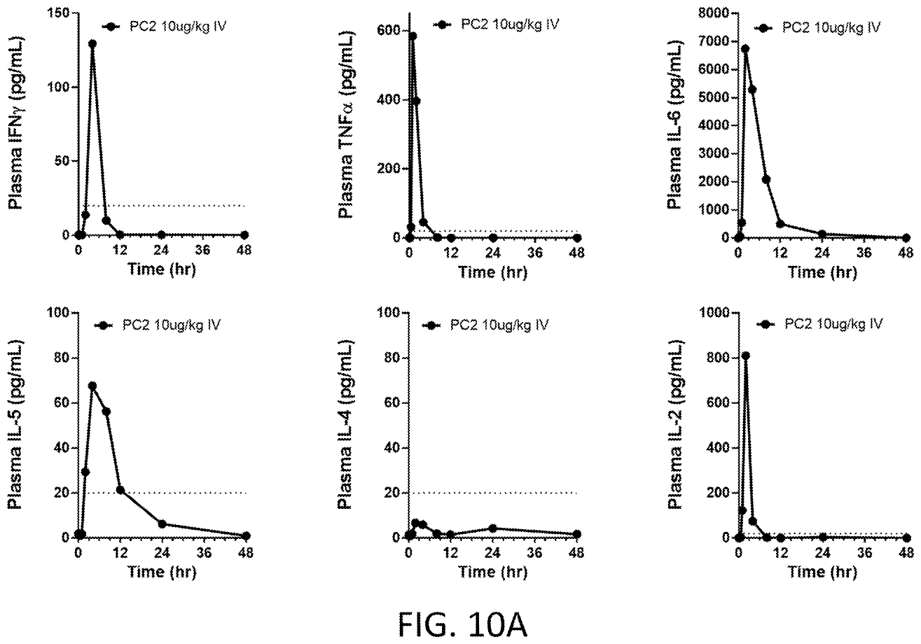

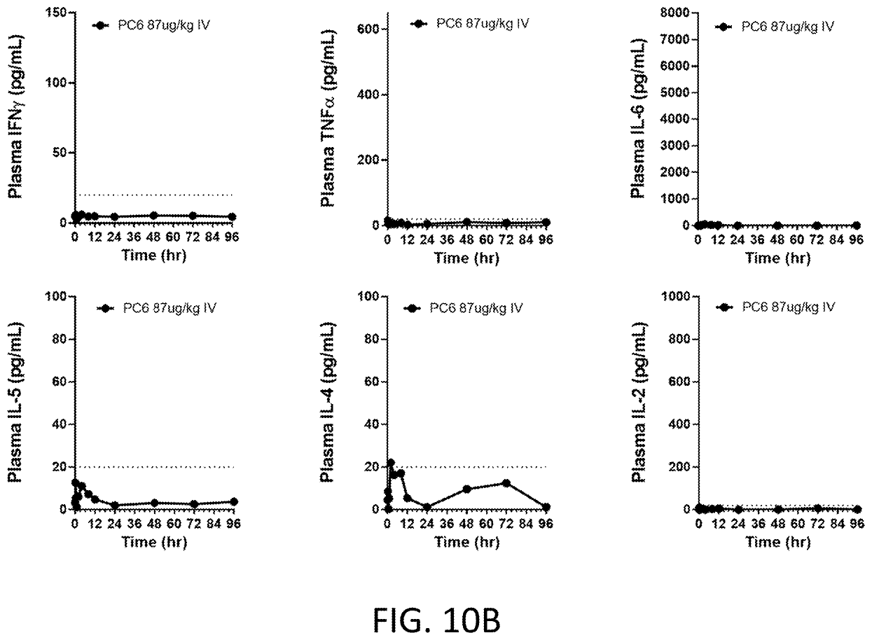

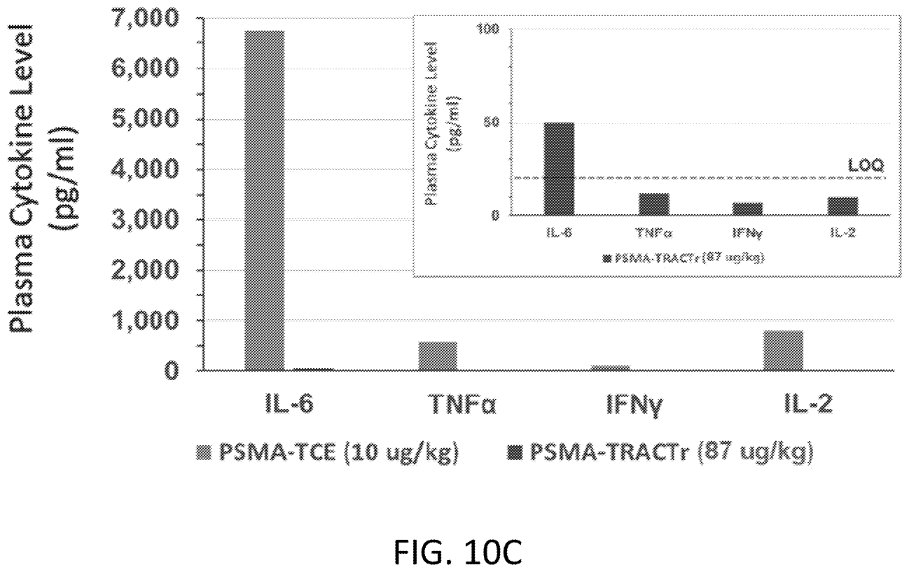

Cytokine release after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for cytokines using a non-human primate cytometric Th1/Th2 bead array kit from BD biosciences following the manufacturer's instructions. Interferon gamma, tumor necrosis factor alpha, interleukin 6, interleukin 5, interleukin 4, and interleukin 2 levels in plasma were calculated relative to reference standards provided with the bead array kit. Data is seen in FIGS. 10A-10C.

### Example 6: Polypeptide Complexes in Cynomolgus Toxicity

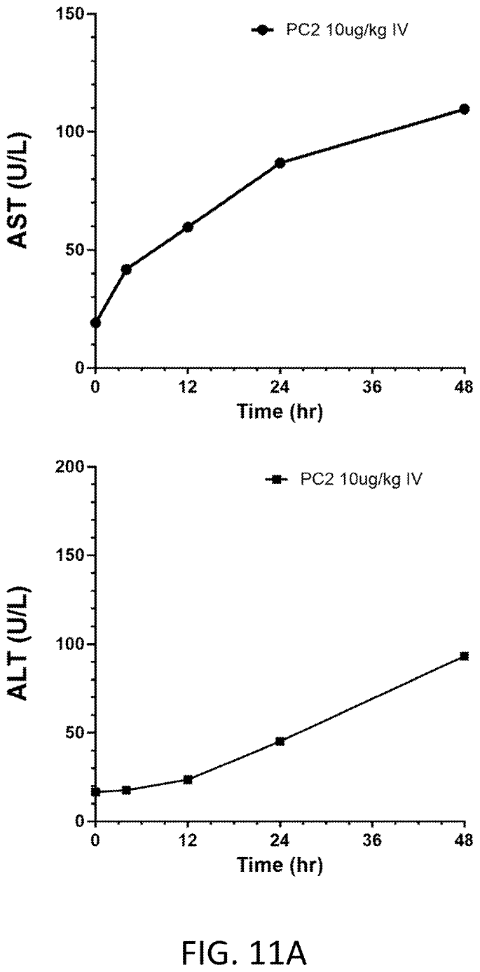

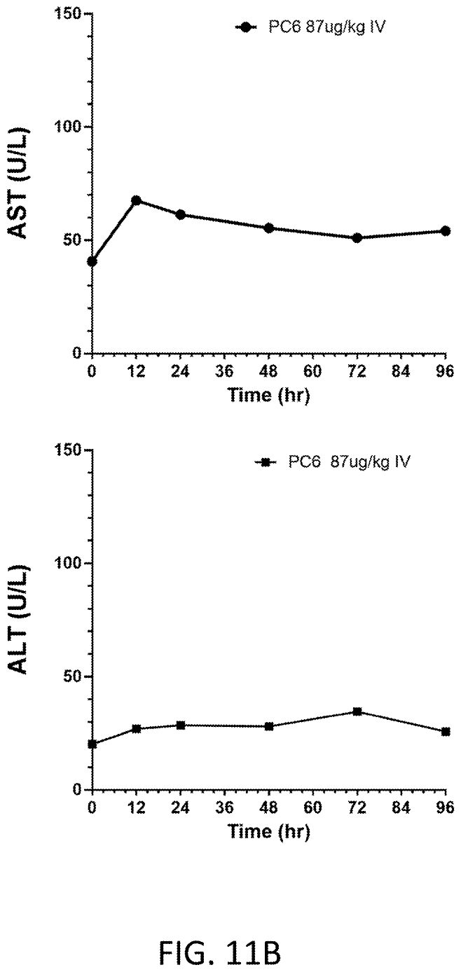

Systemic liver enzymes after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for the presence of liver enzymes aspartate transaminase (AST) and alanine aminotransferase (ALT) as signs of potential liver toxicity. AST and ALT levels remained within the normal ranges for all timepoints tested after dosing suggesting a lack of liver toxicity. AST and ALT were quantified following the instructions provided in a commercially available kit from Millipore. AST and ALT levels were calculated according to manufacturer's instructions relative to a positive control reference standard. Data is seen in FIGS. 11A-11B.

### Example 7: Optimized Phage Library Construction—CD3 scFv Peptides

Sequence activity relationships (SAR) were established for Peptide-A and Peptide-B by mutating each individual residue within the peptide to alanine and measuring binding and inhibition against SP34.185 scFv. Peptide residues whose alanine mutations significantly weakened binding and inhibition can be considered critical residues where mutations were not tolerated. Peptide residues whose alanine mutations performed similarly to the non-mutated sequence can be considered non-critical sites where mutations were indeed tolerated. Using the peptide SAR, DNA oligo libraries were constructed where codons encoding critical residues within each peptide sequence were minimally mutated and codons encoding non-critical residues were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

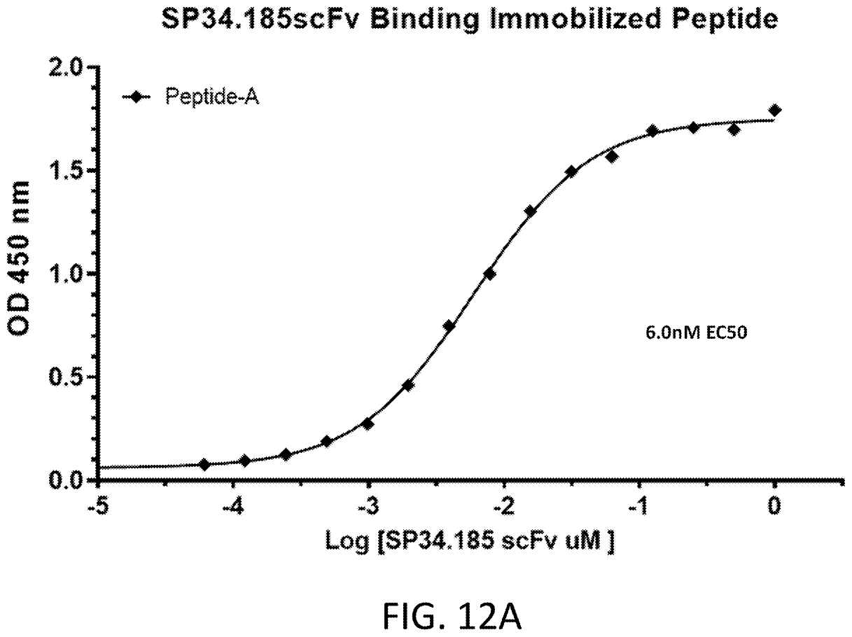

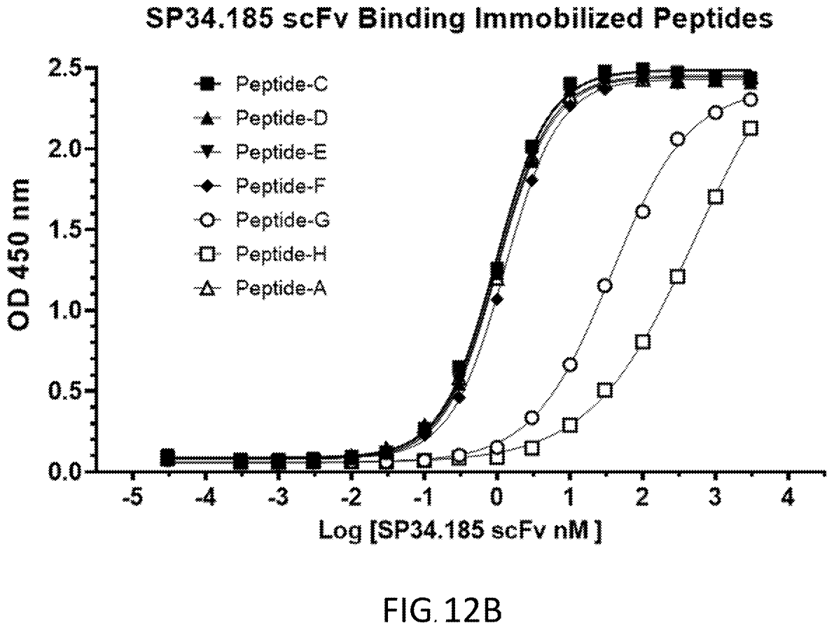

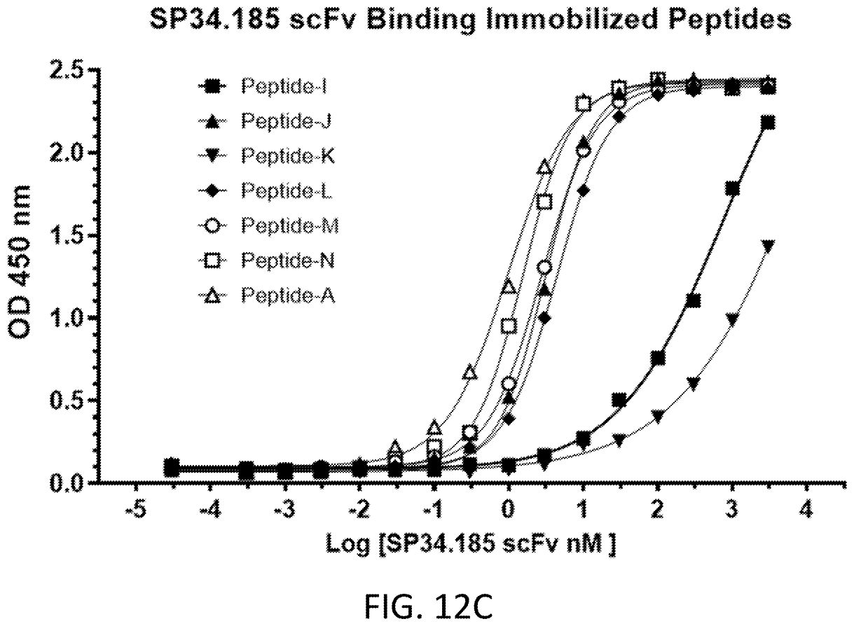

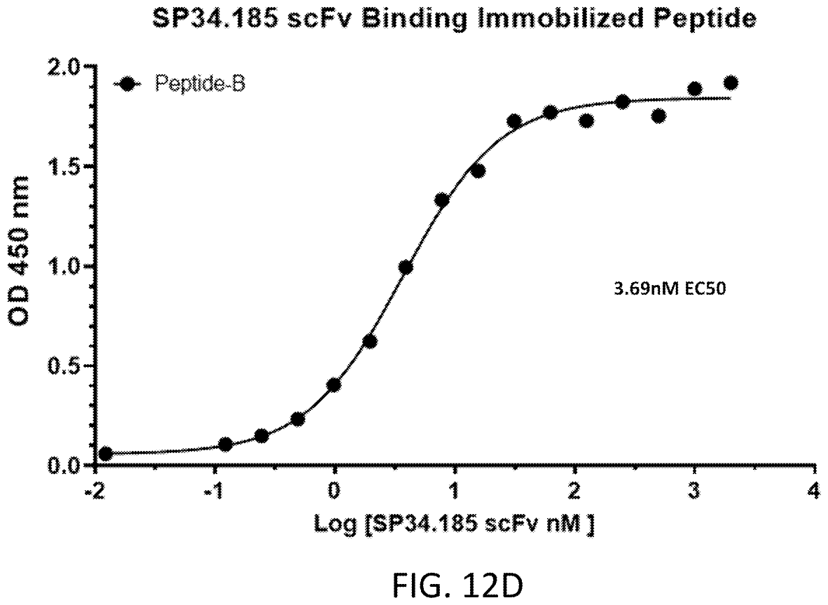

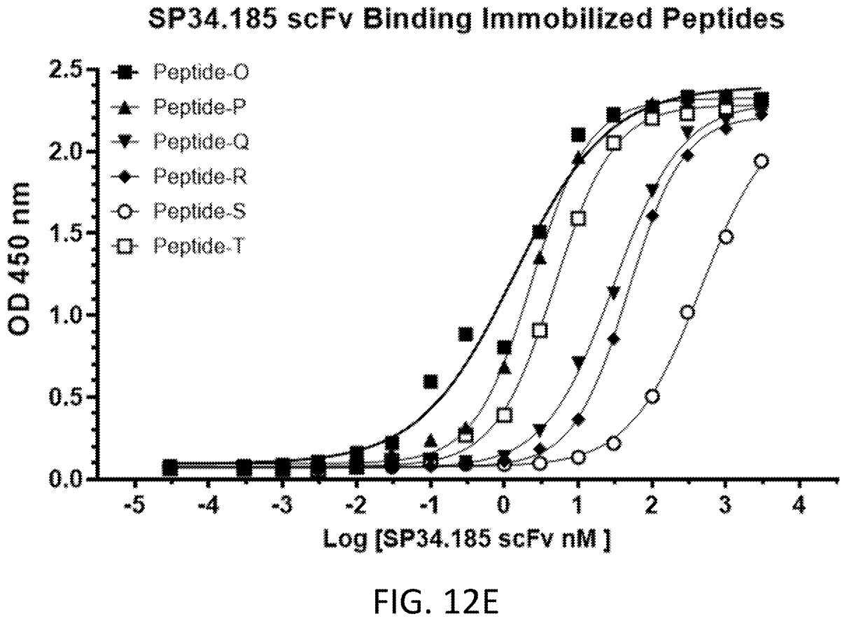

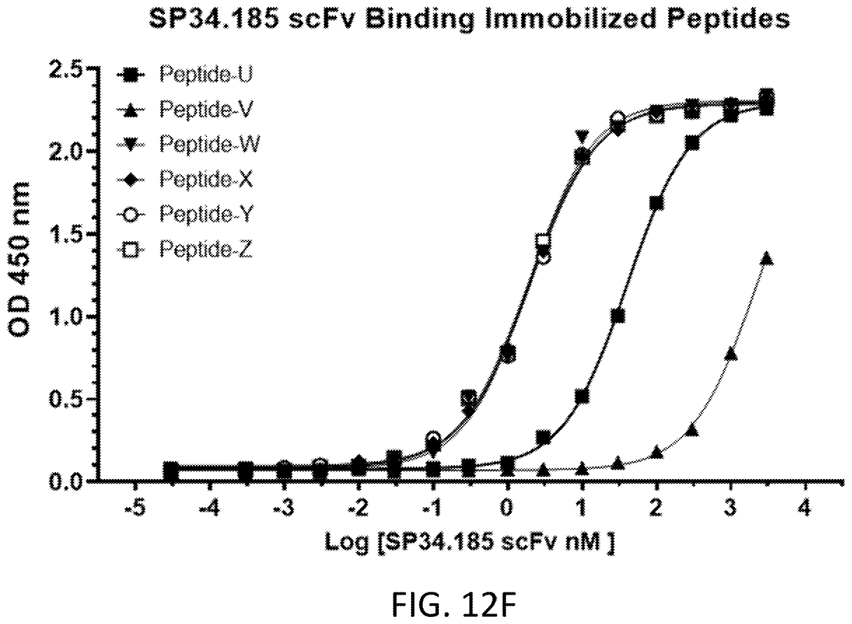

Peptides were evaluated for their ability to bind SP34.185 scFv by standard enzyme linked immunosorbent assays (ELISAs). Briefly, biotinylated peptides were captured on neutravidin coated plates, quenched with biocytin followed by a washing step. SP34.185 scFv was then titrated onto the peptide captured plates. Plates were then washed and bound SP34.185 scFv was detected using a secondary horse radish peroxidase antibody conjugate. After washing again, plates were developed using standard ELISA techniques and stopped using acid. The concentration of SP34.185 scFv required to achieve 50% maximal signal or EC50 was calculated using Graphpad prism. Data is shown in FIGS. 12A-12F and summarized in Tables 17A-17D. Peptide Sequences of CD3 Ala Scan Peptides for Peptide A and Peptide-B are shown in Table 19.

TABLE 17A
Summary of FIG. 12B

| ELISA | Peptide-A | Peptide-C | Peptide-D | Peptide-E | Peptide-F | Peptide-G | Peptide-H |
|---|---|---|---|---|---|---|---|
| EC50 nM | 1.013 | 0.9429 | 1.018 | 0.9738 | 1.27 | 47.5 | 346.2 |

TABLE 17B
Summary of FIG. 12C

| ELISA | Peptide-A | Peptide-I | Peptide-J | Peptide-K | Peptide-L | Peptide-M | Peptide-N |
|---|---|---|---|---|---|---|---|
| EC50 nM | 0.986 | 310.8 | 3.134 | 1,960 | 4.363 | 2.76 | 1.546 |

TABLE 17C
Summary of FIG. 12E

| ELISA | Peptide-O | Peptide-P | Peptide-Q | Peptide-R | Peptide-S | Peptide-T |
|---|---|---|---|---|---|---|
| EC50 nM | 1.356 | 2.359 | 30.04 | 47.50 | 457.1 | 4.762 |

TABLE 17D
Summary of FIG. 12F

| ELISA | Peptide-U | Peptide-V | Peptide-W | Peptide-X | Peptide-Y | Peptide-Z |
|---|---|---|---|---|---|---|
| EC50 nM | 39.90 | 2168 | 1.916 | 1.948 | 2.012 | 1.833 |

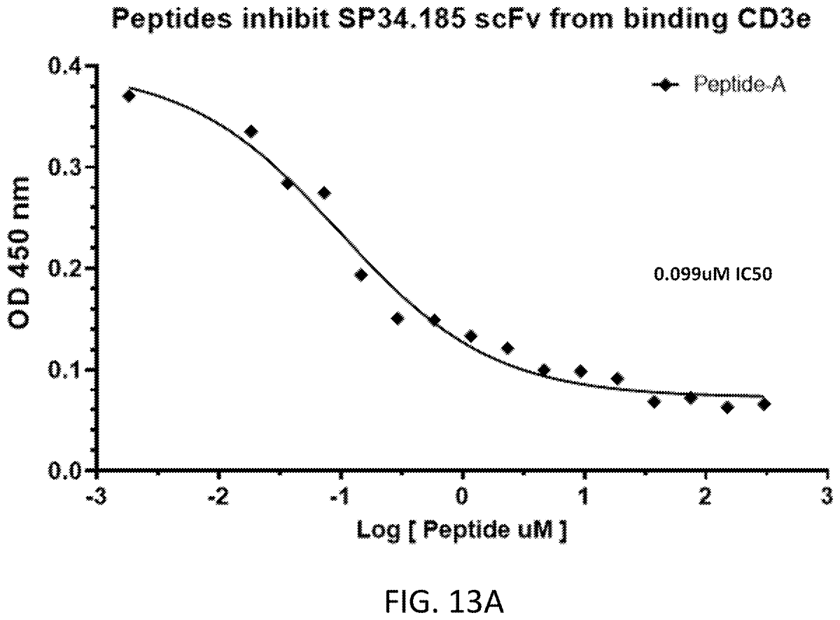

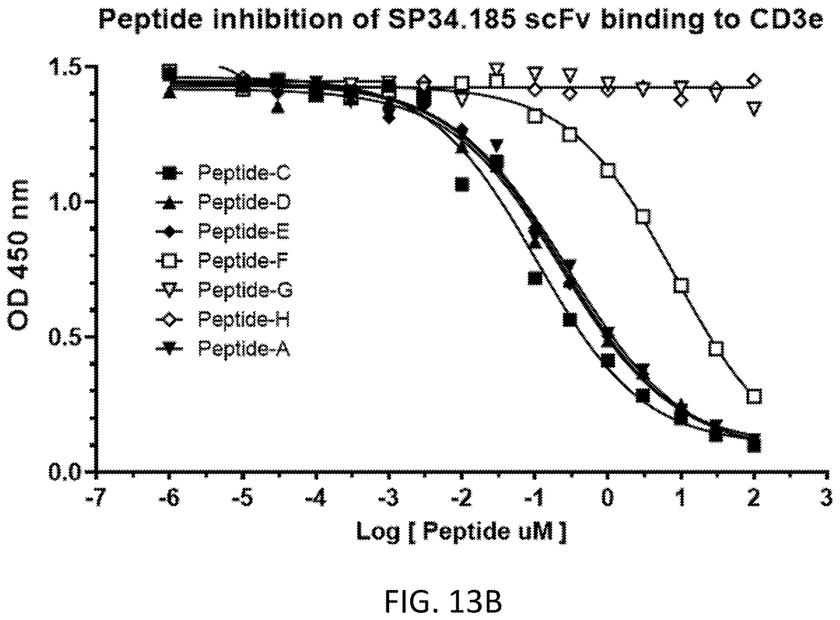

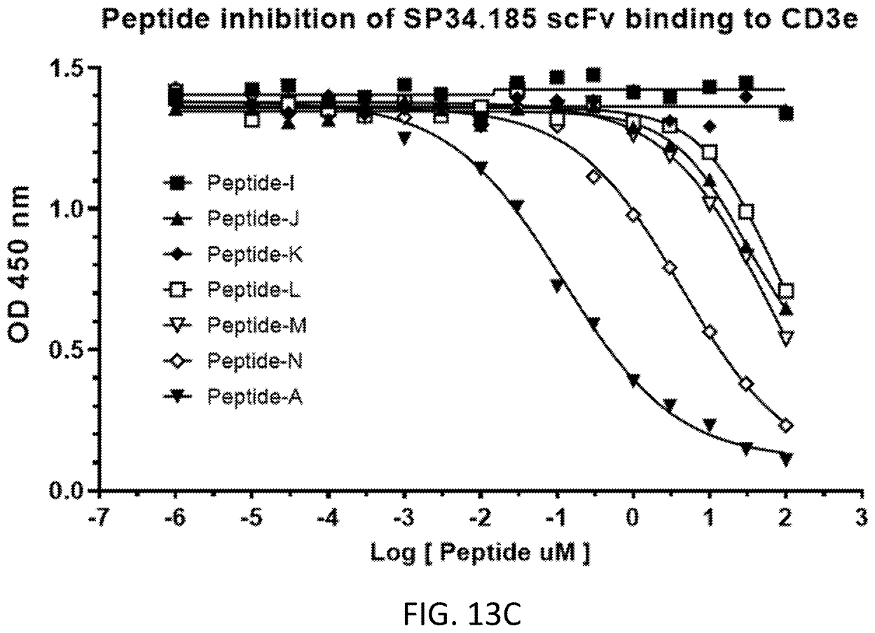

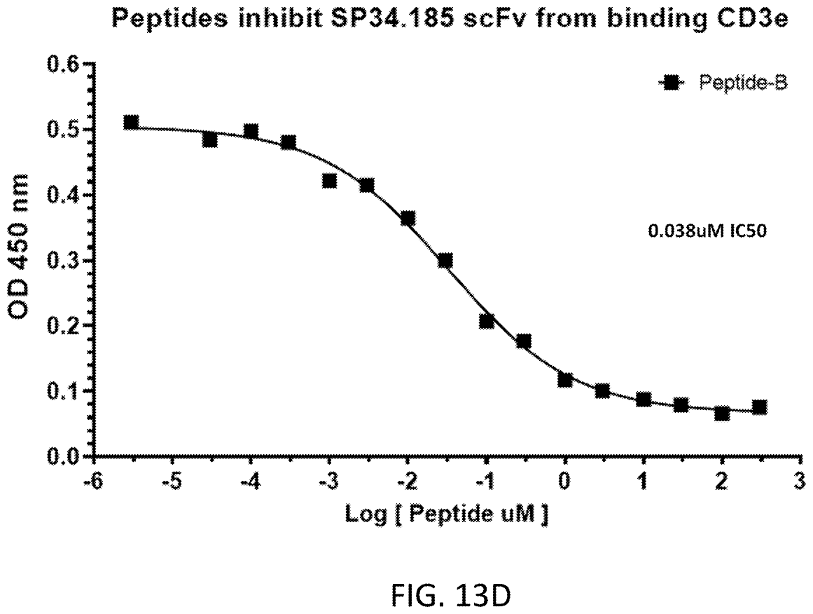

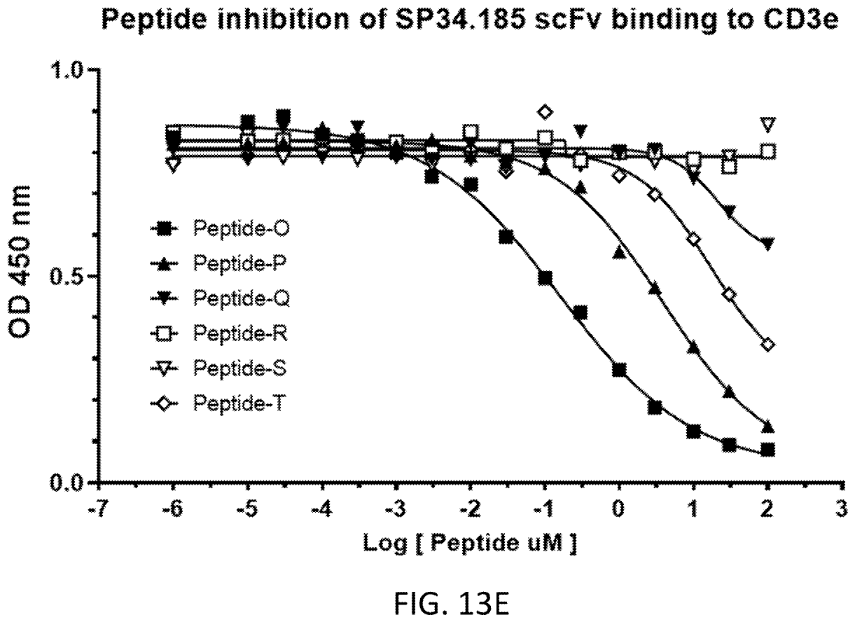

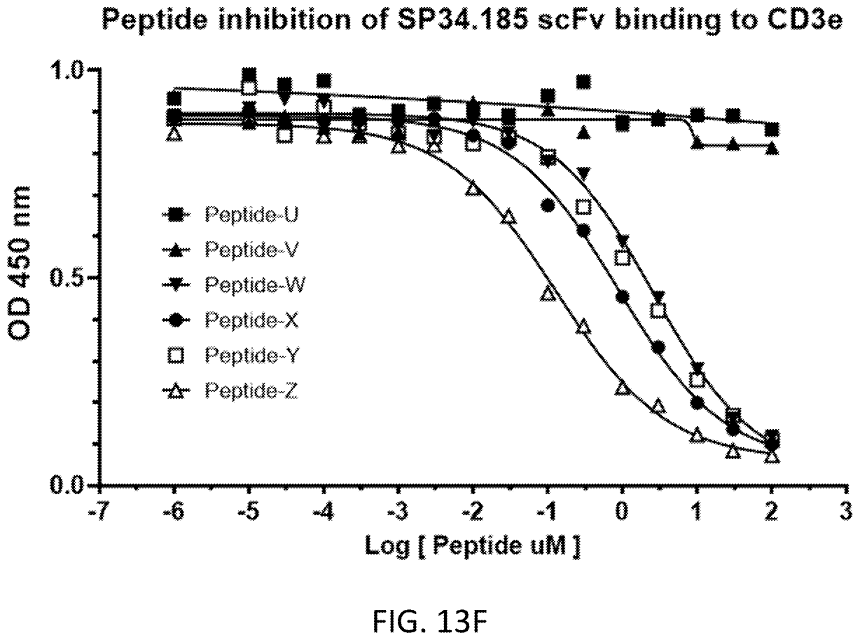

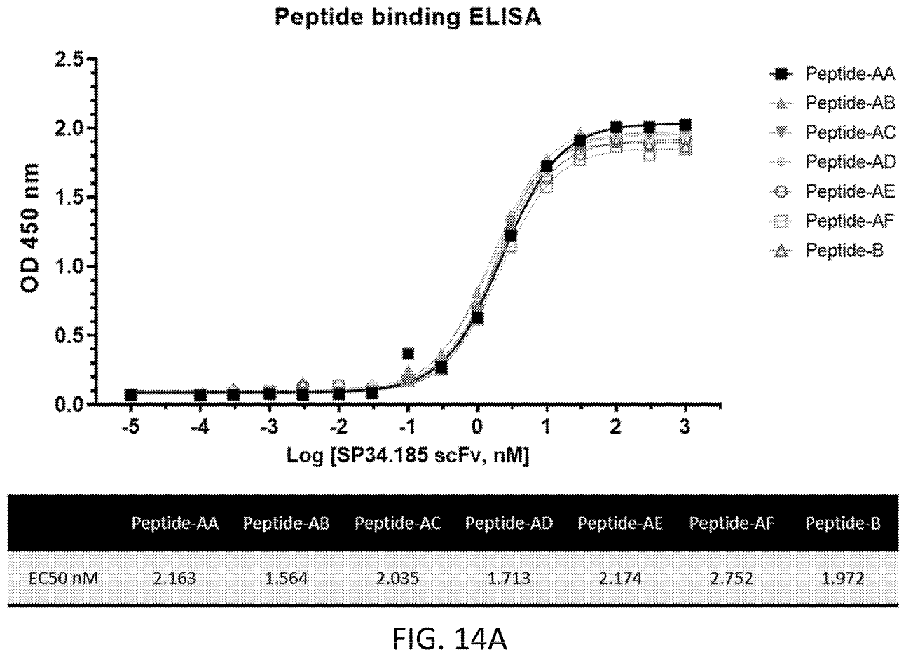

Peptides were evaluated for their ability to inhibit SP34.185 scFv from binding CD3e by standard enzyme linked immunosorbent assays (ELISAs). Briefly, a fixed concentration of SP34.185 scFv was incubated with varying concentrations of peptides in solution. SP34.185scFv and peptide solutions were incubated for 1 hr prior to addition to CD3 coated plates. Binding was allowed to proceed for 30 min prior to washing. After washing, bound SP34.185 scFv using a secondary horse radish peroxidase antibody conjugate. After washing again, plates were developed using standard ELISA techniques and stopped using acid. The concentration of peptide required to inhibit 50% of the SP34.185 scFv CD3 binding signal (IC50) was calculated using Graphpad prism. Data is show in FIGS. 13A-13F and summarized in Tables 18A-18D.

TABLE 18A
Summary of FIG. 13B

| ELISA | Peptide-A | Peptide-C | Peptide-D | Peptide-E | Peptide-F | Peptide-G | Peptide-H |
|---|---|---|---|---|---|---|---|
| IC50 uM | 0.1926 | 0.1025 | 0.2318 | 0.1905 | 5.484 | >100 | >100 |

TABLE 18B
Summary of FIG. 13C

| ELISA | Peptide-A | Peptide-I | Peptide-10 | Peptide-K | Peptide-L | Peptide-M | Peptide-N |
|---|---|---|---|---|---|---|---|
| IC50 uM | 0.1138 | >100 | 63.18 | >100 | 86.78 | 36.66 | 3.009 |

TABLE 18C
Summary of FIG. 13E

| ELISA | Peptide-O | Peptide-P | Peptide-Q | Peptide-R | Peptide-S | Peptide-T |
|---|---|---|---|---|---|---|
| IC50 uM | 0.1473 | 3.333 | >100 | >100 | >100 | 41.46 |

TABLE 18D
Summary of FIG. 13F

| ELISA | Peptide-U | Peptide-V | Peptide-W | Peptide-X | Peptide-Y | Peptide-Z |
|---|---|---|---|---|---|---|
| IC50 uM | >100 | >100 | 1.912 | 0.6992 | 1.456 | 0.1180 |

TABLE 19
CD3 Ala Scan Sequences -
Peptide A and Peptide-B

| | anti-CD3 | | SEQ |
|---|---|---|---|
| Peptide- | Panned | | ID |
| ID | target | Sequence | NO: |
| Peptide-A | SP34.185 | GSQCLGPEWEVCPY | 79 |
| Peptide-C | SP34.185 | ASQCLGPEWEVCPY | 80 |
| Peptide-D | SP34.185 | GAQCLGPEWEVCPY | 81 |
| Peptide-E | SP34.185 | GSACLGPEWEVCPY | 82 |
| Peptide-F | SP34.185 | GSQCAGPEWEVCPY | 83 |
| Peptide-G | SP34.185 | GSQCLAPEWEVCPY | 84 |
| Peptide-H | SP34.185 | GSQCLGAEWEVCPY | 85 |
| Peptide-I | SP34.185 | GSQCLGPAWEVCPY | 86 |
| Peptide-J | SP34.185 | GSQCLGPEAEVCPY | 87 |
| Peptide-K | SP34.185 | GSQCLGPEWAVCPY | 88 |
| Peptide-L | SP34.185 | GSQCLGPEWEACPY | 89 |
| Peptide-M | SP34.185 | GSQCLGPEWEVCAY | 90 |
| Peptide-N | SP34.185 | GSQCLGPEWEVCPA | 91 |
| Peptide-A | SP34.185 | GSQCLGPEWEVCPY | 92 |
| Peptide-B | SP34.185 | VYCGPEFDESVGCM | 93 |
| Peptide-O | SP34.185 | AYCGPEFDESVGCM | 94 |
| Peptide-P | SP34.185 | VACGPEFDESVGCM | 95 |
| Peptide-Q | SP34.185 | VYCAPEFDESVGCM | 96 |
| Peptide-R | SP34.185 | VYCGAEFDESVGCM | 97 |
| Peptide-S | SP34.185 | VYCGPAFDESVGCM | 98 |
| Peptide-T | SP34.185 | VYCGPEADESVGCM | 99 |
| Peptide-U | SP34.185 | VYCGPEFAESVGCM | 100 |
| Peptide-V | SP34.185 | VYCGPEFDASVGCM | 101 |
| Peptide-W | SP34.185 | VYCGPEFDEAVGCM | 102 |
| Peptide-X | SP34.185 | VYCGPEFDESAGCM | 103 |
| Peptide-Y | SP34.185 | VYCGPEFDESVACM | 104 |
| Peptide-Z | SP34.185 | VYCGPEFDESVGCA | 105 |

### Example 8: Panning of the Optimized Phage Library Construction—CD3 scFv Peptides

Once the phage optimization libraries were completed, phage libraries were bio-panned using SP34.185 scFv loaded beads. Multiple rounds of panning were performed where bacteriophage was allowed to bind to SP34.185 scFv loaded beads, washed, eluted, and amplified. Additional selective pressure was included during each round of panning using a fixed concentration of CD3, Peptide-A, or Peptide-B. After panning, phage infected bacteria were plated out and colonies picked into 96 well blocks. Clonal phage was then amplified and separated from bacterial cells via centrifugation. Phage containing supernatants were tested in binding ELISAs against SP34.185 scFv coated plates in the presence or absence of saturating concentration of CD3. Phage able to bind SP34.185 scFv were selected for sequence analysis if the binding signal was reduced in the presence of CD3.

### Example 9: Panning ELISAs—CD3 scFv Peptides

Clonal phages were harvested as crude supernatants and screened via standard enzyme linked immunosorbent assays (ELISAs). Briefly, biotinylated SP34.185 scFv was captured on neutravidin coated plates. Prior to the addition of clonal phage, wells were incubated with blocking buffer and CD3 or blocking buffer alone. Without washing or aspirating, clonal phage supernatants were then added to the wells and incubated for a short time. Wells were then washed followed by detection of bound phage using a horse radish peroxidase conjugated anti-M13 antibody. Clonal phage of interest was then sent for sequence analysis.

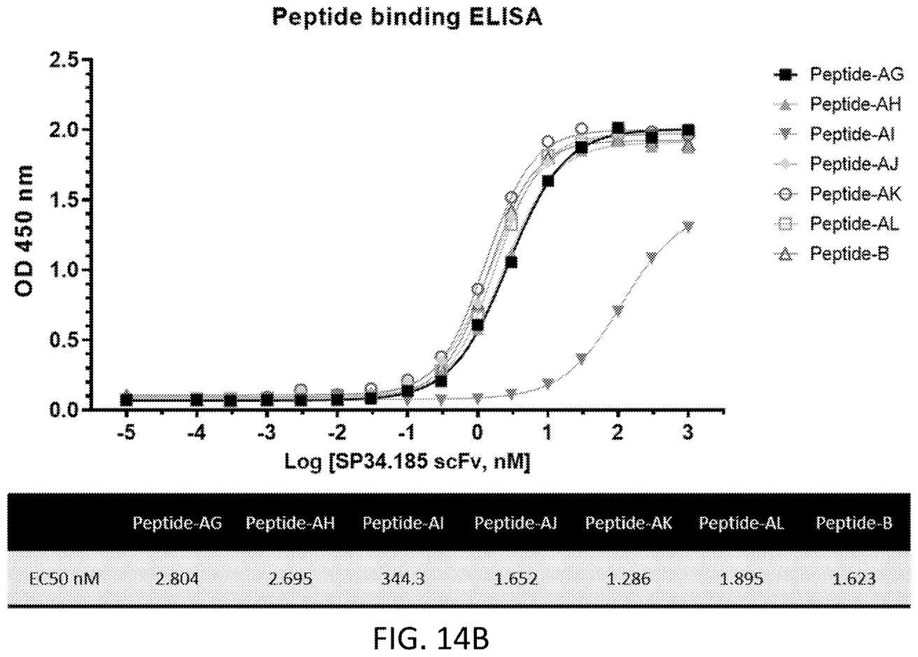

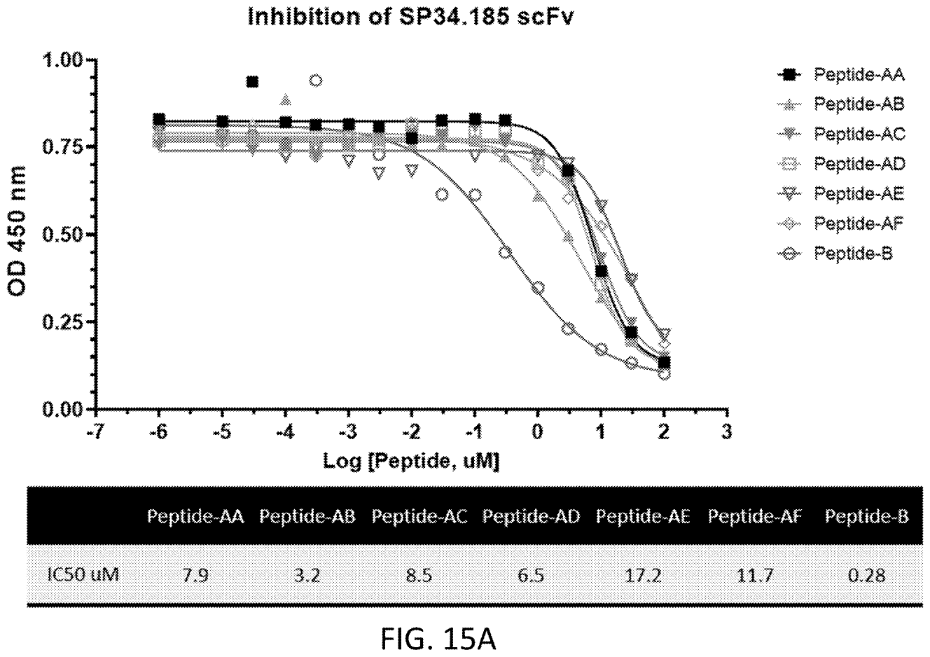

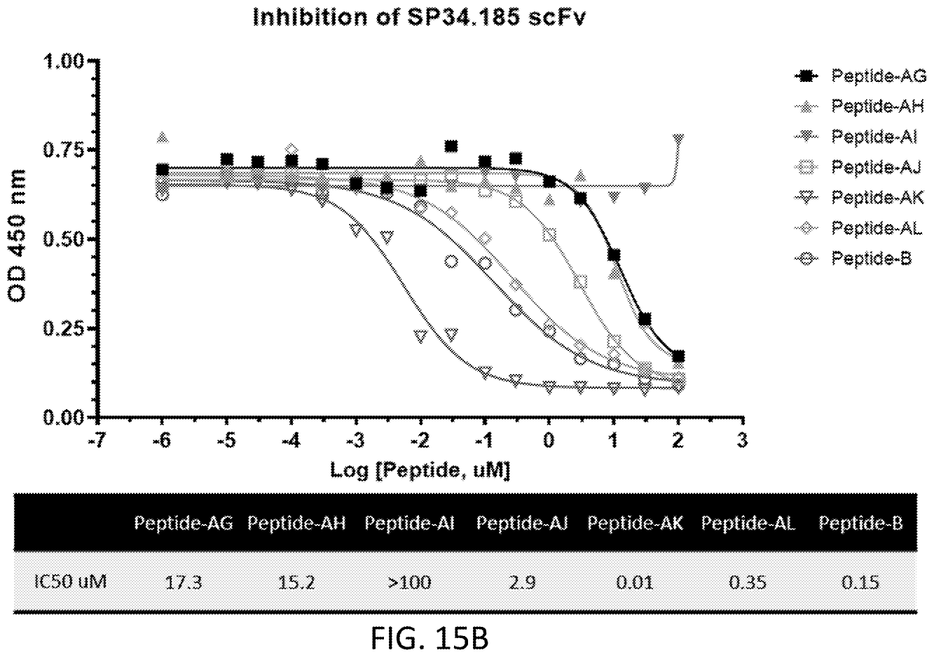

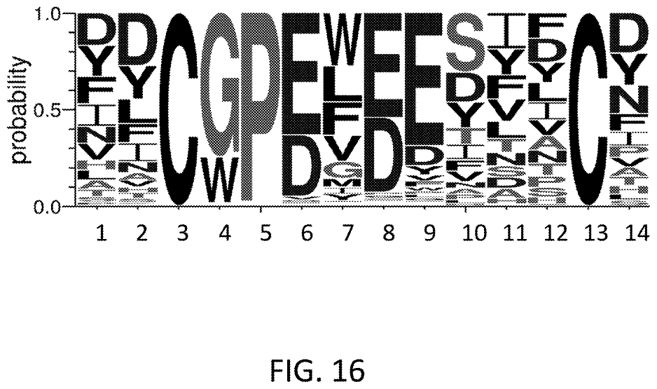

Phage panning results of CD3 scFv Peptide-A library sequences are shown in Table 20. The sequences of those peptides selected for synthesis are shown in Table 21, and further evaluated for binding to anti-CD3 scFv (FIGS. 14A-14B) and inhibition of anti-CD3 scFv binding to CD3 (FIGS. 15A-15B). The consensus sequence shown in FIG. 16 was calculated from all the sequences shown in Table 20 and was generated using WebLogo 3.7.4.

TABLE 20
Clonal Phage Peptide Sequences from the Peptide-B Optimization Library Panning (-)
indicates same amino acid as in CD3 scFv Peptide-B corresponding position (e.g. Phage-1 position).

| | | | | | | | | | | | | | | | Phage binding ELISA | | | |
|---|---|---|---|---|---|---|---|---|---|---|---|---|---|---|---|---|---|---|
| | | | | | | | | | | | | | | | | | SP34.185 | |
| | | | | | | | | | | | | | | | | SP34.185 | scFv signal | SEQ |
| Phage | Amino acid position sequence | | | | | | | | | | | | | | Backgroud | scFv | in presence | ID |
| ID | 1 | 2 | 3 | 4 | 5 | 6 | 7 | 8 | 9 | 10 | 11 | 12 | 13 | 14 | signal | signal | of CD3 | NO: |
| Phage-1/ | V | Y | C | G | P | E | F | D | E | S | V | G | C | M | 0.06 | 2.79 | 0.09 | 19 |
| Peptide B | | | | | | | | | | | | | | | | | | |
| Phage-2 | D | D | — | W | — | D | W | E | F | D | F | A | — | A | 0.08 | 2.75 | 0.09 | 106 |
| Phage-3 | Y | I | — | — | L | D | — | P | D | F | L | Y | — | D | 0.08 | 2.88 | 0.10 | 107 |
| Phage-4 | F | D | — | W | — | D | W | E | — | Y | F | V | — | D | 0.08 | 2.79 | 0.09 | 108 |
| Phage-5 | Y | I | — | W | — | D | W | E | — | Y | F | D | — | D | 0.08 | 2.74 | 0.09 | 109 |
| Phage-6 | N | I | — | W | — | D | W | E | D | D | Y | F | — | F | 0.09 | 2.54 | 0.09 | 110 |
| Phage-7 | N | F | — | W | — | D | W | E | Y | I | Y | P | — | I | 0.07 | 2.77 | 0.09 | 111 |
| Phage-8 | — | D | — | W | — | D | W | E | — | D | F | L | — | I | 0.08 | 2.54 | 0.08 | 112 |
| Phage-9 | H | A | — | W | — | D | W | E | — | Y | F | P | — | N | 0.08 | 2.85 | 0.09 | 113 |
| Phage-10 | Y | D | — | — | — | D | V | — | — | — | Y | V | — | V | 0.09 | 2.63 | 0.10 | 114 |
| Phage-11 | I | D | — | W | — | D | W | E | D | D | T | F | — | Y | 0.09 | 2.73 | 0.08 | 115 |
| Phage-12 | Y | L | — | — | — | D | G | — | — | T | L | A | — | Y | 0.08 | 2.66 | 0.15 | 116 |
| Phage-13 | — | D | — | — | — | D | G | — | — | — | I | L | — | Y | 0.11 | 2.13 | 0.08 | 117 |
| Phage-14 | F | I | — | W | — | D | W | E | — | D | Y | F | — | A | 0.07 | 2.44 | 0.09 | 119 |
| Phage-15 | G | D | — | W | — | D | W | E | W | D | F | Y | — | D | 0.07 | 2.71 | 0.07 | 120 |
| Phage-16 | Y | L | — | W | — | D | W | E | Y | I | D | L | — | D | 0.12 | 2.67 | 0.08 | 121 |
| Phage-17 | S | F | — | W | — | D | W | E | — | Y | F | D | — | D | 0.10 | 2.60 | 0.07 | 122 |
| Phage-18 | D | D | — | W | — | D | W | E | — | Y | A | S | — | D | 0.09 | 2.57 | 0.07 | 123 |
| Phage-19 | N | L | — | W | — | D | W | E | Y | P | F | F | — | D | 0.09 | 2.52 | 0.09 | 124 |
| Phage-20 | F | D | — | W | — | D | W | E | — | — | F | V | — | D | 0.08 | 2.34 | 0.09 | 125 |
| Phage-21 | D | I | — | — | — | D | G | — | — | T | I | I | — | D | 0.13 | 2.30 | 0.10 | 126 |
| Phage-22 | D | D | — | W | — | D | W | E | Y | Y | A | V | — | D | 0.09 | 2.28 | 0.09 | 127 |
| Phage-23 | Y | D | — | W | — | D | W | E | — | Y | S | N | — | D | 0.10 | 2.17 | 0.08 | 128 |
| Phage-24 | I | N | — | W | — | D | W | E | D | Y | F | F | — | D | 0.07 | 2.16 | 0.07 | 129 |
| Phage-25 | N | I | — | W | — | D | W | E | D | D | T | F | — | F | 0.06 | 2.87 | 0.07 | 130 |
| Phage-26 | N | I | — | W | — | D | W | E | P | N | S | F | — | F | 0.09 | 2.87 | 0.08 | 131 |
| Phage-27 | Y | D | — | — | — | — | M | — | — | — | I | D | — | F | 0.09 | 2.39 | 0.08 | 132 |
| Phage-28 | D | F | — | W | — | D | W | E | F | P | F | I | — | H | 0.11 | 2.73 | 0.12 | 133 |
| Phage-29 | D | F | — | — | — | — | M | — | — | — | I | T | — | I | 0.07 | 2.36 | 0.08 | 134 |
| Phage-30 | Y | D | — | — | — | — | — | — | — | — | T | V | — | I | 0.10 | 2.32 | 0.08 | 135 |
| Phage-31 | H | D | — | W | — | D | W | E | W | D | I | F | — | I | 0.07 | 2.26 | 0.08 | 136 |
| Phage-32 | H | A | — | W | — | D | W | E | — | Y | N | P | — | N | 0.11 | 2.71 | 0.11 | 137 |
| Phage-33 | D | V | — | W | — | D | W | E | W | D | F | F | — | N | 0.08 | 2.65 | 0.08 | 138 |
| Phage-34 | N | I | — | W | — | D | W | E | Y | Y | I | P | — | N | 0.10 | 2.57 | 0.08 | 139 |
| Phage-35 | I | I | — | W | — | D | W | E | F | I | D | Y | — | N | 0.08 | 2.10 | 0.07 | 140 |
| Phage-36 | S | L | — | W | — | D | W | E | Y | D | I | A | — | P | 0.07 | 2.53 | 0.08 | 141 |
| Phage-37 | D | L | — | — | — | — | — | — | — | — | I | F | — | P | 0.08 | 2.49 | 0.09 | 142 |
| Phage-38 | T | N | — | W | — | D | W | E | W | V | L | P | — | P | 0.14 | 2.47 | 0.10 | 143 |
| Phage-39 | I | E | — | W | — | D | W | E | P | N | Y | F | — | P | 0.13 | 2.29 | 0.09 | 144 |
| Phage-40 | I | F | — | W | — | D | W | E | D | Y | — | D | — | P | 0.07 | 2.28 | 0.07 | 145 |
| Phage-41 | I | D | — | W | — | D | W | E | Y | D | F | F | — | P | 0.07 | 2.26 | 0.08 | 146 |
| Phage-42 | L | F | — | W | — | D | W | E | D | — | F | F | — | P | 0.18 | 2.11 | 0.13 | 147 |
| Phage-43 | — | D | — | W | — | D | W | E | D | Y | A | D | — | T | 0.11 | 2.20 | 0.10 | 148 |
| Phage-44 | — | T | — | W | — | D | W | E | Q | Y | F | P | — | V | 0.11 | 2.34 | 0.09 | 149 |
| Phage-45 | I | E | — | W | — | D | W | E | P | I | Y | P | — | Y | 0.09 | 2.85 | 0.09 | 150 |
| Phage-46 | I | T | — | W | — | D | W | E | V | Y | F | P | — | Y | 0.07 | 2.55 | 0.08 | 151 |
| Phage-47 | I | D | — | W | — | D | W | E | Y | I | H | P | — | Y | 0.06 | 2.51 | 0.09 | 152 |
| Phage-48 | I | D | — | W | — | D | W | E | Y | I | N | P | — | Y | 0.12 | 2.50 | 0.12 | 153 |
| Phage-49 | A | D | — | W | — | D | W | E | — | A | F | P | — | Y | 0.09 | 2.44 | 0.09 | 154 |
| Phage-50 | I | D | — | W | — | D | W | E | Y | I | Y | P | — | Y | 0.09 | 2.31 | 0.07 | 155 |
| Phage-51 | N | I | — | W | — | D | W | E | D | D | N | F | — | F | 0.09 | 2.08 | 0.09 | 156 |
| Phage-52 | Y | D | — | W | — | D | W | E | Y | V | D | A | — | Y | 0.09 | 2.06 | 0.09 | 157 |
| Phage-53 | F | — | — | — | — | D | G | — | — | — | Y | V | — | D | 0.09 | 2.03 | 0.11 | 158 |
| Phage-54 | D | T | — | W | — | D | W | E | Y | I | N | I | — | S | 0.11 | 2.02 | 0.11 | 159 |
| Phage-55 | F | V | — | W | — | D | W | E | D | F | N | F | — | D | 0.07 | 2.01 | 0.08 | 160 |
| Phage-56 | F | A | — | W | — | D | W | E | D | Y | — | A | — | D | 0.07 | 2.01 | 0.09 | 161 |
| Phage-57 | D | N | — | W | — | D | W | E | Y | D | F | F | — | V | 0.08 | 1.99 | 0.09 | 162 |
| Phage-58 | Y | D | — | W | — | D | W | E | — | Y | N | D | — | A | 0.09 | 1.96 | 0.11 | 163 |
| Phage-59 | D | D | — | — | — | D | G | — | — | T | I | I | — | V | 0.07 | 1.91 | 0.09 | 164 |
| Phage-60 | F | P | — | W | — | D | W | E | — | Y | A | J | — | D | 0.10 | 1.89 | 0.10 | 165 |
| Phage-61 | P | D | — | — | — | D | G | V | — | V | L | F | — | T | 0.12 | 1.86 | 0.07 | 166 |
| Phage-62 | D | N | — | W | — | D | W | E | Y | D | Y | F | — | V | 0.07 | 1.83 | 0.07 | 167 |
| Phage-63 | I | F | — | W | — | D | W | E | — | F | Y | D | — | Y | 0.12 | 1.82 | 0.08 | 168 |
| Phage-64 | A | D | — | W | — | D | W | E | — | Y | F | P | — | N | 0.08 | 1.82 | 0.08 | 169 |
| Phage-65 | H | T | — | W | — | D | W | E | D | D | I | F | — | N | 0.12 | 1.81 | 0.10 | 170 |
| Phage-66 | F | A | — | W | — | D | W | E | — | A | F | L | — | L | 0.09 | 1.80 | 0.09 | 171 |
| Phage-67 | Y | D | — | — | — | — | L | — | — | — | I | A | — | D | 0.08 | 1.77 | 0.08 | 172 |
| Phage-68 | N | S | — | W | — | D | W | E | Y | D | I | I | — | D | 0.08 | 1.77 | 0.10 | 173 |
| Phage-69 | F | A | — | W | — | D | W | E | — | V | A | P | — | Y | 0.07 | 1.75 | 0.07 | 174 |
| Phage-70 | L | D | — | — | — | D | G | — | — | T | L | T | — | Y | 0.10 | 1.75 | 0.12 | 175 |
| Phage-71 | — | L | — | W | — | D | W | E | — | F | Y | D | — | P | 0.07 | 1.74 | 0.09 | 176 |
| Phage-72 | H | A | — | W | — | V | W | E | — | Y | F | P | — | N | 0.07 | 1.72 | 0.08 | 177 |
| Phage-73 | N | E | — | W | — | N | G | E | P | T | F | P | — | T | 0.08 | 1.71 | 0.07 | 178 |
| Phage-74 | L | T | — | — | — | D | G | — | — | T | L | Y | — | D | 0.08 | 1.70 | 0.07 | 179 |
| Phage-75 | Y | D | — | — | — | — | Y | — | — | — | — | P | — | I | 0.13 | 1.67 | 0.09 | 180 |
| Phage-76 | I | E | — | W | — | D | W | E | P | N | S | F | — | D | 0.09 | 1.66 | 0.08 | 181 |
| Phage-77 | Y | D | — | — | — | — | L | — | — | — | I | H | — | Y | 0.12 | 1.66 | 0.09 | 182 |
| Phage-78 | I | — | — | — | — | — | — | — | — | — | T | I | — | N | 0.08 | 1.63 | 0.08 | 183 |
| Phage-79 | I | — | — | — | — | — | V | E | — | A | Y | L | — | Y | 0.09 | 1.62 | 0.10 | 184 |
| Phage-80 | F | D | — | — | — | D | G | — | — | T | — | Y | — | D | 0.09 | 1.61 | 0.08 | 185 |
| Phage-81 | I | D | — | — | — | D | G | — | — | T | I | S | — | Y | 0.08 | 1.57 | 0.11 | 186 |
| Phage-82 | N | I | — | — | — | — | — | — | — | — | S | T | — | L | 0.10 | 1.55 | 0.11 | 187 |
| Phage-83 | Y | D | — | — | — | D | G | — | — | — | Y | F | — | D | 0.08 | 1.53 | 0.08 | 188 |
| Phage-84 | N | F | — | W | — | D | W | E | Y | F | N | D | — | N | 0.09 | 1.53 | 0.09 | 189 |
| Phage-85 | — | L | — | W | — | D | W | E | A | F | F | D | — | D | 0.07 | 1.47 | 0.07 | 190 |
| Phage-86 | I | — | — | — | — | — | W | E | W | P | — | A | — | N | 0.16 | 1.47 | 0.10 | 191 |
| Phage-87 | — | F | — | W | — | D | W | E | D | N | F | F | — | N | 0.08 | 1.46 | 0.10 | 192 |
| Phage-88 | — | V | — | W | — | D | W | E | T | F | F | P | — | D | 0.08 | 1.46 | 0.08 | 193 |
| Phage-89 | D | N | — | — | — | D | G | — | — | T | Y | I | — | N | 0.10 | 1.45 | 0.09 | 194 |
| Phage-90 | D | N | — | W | — | D | W | E | Y | N | F | F | — | V | 0.07 | 1.45 | 0.08 | 195 |
| Phage-91 | F | — | — | — | — | — | V | E | — | D | Y | L | — | I | 0.10 | 1.43 | 0.10 | 196 |
| Phage-92 | D | N | — | W | — | D | W | E | Y | D | I | F | — | V | 0.07 | 1.43 | 0.07 | 197 |
| Phage-93 | T | D | — | — | — | — | — | — | — | — | I | A | — | P | 0.08 | 1.42 | 0.08 | 198 |
| Phage-94 | Y | F | — | — | — | — | V | E | — | Y | T | L | — | F | 0.10 | 1.42 | 0.10 | 199 |
| Phage-95 | F | — | — | — | — | — | — | — | — | — | A | P | — | N | 0.06 | 1.37 | 0.08 | 200 |
| Phage-96 | F | D | — | — | — | — | V | E | — | Y | F | Y | — | A | 0.11 | 1.36 | 0.08 | 201 |
| Phage-97 | D | F | — | W | — | D | W | E | D | F | F | F | — | A | 0.18 | 1.35 | 0.12 | 202 |
| Phage-98 | F | F | — | — | — | D | G | — | — | T | L | S | — | N | 0.08 | 1.35 | 0.09 | 203 |
| Phage-99 | F | I | — | — | — | — | — | — | — | — | — | A | — | L | 0.14 | 1.35 | 0.09 | 204 |
| Phage- | Y | D | — | — | — | — | — | — | — | A | I | — | — | Y | 0.09 | 1.32 | 0.10 | 205 |
| 100 | | | | | | | | | | | | | | | | | | |
| Phage- | Y | I | — | W | — | D | W | E | — | Y | L | Y | — | P | 0.10 | 1.32 | 0.15 | 206 |
| 101 | | | | | | | | | | | | | | | | | | |
| Phage- | F | D | — | W | — | D | W | E | — | P | T | T | — | H | 0.08 | 1.31 | 0.08 | 207 |
| 102 | | | | | | | | | | | | | | | | | | |
| Phage- | Y | D | — | W | — | D | W | E | D | F | P | I | — | D | 0.14 | 1.31 | 0.10 | 208 |
| 103 | | | | | | | | | | | | | | | | | | |
| Phage- | — | V | — | W | — | D | W | E | Y | I | D | D | — | S | 0.08 | 1.30 | 0.07 | 209 |
| 104 | | | | | | | | | | | | | | | | | | |
| Phage- | I | N | — | W | — | D | W | E | V | I | S | F | — | D | 0.12 | 1.30 | 0.08 | 210 |
| 105 | | | | | | | | | | | | | | | | | | |
| Phage- | L | S | — | W | — | D | W | E | — | V | T | P | — | L | 0.10 | 1.29 | 0.10 | 211 |
| 106 | | | | | | | | | | | | | | | | | | |
| Phage- | F | A | — | W | — | D | W | E | — | V | D | I | — | Y | 0.09 | 1.28 | 0.08 | 212 |
| 107 | | | | | | | | | | | | | | | | | | |
| Phage- | Y | D | — | — | — | — | M | — | — | — | I | V | — | D | 0.10 | 1.25 | 0.08 | 213 |
| 108 | | | | | | | | | | | | | | | | | | |
| Phage- | Y | D | — | W | — | D | W | E | V | F | I | V | — | D | 0.06 | 1.25 | 0.07 | 214 |
| 109 | | | | | | | | | | | | | | | | | | |
| Phage- | D | N | — | W | — | D | W | E | H | N | F | F | — | V | 0.10 | 1.25 | 0.08 | 215 |
| 110 | | | | | | | | | | | | | | | | | | |
| Phage- | Y | D | — | — | — | D | G | — | — | — | I | Y | — | P | 0.07 | 1.23 | 0.08 | 216 |
| 111 | | | | | | | | | | | | | | | | | | |
| Phage- | Y | D | — | — | — | — | — | E | F | P | Y | Y | — | F | 0.12 | 1.23 | 0.12 | 217 |
| 112 | | | | | | | | | | | | | | | | | | |
| Phage- | A | D | — | — | — | — | Y | — | — | — | — | P | — | V | 0.11 | 1.22 | 0.09 | 218 |
| 113 | | | | | | | | | | | | | | | | | | |
| Phage- | F | L | — | — | — | — | V | E | — | V | H | Y | — | S | 0.08 | 1.22 | 0.10 | 219 |
| 114 | | | | | | | | | | | | | | | | | | |
| Phage- | T | D | — | W | — | D | W | E | Y | I | T | S | — | S | 0.08 | 1.22 | 0.08 | 220 |
| 115 | | | | | | | | | | | | | | | | | | |
| Phage- | A | F | — | — | — | — | L | — | — | — | I | T | — | D | 0.09 | 1.21 | 0.09 | 221 |
| 116 | | | | | | | | | | | | | | | | | | |
| Phage- | N | D | — | W | — | D | W | E | — | Y | F | S | — | Y | 0.09 | 1.19 | 0.09 | 222 |
| 117 | | | | | | | | | | | | | | | | | | |
| Phage- | F | D | — | — | — | — | W | E | I | V | T | D | — | Y | 0.08 | 1.19 | 0.09 | 223 |
| 118 | | | | | | | | | | | | | | | | | | |
| Phage- | N | L | — | — | — | — | M | — | — | — | I | I | — | P | 0.13 | 1.19 | 0.11 | 224 |
| 119 | | | | | | | | | | | | | | | | | | |
| Phage- | D | L | — | — | — | — | M | — | — | — | I | Y | — | D | 0.10 | 1.19 | 0.14 | 225 |
| 120 | | | | | | | | | | | | | | | | | | |
| Phage- | F | D | — | — | — | D | G | V | — | D | Y | I | — | D | 0.09 | 1.18 | 0.09 | 226 |
| 121 | | | | | | | | | | | | | | | | | | |
| Phage- | Y | A | — | W | — | D | W | E | — | D | F | A | — | Y | 0.11 | 1.18 | 0.08 | 227 |
| 122 | | | | | | | | | | | | | | | | | | |
| Phage- | H | D | — | — | — | — | M | — | — | — | I | V | — | V | 0.10 | 1.17 | 0.10 | 228 |
| 123 | | | | | | | | | | | | | | | | | | |
| Phage- | — | F | — | — | — | — | — | E | F | I | F | L | — | A | 0.07 | 1.17 | 0.08 | 229 |
| 124 | | | | | | | | | | | | | | | | | | |
| Phage- | Y | D | — | — | — | — | L | I | — | — | I | L | — | D | 0.08 | 1.16 | 0.09 | 230 |
| 125 | | | | | | | | | | | | | | | | | | |
| Phage- | S | V | — | W | — | D | W | E | — | F | Y | S | — | D | 0.11 | 1.16 | 0.10 | 231 |
| 126 | | | | | | | | | | | | | | | | | | |
| Phage- | P | — | — | — | — | D | G | — | — | T | A | I | — | T | 0.13 | 1.16 | 0.10 | 232 |
| 127 | | | | | | | | | | | | | | | | | | |
| Phage- | D | D | — | — | — | — | L | E | W | Y | Y | P | — | Y | 0.09 | 1.16 | 0.08 | 233 |
| 128 | | | | | | | | | | | | | | | | | | |
| Phage- | F | I | — | — | — | — | — | — | — | — | L | P | — | N | 0.08 | 1.14 | 0.09 | 234 |
| 129 | | | | | | | | | | | | | | | | | | |
| Phage- | I | D | — | — | — | — | — | — | — | — | L | P | — | D | 0.11 | 1.14 | 0.33 | 235 |
| 130 | | | | | | | | | | | | | | | | | | |
| Phage- | F | L | — | — | — | — | — | E | — | D | A | P | — | Y | 0.08 | 1.13 | 0.08 | 236 |
| 131 | | | | | | | | | | | | | | | | | | |
| Phage- | I | F | — | — | — | D | G | — | — | T | H | I | — | H | 0.10 | 1.13 | 0.07 | 237 |
| 132 | | | | | | | | | | | | | | | | | | |
| Phage- | — | F | — | W | — | D | W | E | Y | I | D | F | — | N | 0.10 | 1.11 | 0.21 | 238 |
| 133 | | | | | | | | | | | | | | | | | | |
| Phage- | I | F | — | — | — | — | Y | — | — | — | L | H | — | I | 0.12 | 1.11 | 0.11 | 239 |
| 134 | | | | | | | | | | | | | | | | | | |
| Phage- | H | L | — | W | — | D | W | E | W | Y | — | D | — | P | 0.08 | 1.11 | 0.10 | 240 |
| 135 | | | | | | | | | | | | | | | | | | |
| Phage- | F | I | — | — | — | — | M | — | — | — | I | A | — | N | 0.08 | 1.11 | 0.09 | 241 |
| 136 | | | | | | | | | | | | | | | | | | |
| Phage- | I | F | — | — | — | — | V | E | M | I | F | L | — | N | 0.09 | 1.10 | 0.08 | 242 |
| 137 | | | | | | | | | | | | | | | | | | |
| Phage- | Y | D | — | — | — | — | W | E | F | P | — | D | — | I | 0.11 | 1.09 | 0.11 | 243 |
| 138 | | | | | | | | | | | | | | | | | | |
| Phage- | N | L | — | — | — | — | — | — | — | | I | T | — | F | 0.10 | 1.09 | 0.08 | 244 |
| 139 | | | | | | | | | | | | | | | | | | |
| Phage- | F | — | — | — | — | — | V | E | D | F | Y | F | — | Y | 0.08 | 1.09 | 0.08 | 245 |
| 140 | | | | | | | | | | | | | | | | | | |
| Phage- | D | — | — | — | — | — | — | — | — | — | L | I | — | N | 0.11 | 1.07 | 0.11 | 246 |
| 141 | | | | | | | | | | | | | | | | | | |
| Phage- | D | — | — | — | — | — | — | — | — | — | L | P | — | D | 0.08 | 1.07 | 0.08 | 247 |
| 142 | | | | | | | | | | | | | | | | | | |
| Phage- | A | I | — | — | — | — | L | — | — | — | I | A | — | P | 0.09 | 1.07 | 0.09 | 248 |
| 143 | | | | | | | | | | | | | | | | | | |
| Phage- | — | I | — | — | — | — | V | E | D | Y | N | L | — | Y | 0.08 | 1.07 | 0.09 | 249 |
| 144 | | | | | | | | | | | | | | | | | | |
| Phage- | H | T | — | W | — | D | W | E | D | Y | T | V | — | P | 0.10 | 1.06 | 0.09 | 250 |
| 145 | | | | | | | | | | | | | | | | | | |
| Phage- | S | D | — | W | — | D | W | E | Y | F | Y | D | — | N | 0.10 | 1.06 | 0.08 | 251 |
| 146 | | | | | | | | | | | | | | | | | | |
| Phage- | — | F | — | — | — | D | G | — | — | T | — | H | — | D | 0.09 | 1.05 | 0.08 | 252 |
| 147 | | | | | | | | | | | | | | | | | | |
| Phage- | D | — | — | — | — | — | Y | — | — | — | — | H | — | I | 0.09 | 1.05 | 0.08 | 253 |
| 148 | | | | | | | | | | | | | | | | | | |
| Phage- | A | D | — | — | — | D | G | — | — | — | I | I | — | H | 0.07 | 1.05 | 0.08 | 254 |
| 149 | | | | | | | | | | | | | | | | | | |
| Phage- | F | — | — | — | — | — | L | — | — | — | L | T | — | V | 0.10 | 1.05 | 0.08 | 255 |
| 150 | | | | | | | | | | | | | | | | | | |
| Phage- | I | L | — | — | — | — | V | E | — | D | Y | Y | — | Y | 0.11 | 1.04 | 0.09 | 256 |
| 151 | | | | | | | | | | | | | | | | | | |
| Phage- | H | L | — | W | — | D | W | E | — | Y | H | S | — | D | 0.09 | 1.04 | 0.09 | 257 |
| 152 | | | | | | | | | | | | | | | | | | |
| Phage- | I | F | — | W | — | D | W | E | D | Y | N | F | — | T | 0.08 | 1.04 | 0.11 | 258 |
| 153 | | | | | | | | | | | | | | | | | | |
| Phage- | I | V | — | — | — | D | G | — | — | T | L | I | — | H | 0.12 | 1.04 | 0.11 | 259 |
| 154 | | | | | | | | | | | | | | | | | | |
| Phage- | A | D | — | W | — | D | W | E | W | D | Y | T | — | D | 0.12 | 1.03 | 0.11 | 260 |
| 155 | | | | | | | | | | | | | | | | | | |
| Phage- | I | T | — | — | — | — | — | — | — | — | T | T | — | N | 0.20 | 1.02 | 0.21 | 261 |
| 156 | | | | | | | | | | | | | | | | | | |
| Phage- | Y | H | — | W | — | D | W | E | — | Y | T | S | — | D | 0.20 | 1.02 | 0.09 | 262 |
| 157 | | | | | | | | | | | | | | | | | | |
| Phage- | N | — | — | — | — | — | V | E | — | Y | A | L | — | T | 0.11 | 1.01 | 0.10 | 263 |
| 158 | | | | | | | | | | | | | | | | | | |
| Phage- | F | I | — | — | — | — | M | — | — | — | I | H | — | D | 0.15 | 1.00 | 0.19 | 264 |
| 159 | | | | | | | | | | | | | | | | | | |
| Phage- | D | N | — | W | — | D | W | E | — | F | A | V | — | P | 0.14 | 1.00 | 0.10 | 265 |
| 160 | | | | | | | | | | | | | | | | | | |
| Phage- | Y | D | — | — | — | — | L | — | — | T | — | V | — | D | 0.10 | 1.00 | 0.09 | 266 |
| 161 | | | | | | | | | | | | | | | | | | |
| Phage- | Y | D | — | — | — | — | — | — | — | — | I | A | — | Y | 0.08 | 0.99 | 0.08 | 267 |
| 162 | | | | | | | | | | | | | | | | | | |
| Phage- | I | D | — | W | — | D | W | E | Y | T | — | H | — | D | 0.07 | 0.97 | 0.09 | 268 |
| 163 | | | | | | | | | | | | | | | | | | |
| Phage- | D | D | — | — | — | — | L | — | — | — | I | I | — | I | 0.09 | 0.96 | 0.09 | 269 |
| 164 | | | | | | | | | | | | | | | | | | |
| Phage- | — | — | — | — | — | — | Y | — | — | — | S | F | — | F | 0.09 | 0.91 | 0.08 | 270 |
| 165 | | | | | | | | | | | | | | | | | | |
| Phage- | F | N | — | W | — | D | W | E | D | P | Y | F | — | V | 0.09 | 0.86 | 0.07 | 271 |
| 166 | | | | | | | | | | | | | | | | | | |
| Phage- | Y | D | — | — | — | — | Y | — | — | — | S | Y | — | S | 0.08 | 0.82 | 0.07 | 272 |
| 167 | | | | | | | | | | | | | | | | | | |
| Phage- | — | A | — | W | — | D | W | E | Y | T | D | S | — | F | 0.13 | 0.79 | 0.09 | 273 |
| 168 | | | | | | | | | | | | | | | | | | |
| Phage- | T | D | — | — | — | — | — | — | — | — | — | A | — | Y | 0.10 | 0.77 | 0.09 | 274 |
| 169 | | | | | | | | | | | | | | | | | | |
| Phage- | T | D | — | W | — | D | W | E | F | Y | A | D | — | D | 0.07 | 0.75 | 0.08 | 275 |
| 170 | | | | | | | | | | | | | | | | | | |
| Phage- | Y | D | — | — | — | — | L | — | — | — | — | I | — | H | 0.09 | 0.69 | 0.09 | 276 |
| 171 | | | | | | | | | | | | | | | | | | |
| Phage- | S | D | — | — | — | D | G | — | — | — | I | I | — | T | 0.07 | 0.69 | 0.07 | 277 |
| 172 | | | | | | | | | | | | | | | | | | |
| Phage- | Y | — | — | — | — | — | — | — | — | — | I | D | — | D | 0.08 | 0.67 | 0.09 | 278 |
| 173 | | | | | | | | | | | | | | | | | | |
| Phage- | F | F | — | — | — | — | I | — | — | — | I | A | — | V | 0.08 | 0.62 | 0.09 | 279 |
| 174 | | | | | | | | | | | | | | | | | | |
| Phage- | D | — | — | — | — | — | — | — | — | — | T | F | — | D | 0.16 | 0.60 | 0.10 | 280 |
| 175 | | | | | | | | | | | | | | | | | | |
| Phage- | Y | D | — | — | — | — | W | E | W | P | I | D | — | V | 0.10 | 0.59 | 0.10 | 281 |
| 176 | | | | | | | | | | | | | | | | | | |
| Phage- | F | — | — | — | — | — | T | E | L | F | S | F | — | Y | 0.13 | 0.59 | 0.11 | 282 |
| 177 | | | | | | | | | | | | | | | | | | |
| Phage- | Y | — | — | — | — | — | V | — | — | — | I | T | — | P | 0.15 | 0.42 | 0.11 | 283 |
| 178 | | | | | | | | | | | | | | | | | | |
| Phage- | I | L | — | — | — | — | — | — | — | — | I | N | — | N | 0.09 | 0.37 | 0.25 | 284 |
| 179 | | | | | | | | | | | | | | | | | | |
| Phage- | — | V | — | — | — | A | M | G | Q | H | Y | L | — | D | 0.08 | 0.09 | 0.08 | 285 |
| 180 | | | | | | | | | | | | | | | | | | |
| Phage- | — | V | — | — | T | K | M | G | — | H | Y | L | — | S | 0.08 | 0.08 | 0.08 | 286 |
| 181 | | | | | | | | | | | | | | | | | | |
| Phage- | Y | D | — | W | — | D | W | E | Y | V | Y | A | — | Y | 0.08 | 0.98 | 0.08 | 287 |
| 182 | | | | | | | | | | | | | | | | | | |
| Phage- | D | L | — | — | — | — | L | — | — | — | — | N | — | D | 0.09 | 0.98 | 0.08 | 288 |
| 183 | | | | | | | | | | | | | | | | | | |
| Phage- | Y | — | — | — | — | — | — | — | — | — | T | V | — | Y | 0.14 | 0.97 | 0.16 | 289 |
| 184 | | | | | | | | | | | | | | | | | | |
| Phage- | L | D | — | W | — | D | W | E | W | P | Y | S | — | N | 0.08 | 0.96 | 0.09 | 290 |
| 185 | | | | | | | | | | | | | | | | | | |
| Phage- | F | I | — | W | — | D | W | E | D | D | F | F | — | Y | 0.08 | 0.96 | 0.09 | 291 |
| 186 | | | | | | | | | | | | | | | | | | |
| Phage- | D | L | — | — | — | — | V | E | W | Y | F | F | — | N | 0.11 | 0.95 | 0.10 | 292 |
| 187 | | | | | | | | | | | | | | | | | | |
| Phage- | Y | D | — | — | — | — | L | — | — | — | I | V | — | F | 0.07 | 0.94 | 0.08 | 293 |
| 188 | | | | | | | | | | | | | | | | | | |
| Phage- | L | N | — | W | — | V | W | E | D | D | — | F | — | Y | 0.09 | 0.92 | 0.09 | 294 |
| 189 | | | | | | | | | | | | | | | | | | |
| Phage- | F | N | — | W | — | D | W | E | D | P | N | F | — | V | 0.09 | 0.91 | 0.09 | 295 |
| 190 | | | | | | | | | | | | | | | | | | |
| Phage- | — | I | — | W | — | D | W | E | D | D | Y | F | — | P | 0.10 | 0.91 | 0.13 | 296 |
| 191 | | | | | | | | | | | | | | | | | | |
| Phage- | F | L | — | — | — | — | — | — | — | — | S | V | — | Y | 0.10 | 0.91 | 0.08 | 297 |
| 192 | | | | | | | | | | | | | | | | | | |
| Phage- | Y | D | — | — | — | — | L | — | — | — | I | F | — | Y | 0.10 | 0.91 | 0.09 | 298 |
| 193 | | | | | | | | | | | | | | | | | | |
| Phage- | H | L | — | — | — | D | G | — | — | — | F | T | — | F | 0.11 | 0.90 | 0.10 | 299 |
| 194 | | | | | | | | | | | | | | | | | | |
| Phage- | Y | F | — | — | — | — | M | — | — | — | L | Y | — | I | 0.08 | 0.90 | 0.08 | 300 |
| 195 | | | | | | | | | | | | | | | | | | |
| Phage- | Y | — | — | — | — | — | V | E | — | Y | A | N | — | Y | 0.07 | 0.90 | 0.07 | 301 |
| 196 | | | | | | | | | | | | | | | | | | |
| Phage- | N | T | — | — | — | — | — | — | — | — | T | A | — | Y | 0.16 | 0.90 | 0.58 | 302 |
| 197 | | | | | | | | | | | | | | | | | | |
| Phage- | I | D | — | W | — | D | W | E | — | A | F | N | — | Y | 0.09 | 0.90 | 0.08 | 303 |
| 198 | | | | | | | | | | | | | | | | | | |
| Phage- | A | — | — | — | — | — | L | E | — | F | F | L | — | T | 0.09 | 0.89 | 0.08 | 304 |
| 199 | | | | | | | | | | | | | | | | | | |
| Phage- | I | — | — | — | — | — | V | E | — | V | H | H | — | Y | 0.08 | 0.89 | 0.08 | 305 |
| 200 | | | | | | | | | | | | | | | | | | |
| Phage- | F | F | — | — | — | — | — | — | — | — | — | A | — | D | 0.10 | 0.89 | 0.11 | 306 |
| 201 | | | | | | | | | | | | | | | | | | |
| Phage- | Y | D | — | — | — | — | L | — | — | T | I | I | — | A | 0.08 | 0.89 | 0.08 | 307 |
| 202 | | | | | | | | | | | | | | | | | | |
| Phage- | I | L | — | — | — | — | W | E | Y | P | L | D | — | S | 0.09 | 0.89 | 0.10 | 308 |
| 203 | | | | | | | | | | | | | | | | | | |
| Phage- | F | I | — | — | — | — | — | — | — | — | T | T | — | N | 0.09 | 0.88 | 0.10 | 309 |
| 204 | | | | | | | | | | | | | | | | | | |
| Phage- | F | — | — | — | — | — | L | — | — | — | — | S | — | D | 0.17 | 0.88 | 0.15 | 310 |
| 205 | | | | | | | | | | | | | | | | | | |
| Phage- | H | L | — | — | — | — | L | — | — | — | — | T | — | F | 0.10 | 0.87 | 0.10 | 311 |
| 206 | | | | | | | | | | | | | | | | | | |
| Phage- | L | I | — | — | — | — | V | E | D | Y | S | L | — | H | 0.09 | 0.87 | 0.09 | 312 |
| 207 | | | | | | | | | | | | | | | | | | |
| Phage- | Y | F | — | — | — | — | M | — | — | — | — | Y | — | D | 0.08 | 0.87 | 0.08 | 313 |
| 208 | | | | | | | | | | | | | | | | | | |
| Phage- | H | — | — | — | — | — | M | — | — | — | I | Y | — | I | 0.13 | 0.87 | 0.09 | 314 |
| 209 | | | | | | | | | | | | | | | | | | |
| Phage- | F | D | — | — | — | — | L | — | — | — | I | N | — | D | 0.08 | 0.87 | 0.09 | 315 |
| 210 | | | | | | | | | | | | | | | | | | |
| Phage- | Y | — | — | — | — | — | V | E | — | Y | T | Y | — | T | 0.07 | 0.87 | 0.08 | 316 |
| 211 | | | | | | | | | | | | | | | | | | |
| Phage- | L | A | — | W | — | V | R | E | — | I | N | A | — | I | 0.08 | 0.85 | 0.07 | 317 |
| 212 | | | | | | | | | | | | | | | | | | |
| Phage- | I | D | — | W | — | D | W | E | D | I | T | F | — | D | 0.08 | 0.85 | 0.08 | 318 |
| 213 | | | | | | | | | | | | | | | | | | |
| Phage- | T | V | — | — | — | — | L | — | — | — | I | T | — | P | 0.11 | 0.85 | 0.15 | 319 |
| 214 | | | | | | | | | | | | | | | | | | |
| Phage- | F | — | — | — | — | — | — | E | L | P | A | D | — | D | 0.08 | 0.85 | 0.09 | 320 |
| 215 | | | | | | | | | | | | | | | | | | |
| Phage- | F | D | — | — | — | — | — | — | — | — | N | P | — | F | 0.10 | 0.85 | 0.09 | 321 |
| 216 | | | | | | | | | | | | | | | | | | |
| Phage- | D | A | — | W | — | D | W | E | — | Y | S | S | — | D | 0.10 | 0.83 | 0.10 | 322 |
| 217 | | | | | | | | | | | | | | | | | | |
| Phage- | D | H | — | W | — | D | W | E | P | N | Y | F | — | V | 0.08 | 0.83 | 0.09 | 323 |
| 218 | | | | | | | | | | | | | | | | | | |
| Phage- | D | — | — | W | — | D | W | E | I | N | Y | I | — | F | 0.09 | 0.83 | 0.10 | 324 |
| 219 | | | | | | | | | | | | | | | | | | |
| Phage- | I | — | — | W | — | D | W | E | Y | V | Y | A | — | N | 0.10 | 0.82 | 0.09 | 325 |
| 220 | | | | | | | | | | | | | | | | | | |
| Phage- | D | F | — | — | — | — | V | E | — | D | Y | L | — | D | 0.07 | 0.82 | 0.08 | 326 |
| 221 | | | | | | | | | | | | | | | | | | |
| Phage- | H | D | — | — | — | D | G | R | — | D | Y | D | — | A | 0.11 | 0.82 | 0.09 | 327 |
| 222 | | | | | | | | | | | | | | | | | | |
| Phage- | L | A | — | W | — | D | W | E | D | D | Y | F | — | V | 0.08 | 0.82 | 0.09 | 328 |
| 223 | | | | | | | | | | | | | | | | | | |
| Phage- | D | I | — | W | — | D | W | E | D | Y | L | P | — | V | 0.10 | 0.82 | 0.10 | 329 |
| 224 | | | | | | | | | | | | | | | | | | |
| Phage- | I | L | — | — | — | — | I | E | V | Y | A | L | — | P | 0.08 | 0.81 | 0.10 | 330 |
| 225 | | | | | | | | | | | | | | | | | | |
| Phage- | I | F | — | — | — | — | W | E | F | — | — | L | — | N | 0.10 | 0.81 | 0.11 | 331 |
| 226 | | | | | | | | | | | | | | | | | | |
| Phage- | T | — | — | — | — | — | V | E | D | F | S | L | — | V | 0.07 | 0.80 | 0.08 | 332 |
| 227 | | | | | | | | | | | | | | | | | | |
| Phage- | F | I | — | — | — | — | W | E | F | V | D | A | — | F | 0.11 | 0.80 | 0.09 | 333 |
| 228 | | | | | | | | | | | | | | | | | | |
| Phage- | F | A | — | W | — | D | W | E | — | D | S | P | — | D | 0.06 | 0.80 | 0.07 | 334 |
| 229 | | | | | | | | | | | | | | | | | | |
| Phage- | I | L | — | — | — | — | V | E | — | L | I | F | — | P | 0.12 | 0.80 | 0.08 | 335 |
| 230 | | | | | | | | | | | | | | | | | | |
| Phage- | F | — | — | — | — | — | V | E | — | Y | I | Y | — | Y | 0.08 | 0.80 | 0.08 | 336 |
| 231 | | | | | | | | | | | | | | | | | | |
| Phage- | D | S | — | — | — | — | L | — | — | — | I | I | — | D | 0.10 | 0.79 | 0.09 | 337 |
| 232 | | | | | | | | | | | | | | | | | | |
| Phage- | F | L | — | — | — | D | G | — | — | T | S | V | — | D | 0.11 | 0.79 | 0.08 | 338 |
| 233 | | | | | | | | | | | | | | | | | | |
| Phage- | F | N | — | W | — | N | G | E | P | T | Y | F | — | V | 0.11 | 0.79 | 0.08 | 339 |
| 234 | | | | | | | | | | | | | | | | | | |
| Phage- | L | A | — | W | — | V | W | E | Y | P | — | T | — | I | 0.09 | 0.78 | 0.09 | 340 |
| 235 | | | | | | | | | | | | | | | | | | |
| Phage- | D | — | — | — | — | — | V | E | — | D | — | Y | — | Y | 0.09 | 0.78 | 0.09 | 341 |
| 236 | | | | | | | | | | | | | | | | | | |
| Phage- | I | T | — | W | — | D | W | E | — | Y | A | N | — | T | 0.08 | 0.77 | 0.07 | 342 |
| 237 | | | | | | | | | | | | | | | | | | |
| Phage- | F | F | — | — | — | D | G | — | — | T | Y | S | — | I | 0.15 | 0.77 | 0.13 | 343 |
| 238 | | | | | | | | | | | | | | | | | | |
| Phage- | T | D | — | W | — | D | W | E | Y | A | T | S | — | D | 0.09 | 0.76 | 0.09 | 344 |
| 239 | | | | | | | | | | | | | | | | | | |
| Phage- | F | N | — | — | — | D | G | Y | — | D | Y | L | — | D | 0.10 | 0.76 | 0.11 | 345 |
| 240 | | | | | | | | | | | | | | | | | | |
| Phage- | Y | D | — | W | — | D | W | E | V | D | F | H | — | P | 0.11 | 0.76 | 0.08 | 346 |
| 241 | | | | | | | | | | | | | | | | | | |
| Phage- | N | I | — | W | — | D | W | E | D | D | S | F | — | F | 0.08 | 0.76 | 0.08 | 347 |
| 242 | | | | | | | | | | | | | | | | | | |
| Phage- | A | T | — | — | — | — | — | — | — | — | I | — | — | S | 0.13 | 0.75 | 0.09 | 348 |
| 243 | | | | | | | | | | | | | | | | | | |
| Phage- | S | — | — | — | — | — | — | — | — | — | T | F | — | D | 0.10 | 0.74 | 0.09 | 349 |
| 244 | | | | | | | | | | | | | | | | | | |
| Phage- | P | I | — | — | — | — | Y | — | — | — | D | V | — | A | 0.08 | 0.74 | 0.08 | 350 |
| 245 | | | | | | | | | | | | | | | | | | |
| Phage- | Y | — | — | — | — | D | G | — | — | Y | N | S | — | T | 0.11 | 0.74 | 0.11 | 351 |
| 246 | | | | | | | | | | | | | | | | | | |
| Phage- | — | D | — | W | — | D | W | E | V | F | I | A | — | D | 0.11 | 0.74 | 0.10 | 352 |
| 247 | | | | | | | | | | | | | | | | | | |
| Phage- | D | L | — | — | — | — | V | E | — | V | N | L | — | L | 0.12 | 0.74 | 0.10 | 353 |
| 248 | | | | | | | | | | | | | | | | | | |
| Phage- | F | D | — | — | — | — | M | — | — | — | T | T | — | F | 0.09 | 0.74 | 0.09 | 354 |
| 249 | | | | | | | | | | | | | | | | | | |
| Phage- | N | F | — | W | — | D | W | E | P | I | Y | F | — | T | 0.13 | 0.74 | 0.14 | 355 |
| 250 | | | | | | | | | | | | | | | | | | |
| Phage- | — | D | — | — | — | D | G | — | — | — | F | F | — | L | 0.08 | 0.73 | 0.08 | 356 |
| 251 | | | | | | | | | | | | | | | | | | |
| Phage- | — | D | — | — | — | D | G | — | — | T | A | F | — | I | 0.10 | 0.73 | 0.09 | 357 |
| 252 | | | | | | | | | | | | | | | | | | |
| Phage- | N | I | — | — | — | — | M | — | — | — | L | V | — | I | 0.10 | 0.73 | 0.09 | 358 |
| 253 | | | | | | | | | | | | | | | | | | |
| Phage- | I | I | — | — | — | — | — | — | — | — | F | F | — | F | 0.08 | 0.73 | 0.09 | 359 |
| 254 | | | | | | | | | | | | | | | | | | |
| Phage- | N | F | — | — | — | — | Y | — | — | — | I | S | — | I | 0.10 | 0.73 | 0.38 | 360 |
| 255 | | | | | | | | | | | | | | | | | | |
| Phage- | H | L | — | — | — | — | I | E | — | A | D | I | — | N | 0.11 | 0.73 | 0.51 | 361 |
| 256 | | | | | | | | | | | | | | | | | | |
| Phage- | D | — | — | — | — | — | V | E | — | D | Y | L | — | D | 0.08 | 0.72 | 0.09 | 362 |
| 257 | | | | | | | | | | | | | | | | | | |
| Phage- | D | — | — | — | — | — | L | — | — | — | I | N | — | D | 0.11 | 0.72 | 0.10 | 363 |
| 258 | | | | | | | | | | | | | | | | | | |
| Phage- | F | — | — | — | — | — | L | — | — | — | L | F | — | V | 0.09 | 0.72 | 0.08 | 364 |
| 259 | | | | | | | | | | | | | | | | | | |
| Phage- | P | D | — | — | — | — | W | E | F | Y | — | T | — | N | 0.12 | 0.72 | 0.08 | 365 |
| 260 | | | | | | | | | | | | | | | | | | |
| Phage- | F | D | — | — | — | — | — | E | Y | I | Y | A | — | T | 0.09 | 0.72 | 0.08 | 366 |
| 261 | | | | | | | | | | | | | | | | | | |
| Phage- | D | — | — | — | — | — | — | — | — | — | S | I | — | N | 0.12 | 0.72 | 0.11 | 367 |
| 262 | | | | | | | | | | | | | | | | | | |
| Phage- | D | F | — | — | — | — | V | E | — | Y | I | F | — | F | 0.08 | 0.72 | 0.07 | 368 |
| 263 | | | | | | | | | | | | | | | | | | |
| Phage- | P | V | — | W | — | D | W | E | Y | V | S | S | — | D | 0.08 | 0.71 | 0.08 | 369 |
| 264 | | | | | | | | | | | | | | | | | | |
| Phage- | Y | I | — | — | — | — | R | — | — | — | N | L | — | L | 0.09 | 0.71 | 0.09 | 370 |
| 265 | | | | | | | | | | | | | | | | | | |
| Phage- | Y | D | — | — | — | — | L | — | — | — | I | V | — | D | 0.11 | 0.71 | 0.11 | 371 |
| 266 | | | | | | | | | | | | | | | | | | |
| Phage- | H | D | — | W | — | D | W | E | D | F | Y | F | — | V | 0.09 | 0.71 | 0.08 | 372 |
| 267 | | | | | | | | | | | | | | | | | | |
| Phage- | H | — | — | — | — | — | Y | — | — | — | I | D | — | Y | 0.12 | 0.71 | 0.10 | 373 |
| 268 | | | | | | | | | | | | | | | | | | |
| Phage- | L | F | — | — | — | — | M | P | — | D | I | F | — | N | 0.08 | 0.71 | 0.08 | 374 |
| 269 | | | | | | | | | | | | | | | | | | |
| Phage- | H | D | — | — | — | — | L | E | F | H | Y | A | — | Y | 0.10 | 0.71 | 0.12 | 375 |
| 270 | | | | | | | | | | | | | | | | | | |
| Phage- | D | F | — | — | — | — | L | — | — | — | I | N | — | F | 0.08 | 0.70 | 0.08 | 376 |
| 271 | | | | | | | | | | | | | | | | | | |
| Phage- | Y | F | — | — | — | — | L | — | — | — | I | A | — | N | 0.10 | 0.70 | 0.10 | 377 |
| 272 | | | | | | | | | | | | | | | | | | |
| Phage- | T | D | — | W | — | D | W | E | D | D | I | I | — | D | 0.10 | 0.70 | 0.08 | 378 |
| 273 | | | | | | | | | | | | | | | | | | |
| Phage- | Y | D | — | — | — | — | L | — | — | — | I | Y | — | F | 0.09 | 0.70 | 0.08 | 379 |
| 274 | | | | | | | | | | | | | | | | | | |
| Phage- | Y | — | — | W | — | D | W | W | — | Y | — | T | — | D | 0.10 | 0.70 | 0.11 | 380 |
| 275 | | | | | | | | | | | | | | | | | | |
| Phage- | P | I | — | — | — | — | L | E | — | — | Y | L | — | N | 0.13 | 0.69 | 0.52 | 381 |
| 276 | | | | | | | | | | | | | | | | | | |
| Phage- | F | D | — | — | — | — | — | — | — | — | I | V | — | Y | 0.12 | 0.69 | 0.11 | 382 |
| 277 | | | | | | | | | | | | | | | | | | |
| Phage- | — | L | — | — | — | D | G | I | — | F | F | D | — | P | 0.09 | 0.68 | 0.07 | 383 |
| 278 | | | | | | | | | | | | | | | | | | |
| Phage- | A | — | — | — | — | — | Y | — | — | — | L | T | — | V | 0.07 | 0.68 | 0.07 | 384 |
| 279 | | | | | | | | | | | | | | | | | | |
| Phage- | D | F | — | — | — | — | L | — | — | — | I | I | — | A | 0.14 | 0.68 | 0.14 | 385 |
| 280 | | | | | | | | | | | | | | | | | | |
| Phage- | Y | D | — | — | — | — | — | — | — | — | L | D | — | N | 0.08 | 0.68 | 0.08 | 386 |
| 281 | | | | | | | | | | | | | | | | | | |
| Phage- | A | I | — | — | — | — | — | — | — | — | — | A | — | D | 0.14 | 0.67 | 0.08 | 387 |
| 282 | | | | | | | | | | | | | | | | | | |
| Phage- | L | L | — | — | — | D | G | V | — | D | F | F | — | D | 0.10 | 0.67 | 0.10 | 388 |
| 283 | | | | | | | | | | | | | | | | | | |
| Phage- | N | F | — | — | — | — | L | P | — | D | I | F | — | F | 0.12 | 0.66 | 0.13 | 389 |
| 284 | | | | | | | | | | | | | | | | | | |
| Phage- | F | — | — | — | — | — | V | E | — | V | S | L | — | N | 0.08 | 0.66 | 0.08 | 390 |
| 285 | | | | | | | | | | | | | | | | | | |
| Phage- | Y | D | — | — | — | D | G | Y | — | A | F | Y | — | H | 0.12 | 0.66 | 0.10 | 391 |
| 286 | | | | | | | | | | | | | | | | | | |
| Phage- | N | F | — | — | — | — | I | E | F | D | Y | L | — | D | 0.08 | 0.65 | 0.08 | 392 |
| 287 | | | | | | | | | | | | | | | | | | |
| Phage- | D | — | — | — | — | D | G | V | — | D | F | I | — | N | 0.06 | 0.65 | 0.08 | 393 |
| 288 | | | | | | | | | | | | | | | | | | |
| Phage- | T | D | — | W | — | D | W | E | Y | I | Y | S | — | S | 0.08 | 0.65 | 0.07 | 394 |
| 289 | | | | | | | | | | | | | | | | | | |
| Phage- | F | — | — | — | — | — | — | E | — | I | T | N | — | I | 0.14 | 0.65 | 0.11 | 395 |
| 290 | | | | | | | | | | | | | | | | | | |
| Phage- | F | D | — | W | — | D | W | E | — | — | F | F | — | H | 0.07 | 0.64 | 0.08 | 396 |
| 291 | | | | | | | | | | | | | | | | | | |
| Phage- | D | F | — | — | — | D | G | — | — | — | — | F | — | P | 0.08 | 0.63 | 0.08 | 397 |
| 292 | | | | | | | | | | | | | | | | | | |
| Phage- | H | N | — | — | — | — | L | — | — | — | L | V | — | D | 0.13 | 0.63 | 0.09 | 398 |
| 293 | | | | | | | | | | | | | | | | | | |
| Phage- | I | — | — | — | — | D | G | A | — | D | Y | T | — | D | 0.07 | 0.63 | 0.07 | 399 |
| 294 | | | | | | | | | | | | | | | | | | |
| Phage- | F | D | — | — | — | — | — | E | F | P | — | I | — | F | 0.08 | 0.62 | 0.08 | 400 |
| 295 | | | | | | | | | | | | | | | | | | |
| Phage- | Y | N | — | — | — | — | L | — | — | — | — | T | — | D | 0.09 | 0.62 | 0.08 | 401 |
| 296 | | | | | | | | | | | | | | | | | | |
| Phage- | F | D | — | — | — | — | L | — | — | — | I | H | — | A | 0.07 | 0.62 | 0.08 | 402 |
| 297 | | | | | | | | | | | | | | | | | | |
| Phage- | D | I | — | — | — | — | V | E | — | Y | F | L | — | F | 0.15 | 0.61 | 0.10 | 403 |
| 298 | | | | | | | | | | | | | | | | | | |
| Phage- | F | D | — | — | — | — | V | — | — | — | L | T | — | F | 0.10 | 0.61 | 0.09 | 404 |
| 299 | | | | | | | | | | | | | | | | | | |
| Phage- | F | D | — | — | — | — | I | E | — | F | H | L | — | F | 0.08 | 0.61 | 0.08 | 405 |
| 300 | | | | | | | | | | | | | | | | | | |
| Phage- | A | — | — | — | — | — | L | — | — | — | I | I | — | D | 0.12 | 0.61 | 0.10 | 406 |
| 301 | | | | | | | | | | | | | | | | | | |
| Phage- | Y | N | — | — | — | — | L | — | — | — | I | T | — | N | 0.09 | 0.61 | 0.10 | 407 |
| 302 | | | | | | | | | | | | | | | | | | |
| Phage- | F | D | — | W | — | D | W | E | — | P | — | D | — | L | 0.08 | 0.61 | 0.07 | 408 |
| 303 | | | | | | | | | | | | | | | | | | |
| Phage- | D | V | — | — | — | — | L | — | — | — | — | L | — | P | 0.08 | 0.60 | 0.08 | 409 |
| 304 | | | | | | | | | | | | | | | | | | |
| Phage- | N | — | — | — | — | — | L | — | — | — | L | P | — | P | 0.09 | 0.60 | 0.08 | 410 |
| 305 | | | | | | | | | | | | | | | | | | |
| Phage- | Y | — | — | W | — | D | W | E | Y | D | I | F | — | S | 0.08 | 0.60 | 0.10 | 411 |
| 306 | | | | | | | | | | | | | | | | | | |
| Phage- | D | D | — | — | — | — | — | — | — | — | T | Y | — | N | 0.08 | 0.60 | 0.09 | 412 |
| 307 | | | | | | | | | | | | | | | | | | |
| Phage- | F | — | — | — | — | — | — | E | — | V | F | H | — | Y | 0.09 | 0.60 | 0.09 | 413 |
| 308 | | | | | | | | | | | | | | | | | | |
| Phage- | T | D | — | W | — | D | W | E | — | Y | F | L | — | D | 0.07 | 0.60 | 0.09 | 414 |
| 309 | | | | | | | | | | | | | | | | | | |
| Phage- | — | — | — | — | — | — | W | E | — | — | Y | L | — | P | 0.09 | 0.60 | 0.09 | 415 |
| 310 | | | | | | | | | | | | | | | | | | |
| Phage- | D | D | — | — | — | N | G | Y | A | T | F | I | — | Y | 0.06 | 0.59 | 0.08 | 416 |
| 311 | | | | | | | | | | | | | | | | | | |
| Phage- | F | L | — | — | — | — | I | E | D | D | T | H | — | Y | 0.16 | 0.59 | 0.38 | 417 |
| 312 | | | | | | | | | | | | | | | | | | |
| Phage- | F | A | — | W | — | D | W | E | — | T | I | P | — | H | 0.08 | 0.59 | 0.08 | 418 |
| 313 | | | | | | | | | | | | | | | | | | |
| Phage- | — | L | — | — | — | — | — | — | — | — | Y | N | — | Y | 0.10 | 0.58 | 0.08 | 419 |
| 314 | | | | | | | | | | | | | | | | | | |
| Phage- | Y | D | — | — | — | — | — | — | — | — | I | S | — | I | 0.09 | 0.58 | 0.09 | 420 |
| 315 | | | | | | | | | | | | | | | | | | |
| Phage- | Y | D | — | — | — | — | — | — | — | — | — | N | — | Y | 0.12 | 0.57 | 0.10 | 421 |
| 316 | | | | | | | | | | | | | | | | | | |
| Phage- | A | I | — | W | — | D | W | E | — | F | — | D | — | Y | 0.10 | 0.57 | 0.09 | 422 |
| 317 | | | | | | | | | | | | | | | | | | |
| Phage- | L | T | — | W | — | V | R | E | — | I | F | A | — | D | 0.07 | 0.57 | 0.08 | 423 |
| 318 | | | | | | | | | | | | | | | | | | |
| Phage- | — | L | — | — | — | — | — | — | — | — | Y | Y | — | N | 0.09 | 0.57 | 0.09 | 424 |
| 319 | | | | | | | | | | | | | | | | | | |
| Phage- | N | V | — | — | — | — | Y | — | — | — | A | P | — | N | 0.07 | 0.56 | 0.08 | 425 |
| 320 | | | | | | | | | | | | | | | | | | |
| Phage- | H | D | — | — | — | — | — | — | — | — | I | S | — | V | 0.11 | 0.55 | 0.09 | 426 |
| 321 | | | | | | | | | | | | | | | | | | |
| Phage- | F | D | — | — | — | — | L | — | — | T | — | D | — | N | 0.12 | 0.55 | 0.41 | 427 |
| 322 | | | | | | | | | | | | | | | | | | |
| Phage- | Y | F | — | — | — | — | V | E | — | H | F | Y | — | Y | 0.09 | 0.55 | 0.08 | 428 |
| 323 | | | | | | | | | | | | | | | | | | |
| Phage- | D | — | — | — | — | — | L | — | — | — | I | I | — | H | 0.09 | 0.54 | 0.09 | 429 |
| 324 | | | | | | | | | | | | | | | | | | |
| Phage- | D | D | — | — | — | — | V | P | — | D | I | T | — | Y | 0.11 | 0.54 | 0.08 | 430 |
| 325 | | | | | | | | | | | | | | | | | | |
| Phage- | D | N | — | — | — | — | L | — | — | — | — | V | — | D | 0.10 | 0.54 | 0.08 | 431 |
| 326 | | | | | | | | | | | | | | | | | | |
| Phage- | — | H | — | W | — | D | W | E | P | N | Y | V | — | D | 0.10 | 0.54 | 0.08 | 432 |
| 327 | | | | | | | | | | | | | | | | | | |
| Phage- | D | — | — | — | — | — | L | — | — | — | L | F | — | L | 0.09 | 0.53 | 0.09 | 433 |
| 328 | | | | | | | | | | | | | | | | | | |
| Phage- | D | D | — | — | — | — | L | — | — | — | — | V | — | A | 0.08 | 0.53 | 0.11 | 434 |
| 329 | | | | | | | | | | | | | | | | | | |
| Phage- | A | A | — | — | — | — | L | — | — | — | I | V | — | D | 0.13 | 0.53 | 0.09 | 435 |
| 330 | | | | | | | | | | | | | | | | | | |
| Phage- | D | F | — | — | — | — | — | E | — | I | N | N | — | F | 0.14 | 0.52 | 0.48 | 436 |
| 331 | | | | | | | | | | | | | | | | | | |
| Phage- | Y | — | — | — | — | — | — | — | — | — | N | A | — | Y | 0.08 | 0.52 | 0.07 | 437 |
| 332 | | | | | | | | | | | | | | | | | | |
| Phage- | — | L | — | — | — | — | — | — | — | — | N | S | — | Y | 0.10 | 0.52 | 0.11 | 438 |
| 333 | | | | | | | | | | | | | | | | | | |
| Phage- | Y | D | — | — | — | — | — | — | — | I | T | D | — | D | 0.09 | 0.52 | 0.09 | 439 |
| 334 | | | | | | | | | | | | | | | | | | |
| Phage- | D | S | — | — | — | — | — | E | F | Y | Y | V | — | F | 0.11 | 0.52 | 0.14 | 440 |
| 335 | | | | | | | | | | | | | | | | | | |
| Phage- | Y | I | — | — | — | — | L | — | — | — | L | I | — | H | 0.10 | 0.52 | 0.08 | 441 |
| 336 | | | | | | | | | | | | | | | | | | |
| Phage- | F | D | — | — | — | — | V | E | — | D | Y | F | — | Y | 0.11 | 0.52 | 0.10 | 442 |
| 337 | | | | | | | | | | | | | | | | | | |
| Phage- | F | D | — | — | — | — | Y | — | — | — | L | Y | — | F | 0.08 | 0.52 | 0.07 | 443 |
| 338 | | | | | | | | | | | | | | | | | | |
| Phage- | — | L | — | — | — | D | G | — | — | Y | S | F | — | H | 0.10 | 0.51 | 0.10 | 444 |
| 339 | | | | | | | | | | | | | | | | | | |
| Phage- | I | P | — | — | — | — | M | — | — | — | — | V | — | N | 0.12 | 0.51 | 0.09 | 445 |
| 340 | | | | | | | | | | | | | | | | | | |
| Phage- | I | I | — | — | — | D | G | Y | — | D | F | T | — | D | 0.12 | 0.51 | 0.09 | 446 |
| 341 | | | | | | | | | | | | | | | | | | |
| Phage- | I | F | — | — | — | — | L | — | — | — | I | I | — | Y | 0.11 | 0.51 | 0.08 | 447 |
| 342 | | | | | | | | | | | | | | | | | | |
| Phage- | — | D | — | — | — | — | — | — | — | — | Y | D | — | Y | 0.18 | 0.51 | 0.18 | 448 |
| 343 | | | | | | | | | | | | | | | | | | |
| Phage- | L | S | — | — | — | — | M | — | — | — | L | Y | — | D | 0.12 | 0.51 | 0.08 | 449 |
| 344 | | | | | | | | | | | | | | | | | | |
| Phage- | Y | D | — | W | — | D | W | E | Y | N | I | D | — | H | 0.08 | 0.51 | 0.09 | 450 |
| 345 | | | | | | | | | | | | | | | | | | |
| Phage- | N | H | — | — | — | D | G | — | — | T | I | V | — | F | 0.09 | 0.51 | 0.08 | 451 |
| 346 | | | | | | | | | | | | | | | | | | |
| Phage- | N | F | — | — | — | — | L | — | — | — | I | P | — | H | 0.11 | 0.50 | 0.12 | 452 |
| 347 | | | | | | | | | | | | | | | | | | |
| Phage- | F | H | — | — | — | — | I | E | — | Y | A | L | — | D | 0.08 | 0.50 | 0.09 | 453 |
| 348 | | | | | | | | | | | | | | | | | | |
| Phage- | — | — | — | — | — | — | V | E | D | Y | N | L | — | Y | 0.07 | 0.50 | 0.08 | 454 |
| 349 | | | | | | | | | | | | | | | | | | |
| Phage- | — | — | — | — | — | D | G | — | — | L | A | N | — | Y | 0.09 | 0.50 | 0.08 | 455 |
| 350 | | | | | | | | | | | | | | | | | | |
| Phage- | L | I | — | — | — | V | I | A | — | D | L | P | — | N | 0.17 | 0.50 | 0.26 | 456 |
| 351 | | | | | | | | | | | | | | | | | | |
| Phage- | D | I | — | — | — | — | I | P | — | D | — | S | — | D | 0.10 | 0.50 | 0.08 | 457 |
| 352 | | | | | | | | | | | | | | | | | | |
| Phage- | I | — | — | — | — | — | W | E | — | A | D | Y | — | D | 0.11 | 0.50 | 0.45 | 458 |
| 353 | | | | | | | | | | | | | | | | | | |
| Phage- | I | D | — | W | — | D | W | E | D | D | S | I | — | Y | 0.10 | 0.50 | 0.10 | 459 |
| 354 | | | | | | | | | | | | | | | | | | |
| Phage- | — | L | — | — | — | — | V | E | D | F | T | L | — | D | 0.09 | 0.50 | 0.11 | 460 |
| 355 | | | | | | | | | | | | | | | | | | |
| Phage- | L | — | — | — | — | V | I | E | — | I | Y | Y | — | Y | 0.09 | 0.49 | 0.09 | 461 |
| 356 | | | | | | | | | | | | | | | | | | |
| Phage- | F | F | — | — | — | — | — | E | V | H | S | D | — | N | 0.14 | 0.49 | 0.38 | 462 |
| 357 | | | | | | | | | | | | | | | | | | |
| Phage- | N | D | — | — | — | — | V | E | L | V | S | D | — | N | 0.10 | 0.49 | 0.08 | 463 |
| 358 | | | | | | | | | | | | | | | | | | |
| Phage- | D | L | — | — | — | — | L | — | — | — | T | V | — | D | 0.09 | 0.49 | 0.08 | 464 |
| 359 | | | | | | | | | | | | | | | | | | |
| Phage- | I | P | — | — | — | — | V | E | D | Y | N | L | — | N | 0.08 | 0.49 | 0.08 | 465 |
| 360 | | | | | | | | | | | | | | | | | | |
| Phage- | Y | — | — | — | — | — | L | E | W | P | — | V | — | N | 0.10 | 0.49 | 0.10 | 466 |
| 361 | | | | | | | | | | | | | | | | | | |
| Phage- | Y | D | — | — | — | — | L | — | — | — | — | I | — | N | 0.08 | 0.49 | 0.10 | 467 |
| 362 | | | | | | | | | | | | | | | | | | |
| Phage- | — | — | — | — | — | D | G | — | — | — | F | D | — | A | 0.08 | 0.49 | 0.08 | 468 |
| 363 | | | | | | | | | | | | | | | | | | |
| Phage- | N | D | — | — | — | — | W | E | D | T | Y | F | — | L | 0.08 | 0.49 | 0.10 | 469 |
| 364 | | | | | | | | | | | | | | | | | | |
| Phage- | P | — | — | — | — | — | M | E | — | L | S | N | — | S | 0.13 | 0.48 | 0.16 | 470 |
| 365 | | | | | | | | | | | | | | | | | | |
| Phage- | D | D | — | — | — | — | — | E | V | I | S | D | — | Y | 0.15 | 0.48 | 0.10 | 471 |
| 366 | | | | | | | | | | | | | | | | | | |
| Phage- | D | L | — | — | — | — | — | P | — | D | — | P | — | D | 0.08 | 0.48 | 0.08 | 472 |
| 367 | | | | | | | | | | | | | | | | | | |
| Phage- | I | — | — | — | — | — | — | — | — | — | F | V | — | Y | 0.11 | 0.48 | 0.10 | 473 |
| 368 | | | | | | | | | | | | | | | | | | |
| Phage- | A | — | — | — | — | — | Y | E | V | F | A | D | — | N | 0.10 | 0.48 | 0.11 | 474 |
| 369 | | | | | | | | | | | | | | | | | | |
| Phage- | I | D | — | — | — | — | Y | — | — | — | — | D | — | L | 0.09 | 0.48 | 0.09 | 475 |
| 370 | | | | | | | | | | | | | | | | | | |
| Phage- | H | I | — | W | — | D | W | E | — | F | H | D | — | N | 0.07 | 0.48 | 0.08 | 476 |
| 371 | | | | | | | | | | | | | | | | | | |
| Phage- | Y | D | — | — | — | — | L | — | — | T | I | T | — | L | 0.08 | 0.48 | 0.08 | 477 |
| 372 | | | | | | | | | | | | | | | | | | |
| Phage- | Y | L | — | — | — | — | L | — | — | T | I | L | — | N | 0.09 | 0.48 | 0.08 | 478 |
| 373 | | | | | | | | | | | | | | | | | | |
| Phage- | F | F | — | — | — | — | — | E | — | A | F | L | — | F | 0.10 | 0.48 | 0.14 | 479 |
| 374 | | | | | | | | | | | | | | | | | | |
| Phage- | I | L | — | — | — | — | L | — | — | — | F | T | — | A | 0.07 | 0.47 | 0.08 | 480 |
| 375 | | | | | | | | | | | | | | | | | | |
| Phage- | F | H | — | — | — | — | V | E | L | Y | T | D | — | N | 0.09 | 0.47 | 0.08 | 481 |
| 376 | | | | | | | | | | | | | | | | | | |
| Phage- | N | L | — | — | — | — | V | E | — | Y | N | F | — | Y | 0.08 | 0.47 | 0.08 | 482 |
| 377 | | | | | | | | | | | | | | | | | | |
| Phage- | F | D | — | — | — | — | V | E | — | T | Y | Y | — | F | 0.21 | 0.47 | 0.09 | 483 |
| 378 | | | | | | | | | | | | | | | | | | |
| Phage- | — | F | — | — | — | — | — | E | — | D | H | Y | — | Y | 0.12 | 0.47 | 0.37 | 484 |
| 379 | | | | | | | | | | | | | | | | | | |
| Phage- | A | I | — | — | — | — | W | E | V | V | A | D | — | N | 0.11 | 0.47 | 0.11 | 485 |
| 380 | | | | | | | | | | | | | | | | | | |
| Phage- | F | I | — | W | — | D | W | E | — | D | N | Y | — | N | 0.12 | 0.47 | 0.25 | 486 |
| 381 | | | | | | | | | | | | | | | | | | |
| Phage- | I | — | — | — | — | — | — | — | — | — | F | I | — | D | 0.08 | 0.47 | 0.10 | 487 |
| 382 | | | | | | | | | | | | | | | | | | |
| Phage- | N | L | — | — | — | — | V | E | D | V | Y | D | — | H | 0.12 | 0.47 | 0.43 | 488 |
| 383 | | | | | | | | | | | | | | | | | | |
| Phage- | H | — | — | — | — | — | V | E | — | Y | H | N | — | N | 0.09 | 0.47 | 0.09 | 489 |
| 384 | | | | | | | | | | | | | | | | | | |
| Phage- | D | I | — | — | — | — | Y | — | — | — | Y | S | — | T | 0.09 | 0.47 | 0.09 | 490 |
| 385 | | | | | | | | | | | | | | | | | | |
| Phage- | D | — | — | — | — | V | L | — | — | T | L | I | — | A | 0.13 | 0.46 | 0.10 | 491 |
| 386 | | | | | | | | | | | | | | | | | | |
| Phage- | I | A | — | — | — | — | M | P | — | D | I | D | — | Y | 0.12 | 0.46 | 0.09 | 492 |
| 387 | | | | | | | | | | | | | | | | | | |
| Phage- | I | D | — | — | — | — | L | — | — | — | I | F | — | D | 0.10 | 0.46 | 0.10 | 493 |
| 388 | | | | | | | | | | | | | | | | | | |
| Phage- | Y | F | — | — | — | D | V | E | — | D | F | A | — | D | 0.11 | 0.46 | 0.11 | 494 |
| 389 | | | | | | | | | | | | | | | | | | |
| Phage- | Y | N | — | — | — | — | W | E | Y | A | I | L | — | D | 0.12 | 0.46 | 0.34 | 495 |
| 390 | | | | | | | | | | | | | | | | | | |
| Phage- | I | — | — | — | — | — | V | E | D | Y | I | V | — | N | 0.14 | 0.46 | 0.22 | 496 |
| 391 | | | | | | | | | | | | | | | | | | |
| Phage- | Y | D | — | — | — | — | I | — | — | — | T | P | — | A | 0.07 | 0.46 | 0.07 | 497 |
| 392 | | | | | | | | | | | | | | | | | | |
| Phage- | D | T | — | W | — | D | W | E | H | I | Y | A | — | D | 0.09 | 0.46 | 0.09 | 498 |
| 393 | | | | | | | | | | | | | | | | | | |
| Phage- | D | I | — | — | — | — | M | — | — | — | — | T | — | N | 0.13 | 0.45 | 0.12 | 499 |
| 394 | | | | | | | | | | | | | | | | | | |
| Phage- | Y | D | — | W | — | D | W | E | R | Y | F | P | — | I | 0.10 | 0.45 | 0.09 | 500 |
| 395 | | | | | | | | | | | | | | | | | | |
| Phage- | H | L | — | — | — | — | L | — | — | — | — | A | — | S | 0.13 | 0.45 | 0.11 | 501 |
| 396 | | | | | | | | | | | | | | | | | | |
| Phage- | Y | D | — | — | — | D | G | — | — | T | T | I | — | A | 0.09 | 0.45 | 0.09 | 502 |
| 397 | | | | | | | | | | | | | | | | | | |
| Phage- | Y | — | — | — | — | — | Y | E | D | V | L | D | — | F | 0.07 | 0.45 | 0.08 | 503 |
| 398 | | | | | | | | | | | | | | | | | | |
| Phage- | D | F | — | — | — | — | M | — | — | T | I | S | — | D | 0.14 | 0.45 | 0.10 | 504 |
| 399 | | | | | | | | | | | | | | | | | | |
| Phage- | I | L | — | — | — | — | L | — | — | — | L | V | — | D | 0.08 | 0.44 | 0.07 | 505 |
| 400 | | | | | | | | | | | | | | | | | | |
| Phage- | L | I | — | — | — | — | W | E | V | I | T | N | — | D | 0.12 | 0.44 | 0.40 | 506 |
| 401 | | | | | | | | | | | | | | | | | | |
| Phage- | Y | D | — | — | — | — | — | — | — | Y | F | — | — | P | 0.09 | 0.44 | 0.09 | 507 |
| 402 | | | | | | | | | | | | | | | | | | |
| Phage- | A | L | — | — | — | — | V | E | V | Y | D | V | — | V | 0.08 | 0.44 | 0.08 | 508 |
| 403 | | | | | | | | | | | | | | | | | | |
| Phage- | Y | H | — | W | — | D | W | E | D | V | N | F | — | Y | 0.10 | 0.44 | 0.09 | 509 |
| 404 | | | | | | | | | | | | | | | | | | |
| Phage- | F | L | — | — | — | M | G | G | L | T | F | Y | — | Y | 0.09 | 0.44 | 0.08 | 510 |
| 405 | | | | | | | | | | | | | | | | | | |
| Phage- | I | I | — | — | — | — | — | — | — | Y | — | — | — | F | 0.09 | 0.43 | 0.19 | 511 |
| 406 | | | | | | | | | | | | | | | | | | |
| Phage- | F | F | — | — | — | — | M | — | — | — | — | H | — | F | 0.11 | 0.43 | 0.09 | 512 |
| 407 | | | | | | | | | | | | | | | | | | |
| Phage- | A | F | — | — | — | — | — | — | — | — | L | F | — | A | 0.09 | 0.43 | 0.09 | 513 |
| 408 | | | | | | | | | | | | | | | | | | |
| Phage- | N | — | — | — | — | D | G | — | — | T | N | I | — | D | 0.08 | 0.43 | 0.08 | 514 |
| 409 | | | | | | | | | | | | | | | | | | |
| Phage- | Y | L | — | — | — | — | W | E | W | V | H | N | — | L | 0.13 | 0.43 | 0.09 | 515 |
| 410 | | | | | | | | | | | | | | | | | | |
| Phage- | A | T | — | — | — | D | G | — | — | — | H | I | — | A | 0.08 | 0.43 | 0.09 | 516 |
| 411 | | | | | | | | | | | | | | | | | | |
| Phage- | — | — | — | — | — | — | V | E | V | L | D | Y | — | D | 0.07 | 0.42 | 0.08 | 517 |
| 412 | | | | | | | | | | | | | | | | | | |
| Phage- | I | H | — | — | — | — | W | E | F | Y | T | D | — | D | 0.08 | 0.42 | 0.08 | 518 |
| 413 | | | | | | | | | | | | | | | | | | |
| Phage- | D | D | — | — | — | — | L | — | — | T | — | A | — | D | 0.13 | 0.42 | 0.32 | 519 |
| 414 | | | | | | | | | | | | | | | | | | |
| Phage- | Y | L | — | — | — | — | — | — | — | — | I | D | — | N | 0.11 | 0.42 | 0.17 | 520 |
| 415 | | | | | | | | | | | | | | | | | | |
| Phage- | L | L | — | — | — | — | V | E | D | V | F | A | — | Y | 0.09 | 0.42 | 0.09 | 521 |
| 416 | | | | | | | | | | | | | | | | | | |
| Phage- | Y | D | — | — | — | — | L | — | — | — | L | T | — | D | 0.08 | 0.42 | 0.08 | 522 |
| 417 | | | | | | | | | | | | | | | | | | |
| Phage- | F | D | — | — | — | — | L | — | — | T | — | N | — | Y | 0.09 | 0.41 | 0.09 | 523 |
| 418 | | | | | | | | | | | | | | | | | | |
| Phage- | F | A | — | W | — | D | W | E | — | I | N | D | — | H | 0.08 | 0.41 | 0.09 | 524 |
| 419 | | | | | | | | | | | | | | | | | | |
| Phage- | Y | — | — | — | — | — | Y | E | — | D | I | Y | — | N | 0.09 | 0.41 | 0.09 | 525 |
| 420 | | | | | | | | | | | | | | | | | | |
| Phage- | N | V | — | — | — | — | V | E | D | Y | T | F | — | Y | 0.09 | 0.40 | 0.08 | 526 |
| 421 | | | | | | | | | | | | | | | | | | |
| Phage- | A | — | — | — | — | — | L | E | — | Y | D | F | — | T | 0.12 | 0.40 | 0.08 | 527 |
| 422 | | | | | | | | | | | | | | | | | | |
| Phage- | F | D | — | — | — | — | I | — | — | — | T | I | — | T | 0.08 | 0.40 | 0.09 | 528 |
| 423 | | | | | | | | | | | | | | | | | | |
| Phage- | N | L | — | — | — | — | L | — | — | T | L | V | — | A | 0.10 | 0.40 | 0.09 | 529 |
| 424 | | | | | | | | | | | | | | | | | | |
| Phage- | Y | S | — | W | — | D | W | E | — | Y | L | A | — | N | 0.08 | 0.40 | 0.08 | 530 |
| 425 | | | | | | | | | | | | | | | | | | |
| Phage- | G | I | — | — | — | — | V | E | D | Y | N | Y | — | D | 0.09 | 0.40 | 0.10 | 531 |
| 426 | | | | | | | | | | | | | | | | | | |
| Phage- | F | F | — | — | — | — | L | — | — | — | — | N | — | H | 0.07 | 0.40 | 0.07 | 532 |
| 427 | | | | | | | | | | | | | | | | | | |
| Phage- | Y | — | — | — | — | — | Y | E | — | D | F | Y | — | F | 0.11 | 0.40 | 0.09 | 533 |
| 428 | | | | | | | | | | | | | | | | | | |
| Phage- | L | — | — | — | — | — | Y | — | — | — | T | D | — | Y | 0.11 | 0.40 | 0.10 | 534 |
| 429 | | | | | | | | | | | | | | | | | | |
| Phage- | D | — | — | — | — | — | V | E | — | D | F | L | — | Y | 0.10 | 0.40 | 0.08 | 535 |
| 430 | | | | | | | | | | | | | | | | | | |
| Phage- | T | L | — | — | — | — | V | E | L | Y | I | F | — | D | 0.08 | 0.40 | 0.09 | 536 |
| 431 | | | | | | | | | | | | | | | | | | |
| Phage- | F | — | — | — | — | — | — | E | Q | I | A | D | — | Y | 0.11 | 0.40 | 0.09 | 537 |
| 432 | | | | | | | | | | | | | | | | | | |
| Phage- | D | D | — | — | — | — | V | E | — | Y | H | L | — | D | 0.11 | 0.40 | 0.18 | 538 |
| 433 | | | | | | | | | | | | | | | | | | |
| Phage- | D | — | — | — | — | — | L | E | D | V | T | L | — | H | 0.13 | 0.39 | 0.09 | 539 |
| 434 | | | | | | | | | | | | | | | | | | |
| Phage- | — | L | — | — | — | — | V | E | D | V | N | L | — | Y | 0.09 | 0.39 | 0.08 | 540 |
| 435 | | | | | | | | | | | | | | | | | | |
| Phage- | D | T | — | — | — | — | L | — | — | T | I | D | — | Y | 0.09 | 0.39 | 0.09 | 541 |
| 436 | | | | | | | | | | | | | | | | | | |
| Phage- | T | — | — | — | — | — | V | E | — | D | I | N | — | Y | 0.08 | 0.39 | 0.08 | 542 |
| 437 | | | | | | | | | | | | | | | | | | |
| Phage- | I | V | — | W | — | D | W | E | — | Y | P | N | — | D | 0.08 | 0.39 | 0.08 | 543 |
| 438 | | | | | | | | | | | | | | | | | | |
| Phage- | S | D | — | — | — | — | L | — | — | — | I | I | — | T | 0.11 | 0.39 | 0.10 | 544 |
| 439 | | | | | | | | | | | | | | | | | | |
| Phage- | Y | D | — | — | — | — | L | P | — | D | Y | D | — | N | 0.15 | 0.39 | 0.10 | 545 |
| 440 | | | | | | | | | | | | | | | | | | |
| Phage- | N | L | — | W | — | D | W | E | — | Y | Y | A | — | D | 0.12 | 0.39 | 0.17 | 546 |
| 441 | | | | | | | | | | | | | | | | | | |
| Phage- | D | D | — | — | — | — | L | — | — | — | L | P | — | H | 0.10 | 0.39 | 0.08 | 547 |
| 442 | | | | | | | | | | | | | | | | | | |
| Phage- | S | L | — | — | — | D | G | Q | — | D | Y | T | — | F | 0.08 | 0.39 | 0.08 | 548 |
| 443 | | | | | | | | | | | | | | | | | | |
| Phage- | L | I | — | W | — | D | W | E | — | Y | N | F | — | T | 0.13 | 0.39 | 0.11 | 549 |
| 444 | | | | | | | | | | | | | | | | | | |
| Phage- | F | H | — | — | — | D | G | — | — | T | — | P | — | I | 0.08 | 0.39 | 0.08 | 550 |
| 445 | | | | | | | | | | | | | | | | | | |
| Phage- | F | D | — | — | — | — | W | E | W | I | Y | D | — | F | 0.08 | 0.38 | 0.08 | 551 |
| 446 | | | | | | | | | | | | | | | | | | |
| Phage- | I | — | — | — | — | — | W | I | I | — | L | D | — | D | 0.08 | 0.38 | 0.09 | 552 |
| 447 | | | | | | | | | | | | | | | | | | |
| Phage- | L | I | — | — | — | — | I | — | — | — | A | S | — | N | 0.10 | 0.38 | 0.11 | 553 |
| 448 | | | | | | | | | | | | | | | | | | |
| Phage- | T | S | — | W | V | D | W | E | — | F | S | D | — | I | 0.11 | 0.38 | 0.34 | 554 |
| 449 | | | | | | | | | | | | | | | | | | |
| Phage- | Y | — | — | — | — | — | V | E | — | D | Y | V | — | D | 0.10 | 0.38 | 0.08 | 555 |
| 450 | | | | | | | | | | | | | | | | | | |
| Phage- | D | D | — | — | — | — | Q | E | F | T | Y | A | — | T | 0.09 | 0.38 | 0.08 | 556 |
| 451 | | | | | | | | | | | | | | | | | | |
| Phage- | A | D | — | W | — | D | W | E | — | Y | A | D | — | Y | 0.11 | 0.38 | 0.10 | 557 |
| 452 | | | | | | | | | | | | | | | | | | |
| Phage- | Y | — | — | — | — | — | — | — | — | — | I | H | — | I | 0.08 | 0.38 | 0.07 | 558 |
| 453 | | | | | | | | | | | | | | | | | | |
| Phage- | S | E | — | W | — | D | W | E | P | F | F | D | — | N | 0.08 | 0.37 | 0.09 | 559 |
| 454 | | | | | | | | | | | | | | | | | | |
| Phage- | Y | H | — | — | — | — | M | — | — | — | L | I | — | T | 0.07 | 0.37 | 0.07 | 560 |
| 455 | | | | | | | | | | | | | | | | | | |
| Phage- | S | D | — | W | — | D | W | E | D | A | Y | F | — | I | 0.09 | 0.37 | 0.07 | 561 |
| 456 | | | | | | | | | | | | | | | | | | |
| Phage- | — | D | — | — | — | — | V | E | — | Y | Y | H | — | D | 0.08 | 0.37 | 0.08 | 562 |
| 457 | | | | | | | | | | | | | | | | | | |
| Phage- | D | N | — | — | — | — | Y | — | — | — | I | A | — | N | 0.10 | 0.37 | 0.10 | 563 |
| 458 | | | | | | | | | | | | | | | | | | |
| Phage- | L | — | — | — | — | V | — | E | F | Y | D | Y | — | Y | 0.08 | 0.37 | 0.10 | 564 |
| 459 | | | | | | | | | | | | | | | | | | |
| Phage- | Y | T | — | — | — | — | M | — | — | — | — | T | — | I | 0.09 | 0.37 | 0.08 | 565 |
| 460 | | | | | | | | | | | | | | | | | | |
| Phage- | N | F | — | W | — | D | W | E | V | N | S | F | — | D | 0.09 | 0.37 | 0.08 | 566 |
| 461 | | | | | | | | | | | | | | | | | | |
| Phage- | N | A | — | W | — | D | W | E | Y | I | D | F | — | N | 0.12 | 0.36 | 0.09 | 567 |
| 462 | | | | | | | | | | | | | | | | | | |
| Phage- | Y | N | — | — | — | — | M | — | — | — | I | F | — | S | 0.09 | 0.36 | 0.07 | 568 |
| 463 | | | | | | | | | | | | | | | | | | |
| Phage- | L | D | — | — | — | — | L | — | — | — | I | T | — | Y | 0.08 | 0.36 | 0.09 | 569 |
| 464 | | | | | | | | | | | | | | | | | | |
| Phage- | H | — | — | — | — | — | — | — | — | — | I | N | — | D | 0.11 | 0.36 | 0.08 | 570 |
| 465 | | | | | | | | | | | | | | | | | | |
| Phage- | I | I | — | — | — | — | L | P | — | D | Y | V | — | T | 0.08 | 0.36 | 0.08 | 571 |
| 466 | | | | | | | | | | | | | | | | | | |
| Phage- | D | I | — | — | — | — | — | — | — | — | I | D | — | S | 0.08 | 0.36 | 0.08 | 572 |
| 467 | | | | | | | | | | | | | | | | | | |
| Phage- | P | — | — | — | — | — | — | — | — | — | — | L | — | F | 0.10 | 0.36 | 0.09 | 573 |
| 468 | | | | | | | | | | | | | | | | | | |
| Phage- | — | F | — | — | — | — | — | — | — | — | — | D | — | Y | 0.07 | 0.36 | 0.08 | 574 |
| 469 | | | | | | | | | | | | | | | | | | |
| Phage- | Y | D | — | W | — | D | W | E | — | A | L | P | — | A | 0.08 | 0.36 | 0.08 | 575 |
| 470 | | | | | | | | | | | | | | | | | | |
| Phage- | D | D | — | W | — | D | W | E | D | Y | — | F | — | F | 0.10 | 0.36 | 0.10 | 576 |
| 471 | | | | | | | | | | | | | | | | | | |
| Phage- | H | F | — | — | — | — | W | E | L | F | S | D | — | Y | 0.11 | 0.36 | 0.11 | 577 |
| 472 | | | | | | | | | | | | | | | | | | |
| Phage- | I | T | — | W | — | D | W | E | V | N | F | P | — | Y | 0.07 | 0.35 | 0.07 | 578 |
| 473 | | | | | | | | | | | | | | | | | | |
| Phage- | P | D | — | — | — | — | L | — | — | — | I | T | — | N | 0.20 | 0.35 | 0.16 | 579 |
| 474 | | | | | | | | | | | | | | | | | | |
| Phage- | N | L | — | W | — | D | W | E | A | F | F | P | — | Y | 0.08 | 0.35 | 0.07 | 580 |
| 475 | | | | | | | | | | | | | | | | | | |
| Phage- | F | — | — | — | — | — | — | E | Y | I | R | D | — | Y | 0.08 | 0.35 | 0.07 | 581 |
| 476 | | | | | | | | | | | | | | | | | | |
| Phage- | F | F | — | — | — | — | — | — | — | — | I | I | — | D | 0.09 | 0.35 | 0.10 | 582 |
| 477 | | | | | | | | | | | | | | | | | | |
| Phage- | — | L | — | — | — | K | G | G | P | T | Y | N | — | S | 0.08 | 0.35 | 0.10 | 583 |
| 478 | | | | | | | | | | | | | | | | | | |
| Phage- | L | A | — | W | — | V | W | E | — | P | G | H | — | D | 0.11 | 0.35 | 0.10 | 584 |
| 479 | | | | | | | | | | | | | | | | | | |
| Phage- | D | — | — | — | — | — | V | E | D | V | N | D | — | Y | 0.07 | 0.35 | 0.08 | 585 |
| 480 | | | | | | | | | | | | | | | | | | |
| Phage- | D | — | — | — | — | — | — | E | — | A | H | Y | — | N | 0.08 | 0.35 | 0.07 | 586 |
| 481 | | | | | | | | | | | | | | | | | | |
| Phage- | — | L | — | — | — | — | L | — | — | T | L | T | — | I | 0.08 | 0.35 | 0.07 | 587 |
| 482 | | | | | | | | | | | | | | | | | | |
| Phage- | Y | — | — | — | — | — | I | E | D | Y | N | L | — | N | 0.10 | 0.34 | 0.09 | 588 |
| 483 | | | | | | | | | | | | | | | | | | |
| Phage- | Y | I | — | — | — | — | V | E | — | Y | Y | N | — | F | 0.13 | 0.34 | 0.14 | 589 |
| 484 | | | | | | | | | | | | | | | | | | |
| Phage- | D | I | — | — | — | — | L | — | — | — | I | F | — | F | 0.08 | 0.34 | 0.09 | 590 |
| 485 | | | | | | | | | | | | | | | | | | |
| Phage- | D | I | — | — | — | — | V | E | — | D | Y | L | — | Y | 0.07 | 0.34 | 0.08 | 591 |
| 486 | | | | | | | | | | | | | | | | | | |
| Phage- | T | L | — | — | — | — | — | E | — | D | A | P | — | I | 0.10 | 0.34 | 0.08 | 592 |
| 487 | | | | | | | | | | | | | | | | | | |
| Phage- | N | — | — | W | — | D | W | E | Y | I | N | S | — | V | 0.14 | 0.34 | 0.09 | 593 |
| 488 | | | | | | | | | | | | | | | | | | |
| Phage- | N | D | — | — | — | — | V | E | — | Y | Y | Y | — | T | 0.07 | 0.34 | 0.09 | 594 |
| 489 | | | | | | | | | | | | | | | | | | |
| Phage- | I | T | — | — | — | — | M | — | — | — | I | D | — | N | 0.08 | 0.34 | 0.08 | 595 |
| 490 | | | | | | | | | | | | | | | | | | |
| Phage- | Y | — | — | — | — | — | M | A | — | D | L | I | — | D | 0.10 | 0.34 | 0.29 | 596 |
| 491 | | | | | | | | | | | | | | | | | | |
| Phage- | I | D | — | — | — | — | L | — | — | — | I | V | — | T | 0.11 | 0.33 | 0.09 | 597 |
| 492 | | | | | | | | | | | | | | | | | | |
| Phage- | H | T | — | W | — | D | W | E | W | D | — | Y | — | D | 0.08 | 0.33 | 0.07 | 598 |
| 493 | | | | | | | | | | | | | | | | | | |
| Phage- | I | H | — | — | — | — | W | E | L | I | D | D | — | L | 0.08 | 0.33 | 0.10 | 599 |
| 494 | | | | | | | | | | | | | | | | | | |
| Phage- | Y | T | — | — | — | — | L | — | — | — | T | T | — | T | 0.08 | 0.33 | 0.08 | 600 |
| 495 | | | | | | | | | | | | | | | | | | |
| Phage- | F | H | — | — | — | — | V | E | — | T | — | Y | — | F | 0.11 | 0.33 | 0.09 | 601 |
| 496 | | | | | | | | | | | | | | | | | | |
| Phage- | D | — | — | — | — | — | L | — | — | — | L | I | — | N | 0.07 | 0.33 | 0.08 | 602 |
| 497 | | | | | | | | | | | | | | | | | | |
| Phage- | I | — | — | — | — | — | — | — | — | — | D | Y | — | I | 0.08 | 0.33 | 0.10 | 603 |
| 498 | | | | | | | | | | | | | | | | | | |
| Phage- | D | L | — | — | — | — | I | E | — | D | L | V | — | T | 0.09 | 0.33 | 0.09 | 604 |
| 499 | | | | | | | | | | | | | | | | | | |
| Phage- | N | I | — | — | — | — | L | Q | — | D | I | V | — | P | 0.09 | 0.33 | 0.09 | 605 |
| 500 | | | | | | | | | | | | | | | | | | |
| Phage- | N | N | — | — | — | — | M | — | — | — | I | T | — | Y | 0.08 | 0.33 | 0.08 | 606 |
| 501 | | | | | | | | | | | | | | | | | | |
| Phage- | H | T | — | — | — | — | L | — | — | — | I | V | — | V | 0.08 | 0.33 | 0.08 | 607 |
| 502 | | | | | | | | | | | | | | | | | | |
| Phage- | Y | — | — | — | — | — | I | E | D | I | L | V | — | T | 0.12 | 0.33 | 0.23 | 608 |
| 503 | | | | | | | | | | | | | | | | | | |
| Phage- | N | T | — | — | — | — | — | E | F | V | H | L | — | P | 0.07 | 0.33 | 0.11 | 609 |
| 504 | | | | | | | | | | | | | | | | | | |
| Phage- | D | I | — | — | — | — | M | — | — | — | T | V | — | D | 0.10 | 0.33 | 0.09 | 610 |
| 505 | | | | | | | | | | | | | | | | | | |
| Phage- | A | I | — | — | — | — | V | E | I | V | N | Y | — | Y | 0.09 | 0.32 | 0.07 | 611 |
| 506 | | | | | | | | | | | | | | | | | | |
| Phage- | H | L | — | — | — | — | V | E | D | P | T | A | — | V | 0.10 | 0.32 | 0.28 | 612 |
| 507 | | | | | | | | | | | | | | | | | | |
| Phage- | A | D | — | — | — | — | L | — | — | — | I | S | — | T | 0.10 | 0.32 | 0.09 | 613 |
| 508 | | | | | | | | | | | | | | | | | | |
| Phage- | F | D | — | — | — | — | L | — | — | — | — | I | — | D | 0.07 | 0.32 | 0.09 | 614 |
| 509 | | | | | | | | | | | | | | | | | | |
| Phage- | D | V | — | — | — | — | — | — | — | — | I | D | — | N | 0.10 | 0.32 | 0.08 | 615 |
| 510 | | | | | | | | | | | | | | | | | | |
| Phage- | Y | L | — | — | — | — | V | E | — | I | S | I | — | F | 0.08 | 0.32 | 0.07 | 616 |
| 511 | | | | | | | | | | | | | | | | | | |
| Phage- | S | A | — | — | — | — | — | — | — | — | L | H | — | V | 0.10 | 0.31 | 0.30 | 617 |
| 512 | | | | | | | | | | | | | | | | | | |
| Phage- | H | L | — | W | — | D | W | E | — | D | S | A | — | N | 0.08 | 0.31 | 0.08 | 618 |
| 513 | | | | | | | | | | | | | | | | | | |
| Phage- | H | T | — | W | — | D | W | E | Y | D | Y | D | — | F | 0.10 | 0.31 | 0.08 | 619 |
| 514 | | | | | | | | | | | | | | | | | | |
| Phage- | Y | D | — | — | — | — | W | E | — | V | A | L | — | N | 0.10 | 0.31 | 0.10 | 620 |
| 515 | | | | | | | | | | | | | | | | | | |
| Phage- | T | L | — | — | — | — | I | E | — | Y | I | V | — | Y | 0.09 | 0.31 | 0.23 | 621 |
| 516 | | | | | | | | | | | | | | | | | | |
| Phage- | S | — | — | — | — | — | — | — | — | — | I | F | — | T | 0.09 | 0.31 | 0.09 | 622 |
| 517 | | | | | | | | | | | | | | | | | | |
| Phage- | D | I | — | — | — | — | — | — | — | — | L | H | — | Y | 0.08 | 0.31 | 0.08 | 623 |
| 518 | | | | | | | | | | | | | | | | | | |
| Phage- | L | F | — | — | — | — | — | E | — | A | Y | L | — | I | 0.08 | 0.31 | 0.08 | 624 |
| 519 | | | | | | | | | | | | | | | | | | |
| Phage- | I | F | — | — | — | — | I | E | — | D | F | V | — | T | 0.10 | 0.31 | 0.10 | 625 |
| 520 | | | | | | | | | | | | | | | | | | |
| Phage- | D | D | — | — | — | — | W | E | Y | Y | — | A | — | V | 0.08 | 0.31 | 0.08 | 626 |
| 521 | | | | | | | | | | | | | | | | | | |
| Phage- | D | D | — | — | — | — | L | — | — | T | T | I | — | Y | 0.10 | 0.31 | 0.09 | 627 |
| 522 | | | | | | | | | | | | | | | | | | |
| Phage- | A | S | — | — | — | — | L | — | — | — | T | A | — | D | 0.10 | 0.31 | 0.09 | 628 |
| 523 | | | | | | | | | | | | | | | | | | |
| Phage- | Y | L | — | — | — | — | V | E | D | Y | D | Y | — | Y | 0.08 | 0.31 | 0.09 | 629 |
| 524 | | | | | | | | | | | | | | | | | | |
| Phage- | D | L | — | — | — | — | W | E | — | T | I | F | — | A | 0.12 | 0.30 | 0.09 | 630 |
| 525 | | | | | | | | | | | | | | | | | | |
| Phage- | D | F | — | — | — | D | G | E | — | F | Y | I | — | P | 0.12 | 0.30 | 0.11 | 631 |
| 526 | | | | | | | | | | | | | | | | | | |
| Phage- | — | — | — | — | — | — | V | E | — | N | I | L | — | H | 0.15 | 0.30 | 0.17 | 632 |
| 527 | | | | | | | | | | | | | | | | | | |
| Phage- | D | — | — | — | — | — | V | E | — | N | Y | F | — | F | 0.12 | 0.30 | 0.21 | 633 |
| 528 | | | | | | | | | | | | | | | | | | |
| Phage- | N | D | — | W | — | D | W | Y | — | F | L | S | — | D | 0.10 | 0.30 | 0.10 | 634 |
| 529 | | | | | | | | | | | | | | | | | | |
| Phage- | R | D | — | W | — | D | W | E | V | P | Y | F | — | D | 0.08 | 0.30 | 0.09 | 635 |
| 530 | | | | | | | | | | | | | | | | | | |
| Phage- | N | L | — | — | — | — | V | E | — | A | — | Y | — | Y | 0.10 | 0.30 | 0.13 | 636 |
| 531 | | | | | | | | | | | | | | | | | | |
| Phage- | F | D | — | W | — | D | G | E | L | N | Y | L | — | T | 0.23 | 0.30 | 0.08 | 637 |
| 532 | | | | | | | | | | | | | | | | | | |
| Phage- | Y | — | — | — | — | — | V | E | D | V | N | L | — | I | 0.18 | 0.30 | 0.10 | 638 |
| 533 | | | | | | | | | | | | | | | | | | |
| Phage- | D | N | — | — | — | — | Y | — | — | — | I | T | — | L | 0.11 | 0.29 | 0.09 | 639 |
| 534 | | | | | | | | | | | | | | | | | | |
| Phage- | L | N | — | W | — | D | W | E | — | D | Y | S | — | N | 0.09 | 0.29 | 0.09 | 640 |
| 535 | | | | | | | | | | | | | | | | | | |
| Phage- | P | T | — | — | — | — | V | E | — | L | L | S | — | N | 0.12 | 0.29 | 0.26 | 641 |
| 536 | | | | | | | | | | | | | | | | | | |
| Phage- | D | H | — | — | — | — | V | E | L | I | F | Y | — | H | 0.11 | 0.29 | 0.08 | 642 |
| 537 | | | | | | | | | | | | | | | | | | |
| Phage- | F | H | — | — | — | — | L | — | — | — | I | F | — | Y | 0.08 | 0.29 | 0.08 | 643 |
| 538 | | | | | | | | | | | | | | | | | | |
| Phage- | F | D | — | — | — | — | L | E | — | T | — | V | — | P | 0.16 | 0.29 | 0.16 | 644 |
| 539 | | | | | | | | | | | | | | | | | | |
| Phage- | D | F | — | — | — | — | L | — | — | — | L | P | — | A | 0.08 | 0.29 | 0.08 | 645 |
| 540 | | | | | | | | | | | | | | | | | | |
| Phage- | D | S | — | — | — | — | L | — | — | — | I | Y | — | D | 0.09 | 0.29 | 0.09 | 646 |
| 541 | | | | | | | | | | | | | | | | | | |
| Phage- | A | D | — | W | — | D | W | E | — | F | L | L | — | F | 0.12 | 0.29 | 0.10 | 647 |
| 542 | | | | | | | | | | | | | | | | | | |
| Phage- | I | L | — | — | — | — | V | E | — | L | D | F | — | N | 0.10 | 0.28 | 0.08 | 648 |
| 543 | | | | | | | | | | | | | | | | | | |
| Phage- | F | F | — | — | — | — | — | E | — | I | F | L | — | Y | 0.09 | 0.28 | 0.07 | 649 |
| 544 | | | | | | | | | | | | | | | | | | |
| Phage- | Y | N | — | — | — | D | G | — | — | — | Y | D | — | H | 0.07 | 0.28 | 0.07 | 650 |
| 545 | | | | | | | | | | | | | | | | | | |
| Phage- | D | N | — | — | — | — | L | — | — | T | I | T | — | F | 0.11 | 0.28 | 0.11 | 651 |
| 546 | | | | | | | | | | | | | | | | | | |
| Phage- | H | N | — | — | — | D | G | — | — | A | F | I | — | N | 0.09 | 0.28 | 0.23 | 652 |
| 547 | | | | | | | | | | | | | | | | | | |
| Phage- | D | — | — | — | — | — | V | E | — | D | L | V | — | P | 0.10 | 0.28 | 0.23 | 653 |
| 548 | | | | | | | | | | | | | | | | | | |
| Phage- | D | — | — | — | — | — | L | — | — | — | L | N | — | F | 0.08 | 0.28 | 0.07 | 654 |
| 549 | | | | | | | | | | | | | | | | | | |
| Phage- | D | — | — | — | — | — | — | Q | — | D | F | H | — | H | 0.11 | 0.28 | 0.27 | 655 |
| 550 | | | | | | | | | | | | | | | | | | |
| Phage- | Y | D | — | — | — | — | W | E | F | T | D | D | — | I | 0.10 | 0.28 | 0.18 | 656 |
| 551 | | | | | | | | | | | | | | | | | | |
| Phage- | D | I | — | — | — | — | Y | E | — | D | I | I | — | Y | 0.14 | 0.28 | 0.19 | 657 |
| 552 | | | | | | | | | | | | | | | | | | |
| Phage- | F | D | — | — | — | — | L | — | — | T | — | P | — | P | 0.11 | 0.28 | 0.08 | 658 |
| 553 | | | | | | | | | | | | | | | | | | |
| Phage- | N | — | — | — | — | — | L | — | — | T | S | V | — | D | 0.09 | 0.28 | 0.26 | 659 |
| 554 | | | | | | | | | | | | | | | | | | |
| Phage- | Y | — | — | — | — | — | W | E | F | — | F | D | — | D | 0.17 | 0.28 | 0.11 | 660 |
| 555 | | | | | | | | | | | | | | | | | | |
| Phage- | Y | A | — | — | — | — | L | — | — | — | — | T | — | D | 0.09 | 0.28 | 0.08 | 661 |
| 556 | | | | | | | | | | | | | | | | | | |
| Phage- | F | L | — | — | — | — | V | E | Q | D | Y | F | — | V | 0.08 | 0.28 | 0.10 | 662 |
| 557 | | | | | | | | | | | | | | | | | | |
| Phage- | D | N | — | — | — | — | — | — | — | — | — | R | — | D | 0.09 | 0.27 | 0.26 | 663 |
| 558 | | | | | | | | | | | | | | | | | | |
| Phage- | N | D | — | — | — | D | G | I | — | T | — | D | — | Y | 0.10 | 0.27 | 0.24 | 664 |
| 559 | | | | | | | | | | | | | | | | | | |
| Phage- | Y | F | — | — | — | — | V | E | D | Y | N | D | — | F | 0.08 | 0.27 | 0.09 | 665 |
| 560 | | | | | | | | | | | | | | | | | | |
| Phage- | N | L | — | — | — | — | — | — | — | — | I | F | — | Y | 0.10 | 0.27 | 0.07 | 666 |
| 561 | | | | | | | | | | | | | | | | | | |
| Phage- | I | D | — | W | — | D | W | E | — | Y | I | P | — | T | 0.10 | 0.27 | 0.08 | 667 |
| 562 | | | | | | | | | | | | | | | | | | |
| Phage- | I | — | — | — | — | D | G | — | — | — | F | I | — | A | 0.07 | 0.27 | 0.08 | 668 |
| 563 | | | | | | | | | | | | | | | | | | |
| Phage- | D | — | — | — | — | — | — | — | — | — | — | V | — | Y | 0.08 | 0.27 | 0.07 | 669 |
| 564 | | | | | | | | | | | | | | | | | | |
| Phage- | — | F | — | — | — | — | W | E | D | I | T | D | — | D | 0.13 | 0.27 | 0.09 | 670 |
| 565 | | | | | | | | | | | | | | | | | | |
| Phage- | L | D | — | — | — | — | V | — | — | T | F | T | — | H | 0.08 | 0.26 | 0.08 | 671 |
| 566 | | | | | | | | | | | | | | | | | | |
| Phage- | D | D | — | — | — | — | Y | — | — | — | F | A | — | H | 0.13 | 0.26 | 0.10 | 672 |
| 567 | | | | | | | | | | | | | | | | | | |
| Phage- | I | — | — | — | — | — | Y | Q | — | D | L | P | — | N | 0.12 | 0.26 | 0.11 | 673 |
| 568 | | | | | | | | | | | | | | | | | | |
| Phage- | L | D | — | — | — | — | V | E | — | Y | N | Y | — | V | 0.09 | 0.26 | 0.08 | 674 |
| 569 | | | | | | | | | | | | | | | | | | |
| Phage- | Y | V | — | — | — | — | — | — | — | — | S | A | — | N | 0.14 | 0.26 | 0.08 | 675 |
| 570 | | | | | | | | | | | | | | | | | | |
| Phage- | T | P | — | — | — | — | L | E | — | A | I | — | — | Y | 0.10 | 0.26 | 0.10 | 676 |
| 571 | | | | | | | | | | | | | | | | | | |
| Phage- | Y | F | — | — | — | — | — | — | — | — | A | D | — | N | 0.08 | 0.26 | 0.08 | 677 |
| 572 | | | | | | | | | | | | | | | | | | |
| Phage- | D | D | — | — | — | — | — | E | — | D | I | I | — | D | 0.12 | 0.26 | 0.25 | 678 |
| 573 | | | | | | | | | | | | | | | | | | |
| Phage- | L | — | — | — | — | V | V | E | — | L | N | H | — | N | 0.08 | 0.26 | 0.09 | 679 |
| 574 | | | | | | | | | | | | | | | | | | |
| Phage- | — | I | — | — | — | D | G | E | — | L | I | A | — | A | 0.09 | 0.26 | 0.27 | 680 |
| 575 | | | | | | | | | | | | | | | | | | |
| Phage- | F | A | — | W | — | D | W | Q | — | T | Y | V | — | N | 0.09 | 0.25 | 0.08 | 681 |
| 576 | | | | | | | | | | | | | | | | | | |
| Phage- | Y | I | — | — | — | — | V | E | F | L | F | F | — | N | 0.08 | 0.25 | 0.08 | 682 |
| 577 | | | | | | | | | | | | | | | | | | |
| Phage- | T | Q | — | — | — | K | G | E | P | T | Y | H | — | Y | 0.12 | 0.25 | 0.12 | 683 |
| 578 | | | | | | | | | | | | | | | | | | |
| Phage- | N | — | — | — | — | — | V | E | — | Y | H | N | — | D | 0.10 | 0.25 | 0.17 | 684 |
| 579 | | | | | | | | | | | | | | | | | | |
| Phage- | — | — | — | — | — | — | W | E | F | F | S | D | — | A | 0.08 | 0.25 | 0.07 | 685 |
| 580 | | | | | | | | | | | | | | | | | | |
| Phage- | F | — | — | — | — | — | L | E | — | — | F | F | — | Y | 0.10 | 0.25 | 0.22 | 686 |
| 581 | | | | | | | | | | | | | | | | | | |
| Phage- | D | — | — | — | — | — | I | E | — | N | F | Y | — | Y | 0.13 | 0.25 | 0.09 | 687 |
| 582 | | | | | | | | | | | | | | | | | | |
| Phage- | Y | A | — | — | — | — | V | E | — | Y | — | Y | — | A | 0.08 | 0.25 | 0.09 | 688 |
| 583 | | | | | | | | | | | | | | | | | | |
| Phage- | — | D | — | — | — | — | — | L | — | — | — | I | — | D | 0.08 | 0.25 | 0.07 | 689 |
| 584 | | | | | | | | | | | | | | | | | | |
| Phage- | N | D | — | — | — | — | M | — | — | — | I | A | — | Y | 0.07 | 0.25 | 0.07 | 690 |
| 585 | | | | | | | | | | | | | | | | | | |
| Phage- | — | — | — | — | — | — | — | — | — | Y | L | A | — | A | 0.09 | 0.25 | 0.21 | 691 |
| 586 | | | | | | | | | | | | | | | | | | |
| Phage- | — | I | — | — | — | — | — | — | — | — | A | N | — | D | 0.08 | 0.25 | 0.09 | 692 |
| 587 | | | | | | | | | | | | | | | | | | |
| Phage- | L | T | — | W | — | D | W | E | — | D | F | F | — | N | 0.07 | 0.24 | 0.07 | 693 |
| 588 | | | | | | | | | | | | | | | | | | |
| Phage- | F | — | — | — | — | — | Q | E | — | I | N | Y | — | Y | 0.10 | 0.24 | 0.23 | 694 |
| 589 | | | | | | | | | | | | | | | | | | |
| Phage- | T | — | — | — | — | — | L | E | — | F | F | L | — | Y | 0.13 | 0.24 | 0.08 | 695 |
| 590 | | | | | | | | | | | | | | | | | | |
| Phage- | P | D | — | — | — | — | L | — | — | — | — | A | — | H | 0.12 | 0.24 | 0.09 | 696 |
| 591 | | | | | | | | | | | | | | | | | | |
| Phage- | N | D | — | — | — | — | — | E | — | I | I | F | — | V | 0.09 | 0.24 | 0.24 | 697 |
| 592 | | | | | | | | | | | | | | | | | | |
| Phage- | Y | I | — | — | — | — | — | — | — | — | F | Y | — | N | 0.25 | 0.24 | 0.08 | 698 |
| 593 | | | | | | | | | | | | | | | | | | |
| Phage- | H | A | — | — | — | — | L | — | — | — | L | L | — | N | 0.20 | 0.24 | 0.07 | 699 |
| 594 | | | | | | | | | | | | | | | | | | |
| Phage- | Y | — | — | — | — | — | W | E | — | A | — | L | — | A | 0.09 | 0.24 | 0.21 | 700 |
| 595 | | | | | | | | | | | | | | | | | | |
| Phage- | A | F | — | — | — | — | V | E | — | Y | D | L | — | N | 0.10 | 0.24 | 0.16 | 701 |
| 596 | | | | | | | | | | | | | | | | | | |
| Phage- | Y | N | — | — | — | — | — | — | — | — | — | A | — | S | 0.15 | 0.23 | 0.09 | 702 |
| 597 | | | | | | | | | | | | | | | | | | |
| Phage- | Y | L | — | — | — | — | V | E | D | A | T | L | — | A | 0.08 | 0.23 | 0.09 | 703 |
| 598 | | | | | | | | | | | | | | | | | | |
| Phage- | A | V | — | — | — | — | — | — | — | — | — | N | — | D | 0.08 | 0.23 | 0.08 | 704 |
| 599 | | | | | | | | | | | | | | | | | | |
| Phage- | N | — | — | — | — | — | W | E | V | Y | S | L | — | P | 0.13 | 0.23 | 0.08 | 705 |
| 600 | | | | | | | | | | | | | | | | | | |
| Phage- | D | F | — | — | — | — | V | E | — | D | T | Y | — | H | 0.07 | 0.23 | 0.07 | 706 |
| 601 | | | | | | | | | | | | | | | | | | |
| Phage- | I | S | — | — | — | — | Y | E | W | D | Y | A | — | N | 0.08 | 0.23 | 0.10 | 707 |
| 602 | | | | | | | | | | | | | | | | | | |
| Phage- | — | N | — | — | — | — | L | — | — | — | I | I | — | Y | 0.08 | 0.23 | 0.08 | 708 |
| 603 | | | | | | | | | | | | | | | | | | |
| Phage- | Y | D | — | — | — | — | — | — | — | T | A | P | — | Y | 0.07 | 0.23 | 0.08 | 709 |
| 604 | | | | | | | | | | | | | | | | | | |
| Phage- | Y | L | — | — | — | — | — | E | — | N | F | L | — | T | 0.09 | 0.23 | 0.22 | 710 |
| 605 | | | | | | | | | | | | | | | | | | |
| Phage- | F | D | — | — | — | — | V | — | — | — | — | D | — | A | 0.08 | 0.22 | 0.09 | 711 |
| 606 | | | | | | | | | | | | | | | | | | |
| Phage- | Y | D | — | — | — | — | Q | E | — | I | S | F | — | N | 0.09 | 0.22 | 0.09 | 712 |
| 607 | | | | | | | | | | | | | | | | | | |
| Phage- | I | D | — | — | — | — | T | E | L | Y | D | D | — | F | 0.09 | 0.22 | 0.09 | 713 |
| 608 | | | | | | | | | | | | | | | | | | |
| Phage- | T | F | — | — | — | — | L | — | — | — | — | Y | — | Y | 0.08 | 0.22 | 0.07 | 714 |
| 609 | | | | | | | | | | | | | | | | | | |
| Phage- | F | F | — | — | — | — | I | — | — | — | N | A | — | V | 0.09 | 0.22 | 0.07 | 715 |
| 610 | | | | | | | | | | | | | | | | | | |
| Phage- | Y | H | — | W | — | D | W | E | P | I | Y | I | — | I | 0.12 | 0.22 | 0.10 | 716 |
| 611 | | | | | | | | | | | | | | | | | | |
| Phage- | A | I | — | — | — | — | Y | E | — | D | H | Y | — | Y | 0.08 | 0.22 | 0.08 | 717 |
| 612 | | | | | | | | | | | | | | | | | | |
| Phage- | P | L | — | — | — | D | G | F | — | N | Y | N | — | F | 0.12 | 0.22 | 0.08 | 718 |
| 613 | | | | | | | | | | | | | | | | | | |
| Phage- | F | P | — | W | — | D | W | E | W | D | N | N | — | H | 0.09 | 0.22 | 0.09 | 719 |
| 614 | | | | | | | | | | | | | | | | | | |
| Phage- | — | D | — | — | — | D | G | — | — | L | A | A | — | H | 0.10 | 0.22 | 0.11 | 720 |
| 615 | | | | | | | | | | | | | | | | | | |
| Phage- | — | D | — | W | — | D | W | E | — | Y | Y | S | — | D | 0.08 | 0.22 | 0.07 | 721 |
| 616 | | | | | | | | | | | | | | | | | | |
| Phage- | — | — | — | — | — | — | Y | — | — | — | Y | D | — | T | 0.07 | 0.21 | 0.10 | 722 |
| 617 | | | | | | | | | | | | | | | | | | |
| Phage- | N | L | — | — | — | — | W | E | N | F | A | D | — | F | 0.08 | 0.21 | 0.08 | 723 |
| 618 | | | | | | | | | | | | | | | | | | |
| Phage- | Y | L | — | — | — | — | L | E | V | F | F | V | — | D | 0.12 | 0.21 | 0.10 | 724 |
| 619 | | | | | | | | | | | | | | | | | | |
| Phage- | I | F | — | — | — | — | L | E | D | Y | S | I | — | F | 0.09 | 0.21 | 0.08 | 725 |
| 620 | | | | | | | | | | | | | | | | | | |
| Phage- | D | — | — | — | — | — | L | E | Q | Y | D | L | — | F | 0.09 | 0.21 | 0.08 | 726 |
| 621 | | | | | | | | | | | | | | | | | | |
| Phage- | L | L | — | — | — | V | N | E | D | P | L | D | — | Y | 0.11 | 0.21 | 0.13 | 727 |
| 622 | | | | | | | | | | | | | | | | | | |
| Phage- | I | D | — | — | — | — | — | — | — | — | — | F | — | Y | 0.08 | 0.21 | 0.08 | 728 |
| 623 | | | | | | | | | | | | | | | | | | |
| Phage- | I | I | — | — | — | — | V | E | — | I | D | I | — | S | 0.08 | 0.21 | 0.08 | 729 |
| 624 | | | | | | | | | | | | | | | | | | |
| Phage- | I | A | — | W | — | D | W | E | D | Y | S | S | — | P | 0.08 | 0.21 | 0.11 | 730 |
| 625 | | | | | | | | | | | | | | | | | | |
| Phage- | Y | — | — | — | — | — | V | E | D | I | N | D | — | I | 0.09 | 0.21 | 0.07 | 731 |
| 626 | | | | | | | | | | | | | | | | | | |
| Phage- | N | I | — | — | — | — | M | — | — | — | I | D | — | I | 0.08 | 0.21 | 0.07 | 732 |
| 627 | | | | | | | | | | | | | | | | | | |
| Phage- | F | D | — | W | — | D | W | E | — | L | — | S | — | Y | 0.07 | 0.21 | 0.08 | 733 |
| 628 | | | | | | | | | | | | | | | | | | |
| Phage- | Y | F | — | — | — | — | W | E | D | H | F | F | — | D | 0.09 | 0.21 | 0.19 | 734 |
| 629 | | | | | | | | | | | | | | | | | | |
| Phage- | T | — | — | — | — | — | — | E | — | D | S | Y | — | D | 0.12 | 0.20 | 0.09 | 735 |
| 630 | | | | | | | | | | | | | | | | | | |
| Phage- | N | L | — | — | — | — | V | E | L | I | D | I | — | S | 0.11 | 0.20 | 0.09 | 736 |
| 631 | | | | | | | | | | | | | | | | | | |
| Phage- | D | N | — | — | — | — | W | E | — | V | Y | L | — | N | 0.08 | 0.20 | 0.08 | 737 |
| 632 | | | | | | | | | | | | | | | | | | |
| Phage- | F | L | — | — | — | — | — | — | — | — | D | L | — | F | 0.08 | 0.20 | 0.09 | 738 |
| 633 | | | | | | | | | | | | | | | | | | |
| Phage- | H | I | — | — | — | — | Q | — | — | — | I | — | — | T | 0.09 | 0.20 | 0.19 | 739 |
| 634 | | | | | | | | | | | | | | | | | | |
| Phage- | F | D | — | W | — | D | W | E | D | N | S | Y | — | D | 0.10 | 0.20 | 0.09 | 740 |
| 635 | | | | | | | | | | | | | | | | | | |
| Phage- | T | A | — | — | — | — | W | E | F | D | F | N | — | D | 0.08 | 0.20 | 0.07 | 741 |
| 636 | | | | | | | | | | | | | | | | | | |
| Phage- | H | H | — | W | — | D | W | E | D | Y | S | T | — | P | 0.10 | 0.20 | 0.11 | 742 |
| 637 | | | | | | | | | | | | | | | | | | |
| Phage- | Y | — | — | — | — | — | — | — | — | — | — | N | — | F | 0.07 | 0.20 | 0.08 | 743 |
| 638 | | | | | | | | | | | | | | | | | | |
| Phage- | L | H | — | W | — | D | W | E | — | I | D | I | — | D | 0.08 | 0.20 | 0.09 | 744 |
| 639 | | | | | | | | | | | | | | | | | | |
| Phage- | D | I | — | — | — | D | G | Q | — | D | F | V | — | S | 0.08 | 0.20 | 0.09 | 745 |
| 640 | | | | | | | | | | | | | | | | | | |
| Phage- | D | V | — | W | — | D | W | E | V | N | Y | F | — | D | 0.09 | 0.20 | 0.07 | 746 |
| 641 | | | | | | | | | | | | | | | | | | |
| Phage- | — | N | — | — | — | — | M | — | — | — | I | D | — | A | 0.12 | 0.20 | 0.10 | 747 |
| 642 | | | | | | | | | | | | | | | | | | |
| Phage- | D | N | — | — | — | — | — | — | — | A | T | V | — | N | 0.11 | 0.19 | 0.19 | 748 |
| 643 | | | | | | | | | | | | | | | | | | |
| Phage- | D | L | — | — | — | — | — | E | — | V | H | N | — | N | 0.08 | 0.19 | 0.08 | 749 |
| 644 | | | | | | | | | | | | | | | | | | |
| Phage- | — | N | — | — | — | — | — | — | — | — | S | Y | — | F | 0.13 | 0.19 | 0.09 | 750 |
| 645 | | | | | | | | | | | | | | | | | | |
| Phage- | N | I | — | W | — | D | W | E | — | D | N | F | — | S | 0.08 | 0.19 | 0.08 | 751 |
| 646 | | | | | | | | | | | | | | | | | | |
| Phage- | F | V | — | — | — | — | W | E | V | Y | D | D | — | D | 0.08 | 0.19 | 0.08 | 752 |
| 647 | | | | | | | | | | | | | | | | | | |
| Phage- | A | — | — | — | — | — | L | E | V | V | H | L | — | V | 0.10 | 0.19 | 0.17 | 753 |
| 648 | | | | | | | | | | | | | | | | | | |
| Phage- | P | F | — | — | — | — | M | — | — | T | I | D | — | Y | 0.07 | 0.19 | 0.09 | 754 |
| 649 | | | | | | | | | | | | | | | | | | |
| Phage- | L | L | — | — | — | V | M | E | D | V | F | A | — | Y | 0.08 | 0.19 | 0.08 | 755 |
| 650 | | | | | | | | | | | | | | | | | | |
| Phage- | D | L | — | — | — | — | — | — | — | — | T | N | — | Y | 0.07 | 0.19 | 0.08 | 756 |
| 651 | | | | | | | | | | | | | | | | | | |
| Phage- | H | D | — | — | — | — | M | E | — | Y | Y | L | — | P | 0.10 | 0.18 | 0.10 | 757 |
| 652 | | | | | | | | | | | | | | | | | | |
| Phage- | T | D | — | — | — | — | Y | — | — | — | I | I | — | P | 0.08 | 0.18 | 0.09 | 758 |
| 653 | | | | | | | | | | | | | | | | | | |
| Phage- | — | L | — | W | — | D | W | E | D | Y | A | D | — | N | 0.09 | 0.18 | 0.08 | 759 |
| 654 | | | | | | | | | | | | | | | | | | |
| Phage- | N | D | — | — | — | — | L | — | — | — | L | T | — | D | 0.07 | 0.18 | 0.09 | 760 |
| 655 | | | | | | | | | | | | | | | | | | |
| Phage- | I | — | — | — | — | — | L | — | — | — | I | A | — | Y | 0.11 | 0.18 | 0.08 | 761 |
| 656 | | | | | | | | | | | | | | | | | | |
| Phage- | N | — | — | — | — | — | V | E | — | F | N | F | — | H | 0.11 | 0.18 | 0.14 | 762 |
| 657 | | | | | | | | | | | | | | | | | | |
| Phage- | D | V | — | — | — | — | I | E | — | Y | S | F | — | I | 0.08 | 0.18 | 0.09 | 763 |
| 658 | | | | | | | | | | | | | | | | | | |
| Phage- | D | L | — | — | — | — | V | E | — | I | T | D | — | A | 0.10 | 0.18 | 0.12 | 764 |
| 659 | | | | | | | | | | | | | | | | | | |
| Phage- | H | D | — | — | — | — | — | — | — | — | — | F | — | I | 0.12 | 0.18 | 0.12 | 765 |
| 660 | | | | | | | | | | | | | | | | | | |
| Phage- | P | L | — | — | — | V | L | E | — | D | I | Y | — | Y | 0.10 | 0.18 | 0.13 | 766 |
| 661 | | | | | | | | | | | | | | | | | | |
| Phage- | D | L | — | — | — | — | — | E | D | I | I | D | — | N | 0.10 | 0.18 | 0.11 | 767 |
| 662 | | | | | | | | | | | | | | | | | | |
| Phage- | D | — | — | — | — | — | V | E | V | P | S | N | — | N | 0.10 | 0.18 | 0.18 | 768 |
| 663 | | | | | | | | | | | | | | | | | | |
| Phage- | I | I | — | — | — | — | L | — | — | — | T | A | — | D | 0.10 | 0.18 | 0.09 | 769 |
| 664 | | | | | | | | | | | | | | | | | | |
| Phage- | D | H | — | — | — | — | — | — | — | — | — | N | — | D | 0.10 | 0.18 | 0.14 | 770 |
| 665 | | | | | | | | | | | | | | | | | | |
| Phage- | F | D | — | — | — | — | — | — | — | — | L | Y | — | S | 0.07 | 0.18 | 0.07 | 771 |
| 666 | | | | | | | | | | | | | | | | | | |
| Phage- | F | A | — | W | — | D | W | E | — | V | Y | I | — | Y | 0.08 | 0.18 | 0.08 | 772 |
| 667 | | | | | | | | | | | | | | | | | | |
| Phage- | L | — | — | — | — | — | — | — | — | — | L | D | — | S | 0.08 | 0.18 | 0.09 | 773 |
| 668 | | | | | | | | | | | | | | | | | | |
| Phage- | D | L | — | — | — | — | L | E | — | A | F | L | — | A | 0.09 | 0.18 | 0.08 | 774 |
| 669 | | | | | | | | | | | | | | | | | | |
| Phage- | F | A | — | — | — | — | L | — | — | T | L | T | — | L | 0.10 | 0.18 | 0.08 | 775 |
| 670 | | | | | | | | | | | | | | | | | | |
| Phage- | F | D | — | — | — | — | V | E | — | I | S | N | — | D | 0.17 | 0.18 | 0.10 | 776 |
| 671 | | | | | | | | | | | | | | | | | | |
| Phage- | — | H | — | — | — | — | L | E | Y | P | F | D | — | N | 0.09 | 0.17 | 0.16 | 777 |
| 672 | | | | | | | | | | | | | | | | | | |
| Phage- | A | — | — | — | — | — | — | E | — | H | T | T | — | N | 0.10 | 0.17 | 0.15 | 778 |
| 673 | | | | | | | | | | | | | | | | | | |
| Phage- | L | — | — | — | — | V | S | E | W | F | T | F | — | I | 0.08 | 0.17 | 0.08 | 779 |
| 674 | | | | | | | | | | | | | | | | | | |
| Phage- | D | — | — | — | — | — | L | — | — | — | Y | D | — | N | 0.10 | 0.17 | 0.09 | 780 |
| 675 | | | | | | | | | | | | | | | | | | |
| Phage- | F | — | — | — | — | — | W | E | — | F | D | V | — | I | 0.13 | 0.17 | 0.15 | 781 |
| 676 | | | | | | | | | | | | | | | | | | |
| Phage- | F | T | — | — | — | — | V | E | — | Y | D | H | — | I | 0.08 | 0.17 | 0.09 | 782 |
| 677 | | | | | | | | | | | | | | | | | | |
| Phage- | Y | N | — | — | — | — | — | — | — | — | — | T | — | F | 0.12 | 0.17 | 0.11 | 783 |
| 678 | | | | | | | | | | | | | | | | | | |
| Phage- | A | V | — | — | — | — | N | — | — | — | N | S | — | A | 0.08 | 0.17 | 0.08 | 784 |
| 679 | | | | | | | | | | | | | | | | | | |
| Phage- | I | — | — | W | — | D | W | E | V | P | N | D | — | A | 0.10 | 0.17 | 0.09 | 785 |
| 680 | | | | | | | | | | | | | | | | | | |
| Phage- | Y | F | — | — | — | — | — | E | — | F | F | H | — | Y | 0.12 | 0.17 | 0.12 | 786 |
| 681 | | | | | | | | | | | | | | | | | | |
| Phage- | Y | V | — | — | — | D | G | — | — | — | S | F | — | D | 0.12 | 0.17 | 0.12 | 787 |
| 682 | | | | | | | | | | | | | | | | | | |
| Phage- | I | S | — | — | — | — | V | E | — | F | F | Y | — | Y | 0.10 | 0.17 | 0.08 | 788 |
| 683 | | | | | | | | | | | | | | | | | | |
| Phage- | L | I | — | — | — | V | — | E | — | D | — | Y | — | D | 0.17 | 0.17 | 0.15 | 789 |
| 684 | | | | | | | | | | | | | | | | | | |
| Phage- | — | — | — | — | — | — | V | E | D | H | N | Y | — | A | 0.14 | 0.17 | 0.16 | 790 |
| 685 | | | | | | | | | | | | | | | | | | |
| Phage- | L | D | — | — | — | — | — | E | F | V | Y | I | — | A | 0.08 | 0.17 | 0.10 | 791 |
| 686 | | | | | | | | | | | | | | | | | | |
| Phage- | Y | D | — | — | — | — | — | E | — | D | L | P | — | I | 0.17 | 0.17 | 0.11 | 792 |
| 687 | | | | | | | | | | | | | | | | | | |
| Phage- | D | V | — | — | — | — | V | E | — | D | Y | Y | — | D | 0.10 | 0.17 | 0.14 | 793 |
| 688 | | | | | | | | | | | | | | | | | | |
| Phage- | N | D | — | W | — | D | W | E | Y | D | N | V | — | V | 0.08 | 0.17 | 0.10 | 794 |
| 689 | | | | | | | | | | | | | | | | | | |
| Phage- | D | L | — | — | — | — | — | E | V | A | N | D | — | N | 0.10 | 0.16 | 0.16 | 795 |
| 690 | | | | | | | | | | | | | | | | | | |
| Phage- | H | D | — | — | — | — | L | — | — | — | I | S | — | N | 0.09 | 0.16 | 0.07 | 796 |
| 691 | | | | | | | | | | | | | | | | | | |
| Phage- | L | D | — | W | — | D | W | E | — | T | T | H | — | D | 0.08 | 0.16 | 0.08 | 797 |
| 692 | | | | | | | | | | | | | | | | | | |
| Phage- | I | I | — | — | — | — | V | E | — | D | D | Y | — | L | 0.09 | 0.16 | 0.09 | 798 |
| 693 | | | | | | | | | | | | | | | | | | |
| Phage- | Y | — | — | W | — | D | W | E | — | V | I | I | — | D | 0.09 | 0.16 | 0.09 | 799 |
| 694 | | | | | | | | | | | | | | | | | | |
| Phage- | F | D | — | — | — | — | I | — | — | Y | T | N | — | N | 0.12 | 0.16 | 0.09 | 800 |
| 695 | | | | | | | | | | | | | | | | | | |
| Phage- | I | T | — | — | — | — | L | — | — | T | I | N | — | D | 0.08 | 0.16 | 0.11 | 801 |
| 696 | | | | | | | | | | | | | | | | | | |
| Phage- | D | S | — | — | — | — | V | E | — | D | I | Y | — | I | 0.07 | 0.16 | 0.08 | 802 |
| 697 | | | | | | | | | | | | | | | | | | |
| Phage- | Y | L | — | — | — | — | — | — | — | — | G | N | — | H | 0.07 | 0.16 | 0.08 | 803 |
| 698 | | | | | | | | | | | | | | | | | | |
| Phage- | D | N | — | — | — | — | L | P | — | D | Y | F | — | D | 0.08 | 0.16 | 0.10 | 804 |
| 699 | | | | | | | | | | | | | | | | | | |
| Phage- | — | L | — | — | — | — | — | E | — | V | S | N | — | N | 0.11 | 0.16 | 0.08 | 805 |
| 700 | | | | | | | | | | | | | | | | | | |
| Phage- | — | D | — | W | — | D | W | E | — | D | I | V | — | D | 0.10 | 0.16 | 0.09 | 806 |
| 701 | | | | | | | | | | | | | | | | | | |
| Phage- | — | — | — | W | — | D | W | E | D | N | F | P | — | Y | 0.07 | 0.16 | 0.07 | 807 |
| 702 | | | | | | | | | | | | | | | | | | |
| Phage- | D | — | — | — | — | — | V | E | — | H | F | N | — | H | 0.08 | 0.16 | 0.08 | 808 |
| 703 | | | | | | | | | | | | | | | | | | |
| Phage- | A | D | — | — | — | — | I | E | — | D | A | Y | — | Y | 0.12 | 0.16 | 0.09 | 809 |
| 704 | | | | | | | | | | | | | | | | | | |
| Phage- | I | L | — | W | — | D | W | E | D | A | T | F | — | Y | 0.09 | 0.16 | 0.07 | 810 |
| 705 | | | | | | | | | | | | | | | | | | |
| Phage- | I | H | — | W | — | D | W | E | D | F | N | I | — | P | 0.09 | 0.16 | 0.08 | 811 |
| 706 | | | | | | | | | | | | | | | | | | |
| Phage- | T | I | — | — | — | — | V | E | D | Y | N | D | — | I | 0.07 | 0.16 | 0.07 | 812 |
| 707 | | | | | | | | | | | | | | | | | | |
| Phage- | D | D | — | — | — | — | L | — | — | — | — | A | — | I | 0.08 | 0.16 | 0.08 | 813 |
| 708 | | | | | | | | | | | | | | | | | | |
| Phage- | D | D | — | W | — | D | W | E | D | H | I | F | — | F | 0.13 | 0.16 | 0.08 | 814 |
| 709 | | | | | | | | | | | | | | | | | | |
| Phage- | — | N | — | — | — | — | V | E | — | I | I | F | — | D | 0.12 | 0.15 | 0.12 | 815 |
| 710 | | | | | | | | | | | | | | | | | | |
| Phage- | I | F | — | W | — | D | W | E | D | D | T | V | — | I | 0.08 | 0.15 | 0.09 | 816 |
| 711 | | | | | | | | | | | | | | | | | | |
| Phage- | I | I | — | — | — | — | — | E | — | I | S | D | — | L | 0.12 | 0.15 | 0.15 | 817 |
| 712 | | | | | | | | | | | | | | | | | | |
| Phage- | F | D | — | — | — | — | V | E | — | Y | N | D | — | D | 0.11 | 0.15 | 0.09 | 818 |
| 713 | | | | | | | | | | | | | | | | | | |
| Phage- | N | D | — | — | — | — | L | — | — | T | L | Y | — | I | 0.08 | 0.15 | 0.10 | 819 |
| 714 | | | | | | | | | | | | | | | | | | |
| Phage- | A | I | — | — | — | — | L | E | — | D | I | S | — | N | 0.12 | 0.15 | 0.17 | 820 |
| 715 | | | | | | | | | | | | | | | | | | |
| Phage- | H | L | — | — | — | — | — | — | — | — | T | N | — | Y | 0.07 | 0.15 | 0.07 | 821 |
| 716 | | | | | | | | | | | | | | | | | | |
| Phage- | S | — | — | — | — | — | L | — | — | — | — | A | — | I | 0.10 | 0.15 | 0.11 | 822 |
| 717 | | | | | | | | | | | | | | | | | | |
| Phage- | L | — | — | — | — | — | — | E | Q | L | A | D | — | T | 0.08 | 0.15 | 0.08 | 823 |
| 718 | | | | | | | | | | | | | | | | | | |
| Phage- | I | D | — | — | — | — | L | — | — | — | I | A | — | N | 0.07 | 0.15 | 0.08 | 824 |
| 719 | | | | | | | | | | | | | | | | | | |
| Phage- | F | D | — | — | — | D | G | Q | — | D | L | V | — | N | 0.10 | 0.15 | 0.08 | 825 |
| 720 | | | | | | | | | | | | | | | | | | |
| Phage- | N | L | — | — | — | — | — | E | — | F | F | D | — | Y | 0.09 | 0.15 | 0.15 | 826 |
| 721 | | | | | | | | | | | | | | | | | | |
| Phage- | S | I | — | — | — | — | L | Q | — | D | I | V | — | P | 0.09 | 0.14 | 0.14 | 827 |
| 722 | | | | | | | | | | | | | | | | | | |
| Phage- | N | P | — | — | — | — | Y | — | — | — | A | H | — | D | 0.08 | 0.14 | 0.08 | 828 |
| 723 | | | | | | | | | | | | | | | | | | |
| Phage- | Y | D | — | — | — | — | L | — | — | Y | Y | N | — | N | 0.12 | 0.14 | 0.11 | 829 |
| 724 | | | | | | | | | | | | | | | | | | |
| Phage- | — | D | — | — | — | — | L | — | — | T | I | F | — | D | 0.07 | 0.14 | 0.08 | 830 |
| 725 | | | | | | | | | | | | | | | | | | |
| Phage- | D | N | — | — | — | — | L | — | — | — | — | T | — | T | 0.09 | 0.14 | 0.10 | 831 |
| 726 | | | | | | | | | | | | | | | | | | |
| Phage- | — | D | — | — | — | — | L | — | — | — | S | Y | — | D | 0.10 | 0.14 | 0.13 | 832 |
| 727 | | | | | | | | | | | | | | | | | | |
| Phage- | Y | — | — | — | — | — | — | E | F | I | D | F | — | F | 0.07 | 0.14 | 0.07 | 833 |
| 728 | | | | | | | | | | | | | | | | | | |
| Phage- | Y | D | — | W | — | D | W | E | V | I | T | Y | — | N | 0.08 | 0.14 | 0.09 | 834 |
| 729 | | | | | | | | | | | | | | | | | | |
| Phage- | F | D | — | — | — | — | I | E | — | D | F | F | — | V | 0.06 | 0.14 | 0.07 | 835 |
| 730 | | | | | | | | | | | | | | | | | | |
| Phage- | I | F | — | W | — | D | W | — | D | I | N | F | — | D | 0.10 | 0.14 | 0.14 | 836 |
| 731 | | | | | | | | | | | | | | | | | | |
| Phage- | N | F | — | — | — | — | L | P | — | D | I | T | — | Y | 0.37 | 0.14 | 0.09 | 837 |
| 732 | | | | | | | | | | | | | | | | | | |
| Phage- | S | L | — | — | — | — | — | E | — | Y | Y | H | — | L | 0.09 | 0.14 | 0.07 | 838 |
| 733 | | | | | | | | | | | | | | | | | | |
| Phage- | A | S | — | — | — | — | L | — | — | — | L | D | — | L | 0.12 | 0.14 | 0.13 | 839 |
| 734 | | | | | | | | | | | | | | | | | | |
| Phage- | S | F | — | — | — | — | R | E | W | D | L | A | — | Y | 0.09 | 0.14 | 0.08 | 840 |
| 735 | | | | | | | | | | | | | | | | | | |
| Phage- | H | L | — | — | — | — | — | E | D | V | L | D | — | I | 0.08 | 0.14 | 0.12 | 841 |
| 736 | | | | | | | | | | | | | | | | | | |
| Phage- | L | D | — | — | — | D | G | — | — | F | Y | Y | — | L | 0.20 | 0.14 | 0.09 | 842 |
| 737 | | | | | | | | | | | | | | | | | | |
| Phage- | D | N | — | W | — | D | W | E | — | D | I | A | — | T | 0.16 | 0.14 | 0.11 | 843 |
| 738 | | | | | | | | | | | | | | | | | | |
| Phage- | S | D | — | — | — | — | L | — | — | T | I | H | — | I | 0.09 | 0.14 | 0.08 | 844 |
| 739 | | | | | | | | | | | | | | | | | | |
| Phage- | N | S | — | — | — | D | G | — | — | — | — | D | — | L | 0.08 | 0.14 | 0.08 | 845 |
| 740 | | | | | | | | | | | | | | | | | | |
| Phage- | D | L | — | — | — | — | L | — | — | — | T | L | — | I | 0.07 | 0.14 | 0.08 | 846 |
| 741 | | | | | | | | | | | | | | | | | | |
| Phage- | F | D | — | — | — | — | S | — | — | — | F | N | — | Y | 0.09 | 0.14 | 0.11 | 847 |
| 742 | | | | | | | | | | | | | | | | | | |
| Phage- | D | L | — | — | — | — | — | E | — | D | D | I | — | Y | 0.12 | 0.14 | 0.13 | 848 |
| 743 | | | | | | | | | | | | | | | | | | |
| Phage- | H | A | — | — | — | — | — | E | — | D | T | Y | — | F | 0.10 | 0.14 | 0.14 | 849 |
| 744 | | | | | | | | | | | | | | | | | | |
| Phage- | S | D | — | — | — | — | L | — | — | — | — | A | — | I | 0.11 | 0.14 | 0.08 | 850 |
| 745 | | | | | | | | | | | | | | | | | | |
| Phage- | I | V | — | — | — | — | L | P | — | D | Y | N | — | Y | 0.09 | 0.13 | 0.11 | 851 |
| 746 | | | | | | | | | | | | | | | | | | |
| Phage- | D | L | — | — | — | — | — | — | — | — | F | I | — | F | 0.09 | 0.13 | 0.08 | 852 |
| 747 | | | | | | | | | | | | | | | | | | |
| Phage- | Y | D | — | — | — | — | — | — | — | T | L | T | — | N | 0.13 | 0.13 | 0.08 | 853 |
| 748 | | | | | | | | | | | | | | | | | | |
| Phage- | Y | D | — | — | — | — | V | E | — | I | — | N | — | D | 0.11 | 0.13 | 0.12 | 854 |
| 749 | | | | | | | | | | | | | | | | | | |
| Phage- | — | F | — | — | — | — | I | E | D | D | H | V | — | I | 0.07 | 0.13 | 0.07 | 855 |
| 750 | | | | | | | | | | | | | | | | | | |
| Phage- | F | I | — | — | — | — | W | E | D | D | Y | A | — | S | 0.12 | 0.13 | 0.12 | 856 |
| 751 | | | | | | | | | | | | | | | | | | |
| Phage- | N | F | — | — | — | — | — | — | — | — | — | — | — | N | 0.10 | 0.13 | 0.09 | 857 |
| 752 | | | | | | | | | | | | | | | | | | |
| Phage- | N | L | — | — | — | — | V | E | — | I | L | I | — | D | 0.07 | 0.13 | 0.07 | 858 |
| 753 | | | | | | | | | | | | | | | | | | |
| Phage- | D | S | — | — | — | — | V | E | — | Y | D | L | — | N | 0.11 | 0.13 | 0.08 | 859 |
| 754 | | | | | | | | | | | | | | | | | | |
| Phage- | T | L | — | — | — | — | — | E | — | I | T | D | — | N | 0.09 | 0.13 | 0.08 | 860 |
| 755 | | | | | | | | | | | | | | | | | | |
| Phage- | T | V | — | — | — | K | M | E | M | N | S | T | — | D | 0.09 | 0.13 | 0.09 | 861 |
| 756 | | | | | | | | | | | | | | | | | | |
| Phage- | — | H | — | W | — | D | W | E | D | A | — | S | — | N | 0.10 | 0.12 | 0.11 | 862 |
| 757 | | | | | | | | | | | | | | | | | | |
| Phage- | D | L | — | — | — | D | G | N | — | L | D | F | — | F | 0.08 | 0.12 | 0.08 | 863 |
| 758 | | | | | | | | | | | | | | | | | | |
| Phage- | D | L | — | — | — | D | G | E | — | H | Y | Y | — | D | 0.09 | 0.12 | 0.07 | 864 |
| 759 | | | | | | | | | | | | | | | | | | |
| Phage- | F | N | — | — | — | — | V | E | — | I | L | L | — | T | 0.11 | 0.12 | 0.12 | 865 |
| 760 | | | | | | | | | | | | | | | | | | |
| Phage- | H | — | — | — | — | — | V | E | N | I | N | D | — | I | 0.09 | 0.12 | 0.07 | 866 |
| 761 | | | | | | | | | | | | | | | | | | |
| Phage- | I | D | — | — | — | — | L | — | — | — | — | I | — | D | 0.09 | 0.12 | 0.08 | 867 |
| 762 | | | | | | | | | | | | | | | | | | |
| Phage- | I | F | — | — | — | — | I | E | Q | P | A | L | — | Y | 0.08 | 0.12 | 0.09 | 868 |
| 763 | | | | | | | | | | | | | | | | | | |
| Phage- | Y | D | — | — | — | — | — | Q | — | D | L | V | — | P | 0.10 | 0.12 | 0.08 | 869 |
| 764 | | | | | | | | | | | | | | | | | | |
| Phage- | H | A | — | W | — | D | W | E | — | P | N | Y | — | D | 0.13 | 0.12 | 0.09 | 870 |
| 765 | | | | | | | | | | | | | | | | | | |
| Phage- | N | V | — | W | — | D | W | E | — | D | Y | N | — | Y | 0.11 | 0.12 | 0.10 | 871 |
| 766 | | | | | | | | | | | | | | | | | | |
| Phage- | S | F | — | — | Q | — | L | G | D | N | Y | D | — | I | 0.09 | 0.12 | 0.12 | 872 |
| 767 | | | | | | | | | | | | | | | | | | |
| Phage- | D | D | — | — | — | — | L | — | — | T | T | V | — | Y | 0.08 | 0.12 | 0.09 | 873 |
| 768 | | | | | | | | | | | | | | | | | | |
| Phage- | N | F | — | — | — | — | W | E | V | A | T | L | — | L | 0.09 | 0.12 | 0.14 | 874 |
| 769 | | | | | | | | | | | | | | | | | | |
| Phage- | D | L | — | — | — | — | V | E | — | D | T | Y | — | N | 0.09 | 0.11 | 0.07 | 875 |
| 770 | | | | | | | | | | | | | | | | | | |
| Phage- | A | L | — | — | — | — | V | E | Q | V | D | L | — | T | 0.08 | 0.11 | 0.08 | 876 |
| 771 | | | | | | | | | | | | | | | | | | |
| Phage- | D | D | — | — | — | — | L | — | — | — | — | N | — | N | 0.08 | 0.11 | 0.08 | 877 |
| 772 | | | | | | | | | | | | | | | | | | |
| Phage- | I | F | — | — | — | — | — | E | Q | I | I | Y | — | D | 0.09 | 0.11 | 0.11 | 878 |
| 773 | | | | | | | | | | | | | | | | | | |
| Phage- | S | D | — | W | — | D | W | E | — | V | Y | Y | — | S | 0.09 | 0.11 | 0.10 | 879 |
| 774 | | | | | | | | | | | | | | | | | | |
| Phage- | F | F | — | — | — | D | G | — | — | V | A | I | — | D | 0.09 | 0.11 | 0.07 | 880 |
| 775 | | | | | | | | | | | | | | | | | | |
| Phage- | D | D | — | W | — | D | W | E | D | D | — | Y | — | Y | 0.11 | 0.11 | 0.07 | 881 |
| 776 | | | | | | | | | | | | | | | | | | |
| Phage- | Y | D | — | — | — | — | — | — | — | T | — | V | — | P | 0.08 | 0.11 | 0.08 | 882 |
| 777 | | | | | | | | | | | | | | | | | | |
| Phage- | S | — | — | — | — | — | L | — | — | — | — | N | — | V | 0.10 | 0.11 | 0.08 | 883 |
| 778 | | | | | | | | | | | | | | | | | | |
| Phage- | A | F | — | V | S | F | Q | Q | S | L | P | H | — | D | 0.09 | 0.11 | 0.10 | 884 |
| 779 | | | | | | | | | | | | | | | | | | |
| Phage- | — | N | — | — | — | — | V | E | — | Y | F | V | — | F | 0.09 | 0.11 | 0.09 | 885 |
| 780 | | | | | | | | | | | | | | | | | | |
| Phage- | T | N | — | W | — | D | W | E | — | D | F | A | — | V | 0.07 | 0.11 | 0.08 | 886 |
| 781 | | | | | | | | | | | | | | | | | | |
| Phage- | D | D | — | — | — | — | — | E | — | I | I | L | — | F | 0.08 | 0.10 | 0.08 | 887 |
| 782 | | | | | | | | | | | | | | | | | | |
| Phage- | N | S | — | — | — | — | L | E | D | Y | H | L | — | P | 0.08 | 0.10 | 0.10 | 888 |
| 783 | | | | | | | | | | | | | | | | | | |
| Phage- | I | H | — | — | — | D | S | — | G | F | D | F | — | D | 0.09 | 0.10 | 0.08 | 889 |
| 784 | | | | | | | | | | | | | | | | | | |
| Phage- | F | D | — | — | — | — | L | E | — | D | H | L | — | F | 0.08 | 0.10 | 0.08 | 890 |
| 785 | | | | | | | | | | | | | | | | | | |
| Phage- | T | V | — | W | — | D | — | E | — | Y | A | D | — | D | 0.10 | 0.10 | 0.08 | 891 |
| 786 | | | | | | | | | | | | | | | | | | |
| Phage- | Y | F | — | W | — | D | W | E | — | A | A | D | — | L | 0.09 | 0.10 | 0.09 | 892 |
| 787 | | | | | | | | | | | | | | | | | | |
| Phage- | — | S | — | — | — | — | — | — | — | — | — | D | — | I | 0.08 | 0.10 | 0.07 | 893 |
| 788 | | | | | | | | | | | | | | | | | | |
| Phage- | A | V | — | — | — | — | L | P | — | D | I | V | — | Y | 0.08 | 0.10 | 0.08 | 894 |
| 789 | | | | | | | | | | | | | | | | | | |
| Phage- | — | L | — | — | — | — | V | E | — | Y | H | L | — | A | 0.08 | 0.10 | 0.30 | 895 |
| 790 | | | | | | | | | | | | | | | | | | |
| Phage- | S | L | — | W | — | D | W | E | — | V | D | N | — | F | 0.12 | 0.10 | 0.11 | 896 |
| 791 | | | | | | | | | | | | | | | | | | |
| Phage- | T | I | — | — | — | D | G | Q | — | D | Y | N | — | H | 0.07 | 0.10 | 0.07 | 897 |
| 792 | | | | | | | | | | | | | | | | | | |
| Phage- | F | L | — | — | — | — | G | E | P | T | Y | L | — | T | 0.12 | 0.10 | 0.09 | 898 |
| 793 | | | | | | | | | | | | | | | | | | |
| Phage- | D | D | — | W | L | — | Q | H | D | I | Y | V | — | A | 0.10 | 0.10 | 0.08 | 899 |
| 794 | | | | | | | | | | | | | | | | | | |
| Phage- | I | F | — | — | — | — | V | E | — | V | A | F | — | F | 0.07 | 0.10 | 0.08 | 900 |
| 795 | | | | | | | | | | | | | | | | | | |
| Phage- | A | L | — | — | — | — | V | E | D | D | Y | D | — | L | 0.09 | 0.10 | 0.09 | 901 |
| 796 | | | | | | | | | | | | | | | | | | |
| Phage- | D | D | — | — | — | — | I | E | L | Y | L | T | — | A | 0.08 | 0.10 | 0.08 | 902 |
| 797 | | | | | | | | | | | | | | | | | | |
| Phage- | T | D | — | W | — | D | W | E | D | D | S | I | — | D | 0.07 | 0.10 | 0.07 | 903 |
| 798 | | | | | | | | | | | | | | | | | | |
| Phage- | S | I | — | — | — | — | L | E | — | I | F | L | — | N | 0.08 | 0.10 | 0.08 | 904 |
| 799 | | | | | | | | | | | | | | | | | | |
| Phage- | N | D | — | — | — | — | L | E | — | D | I | L | — | F | 0.07 | 0.10 | 0.09 | 905 |
| 800 | | | | | | | | | | | | | | | | | | |
| Phage- | D | D | — | — | — | — | L | — | — | T | Y | S | — | Y | 0.09 | 0.09 | 0.08 | 906 |
| 801 | | | | | | | | | | | | | | | | | | |
| Phage- | F | V | — | — | — | — | W | E | — | I | D | L | — | I | 0.10 | 0.09 | 0.09 | 907 |
| 802 | | | | | | | | | | | | | | | | | | |
| Phage- | Y | D | — | — | — | — | L | — | — | — | L | S | — | P | 0.07 | 0.09 | 0.07 | 908 |
| 803 | | | | | | | | | | | | | | | | | | |
| Phage- | N | L | — | — | — | — | L | — | — | — | I | I | — | P | 0.08 | 0.09 | 0.08 | 909 |
| 804 | | | | | | | | | | | | | | | | | | |
| Phage- | I | D | — | W | — | D | W | E | — | F | N | N | — | F | 0.08 | 0.09 | 0.09 | 910 |
| 805 | | | | | | | | | | | | | | | | | | |
| Phage- | A | — | — | — | — | — | L | E | — | H | D | Y | — | Y | 0.08 | 0.09 | 0.09 | 911 |
| 806 | | | | | | | | | | | | | | | | | | |
| Phage- | D | D | — | S | — | Q | — | — | Q | I | D | L | — | D | 0.14 | 0.09 | 0.09 | 912 |
| 807 | | | | | | | | | | | | | | | | | | |
| Phage- | S | — | — | — | — | — | S | — | — | — | I | Y | — | Y | 0.08 | 0.09 | 0.07 | 913 |
| 808 | | | | | | | | | | | | | | | | | | |
| Phage- | S | L | — | — | — | — | — | Q | — | D | A | P | — | N | 0.07 | 0.09 | 0.07 | 914 |
| 809 | | | | | | | | | | | | | | | | | | |
| Phage- | D | A | — | — | — | — | L | — | — | T | T | H | — | D | 0.09 | 0.09 | 0.08 | 915 |
| 810 | | | | | | | | | | | | | | | | | | |
| Phage- | N | D | — | — | — | — | V | E | — | V | A | D | — | F | 0.07 | 0.09 | 0.08 | 916 |
| 811 | | | | | | | | | | | | | | | | | | |
| Phage- | Y | I | — | — | — | — | — | E | Q | D | Y | F | — | F | 0.08 | 0.09 | 0.08 | 917 |
| 812 | | | | | | | | | | | | | | | | | | |
| Phage- | T | D | — | — | — | D | G | — | — | T | N | Y | — | F | 0.11 | 0.09 | 0.08 | 918 |
| 813 | | | | | | | | | | | | | | | | | | |
| Phage- | Y | D | — | — | — | — | V | — | — | — | — | L | — | P | 0.08 | 0.09 | 0.08 | 919 |
| 814 | | | | | | | | | | | | | | | | | | |
| Phage- | I | D | — | — | — | — | — | — | — | — | A | Y | — | T | 0.08 | 0.09 | 0.08 | 920 |
| 815 | | | | | | | | | | | | | | | | | | |
| Phage- | F | D | — | — | — | — | — | E | — | F | F | H | — | Y | 0.09 | 0.09 | 0.09 | 921 |
| 816 | | | | | | | | | | | | | | | | | | |
| Phage- | F | P | — | — | — | — | I | E | — | Y | D | Y | — | V | 0.09 | 0.09 | 0.08 | 922 |
| 817 | | | | | | | | | | | | | | | | | | |
| Phage- | A | D | — | — | — | I | — | E | S | I | D | I | — | V | 0.09 | 0.09 | 0.07 | 923 |
| 818 | | | | | | | | | | | | | | | | | | |
| Phage- | I | S | — | — | — | D | L | W | P | T | D | I | — | T | 0.10 | 0.09 | 0.10 | 924 |
| 819 | | | | | | | | | | | | | | | | | | |
| Phage- | D | D | — | — | — | D | G | — | — | V | H | T | — | N | 0.09 | 0.09 | 0.07 | 925 |
| 820 | | | | | | | | | | | | | | | | | | |
| Phage- | I | D | — | W | — | D | W | E | G | — | F | A | — | N | 0.09 | 0.09 | 0.08 | 926 |
| 821 | | | | | | | | | | | | | | | | | | |
| Phage- | L | N | — | — | — | D | G | — | — | T | F | Y | — | D | 0.08 | 0.08 | 0.07 | 927 |
| 822 | | | | | | | | | | | | | | | | | | |
| Phage- | I | H | — | — | — | — | L | G | A | Y | I | S | — | S | 0.08 | 0.08 | 0.09 | 928 |
| 823 | | | | | | | | | | | | | | | | | | |
| Phage- | T | I | — | W | — | D | W | E | — | D | Y | F | — | Y | 0.08 | 0.08 | 0.08 | 929 |
| 824 | | | | | | | | | | | | | | | | | | |
| Phage- | H | S | — | L | A | Q | — | — | Q | D | L | V | — | I | 0.07 | 0.08 | 0.07 | 930 |
| 825 | | | | | | | | | | | | | | | | | | |
| Phage- | N | N | — | A | S | D | L | S | — | D | N | S | — | I | 0.07 | 0.08 | 0.08 | 931 |
| 826 | | | | | | | | | | | | | | | | | | |
| Phage- | — | N | — | W | — | D | W | E | — | D | — | A | — | N | 0.09 | 0.08 | 0.07 | 932 |
| 827 | | | | | | | | | | | | | | | | | | |
| Phage- | — | — | — | — | — | — | L | — | — | — | F | Y | — | F | 0.08 | 0.08 | 0.07 | 933 |
| 828 | | | | | | | | | | | | | | | | | | |
| Phage- | L | F | — | — | T | V | L | — | — | F | F | D | — | D | 0.07 | 0.08 | 0.07 | 934 |
| 829 | | | | | | | | | | | | | | | | | | |
| Phage- | F | D | — | — | — | — | L | — | — | — | — | S | — | A | 0.08 | 0.08 | 0.07 | 935 |
| 830 | | | | | | | | | | | | | | | | | | |
| Phage- | Y | — | — | — | — | — | V | E | — | N | — | Y | — | I | 0.07 | 0.08 | 0.08 | 936 |
| 831 | | | | | | | | | | | | | | | | | | |
| Phage- | D | — | — | — | — | — | L | — | D | — | T | I | — | H | 0.12 | 0.08 | 0.10 | 937 |
| 832 | | | | | | | | | | | | | | | | | | |
| Phage- | — | F | — | — | — | Q | W | A | — | A | N | A | — | F | 0.09 | 0.08 | 0.07 | 938 |
| 833 | | | | | | | | | | | | | | | | | | |
| Phage- | F | T | — | — | — | — | Y | — | — | — | I | T | — | P | 0.09 | 0.08 | 0.09 | 939 |
| 834 | | | | | | | | | | | | | | | | | | |
| Phage- | L | N | — | — | — | V | N | — | — | — | S | V | — | I | 0.07 | 0.08 | 0.08 | 940 |
| 835 | | | | | | | | | | | | | | | | | | |
| Phage- | A | I | — | W | — | D | W | E | — | F | S | D | — | H | 0.07 | 0.08 | 0.07 | 941 |
| 836 | | | | | | | | | | | | | | | | | | |
| Phage- | Y | — | — | — | V | D | L | G | A | N | — | Y | — | Y | 0.10 | 0.08 | 0.09 | 942 |
| 837 | | | | | | | | | | | | | | | | | | |
| Phage- | H | — | — | — | — | — | V | E | — | D | Y | H | — | D | 0.07 | 0.08 | 0.07 | 943 |
| 838 | | | | | | | | | | | | | | | | | | |
| Phage- | N | D | — | — | S | L | Q | Y | D | I | P | T | — | V | 0.08 | 0.08 | 0.07 | 944 |
| 839 | | | | | | | | | | | | | | | | | | |
| Phage- | Y | V | — | R | — | Q | L | — | V | Y | H | Y | — | N | 0.15 | 0.08 | 0.07 | 945 |
| 840 | | | | | | | | | | | | | | | | | | |
| Phage- | H | D | — | — | — | D | G | — | — | — | I | I | — | S | 0.08 | 0.08 | 0.07 | 946 |
| 841 | | | | | | | | | | | | | | | | | | |
| Phage- | F | D | — | — | — | — | L | — | — | T | I | I | — | P | 0.08 | 0.08 | 0.08 | 947 |
| 842 | | | | | | | | | | | | | | | | | | |
| Phage- | — | D | — | — | S | D | R | G | — | N | A | A | — | H | 0.07 | 0.08 | 0.07 | 948 |
| 843 | | | | | | | | | | | | | | | | | | |
| Phage- | N | I | — | L | A | Q | — | N | — | D | P | T | — | N | 0.07 | 0.08 | 0.08 | 949 |
| 844 | | | | | | | | | | | | | | | | | | |
| Phage- | T | N | — | — | S | K | S | Q | V | — | D | H | — | I | 0.10 | 0.08 | 0.09 | 950 |
| 845 | | | | | | | | | | | | | | | | | | |
| Phage- | N | H | — | H | — | Q | — | W | — | L | T | N | — | N | 0.09 | 0.07 | 0.07 | 951 |
| 846 | | | | | | | | | | | | | | | | | | |
| Phage- | L | L | — | H | — | Q | G | — | L | Y | H | L | — | H | 0.09 | 0.07 | 0.08 | 952 |
| 847 | | | | | | | | | | | | | | | | | | |
| Phage- | T | N | — | D | S | K | L | E | G | D | D | N | — | F | 0.09 | 0.07 | 0.07 | 953 |
| 848 | | | | | | | | | | | | | | | | | | |
| Phage- | N | D | — | — | — | — | M | — | — | — | L | L | — | D | 0.08 | 0.07 | 0.07 | 954 |
| 849 | | | | | | | | | | | | | | | | | | |
| Phage- | F | H | — | — | — | — | V | — | — | — | I | N | — | N | 0.07 | 0.07 | 0.07 | 955 |
| 850 | | | | | | | | | | | | | | | | | | |
| Phage- | D | F | — | — | — | D | G | — | — | T | Y | V | — | S | 0.07 | 0.07 | 0.08 | 956 |
| 851 | | | | | | | | | | | | | | | | | | |
| Phage- | — | S | — | — | — | Q | — | — | — | N | N | T | — | N | 0.07 | 0.07 | 0.08 | 957 |
| 852 | | | | | | | | | | | | | | | | | | |
| Phage- | — | — | — | H | L | — | S | E | Q | F | D | I | — | T | 0.08 | 0.07 | 0.08 | 958 |
| 853 | | | | | | | | | | | | | | | | | | |
| Phage- | D | D | — | — | — | — | W | E | F | V | F | F | — | D | 0.08 | 0.07 | 0.08 | 959 |
| 854 | | | | | | | | | | | | | | | | | | |
| Phage- | Y | N | — | E | Q | Q | Q | — | — | D | P | S | — | I | 0.07 | 0.07 | 0.07 | 960 |
| 855 | | | | | | | | | | | | | | | | | | |
| Phage- | N | T | — | — | T | — | Q | H | — | F | N | — | — | L | 0.08 | 0.07 | 0.08 | 961 |
| 856 | | | | | | | | | | | | | | | | | | |
| Phage- | H | P | — | Q | — | G | — | E | — | V | D | Y | — | V | 0.08 | 0.07 | 0.08 | 962 |
| 857 | | | | | | | | | | | | | | | | | | |
| Phage- | — | A | — | S | R | Q | L | G | — | D | A | Y | — | N | 0.07 | 0.07 | 0.07 | 963 |
| 858 | | | | | | | | | | | | | | | | | | |
| Phage- | D | I | — | — | A | Q | E | V | H | V | Y | T | — | P | 0.07 | 0.07 | 0.07 | 964 |
| 859 | | | | | | | | | | | | | | | | | | |
| Phage- | F | F | — | E | G | N | L | — | A | Y | L | L | — | L | 0.08 | 0.07 | 0.08 | 965 |
| 860 | | | | | | | | | | | | | | | | | | |
| Phage- | Y | — | — | — | — | D | G | E | — | N | I | V | — | D | 0.07 | 0.07 | 0.07 | 966 |
| 861 | | | | | | | | | | | | | | | | | | |

TABLE 21
Sequences of those peptides
selected for synthesis
(CD3 scFv Peptide-B Optimization)

| | | SEQ |
|---|---|---|
| | | ID |
| Peptide-ID | Sequence | NO: |
| Peptide-AA | DDCWPDWEFDFACA | 106 |
| Peptide-AB | YICGLDFPDFLYCD | 107 |
| Peptide-AC | FDCWPDWEEYFVCD | 108 |
| Peptide-AD | YICWPDWEEYFDCD | 109 |
| Peptide-AE | NICWPDWEDDYFCF | 110 |
| Peptide-AF | NFCWPDWEYIYPCI | 111 |
| Peptide-AG | VDCWPDWEEDFLCI | 112 |
| Peptide-AH | HACWPDWEEYFPCN | 113 |
| Peptide-AI | YDCGPDVDESYVCV | 114 |
| Peptide-AJ | IDCWPDWEDDTFCY | 115 |
| Peptide-AK | YLCGPDGDETLACY | 116 |
| Peptide-AL | VDCGPDGDESILCY | 117 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. An isolated recombinant nucleic acid molecule encoding a polypeptide or polypeptide complex according to Formula I: 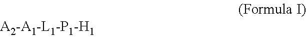
wherein:
A₁ comprises a first antigen recognizing molecule that binds to an effector cell antigen, wherein A₁ comprises an anti-CD3 binding molecule comprising complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1, HC-CDR2: SEQ ID NO: 2, and HC-CDR3: SEQ ID NO: 3; and A₁ comprises CDRs: LC-CDR1, LC-CDR2, and LC-CDR3, wherein the LC-CDR1, the LC-CDR2, and the LC-CDR3 of A₁ comprise LC-CDR1: SEQ ID NO: 4, LC-CDR2: SEQ ID NO:5, and LC-CDR3: SEQ ID NO: 6;
P₁ comprises a peptide that binds to A₁, wherein P₁ comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S;
L₁ comprises a linking moiety that connects A₁ to P₁ and is a substrate for a tumor specific protease;
H₁ comprises a half-life extending molecule; and
A₂ comprises a second antigen recognizing molecule that binds to prostate-specific membrane antigen (PSMA).

2. The isolated recombinant nucleic acid of claim 1, wherein A2 comprises complementarity determining regions (CDRs): HC-CDR1, HC-CDR2, and HC-CDR3, wherein the HC-CDR1, the HC-CDR2, and the HC-CDR3 comprise: HC-CDR1: SEQ ID NO: 8, HC-CDR2: SEQ ID NO: 9, and HC-CDR3: SEQ ID NO: 10; and A2 comprises CDRs: LC-CDR1, LC-CDR2, and LC-CDR3, wherein the LC-CDR1, the LC-CDR2, and the LC-CDR3 of A2 comprise LC-CDR1: SEQ ID NO: 11, LC-CDR2: SEQ ID NO:12, and LC-CDR3: SEQ ID NO: 13.

3. The isolated recombinant nucleic acid of claim 1, wherein the effector cell antigen comprises cluster of differentiation 3 (CD3).

4. The isolated recombinant nucleic acid of claim 1, wherein A₁ comprises an antibody format selected from single chain variable fragment and a Fab or Fab′ fragment.

5. The isolated recombinant nucleic acid of claim 1, wherein A₂ comprises an antibody format selected from single chain variable fragment, a single domain antibody, and a Fab or Fab′ fragment.

6. The isolated recombinant nucleic acid of claim 1, wherein A₁ comprises an antibody format of a single chain variable fragment (scFv), and A₂ comprises an antibody format of a Fab or Fab′.

7. The isolated recombinant nucleic acid of claim 1, wherein P₁ becomes unbound from A₁ when L₁ is cleaved by the tumor specific protease thereby exposing A₁ to the effector cell antigen.

8. The isolated recombinant nucleic acid of claim 1, wherein the tumor specific protease is selected from the group consisting of a matrix metalloprotease (MMP), serine protease, cysteine protease, threonine protease, and aspartic protease.

9. The isolated recombinant nucleic acid of claim 8, wherein the matrix metalloprotease comprises MMP2, MMP7, MMP9, MMP13, or MMP14.

10. The isolated recombinant nucleic acid of claim 8, wherein the serine protease comprises matriptase (MTSP1), urokinase, or hepsin.

11. The isolated recombinant nucleic acid of claim 1, wherein L₁ comprises a urokinase cleavable amino acid sequence, a matriptase cleavable amino acid sequence, a matrix metalloprotease cleavable amino acid sequence, or a legumain cleavable amino acid sequence.

12. The isolated recombinant nucleic acid of claim 1, wherein L₁ comprises an amino acid sequence according to SEQ ID NO: 23.

13. The isolated recombinant nucleic acid of claim 1, wherein L₁ comprises an amino acid sequence according to any one of SEQ ID NOs: 20-49.

14. The isolated recombinant nucleic acid of claim 1, wherein L₁ comprises an amino acid sequence of Linker 25 (ISSGLLSGRSDAG) (SEQ ID NO: 45), Linker 26 (AAGLLAPPGGLSGRSDAG) (SEQ ID NO: 46), Linker 27 (SPLGLSGRSDAG) (SEQ ID NO: 47), or Linker 28 (LSGRSDAGSPLGLAG) (SEQ ID NO: 48), or an amino acid sequence that has 1, 2, or 3 amino acid substitutions, additions, or deletions relative to the amino acid sequences of Linker 25, Linker 26, Linker 27, or Linker 28.

15. The isolated recombinant nucleic acid of claim 1, wherein H₁ comprises serum albumin.

16. The isolated recombinant nucleic acid of claim 15, wherein the albumin is human serum albumin.

17. The isolated recombinant nucleic acid of claim 1, wherein H₁ comprises a single domain antibody.

18. The isolated recombinant nucleic acid of claim 1, wherein H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 54, HC-CDR2: SEQ ID NO: 55, and HC-CDR3: SEQ ID NO: 56.

19. The isolated recombinant nucleic acid of claim 1, wherein H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 58, HC-CDR2: SEQ ID NO: 59, and HC-CDR3: SEQ ID NO: 60.

20. The isolated recombinant nucleic acid of claim 1, wherein U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P.

21. The isolated recombinant nucleic acid of claim 20, wherein U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; U₁₄ is selected from D, Y, M, and N.

22. The isolated recombinant nucleic acid of claim 1, wherein P₁ comprises the amino acid sequence according to any one of SEQ ID NOs: 93-95 and 102-105.

23. The isolated recombinant nucleic acid of claim 1, wherein P₁ comprises the amino acid sequence according to any one of SEQ ID NOs: 106 and 108-117.

24. The isolated recombinant nucleic acid of claim 1, wherein P₁ comprises the amino acid sequence according to SEQ ID NO: 19.

25. The isolated recombinant nucleic acid of claim 1, wherein P₁ comprises the amino acid sequence according to SEQ ID NO: 116.

26. The isolated recombinant nucleic acid of claim 1, wherein the polypeptide or polypeptide complex comprises the amino acid sequences according to SEQ ID NO: 72 and SEQ ID NO: 73.


SEQUENCE LISTING (966 sequences)

SEQ ID NO: 1 - protein, 8 aa, synthetic construct
  REGION 1..8: note = Description of Artificial Sequence: Synthetic peptide
GFTFNKYA

SEQ ID NO: 2 - protein, 10 aa, synthetic construct
  REGION 1..10: note = Description of Artificial Sequence: Synthetic peptide
IRSKYNNYAT

SEQ ID NO: 3 - protein, 16 aa, synthetic construct
  REGION 1..16: note = Description of Artificial Sequence: Synthetic peptide
VRHGNFGNSYISYWAY

SEQ ID NO: 4 - protein, 9 aa, synthetic construct
  REGION 1..9: note = Description of Artificial Sequence: Synthetic peptide
TGAVTSGNY

SEQ ID NO: 5 - skipped

SEQ ID NO: 6 - protein, 9 aa, synthetic construct
  REGION 1..9: note = Description of Artificial Sequence: Synthetic peptide
VLWYSNRWV

SEQ ID NO: 7 - protein, 249 aa, synthetic construct
  REGION 1..249: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 8 - protein, 8 aa, synthetic construct
  REGION 1..8: note = Description of Artificial Sequence: Synthetic peptide
GFAFSRYG

SEQ ID NO: 9 - protein, 8 aa, synthetic construct
  REGION 1..8: note = Description of Artificial Sequence: Synthetic peptide
IWYDGSNK

SEQ ID NO: 10 - protein, 16 aa, synthetic construct
  REGION 1..16: note = Description of Artificial Sequence: Synthetic peptide
ARGGDFLYYYYYGMDV

SEQ ID NO: 11 - protein, 6 aa, synthetic construct
  REGION 1..6: note = Description of Artificial Sequence: Synthetic peptide
QGISNY

SEQ ID NO: 12 - skipped

SEQ ID NO: 13 - protein, 9 aa, synthetic construct
  REGION 1..9: note = Description of Artificial Sequence: Synthetic peptide
QNYNSAPFT

SEQ ID NO: 14 - protein, 214 aa, synthetic construct
  REGION 1..214: note = Description of Artificial Sequence: Synthetic polypeptide
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 226 aa, synthetic construct
  REGION 1..226: note = Description of Artificial Sequence: Synthetic polypeptide
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 16 - protein, 16 aa, synthetic construct
  REGION 1..16: note = Description of Artificial Sequence: Synthetic peptide
GGGSQCLGPEWEVCPY

SEQ ID NO: 17 - protein, 16 aa, synthetic construct
  REGION 1..16: note = Description of Artificial Sequence: Synthetic peptide
GGVYCGPEFDESVGCM

SEQ ID NO: 18 - protein, 14 aa, synthetic construct
  REGION 1..14: note = Description of Artificial Sequence: Synthetic peptide
GSQCLGPEWEVCPY

SEQ ID NO: 19 - protein, 14 aa, synthetic construct
  REGION 1..14: note = Description of Artificial Sequence: Synthetic peptide
VYCGPEFDESVGCM

SEQ ID NO: 20 - protein, 15 aa, synthetic construct
  REGION 1..15: note = Description of Artificial Sequence: Synthetic peptide
GGGGSGGGGSGGGGS

SEQ ID NO: 21 - protein, 5 aa, synthetic construct
  REGION 1..5: note = Description of Artificial Sequence: Synthetic peptide
GGGGS

SEQ ID NO: 22 - protein, 9 aa, synthetic construct
  REGION 1..9: note = Description of Artificial Sequence: Synthetic peptide
GGGGSGGGS

SEQ ID NO: 23 - protein, 28 aa, synthetic construct
  REGION 1..28: note = Description of Artificial Sequence: Synthetic peptide
GGGGSGGGLSGRSDAGSPLGLAGSGGGS

SEQ ID NO: 24 - protein, 18 aa, synthetic construct
  REGION 1..18: note = Description of Artificial Sequence: Synthetic peptide
GGGGSLSGRSDNHGSSGT

SEQ ID NO: 25 - protein, 26 aa, synthetic construct
  REGION 1..26: note = Description of Artificial Sequence: Synthetic peptide
GGGGSSGGSGGSGLSGRSDNHGSSGT

SEQ ID NO: 26 - protein, 8 aa, synthetic construct
  REGION 1..8: note = Description of Artificial Sequence: Synthetic peptide
ASGRSDNH

SEQ ID NO: 27 - protein, 8 aa, synthetic construct
  REGION 1..8: note = Description of Artificial Sequence: Synthetic peptide
LAGRSDNH

SEQ ID NO: 28 - protein, 13 aa, synthetic construct
  REGION 1..13: note = Description of Artificial Sequence: Synthetic peptide
ISSGLASGRSDNH

SEQ ID NO: 29 - protein, 13 aa, synthetic construct
  REGION 1..13: note = Description of Artificial Sequence: Synthetic peptide
ISSGLLAGRSDNH

SEQ ID NO: 30 - protein, 8 aa, synthetic construct
  REGION 1..8: note = Description of Artificial Sequence: Synthetic peptide
LSGRSDNH

SEQ ID NO: 31 - protein, 13 aa, synthetic construct
  REGION 1..13: note = Description of Artificial Sequence: Synthetic peptide
ISSGLLSGRSDNP

SEQ ID NO: 32 - protein, 13 aa, synthetic construct
  REGION 1..13: note = Description of Artificial Sequence: Synthetic peptide
ISSGLLSGRSDNH

SEQ ID NO: 33 - protein, 16 aa, synthetic construct
  REGION 1..16: note = Description of Artificial Sequence: Synthetic peptide
LSGRSDNHSPLGLAGS

SEQ ID NO: 34 - protein, 16 aa, synthetic construct
  REGION 1..16: note = Description of Artificial Sequence: Synthetic peptide
SPLGLAGSLSGRSDNH

SEQ ID NO: 35 - protein, 12 aa, synthetic construct
  REGION 1..12: note = Description of Artificial Sequence: Synthetic peptide
SPLGLSGRSDNH

SEQ ID NO: 36 - protein, 16 aa, synthetic construct
  REGION 1..16: note = Description of Artificial Sequence: Synthetic peptide
LAGRSDNHSPLGLAGS

SEQ ID NO: 37 - protein, 16 aa, synthetic construct
  REGION 1..16: note = Description of Artificial Sequence: Synthetic peptide
LSGRSDNHVPLSLKMG

SEQ ID NO: 38 - protein, 16 aa, synthetic construct
  REGION 1..16: note = Description of Artificial Sequence: Synthetic peptide
LSGRSDNHVPLSLSMG

SEQ ID NO: 39 - protein, 31 aa, synthetic construct
  REGION 1..31: note = Description of Artificial Sequence: Synthetic polypeptide
GSSGGSGGSGGSGISSGLLSGRSDNHGSSGT

SEQ ID NO: 40 - protein, 28 aa, synthetic construct
  REGION 1..28: note = Description of Artificial Sequence: Synthetic peptide
GSSGGSGGSGGISSGLLSGRSDNHGGGS

SEQ ID NO: 41 - protein, 8 aa, synthetic construct
  REGION 1..8: note = Description of Artificial Sequence: Synthetic peptide
ASGRSDNH

SEQ ID NO: 42 - protein, 8 aa, synthetic construct
  REGION 1..8: note = Description of Artificial Sequence: Synthetic peptide
LAGRSDNH

SEQ ID NO: 43 - protein, 13 aa, synthetic construct
  REGION 1..13: note = Description of Artificial Sequence: Synthetic peptide
ISSGLASGRSDNH

SEQ ID NO: 44 - protein, 8 aa, synthetic construct
  REGION 1..8: note = Description of Artificial Sequence: Synthetic peptide
LSGRSDAG

SEQ ID NO: 45 - protein, 13 aa, synthetic construct
  REGION 1..13: note = Description of Artificial Sequence: Synthetic peptide
ISSGLLSGRSDAG

SEQ ID NO: 46 - protein, 18 aa, synthetic construct
  REGION 1..18: note = Description of Artificial Sequence: Synthetic peptide
AAGLLAPPGGLSGRSDAG

SEQ ID NO: 47 - protein, 12 aa, synthetic construct
  REGION 1..12: note = Description of Artificial Sequence: Synthetic peptide
SPLGLSGRSDAG

SEQ ID NO: 48 - protein, 15 aa, synthetic construct
  REGION 1..15: note = Description of Artificial Sequence: Synthetic peptide
LSGRSDAGSPLGLAG

SEQ ID NO: 49 - protein, 28 aa, synthetic construct
  REGION 1..28: note = Description of Artificial Sequence: Synthetic peptide
GGGGSGGGSGGGGSGGASSGAGGSGGGS

SEQ ID NO: 50 - protein, 5 aa, synthetic construct
  REGION 1..5: note = Description of Artificial Sequence: Synthetic peptide
GSGGS

SEQ ID NO: 51 - protein, 4 aa, synthetic construct
  REGION 1..4: note = Description of Artificial Sequence: Synthetic peptide
GGGS

SEQ ID NO: 52 - protein, 5 aa, synthetic construct
  REGION 1..5: note = Description of Artificial Sequence: Synthetic peptide
GGGGS

SEQ ID NO: 53 - protein, 6 aa, synthetic construct
  REGION 1..6: note = Description of Artificial Sequence: Synthetic peptide
GSSGGS

SEQ ID NO: 54 - protein, 8 aa, synthetic construct
  REGION 1..8: note = Description of Artificial Sequence: Synthetic peptide
GSTFYTAV

SEQ ID NO: 55 - protein, 8 aa, synthetic construct
  REGION 1..8: note = Description of Artificial Sequence: Synthetic peptide
IRWTALTT

SEQ ID NO: 56 - protein, 17 aa, synthetic construct
  REGION 1..17: note = Description of Artificial Sequence: Synthetic peptide
AARGTLGLFTTADSYDY

SEQ ID NO: 57 - protein, 124 aa, synthetic construct
  REGION 1..124: note = Description of Artificial Sequence: Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSS

SEQ ID NO: 58 - protein, 8 aa, synthetic construct
  REGION 1..8: note = Description of Artificial Sequence: Synthetic peptide
GFTFSKFG

SEQ ID NO: 59 - protein, 8 aa, synthetic construct
  REGION 1..8: note = Description of Artificial Sequence: Synthetic peptide
ISGSGRDT

SEQ ID NO: 60 - protein, 8 aa, synthetic construct
  REGION 1..8: note = Description of Artificial Sequence: Synthetic peptide
TIGGSLSV

SEQ ID NO: 61 - protein, 115 aa, synthetic construct
  REGION 1..115: note = Description of Artificial Sequence: Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 62 - protein, 214 aa, synthetic construct; identical to SEQ ID NO: 14
  REGION 1..214: note = Description of Artificial Sequence: Synthetic polypeptide

SEQ ID NO: 63 - protein, 480 aa, synthetic construct
  REGION 1..480: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 468 aa, synthetic construct
  REGION 1..468: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 226 aa, synthetic construct; identical to SEQ ID NO: 15
  REGION 1..226: note = Description of Artificial Sequence: Synthetic polypeptide

SEQ ID NO: 66 - protein, 214 aa, synthetic construct; identical to SEQ ID NO: 14
  REGION 1..214: note = Description of Artificial Sequence: Synthetic polypeptide

SEQ ID NO: 67 - protein, 657 aa, synthetic construct
  REGION 1..657: note = Description of Artificial Sequence: Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGGSQCLGPEWEVCPYGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 68 - protein, 645 aa, synthetic construct
  REGION 1..645: note = Description of Artificial Sequence: Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGGSQCLGPEWEVCPYGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 226 aa, synthetic construct; identical to SEQ ID NO: 15
  REGION 1..226: note = Description of Artificial Sequence: Synthetic polypeptide

SEQ ID NO: 70 - protein, 214 aa, synthetic construct; identical to SEQ ID NO: 14
  REGION 1..214: note = Description of Artificial Sequence: Synthetic polypeptide

SEQ ID NO: 71 - protein, 657 aa, synthetic construct
  REGION 1..657: note = Description of Artificial Sequence: Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 72 - protein, 645 aa, synthetic construct
  REGION 1..645: note = Description of Artificial Sequence: Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 226 aa, synthetic construct; identical to SEQ ID NO: 15
  REGION 1..226: note = Description of Artificial Sequence: Synthetic polypeptide

SEQ ID NO: 74 - protein, 636 aa, synthetic construct
  REGION 1..636: note = Description of Artificial Sequence: Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 226 aa, synthetic construct; identical to SEQ ID NO: 15
  REGION 1..226: note = Description of Artificial Sequence: Synthetic polypeptide

SEQ ID NO: 76 - protein, 636 aa, synthetic construct
  REGION 1..636: note = Description of Artificial Sequence: Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGSGGGGSGGASSGAGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 226 aa, synthetic construct; identical to SEQ ID NO: 15
  REGION 1..226: note = Description of Artificial Sequence: Synthetic polypeptide

SEQ ID NO: 78 - protein, 14 aa, synthetic construct
  REGION 1..14: note = Description of Artificial Sequence: Synthetic peptide
YLWGCEWNCAGITT

SEQ ID NO: 79 - protein, 14 aa, synthetic construct
  REGION 1..14: note = Description of Artificial Sequence: Synthetic peptide
GSQCLGPEWEVCPY

SEQ ID NO: 80 - protein, 14 aa, synthetic construct
  REGION 1..14: note = Description of Artificial Sequence: Synthetic peptide
ASQCLGPEWEVCPY

SEQ ID NO: 81 - protein, 14 aa, synthetic construct
  REGION 1..14: note = Description of Artificial Sequence: Synthetic peptide
GAQCLGPEWEVCPY

SEQ ID NO: 82 - protein, 14 aa, synthetic construct
  REGION 1..14: note = Description of Artificial Sequence: Synthetic peptide
GSACLGPEWEVCPY

SEQ ID NO: 83 - protein, 14 aa, synthetic construct
  REGION 1..14: note = Description of Artificial Sequence: Synthetic peptide
GSQCAGPEWEVCPY

SEQ ID NO: 84 - protein, 14 aa, synthetic construct
  REGION 1..14: note = Description of Artificial Sequence: Synthetic peptide
GSQCLAPEWEVCPY

SEQ ID NO: 85 - protein, 14 aa, synthetic construct
  REGION 1..14: note = Description of Artificial Sequence: Synthetic peptide
GSQCLGAEWEVCPY

SEQ ID NO: 86 - protein, 14 aa, synthetic construct
  REGION 1..14: note = Description of Artificial Sequence: Synthetic peptide
GSQCLGPAWEVCPY

SEQ ID NO: 87 - protein, 14 aa, synthetic construct
  REGION 1..14: note = Description of Artificial Sequence: Synthetic peptide
GSQCLGPEAEVCPY

SEQ ID NO: 88 - protein, 14 aa, synthetic construct
  REGION 1..14: note = Description of Artificial Sequence: Synthetic peptide
GSQCLGPEWAVCPY

SEQ ID NO: 89 - protein, 14 aa, synthetic construct
  REGION 1..14: note = Description of Artificial Sequence: Synthetic peptide
GSQCLGPEWEACPY

SEQ ID NO: 90 - protein, 14 aa, synthetic construct
  REGION 1..14: note = Description of Artificial Sequence: Synthetic peptide
GSQCLGPEWEVCAY

SEQ ID NO: 91 - protein, 14 aa, synthetic construct
  REGION 1..14: note = Description of Artificial Sequence: Synthetic peptide
GSQCLGPEWEVCPA

SEQ ID NO: 92 - protein, 14 aa, synthetic construct
  REGION 1..14: note = Description of Artificial Sequence: Synthetic peptide
GSQCLGPEWEVCPY

SEQ ID NO: 93 - protein, 14 aa, synthetic construct
  REGION 1..14: note = Description of Artificial Sequence: Synthetic peptide
VYCGPEFDESVGCM

SEQ ID NO: 94 - protein, 14 aa, synthetic construct
  REGION 1..14: note = Description of Artificial Sequence: Synthetic peptide
AYCGPEFDESVGCM

SEQ ID NO: 95 - protein, 14 aa, synthetic construct
  REGION 1..14: note = Description of Artificial Sequence: Synthetic peptide
VACGPEFDESVGCM

SEQ ID NO: 96 - protein, 14 aa, synthetic construct
  REGION 1..14: note = Description of Artificial Sequence: Synthetic peptide
VYCAPEFDESVGCM

SEQ ID NO: 97 - protein, 14 aa, synthetic construct
  REGION 1..14: note = Description of Artificial Sequence: Synthetic peptide
VYCGAEFDESVGCM

SEQ ID NO: 98 - protein, 14 aa, synthetic construct
  REGION 1..14: note = Description of Artificial Sequence: Synthetic peptide
VYCGPAFDESVGCM

SEQ ID NO: 99 - protein, 14 aa, synthetic construct
  REGION 1..14: note = Description of Artificial Sequence: Synthetic peptide
VYCGPEADESVGCM

SEQ ID NO: 100 - protein, 14 aa, synthetic construct
  REGION 1..14: note = Description of Artificial Sequence: Synthetic peptide
VYCGPEFAESVGCM

SEQ ID NO: 101 - protein, 14 aa, synthetic construct
  REGION 1..14: note = Description of Artificial Sequence: Synthetic peptide
VYCGPEFDASVGCM

SEQ ID NO: 102 - protein, 14 aa, synthetic construct
  REGION 1..14: note = Description of Artificial Sequence: Synthetic peptide
VYCGPEFDEAVGCM

SEQ ID NO: 103 - protein, 14 aa, synthetic construct
  REGION 1..14: note = Description of Artificial Sequence: Synthetic peptide
VYCGPEFDESAGCM

SEQ ID NO: 104 - protein, 14 aa, synthetic construct
  REGION 1..14: note = Description of Artificial Sequence: Synthetic peptide
VYCGPEFDESVACM

SEQ ID NO: 105 - protein, 14 aa, synthetic construct
  REGION 1..14: note = Description of Artificial Sequence: Synthetic peptide
VYCGPEFDESVGCA

SEQ ID NO: 106 - protein, 14 aa, synthetic construct
  REGION 1..14: note = Description of Artificial Sequence: Synthetic peptide
DDCWPDWEFDFACA

SEQ ID NO: 107 - protein, 14 aa, synthetic construct
  REGION 1..14: note = Description of Artificial Sequence: Synthetic peptide
YICGLDFPDFLYCD

SEQ ID NO: 108 - protein, 14 aa, synthetic construct
  REGION 1..14: note = Description of Artificial Sequence: Synthetic peptide
FDCWPDWEEYFVCD

SEQ ID NO: 109 - protein, 14 aa, synthetic construct
  REGION 1..14: note = Description of Artificial Sequence: Synthetic peptide
YICWPDWEEYFDCD

SEQ ID NO: 110 - protein, 14 aa, synthetic construct
  REGION 1..14: note = Description of Artificial Sequence: Synthetic peptide
NICWPDWEDDYFCF

SEQ ID NO: 111 - protein, 14 aa, synthetic construct
  REGION 1..14: note = Description of Artificial Sequence: Synthetic peptide
NFCWPDWEYIYPCI

SEQ ID NO: 112 - protein, 14 aa, synthetic construct
  REGION 1..14: note = Description of Artificial Sequence: Synthetic peptide
VDCWPDWEEDFLCI

SEQ ID NO: 113 - protein, 14 aa, synthetic construct
  REGION 1..14: note = Description of Artificial Sequence: Synthetic peptide
HACWPDWEEYFPCN

SEQ ID NO: 114 - protein, 14 aa, synthetic construct
  REGION 1..14: note = Description of Artificial Sequence: Synthetic peptide
YDCGPDVDESYVCV

SEQ ID NO: 115 - protein, 14 aa, synthetic construct
  REGION 1..14: note = Description of Artificial Sequence: Synthetic peptide
IDCWPDWEDDTFCY

SEQ ID NO: 116 - protein, 14 aa, synthetic construct
  REGION 1..14: note = Description of Artificial Sequence: Synthetic peptide
YLCGPDGDETLACY

SEQ ID NO: 117 - protein, 14 aa, synthetic construct
  REGION 1..14: note = Description of Artificial Sequence: Synthetic peptide
VDCGPDGDESILCY

SEQ ID NO: 118 - protein, 9 aa, synthetic construct
  REGION 1..9: note = Description of Artificial Sequence: Synthetic peptide
  SITE 1..9: note = This sequence may encompass 1-3 "Gly Gly Ser" repeating units
GGSGGSGGS

SEQ ID NO: 119 - protein, 14 aa, synthetic construct
  REGION 1..14: note = Description of Artificial Sequence: Synthetic peptide
FICWPDWEEDYFCA

SEQ ID NO: 120 - protein, 14 aa, synthetic construct
  REGION 1..14: note = Description of Artificial Sequence: Synthetic peptide
GDCWPDWEWDFYCD

SEQ ID NO: 121 - protein, 14 aa, synthetic construct
  REGION 1..14: note = Description of Artificial Sequence: Synthetic peptide
YLCWPDWEYIDLCD

SEQ ID NO: 122 - protein, 14 aa, synthetic construct
  REGION 1..14: note = Description of Artificial Sequence: Synthetic peptide
SFCWPDWEEYFDCD

SEQ ID NO: 123 - protein, 14 aa, synthetic construct
  REGION 1..14: note = Description of Artificial Sequence: Synthetic peptide
DDCWPDWEEYASCD

SEQ ID NO: 124 - protein, 14 aa, synthetic construct
  REGION 1..14: note = Description of Artificial Sequence: Synthetic peptide
NLCWPDWEYPFFCD

SEQ ID NO: 125 - protein, 14 aa, synthetic construct
  REGION 1..14: note = Description of Artificial Sequence: Synthetic peptide
FDCWPDWEESFVCD

SEQ ID NO: 126 - protein, 14 aa, synthetic construct
  REGION 1..14: note = Description of Artificial Sequence: Synthetic peptide
DICGPDGDETIICD

SEQ ID NO: 127 - protein, 14 aa, synthetic construct
  REGION 1..14: note = Description of Artificial Sequence: Synthetic peptide
DDCWPDWEYYAVCD

SEQ ID NO: 128 - protein, 14 aa, synthetic construct
  REGION 1..14: note = Description of Artificial Sequence: Synthetic peptide
YDCWPDWEEYSNCD

SEQ ID NO: 129 - protein, 14 aa, synthetic construct
  REGION 1..14: note = Description of Artificial Sequence: Synthetic peptide
INCWPDWEDYFFCD

SEQ ID NO: 130 - protein, 14 aa, synthetic construct
  REGION 1..14: note = Description of Artificial Sequence: Synthetic peptide
NICWPDWEDDTFCF

SEQ ID NO: 131 - protein, 14 aa, synthetic construct
  REGION 1..14: note = Description of Artificial Sequence: Synthetic peptide
NICWPDWEPNSFCF

SEQ ID NO: 132 - protein, 14 aa, synthetic construct
  REGION 1..14: note = Description of Artificial Sequence: Synthetic peptide
YDCGPEMDESIDCF

SEQ ID NO: 133 - protein, 14 aa, synthetic construct
  REGION 1..14: note = Description of Artificial Sequence: Synthetic peptide
DFCWPDWEFPFICH

SEQ ID NO: 134 - protein, 14 aa, synthetic construct
  REGION 1..14: note = Description of Artificial Sequence: Synthetic peptide
DFCGPEMDESITCI

SEQ ID NO: 135 - protein, 14 aa, synthetic construct
  REGION 1..14: note = Description of Artificial Sequence: Synthetic peptide
YDCGPEFDESTVCI

SEQ ID NO: 136 - protein, 14 aa, synthetic construct
  REGION 1..14: note = Description of Artificial Sequence: Synthetic peptide
HDCWPDWEWDIFCI

SEQ ID NO: 137 - protein, 14 aa, synthetic construct
  REGION 1..14: note = Description of Artificial Sequence: Synthetic peptide
HACWPDWEEYNPCN

SEQ ID NO: 138 - protein, 14 aa, synthetic construct
  REGION 1..14: note = Description of Artificial Sequence: Synthetic peptide
DVCWPDWEWDFFCN

SEQ ID NO: 139 - protein, 14 aa, synthetic construct
  REGION 1..14: note = Description of Artificial Sequence: Synthetic peptide
NYCWPDWEYYIPCN

SEQ ID NO: 140 - protein, 14 aa, synthetic construct
  REGION 1..14: note = Description of Artificial Sequence: Synthetic peptide
IICWPDWEFIDYCN

SEQ ID NO: 141 - protein, 14 aa, synthetic construct
  REGION 1..14: note = Description of Artificial Sequence: Synthetic peptide
SLCWPDWEYDIACP

SEQ ID NO: 142 - protein, 14 aa, synthetic construct
  REGION 1..14: note = Description of Artificial Sequence: Synthetic peptide
DLCGPELDESIFCP

SEQ ID NO: 143 - protein, 14 aa, synthetic construct
  REGION 1..14: note = Description of Artificial Sequence: Synthetic peptide
TNCWPDWEWVLPCP

SEQ ID NO: 144 - protein, 14 aa, synthetic construct
  REGION 1..14: note = Description of Artificial Sequence: Synthetic peptide
IECWPDWEPNYFCP

SEQ ID NO: 145 - protein, 14 aa, synthetic construct
  REGION 1..14: note = Description of Artificial Sequence: Synthetic peptide
IFCWPDWEDYVDCP

SEQ ID NO: 146 - protein, 14 aa, synthetic construct
  REGION 1..14: note = Description of Artificial Sequence: Synthetic peptide
IDCWPDWEYDFFCP

SEQ ID NO: 147 - protein, 14 aa, synthetic construct
  REGION 1..14: note = Description of Artificial Sequence: Synthetic peptide
LFCWPDWEDSFFCP

SEQ ID NO: 148 - protein, 14 aa, synthetic construct
  REGION 1..14: note = Description of Artificial Sequence: Synthetic peptide
VDCWPDWEDYADCT

SEQ ID NO: 149 - protein, 14 aa, synthetic construct
  REGION 1..14: note = Description of Artificial Sequence: Synthetic peptide
VICWPDWEQYFPCV

SEQ ID NO: 150 - protein, 14 aa, synthetic construct
  REGION 1..14: note = Description of Artificial Sequence: Synthetic peptide
IECWPDWEPIYPCY

SEQ ID NO: 151 - protein, 14 aa, synthetic construct
  REGION 1..14: note = Description of Artificial Sequence: Synthetic peptide
ITCWPDWEVYFPCY

SEQ ID NO: 152 - protein, 14 aa, synthetic construct
  REGION 1..14: note = Description of Artificial Sequence: Synthetic peptide
IDCWPDWEYIHPCY

SEQ ID NO: 153 - protein, 14 aa, synthetic construct
  REGION 1..14: note = Description of Artificial Sequence: Synthetic peptide
IDCWPDWEYINPCY

SEQ ID NO: 154 - protein, 14 aa, synthetic construct
  REGION 1..14: note = Description of Artificial Sequence: Synthetic peptide
ADCWPDWEEAFPCY

SEQ ID NO: 155 - protein, 14 aa, synthetic construct
  REGION 1..14: note = Description of Artificial Sequence: Synthetic peptide
IDCWPDWEYIYPCY

SEQ ID NO: 156 - protein, 14 aa, synthetic construct
  REGION 1..14: note = Description of Artificial Sequence: Synthetic peptide
NICWPDWEDDNFCF

SEQ ID NO: 157 - protein, 14 aa, synthetic construct
  REGION 1..14: note = Description of Artificial Sequence: Synthetic peptide
YDCWPDWEYVDACY

SEQ ID NO: 158 - protein, 14 aa, synthetic construct
  REGION 1..14: note = Description of Artificial Sequence: Synthetic peptide
FYCGPDGDESYVCD

SEQ ID NO: 159 - protein, 14 aa, synthetic construct
  REGION 1..14: note = Description of Artificial Sequence: Synthetic peptide
DICWPDWEYINICS

SEQ ID NO: 160 - protein, 14 aa, synthetic construct
  REGION 1..14: note = Description of Artificial Sequence: Synthetic peptide
FVCWPDWEDFNFCD

SEQ ID NO: 161 - protein, 14 aa, synthetic construct
  REGION 1..14: note = Description of Artificial Sequence: Synthetic peptide
FACWPDWEDYVACD

SEQ ID NO: 162 - protein, 14 aa, synthetic construct
  REGION 1..14: note = Description of Artificial Sequence: Synthetic peptide
DNCWPDWEYDFFCV

SEQ ID NO: 163 - protein, 14 aa, synthetic construct
  REGION 1..14: note = Description of Artificial Sequence: Synthetic peptide
YDCWPDWEEYNDCA

SEQ ID NO: 164 - protein, 14 aa, synthetic construct
  REGION 1..14: note = Description of Artificial Sequence: Synthetic peptide
DDCGPDGDETIICV

SEQ ID NO: 165 - protein, 14 aa, synthetic construct
  REGION 1..14: note = Description of Artificial Sequence: Synthetic peptide
FPCWPDWEEYAICD

SEQ ID NO: 166 - protein, 14 aa, synthetic construct
  REGION 1..14: note = Description of Artificial Sequence: Synthetic peptide
PDCGPDGDESLFCT

SEQ ID NO: 167 - protein, 14 aa, synthetic construct
  REGION 1..14: note = Description of Artificial Sequence: Synthetic peptide
DNCWPDWEYDYFCV

SEQ ID NO: 168 - protein, 14 aa, synthetic construct
  REGION 1..14: note = Description of Artificial Sequence: Synthetic peptide
IFCWPDWEEFYDCY

SEQ ID NO: 169 - protein, 14 aa, synthetic construct
  REGION 1..14: note = Description of Artificial Sequence: Synthetic peptide
ADCWPDWEEYFPCN

SEQ ID NO: 170 - protein, 14 aa, synthetic construct
  REGION 1..14: note = Description of Artificial Sequence: Synthetic peptide
HTCWPDWEDDIFCN

SEQ ID NO: 171 - protein, 14 aa, synthetic construct
  REGION 1..14: note = Description of Artificial Sequence: Synthetic peptide
FACWPDWEEAFLCL

SEQ ID NO: 172 - protein, 14 aa, synthetic construct
  REGION 1..14: note = Description of Artificial Sequence: Synthetic peptide
YDCGPELDESIACD

SEQ ID NO: 173 - protein, 14 aa, synthetic construct
  REGION 1..14: note = Description of Artificial Sequence: Synthetic peptide
NSCWPDWEYDIICD

SEQ ID NO: 174 - protein, 14 aa, synthetic construct
  REGION 1..14: note = Description of Artificial Sequence: Synthetic peptide
FACWPDWEEVAPCY

SEQ ID NO: 175 - protein, 14 aa, synthetic construct
  REGION 1..14: note = Description of Artificial Sequence: Synthetic peptide
LDCGPDGDETLTCY

SEQ ID NO: 176 - protein, 14 aa, synthetic construct
  REGION 1..14: note = Description of Artificial Sequence: Synthetic peptide
VLCWPDWEEFYDCP

SEQ ID NO: 177 - protein, 14 aa, synthetic construct
  REGION 1..14: note = Description of Artificial Sequence: Synthetic peptide
HACWPVWEEYFPCN

SEQ ID NO: 178 - protein, 14 aa, synthetic construct
  REGION 1..14: note = Description of Artificial Sequence: Synthetic peptide
NECWPNGEPTFPCT

SEQ ID NO: 179 - protein, 14 aa, synthetic construct
  REGION 1..14: note = Description of Artificial Sequence: Synthetic peptide
LTCGPDGDETLYCD

SEQ ID NO: 180 - protein, 14 aa, synthetic construct
  REGION 1..14: note = Description of Artificial Sequence: Synthetic peptide
YDCGPEYDESVPCI

SEQ ID NO: 181 - protein, 14 aa, synthetic construct
  REGION 1..14: note = Description of Artificial Sequence: Synthetic peptide
IECWPDWEPNSFCD

SEQ ID NO: 182 - protein, 14 aa, synthetic construct
  REGION 1..14: note = Description of Artificial Sequence: Synthetic peptide
YDCGPELDESIHCY

SEQ ID NO: 183 - protein, 14 aa, synthetic construct
  REGION 1..14: note = Description of Artificial Sequence: Synthetic peptide
IYCGPEFDESTICN

SEQ ID NO: 184 - protein, 14 aa, synthetic construct
  REGION 1..14: note = Description of Artificial Sequence: Synthetic peptide
IYCGPEVEEAYLCY

SEQ ID NO: 185 - protein, 14 aa, synthetic construct
  REGION 1..14: note = Description of Artificial Sequence: Synthetic peptide
FDCGPDGDETVYCD

SEQ ID NO: 186 - protein, 14 aa, synthetic construct
  REGION 1..14: note = Description of Artificial Sequence: Synthetic peptide
IDCGPDGDETISCY

SEQ ID NO: 187 - protein, 14 aa, synthetic construct
  REGION 1..14: note = Description of Artificial Sequence: Synthetic peptide
NYCGPEFDESSTCL

SEQ ID NO: 188 - protein, 14 aa, synthetic construct
  REGION 1..14: note = Description of Artificial Sequence: Synthetic peptide
YDCGPDGDESYFCD

SEQ ID NO: 189 - protein, 14 aa, synthetic construct
  REGION 1..14: note = Description of Artificial Sequence: Synthetic peptide
NFCWPDWEYFNDCN

SEQ ID NO: 190 - protein, 14 aa, synthetic construct
  REGION 1..14: note = Description of Artificial Sequence: Synthetic peptide
VLCWPDWEAFFDCD

SEQ ID NO: 191 - protein, 14 aa, synthetic construct
  REGION 1..14: note = Description of Artificial Sequence: Synthetic peptide
IYCGPEWEWPVACN

SEQ ID NO: 192 - protein, 14 aa, synthetic construct
  REGION 1..14: note = Description of Artificial Sequence: Synthetic peptide
VFCWPDWEDNFFCN

SEQ ID NO: 193 - protein, 14 aa, synthetic construct
  REGION 1..14: note = Description of Artificial Sequence: Synthetic peptide
VVCWPDWETFFPCD

SEQ ID NO: 194 - protein, 14 aa, synthetic construct
  REGION 1..14: note = Description of Artificial Sequence: Synthetic peptide
DNCGPDGDETYICN

SEQ ID NO: 195 - protein, 14 aa, synthetic construct
  REGION 1..14: note = Description of Artificial Sequence: Synthetic peptide
DNCWPDWEYNFFCV

SEQ ID NO: 196 - protein, 14 aa, synthetic construct
  REGION 1..14: note = Description of Artificial Sequence: Synthetic peptide
FYCGPEVEEDYLCI

SEQ ID NO: 197 - protein, 14 aa, synthetic construct
  REGION 1..14: note = Description of Artificial Sequence: Synthetic peptide
DNCWPDWEYDIFCV

SEQ ID NO: 198 - protein, 14 aa, synthetic construct
  REGION 1..14: note = Description of Artificial Sequence: Synthetic peptide
IDCGPEFDESIACP

SEQ ID NO: 199 - protein, 14 aa, synthetic construct
  REGION 1..14: note = Description of Artificial Sequence: Synthetic peptide
YFCGPEVEEYTLCF

SEQ ID NO: 200 - protein, 14 aa, synthetic construct
  REGION 1..14: note = Description of Artificial Sequence: Synthetic peptide
FYCGPEFDESAPCN

SEQ ID NO: 201 - protein, 14 aa, synthetic construct
  REGION 1..14: note = Description of Artificial Sequence: Synthetic peptide
FDCGPEVEEYFYCA

SEQ ID NO: 202 - protein, 14 aa, synthetic construct
  REGION 1..14: note = Description of Artificial Sequence: Synthetic peptide
DFCWPDWEDFFFCA

SEQ ID NO: 203 - protein, 14 aa, synthetic construct
  REGION 1..14: note = Description of Artificial Sequence: Synthetic peptide
FFCGPDGDETLSCN

SEQ ID NO: 204 - protein, 14 aa, synthetic construct
  REGION 1..14: note = Description of Artificial Sequence: Synthetic peptide
FICGPEFDESVACL

SEQ ID NO: 205 - protein, 14 aa, synthetic construct
  REGION 1..14: note = Description of Artificial Sequence: Synthetic peptide
YDCGPEFDEAIGCY

SEQ ID NO: 206 - protein, 14 aa, synthetic construct
  REGION 1..14: note = Description of Artificial Sequence: Synthetic peptide
YICWPDWEEYLYCP

SEQ ID NO: 207 - protein, 14 aa, synthetic construct
  REGION 1..14: note = Description of Artificial Sequence: Synthetic peptide
FDCWPDWEEPTTCH

SEQ ID NO: 208 - protein, 14 aa, synthetic construct
  REGION 1..14: note = Description of Artificial Sequence: Synthetic peptide
YDCWPDWEDFPICD

SEQ ID NO: 209 - protein, 14 aa, synthetic construct
  REGION 1..14: note = Description of Artificial Sequence: Synthetic peptide
VVCWPDWEYIDDCS

SEQ ID NO: 210 - protein, 14 aa, synthetic construct
  REGION 1..14: note = Description of Artificial Sequence: Synthetic peptide
INCWPDWEVISFCD

SEQ ID NO: 211 - protein, 14 aa, synthetic construct
  REGION 1..14: note = Description of Artificial Sequence: Synthetic peptide
LSCWPDWEEVTPCL

SEQ ID NO: 212 - protein, 14 aa, synthetic construct
  REGION 1..14: note = Description of Artificial Sequence: Synthetic peptide
FACWPDWEEVDICY

SEQ ID NO: 213 - protein, 14 aa, synthetic construct
  REGION 1..14: note = Description of Artificial Sequence: Synthetic peptide
YDCGPEMDESIVCD

SEQ ID NO: 214 - protein, 14 aa, synthetic construct
  REGION 1..14: note = Description of Artificial Sequence: Synthetic peptide
YDCWPDWEVFIVCD

SEQ ID NO: 215 - protein, 14 aa, synthetic construct
  REGION 1..14: note = Description of Artificial Sequence: Synthetic peptide
DNCWPDWEHNFFCV

SEQ ID NO: 216 - protein, 14 aa, synthetic construct
  REGION 1..14: note = Description of Artificial Sequence: Synthetic peptide
YDCGPDGDESIYCP

SEQ ID NO: 217 - protein, 14 aa, synthetic construct
  REGION 1..14: note = Description of Artificial Sequence: Synthetic peptide
YDCGPEFEFPYYCF

SEQ ID NO: 218 - protein, 14 aa, synthetic construct
  REGION 1..14: note = Description of Artificial Sequence: Synthetic peptide
ADCGPEYDESVPCV

SEQ ID NO: 219 - protein, 14 aa, synthetic construct
  REGION 1..14: note = Description of Artificial Sequence: Synthetic peptide
FLCGPEVEEVHYCS

SEQ ID NO: 220 - protein, 14 aa, synthetic construct
  REGION 1..14: note = Description of Artificial Sequence: Synthetic peptide
TDCWPDWEYITSCS

SEQ ID NO: 221 - protein, 14 aa, synthetic construct
  REGION 1..14: note = Description of Artificial Sequence: Synthetic peptide
AFCGPELDESITCD

SEQ ID NO: 222 - protein, 14 aa, synthetic construct
  REGION 1..14: note = Description of Artificial Sequence: Synthetic peptide
NDCWPDWEEYFSCY

SEQ ID NO: 223 - protein, 14 aa, synthetic construct
  REGION 1..14: note = Description of Artificial Sequence: Synthetic peptide
FDCGPEWEIVTDCY

SEQ ID NO: 224 - protein, 14 aa, synthetic construct
  REGION 1..14: note = Description of Artificial Sequence: Synthetic peptide
NLCGPEMDESIICP

SEQ ID NO: 225 - protein, 14 aa, synthetic construct
  REGION 1..14: note = Description of Artificial Sequence: Synthetic peptide
DLCGPEMDESIYCD

SEQ ID NO: 226 - protein, 14 aa, synthetic construct
  REGION 1..14: note = Description of Artificial Sequence: Synthetic peptide
FDCGPDGVEDYICD

SEQ ID NO: 227 - protein, 14 aa, synthetic construct
  REGION 1..14: note = Description of Artificial Sequence: Synthetic peptide
YACWPDWEEDFACY

SEQ ID NO: 228 - protein, 14 aa, synthetic construct
  REGION 1..14: note = Description of Artificial Sequence: Synthetic peptide
HDCGPEMDESIVCV

SEQ ID NO: 229 - protein, 14 aa, synthetic construct
  REGION 1..14: note = Description of Artificial Sequence: Synthetic peptide
VFCGPEFEFIFLCA

SEQ ID NO: 230 - protein, 14 aa, synthetic construct
  REGION 1..14: note = Description of Artificial Sequence: Synthetic peptide
YDCGPELDESILCD

SEQ ID NO: 231 - protein, 14 aa, synthetic construct
  REGION 1..14: note = Description of Artificial Sequence: Synthetic peptide
SVCWPDWEEFYSCD

SEQ ID NO: 232 - protein, 14 aa, synthetic construct
  REGION 1..14: note = Description of Artificial Sequence: Synthetic peptide
PYCGPDGDETAICT

SEQ ID NO: 233 - protein, 14 aa, synthetic construct
  REGION 1..14: note = Description of Artificial Sequence: Synthetic peptide
DDCGPELEWYYPCY

SEQ ID NO: 234 - protein, 14 aa, synthetic construct
  REGION 1..14: note = Description of Artificial Sequence: Synthetic peptide
FICGPEFDESLPCN

SEQ ID NO: 235 - protein, 14 aa, synthetic construct
  REGION 1..14: note = Description of Artificial Sequence: Synthetic peptide
IDCGPEFDESLPCD

SEQ ID NO: 236 - protein, 14 aa, synthetic construct
  REGION 1..14: note = Description of Artificial Sequence: Synthetic peptide
FLCGPEFEEDAPCY

SEQ ID NO: 237 - protein, 14 aa, synthetic construct
  REGION 1..14: note = Description of Artificial Sequence: Synthetic peptide
IFCGPDGDETHICH

SEQ ID NO: 238 - protein, 14 aa, synthetic construct
  REGION 1..14: note = Description of Artificial Sequence: Synthetic peptide
VFCWPDWEYIDFCN

SEQ ID NO: 239 - protein, 14 aa, synthetic construct
  REGION 1..14: note = Description of Artificial Sequence: Synthetic peptide
IFCGPEYDESLHCI

SEQ ID NO: 240 - protein, 14 aa, synthetic construct
  REGION 1..14: note = Description of Artificial Sequence: Synthetic peptide
HLCWPDWEWYVDCP

SEQ ID NO: 241 - protein, 14 aa, synthetic construct
  REGION 1..14: note = Description of Artificial Sequence: Synthetic peptide
FICGPEMDESIACN

SEQ ID NO: 242 - protein, 14 aa, synthetic construct
  REGION 1..14: note = Description of Artificial Sequence: Synthetic peptide
IFCGPEVEMIFLCN

SEQ ID NO: 243 - protein, 14 aa, synthetic construct
  REGION 1..14: note = Description of Artificial Sequence: Synthetic peptide
YDCGPEWEFPVDCI

SEQ ID NO: 244 - protein, 14 aa, synthetic construct
  REGION 1..14: note = Description of Artificial Sequence: Synthetic peptide
NLCGPELDESITCF

SEQ ID NO: 245 - protein, 14 aa, synthetic construct
  REGION 1..14: note = Description of Artificial Sequence: Synthetic peptide
FYCGPEVEDFYFCY

SEQ ID NO: 246 - protein, 14 aa, synthetic construct
  REGION 1..14: note = Description of Artificial Sequence: Synthetic peptide
DYCGPEFDESLICN

SEQ ID NO: 247 - protein, 14 aa, synthetic construct
  REGION 1..14: note = Description of Artificial Sequence: Synthetic peptide
DYCGPEFDESLPCD

SEQ ID NO: 248 - protein, 14 aa, synthetic construct
  REGION 1..14: note = Description of Artificial Sequence: Synthetic peptide
AICGPELDESIACP

SEQ ID NO: 249 - protein, 14 aa, synthetic construct
  REGION 1..14: note = Description of Artificial Sequence: Synthetic peptide
VICGPEVEDYNLCY

SEQ ID NO: 250 - protein, 14 aa, synthetic construct
  REGION 1..14: note = Description of Artificial Sequence: Synthetic peptide
HTCWPDWEDYTVCP

SEQ ID NO: 251 - protein, 14 aa, synthetic construct
  REGION 1..14: note = Description of Artificial Sequence: Synthetic peptide
SDCWPDWEYFYDCN

SEQ ID NO: 252 - protein, 14 aa, synthetic construct
  REGION 1..14: note = Description of Artificial Sequence: Synthetic peptide
VFCGPDGDETVHCD

SEQ ID NO: 253 - protein, 14 aa, synthetic construct
  REGION 1..14: note = Description of Artificial Sequence: Synthetic peptide
DYCGPEYDESVHCI

SEQ ID NO: 254 - protein, 14 aa, synthetic construct
  REGION 1..14: note = Description of Artificial Sequence: Synthetic peptide
ADCGPDGDESIICH

SEQ ID NO: 255 - protein, 14 aa, synthetic construct
  REGION 1..14: note = Description of Artificial Sequence: Synthetic peptide
FYCGPELDESLTCV

SEQ ID NO: 256 - protein, 14 aa, synthetic construct
  REGION 1..14: note = Description of Artificial Sequence: Synthetic peptide
ILCGPEVEEDYYCY

SEQ ID NO: 257 - protein, 14 aa, synthetic construct
  REGION 1..14: note = Description of Artificial Sequence: Synthetic peptide
HLCWPDWEEYHSCD

SEQ ID NO: 258 - protein, 14 aa, synthetic construct
  REGION 1..14: note = Description of Artificial Sequence: Synthetic peptide
IFCWPDWEDYNFCT

SEQ ID NO: 259 - protein, 14 aa, synthetic construct
  REGION 1..14: note = Description of Artificial Sequence: Synthetic peptide
IVCGPDGDETLICH

SEQ ID NO: 260 - protein, 14 aa, synthetic construct
  REGION 1..14: note = Description of Artificial Sequence: Synthetic peptide
ADCWPDWEWDYTCD

SEQ ID NO: 261 - protein, 14 aa, synthetic construct
  REGION 1..14: note = Description of Artificial Sequence: Synthetic peptide
ITCGPEFDESTTCN

SEQ ID NO: 262 - protein, 14 aa, synthetic construct
  REGION 1..14: note = Description of Artificial Sequence: Synthetic peptide
YHCWPDWEEYTSCD

SEQ ID NO: 263 - protein, 14 aa, synthetic construct
  REGION 1..14: note = Description of Artificial Sequence: Synthetic peptide
NYCGPEVEEYALCT

SEQ ID NO: 264 - protein, 14 aa, synthetic construct
  REGION 1..14: note = Description of Artificial Sequence: Synthetic peptide
FICGPEMDESIHCD

SEQ ID NO: 265 - protein, 14 aa, synthetic construct
  REGION 1..14: note = Description of Artificial Sequence: Synthetic peptide
DNCWPDWEEFAVCP

SEQ ID NO: 266 - protein, 14 aa, synthetic construct
  REGION 1..14: note = Description of Artificial Sequence: Synthetic peptide
YDCGPELDETVVCD

SEQ ID NO: 267 - protein, 14 aa, synthetic construct
  REGION 1..14: note = Description of Artificial Sequence: Synthetic peptide
YDCGPEFDESIACY

SEQ ID NO: 268 - protein, 14 aa, synthetic construct
  REGION 1..14: note = Description of Artificial Sequence: Synthetic peptide
IDCWPDWEYTVHCD

SEQ ID NO: 269 - protein, 14 aa, synthetic construct
  REGION 1..14: note = Description of Artificial Sequence: Synthetic peptide
DDCGPELDESIICI

SEQ ID NO: 270 - protein, 14 aa, synthetic construct
  REGION 1..14: note = Description of Artificial Sequence: Synthetic peptide
VYCGPEYDESSFCF

SEQ ID NO: 271 - protein, 14 aa, synthetic construct
  REGION 1..14: note = Description of Artificial Sequence: Synthetic peptide
FNCWPDWEDPYFCV

SEQ ID NO: 272 - protein, 14 aa, synthetic construct
  REGION 1..14: note = Description of Artificial Sequence: Synthetic peptide
YDCGPEYDESSYCS

SEQ ID NO: 273 - protein, 14 aa, synthetic construct
  REGION 1..14: note = Description of Artificial Sequence: Synthetic peptide
VACWPDWEYTDSCF

SEQ ID NO: 274 - protein, 14 aa, synthetic construct
  REGION 1..14: note = Description of Artificial Sequence: Synthetic peptide
TDCGPEFDESVACY

SEQ ID NO: 275 - protein, 14 aa, synthetic construct
  REGION 1..14: note = Description of Artificial Sequence: Synthetic peptide
TDCWPDWEFYADCD

SEQ ID NO: 276 - protein, 14 aa, synthetic construct
  REGION 1..14: note = Description of Artificial Sequence: Synthetic peptide
YDCGPELDESVICH

SEQ ID NO: 277 - protein, 14 aa, synthetic construct
  REGION 1..14: note = Description of Artificial Sequence: Synthetic peptide
SDCGPDGDESIICT

SEQ ID NO: 278 - protein, 14 aa, synthetic construct
  REGION 1..14: note = Description of Artificial Sequence: Synthetic peptide
YYCGPEFDESIDCD

SEQ ID NO: 279 - protein, 14 aa, synthetic construct
  REGION 1..14: note = Description of Artificial Sequence: Synthetic peptide
FFCGPEIDESIACV

SEQ ID NO: 280 - protein, 14 aa, synthetic construct
  REGION 1..14: note = Description of Artificial Sequence: Synthetic peptide
DYCGPEFDESTFCD

SEQ ID NO: 281 - protein, 14 aa, synthetic construct
  REGION 1..14: note = Description of Artificial Sequence: Synthetic peptide
YDCGPEWEWPIDCV

SEQ ID NO: 282 - protein, 14 aa, synthetic construct
  REGION 1..14: note = Description of Artificial Sequence: Synthetic peptide
FYCGPEIELFSFCY

SEQ ID NO: 283 - protein, 14 aa, synthetic construct
  REGION 1..14: note = Description of Artificial Sequence: Synthetic peptide
YYCGPEVDESITCP

SEQ ID NO: 284 - protein, 14 aa, synthetic construct
  REGION 1..14: note = Description of Artificial Sequence: Synthetic peptide
ILCGPEFDESINCN

SEQ ID NO: 285 - protein, 14 aa, synthetic construct
  REGION 1..14: note = Description of Artificial Sequence: Synthetic peptide
VVCGPAMGQHYLCD

SEQ ID NO: 286 - protein, 14 aa, synthetic construct
  REGION 1..14: note = Description of Artificial Sequence: Synthetic peptide
VVCGTKMGEHYLCS

SEQ ID NO: 287 - protein, 14 aa, synthetic construct
  REGION 1..14: note = Description of Artificial Sequence: Synthetic peptide
YDCWPDWEYVYACY

SEQ ID NO: 288 - protein, 14 aa, synthetic construct
  REGION 1..14: note = Description of Artificial Sequence: Synthetic peptide
DLCGPELDESVNCD

SEQ ID NO: 289 - protein, 14 aa, synthetic construct
  REGION 1..14: note = Description of Artificial Sequence: Synthetic peptide
YYCGPEFDESTVCY

SEQ ID NO: 290 - protein, 14 aa, synthetic construct
  REGION 1..14: note = Description of Artificial Sequence: Synthetic peptide
LDCWPDWEWPYSCN

SEQ ID NO: 291 - protein, 14 aa, synthetic construct
  REGION 1..14: note = Description of Artificial Sequence: Synthetic peptide
FICWPDWEDDFFCY

SEQ ID NO: 292 - protein, 14 aa, synthetic construct
  REGION 1..14: note = Description of Artificial Sequence: Synthetic peptide
DLCGPEVEWYFFCN

SEQ ID NO: 293 - protein, 14 aa, synthetic construct
  REGION 1..14: note = Description of Artificial Sequence: Synthetic peptide
YDCGPELDESIVCF

SEQ ID NO: 294 - protein, 14 aa, synthetic construct
  REGION 1..14: note = Description of Artificial Sequence: Synthetic peptide
LNCWPVWEDDVFCY

SEQ ID NO: 295 - protein, 14 aa, synthetic construct
  REGION 1..14: note = Description of Artificial Sequence: Synthetic peptide
FNCWPDWEDPNFCV

SEQ ID NO: 296 - protein, 14 aa, synthetic construct
  REGION 1..14: note = Description of Artificial Sequence: Synthetic peptide
VICWPDWEDDYFCP

SEQ ID NO: 297 - protein, 14 aa, synthetic construct
  REGION 1..14: note = Description of Artificial Sequence: Synthetic peptide
FLCGPEFDESSVCY

SEQ ID NO: 298 - protein, 14 aa, synthetic construct
  REGION 1..14: note = Description of Artificial Sequence: Synthetic peptide
YDCGPELDESIFCY

SEQ ID NO: 299 - protein, 14 aa, synthetic construct
  REGION 1..14: note = Description of Artificial Sequence: Synthetic peptide
HLCGPDGDESFTCF

SEQ ID NO: 300 - protein, 14 aa, synthetic construct
  REGION 1..14: note = Description of Artificial Sequence: Synthetic peptide
YFCGPEMDESLYCI

SEQ ID NO: 301 - protein, 14 aa, synthetic construct
  REGION 1..14: note = Description of Artificial Sequence: Synthetic peptide
YYCGPEVEEYANCY

SEQ ID NO: 302 - protein, 14 aa, synthetic construct
  REGION 1..14: note = Description of Artificial Sequence: Synthetic peptide
NTCGPEFDESTACY

SEQ ID NO: 303 - protein, 14 aa, synthetic construct
  REGION 1..14: note = Description of Artificial Sequence: Synthetic peptide
IDCWPDWEEAFNCY

SEQ ID NO: 304 - protein, 14 aa, synthetic construct
  REGION 1..14: note = Description of Artificial Sequence: Synthetic peptide
AYCGPELEEFFLCT

SEQ ID NO: 305 - protein, 14 aa, synthetic construct
  REGION 1..14: note = Description of Artificial Sequence: Synthetic peptide
IYCGPEVEEVHHCY

SEQ ID NO: 306 - protein, 14 aa, synthetic construct
  REGION 1..14: note = Description of Artificial Sequence: Synthetic peptide
FFCGPEFDESVACD

SEQ ID NO: 307 - protein, 14 aa, synthetic construct
  REGION 1..14: note = Description of Artificial Sequence: Synthetic peptide
YDCGPELDETIICA

SEQ ID NO: 308 - protein, 14 aa, synthetic construct
  REGION 1..14: note = Description of Artificial Sequence: Synthetic peptide
ILCGPEWEYPLDCS

SEQ ID NO: 309 - protein, 14 aa, synthetic construct
  REGION 1..14: note = Description of Artificial Sequence: Synthetic peptide
FICGPEFDESTTCN

SEQ ID NO: 310 - protein, 14 aa, synthetic construct
  REGION 1..14: note = Description of Artificial Sequence: Synthetic peptide
FYCGPELDESVSCD

SEQ ID NO: 311 - protein, 14 aa, synthetic construct
  REGION 1..14: note = Description of Artificial Sequence: Synthetic peptide
HLCGPELDESVTCF

SEQ ID NO: 312 - protein, 14 aa, synthetic construct
  REGION 1..14: note = Description of Artificial Sequence: Synthetic peptide
LICGPEVEDYSLCH

SEQ ID NO: 313 - protein, 14 aa, synthetic construct
  REGION 1..14: note = Description of Artificial Sequence: Synthetic peptide
YFCGPEMDESVYCD

SEQ ID NO: 314 - protein, 14 aa, synthetic construct
  REGION 1..14: note = Description of Artificial Sequence: Synthetic peptide
HYCGPEMDESIYCI

SEQ ID NO: 315 - protein, 14 aa, synthetic construct
  REGION 1..14: note = Description of Artificial Sequence: Synthetic peptide
FDCGPELDESINCD

SEQ ID NO: 316 - protein, 14 aa, synthetic construct
  REGION 1..14: note = Description of Artificial Sequence: Synthetic peptide
YYCGPEVEEYIYCT

SEQ ID NO: 317 - protein, 14 aa, synthetic construct
  REGION 1..14: note = Description of Artificial Sequence: Synthetic peptide
LACWPVREEINACI

SEQ ID NO: 318 - protein, 14 aa, synthetic construct
  REGION 1..14: note = Description of Artificial Sequence: Synthetic peptide
IDCWPDWEDITFCD

SEQ ID NO: 319 - protein, 14 aa, synthetic construct
  REGION 1..14: note = Description of Artificial Sequence: Synthetic peptide
IVCGPELDESITCP

SEQ ID NO: 320 - protein, 14 aa, synthetic construct
  REGION 1..14: note = Description of Artificial Sequence: Synthetic peptide
FYCGPEFELPADCD

SEQ ID NO: 321 - protein, 14 aa, synthetic construct
  REGION 1..14: note = Description of Artificial Sequence: Synthetic peptide
FDCGPEFDESNPCF

SEQ ID NO: 322 - protein, 14 aa, synthetic construct
  REGION 1..14: note = Description of Artificial Sequence: Synthetic peptide
DACWPDWEEYSSCD

SEQ ID NO: 323 - protein, 14 aa, synthetic construct
  REGION 1..14: note = Description of Artificial Sequence: Synthetic peptide
DHCWPDWEPNYFCV

SEQ ID NO: 324 - protein, 14 aa, synthetic construct
  REGION 1..14: note = Description of Artificial Sequence: Synthetic peptide
DYCWPDWEINYICF

SEQ ID NO: 325 - protein, 14 aa, synthetic construct
  REGION 1..14: note = Description of Artificial Sequence: Synthetic peptide
IYCWPDWEYVYACN

SEQ ID NO: 326 - protein, 14 aa, synthetic construct
  REGION 1..14: note = Description of Artificial Sequence: Synthetic peptide
DFCGPEVEEDYLCD

SEQ ID NO: 327 - protein, 14 aa, synthetic construct
  REGION 1..14: note = Description of Artificial Sequence: Synthetic peptide
HDCGPDGREDYDCA

SEQ ID NO: 328 - protein, 14 aa, synthetic construct
  REGION 1..14: note = Description of Artificial Sequence: Synthetic peptide
LACWPDWEDDYFCV

SEQ ID NO: 329 - protein, 14 aa, synthetic construct
  REGION 1..14: note = Description of Artificial Sequence: Synthetic peptide
DICWPDWEDYLPCV

SEQ ID NO: 330 - protein, 14 aa, synthetic construct
  REGION 1..14: note = Description of Artificial Sequence: Synthetic peptide
ILCGPEIEVYALCP

SEQ ID NO: 331 - protein, 14 aa, synthetic construct
  REGION 1..14: note = Description of Artificial Sequence: Synthetic peptide
IFCGPEWEFSVLCN

SEQ ID NO: 332 - protein, 14 aa, synthetic construct
  REGION 1..14: note = Description of Artificial Sequence: Synthetic peptide
TYCGPEVEDFSLCV

SEQ ID NO: 333 - protein, 14 aa, synthetic construct
  REGION 1..14: note = Description of Artificial Sequence: Synthetic peptide
FICGPEWEFVDACF

SEQ ID NO: 334 - protein, 14 aa, synthetic construct
  REGION 1..14: note = Description of Artificial Sequence: Synthetic peptide
FACWPDWEEDSPCD

SEQ ID NO: 335 - protein, 14 aa, synthetic construct
  REGION 1..14: note = Description of Artificial Sequence: Synthetic peptide
ILCGPEVEELIFCP

SEQ ID NO: 336 - protein, 14 aa, synthetic construct
  REGION 1..14: note = Description of Artificial Sequence: Synthetic peptide
FYCGPEVEEYIYCY

SEQ ID NO: 337 - protein, 14 aa, synthetic construct
  REGION 1..14: note = Description of Artificial Sequence: Synthetic peptide
DSCGPELDESIICD

SEQ ID NO: 338 - protein, 14 aa, synthetic construct
  REGION 1..14: note = Description of Artificial Sequence: Synthetic peptide
FLCGPDGDETSVCD

SEQ ID NO: 339 - protein, 14 aa, synthetic construct
  REGION 1..14: note = Description of Artificial Sequence: Synthetic peptide
FNCWPNGEPTYFCV

SEQ ID NO: 340 - protein, 14 aa, synthetic construct
  REGION 1..14: note = Description of Artificial Sequence: Synthetic peptide
LACWPVWEYPVTCI

SEQ ID NO: 341 - protein, 14 aa, synthetic construct
  REGION 1..14: note = Description of Artificial Sequence: Synthetic peptide
DYCGPEVEEDVYCY

SEQ ID NO: 342 - protein, 14 aa, synthetic construct
  REGION 1..14: note = Description of Artificial Sequence: Synthetic peptide
ITCWPDWEEYANCT

SEQ ID NO: 343 - protein, 14 aa, synthetic construct
  REGION 1..14: note = Description of Artificial Sequence: Synthetic peptide
FFCGPDGDETYSCI

SEQ ID NO: 344 - protein, 14 aa, synthetic construct
  REGION 1..14: note = Description of Artificial Sequence: Synthetic peptide
TDCWPDWEYATSCD

SEQ ID NO: 345 - protein, 14 aa, synthetic construct
  REGION 1..14: note = Description of Artificial Sequence: Synthetic peptide
FNCGPDGYEDYLCD

SEQ ID NO: 346 - protein, 14 aa, synthetic construct
  REGION 1..14: note = Description of Artificial Sequence: Synthetic peptide
YDCWPDWEVDFHCP

SEQ ID NO: 347 - protein, 14 aa, synthetic construct
  REGION 1..14: note = Description of Artificial Sequence: Synthetic peptide
NICWPDWEDDSFCF

SEQ ID NO: 348 - protein, 14 aa, synthetic construct
  REGION 1..14: note = Description of Artificial Sequence: Synthetic peptide
ATCGPEFDESIGCS

SEQ ID NO: 349 - protein, 14 aa, synthetic construct
  REGION 1..14: note = Description of Artificial Sequence: Synthetic peptide
SYCGPEFDESTFCD

SEQ ID NO: 350 - protein, 14 aa, synthetic construct
  REGION 1..14: note = Description of Artificial Sequence: Synthetic peptide
PICGPEYDESDVCA

SEQ ID NO: 351 - protein, 14 aa, synthetic construct
  REGION 1..14: note = Description of Artificial Sequence: Synthetic peptide
YYCGPDGDEYNSCI

SEQ ID NO: 352 - protein, 14 aa, synthetic construct
  REGION 1..14: note = Description of Artificial Sequence: Synthetic peptide
VDCWPDWEVFIACD

SEQ ID NO: 353 - protein, 14 aa, synthetic construct
  REGION 1..14: note = Description of Artificial Sequence: Synthetic peptide
DLCGPEVEEVNLCL

SEQ ID NO: 354 - protein, 14 aa, synthetic construct
  REGION 1..14: note = Description of Artificial Sequence: Synthetic peptide
FDCGPEMDESTTCF

SEQ ID NO: 355 - protein, 14 aa, synthetic construct
  REGION 1..14: note = Description of Artificial Sequence: Synthetic peptide
NFCWPDWEPIYFCT

SEQ ID NO: 356 - protein, 14 aa, synthetic construct
  REGION 1..14: note = Description of Artificial Sequence: Synthetic peptide
VDCGPDGDESFFCL

SEQ ID NO: 357 - protein, 14 aa, synthetic construct
  REGION 1..14: note = Description of Artificial Sequence: Synthetic peptide
VDCGPDGDETAFCI

SEQ ID NO: 358 - protein, 14 aa, synthetic construct
  REGION 1..14: note = Description of Artificial Sequence: Synthetic peptide
NICGPEMDESLVCI

SEQ ID NO: 359 - protein, 14 aa, synthetic construct
  REGION 1..14: note = Description of Artificial Sequence: Synthetic peptide
IICGPEFDESFFCF

SEQ ID NO: 360 - protein, 14 aa, synthetic construct
  REGION 1..14: note = Description of Artificial Sequence: Synthetic peptide
NFCGPEYDESISCI

SEQ ID NO: 361 - protein, 14 aa, synthetic construct
  REGION 1..14: note = Description of Artificial Sequence: Synthetic peptide
HLCGPEIEEADICN

SEQ ID NO: 362 - protein, 14 aa, synthetic construct
  REGION 1..14: note = Description of Artificial Sequence: Synthetic peptide
DYCGPEVEEDYLCD

SEQ ID NO: 363 - protein, 14 aa, synthetic construct
  REGION 1..14: note = Description of Artificial Sequence: Synthetic peptide
DYCGPELDESINCD

SEQ ID NO: 364 - protein, 14 aa, synthetic construct
  REGION 1..14: note = Description of Artificial Sequence: Synthetic peptide
FYCGPELDESLFCV

SEQ ID NO: 365 - protein, 14 aa, synthetic construct
  REGION 1..14: note = Description of Artificial Sequence: Synthetic peptide
PDCGPEWEFYVTCN

SEQ ID NO: 366 - protein, 14 aa, synthetic construct
  REGION 1..14: note = Description of Artificial Sequence: Synthetic peptide
FDCGPEFEYIYACT

SEQ ID NO: 367 - protein, 14 aa, synthetic construct
  REGION 1..14: note = Description of Artificial Sequence: Synthetic peptide
DYCGPEFDESSICN

SEQ ID NO: 368 - protein, 14 aa, synthetic construct
  REGION 1..14: note = Description of Artificial Sequence: Synthetic peptide
DFCGPEVEEYIFCF

SEQ ID NO: 369 - protein, 14 aa, synthetic construct
  REGION 1..14: note = Description of Artificial Sequence: Synthetic peptide
PVCWPDWEYVSSCD

SEQ ID NO: 370 - protein, 14 aa, synthetic construct
  REGION 1..14: note = Description of Artificial Sequence: Synthetic peptide
YICGPERDESNLCL

SEQ ID NO: 371 - protein, 14 aa, synthetic construct
  REGION 1..14: note = Description of Artificial Sequence: Synthetic peptide
YDCGPELDESIVCD

SEQ ID NO: 372 - protein, 14 aa, synthetic construct
  REGION 1..14: note = Description of Artificial Sequence: Synthetic peptide
HDCWPDWEDFYFCV

SEQ ID NO: 373 - protein, 14 aa, synthetic construct
  REGION 1..14: note = Description of Artificial Sequence: Synthetic peptide
HYCGPEYDESIDCY

SEQ ID NO: 374 - protein, 14 aa, synthetic construct
  REGION 1..14: note = Description of Artificial Sequence: Synthetic peptide
LFCGPEMPEDIFCN

SEQ ID NO: 375 - protein, 14 aa, synthetic construct
  REGION 1..14: note = Description of Artificial Sequence: Synthetic peptide
HDCGPELEFHYACY

SEQ ID NO: 376 - protein, 14 aa, synthetic construct
  REGION 1..14: note = Description of Artificial Sequence: Synthetic peptide
DFCGPELDESINCF

SEQ ID NO: 377 - protein, 14 aa, synthetic construct
  REGION 1..14: note = Description of Artificial Sequence: Synthetic peptide
YFCGPELDESIACN

SEQ ID NO: 378 - protein, 14 aa, synthetic construct
  REGION 1..14: note = Description of Artificial Sequence: Synthetic peptide
TDCWPDWEDDIICD

SEQ ID NO: 379 - protein, 14 aa, synthetic construct
  REGION 1..14: note = Description of Artificial Sequence: Synthetic peptide
YDCGPELDESIYCF

SEQ ID NO: 380 - protein, 14 aa, synthetic construct
  REGION 1..14: note = Description of Artificial Sequence: Synthetic peptide
YYCWPDWWEYVTCD

SEQ ID NO: 381 - protein, 14 aa, synthetic construct
  REGION 1..14: note = Description of Artificial Sequence: Synthetic peptide
PICGPELEESYLCN

SEQ ID NO: 382 - protein, 14 aa, synthetic construct
  REGION 1..14: note = Description of Artificial Sequence: Synthetic peptide
FDCGPEFDESIVCY

SEQ ID NO: 383 - protein, 14 aa, synthetic construct
  REGION 1..14: note = Description of Artificial Sequence: Synthetic peptide
VLCGPDGIEFFDCP

SEQ ID NO: 384 - protein, 14 aa, synthetic construct
  REGION 1..14: note = Description of Artificial Sequence: Synthetic peptide
AYCGPEYDESLTCV

SEQ ID NO: 385 - protein, 14 aa, synthetic construct
  REGION 1..14: note = Description of Artificial Sequence: Synthetic peptide
DFCGPELDESIICA

SEQ ID NO: 386 - protein, 14 aa, synthetic construct
  REGION 1..14: note = Description of Artificial Sequence: Synthetic peptide
YDCGPEFDESLDCN

SEQ ID NO: 387 - protein, 14 aa, synthetic construct
  REGION 1..14: note = Description of Artificial Sequence: Synthetic peptide
AICGPEFDESVACD

SEQ ID NO: 388 - protein, 14 aa, synthetic construct
  REGION 1..14: note = Description of Artificial Sequence: Synthetic peptide
LLCGPDGVEDFFCD

SEQ ID NO: 389 - protein, 14 aa, synthetic construct
  REGION 1..14: note = Description of Artificial Sequence: Synthetic peptide
NFCGPELPEDIFCF

SEQ ID NO: 390 - protein, 14 aa, synthetic construct
  REGION 1..14: note = Description of Artificial Sequence: Synthetic peptide
FYCGPEVEEVSLCN

SEQ ID NO: 391 - protein, 14 aa, synthetic construct
  REGION 1..14: note = Description of Artificial Sequence: Synthetic peptide
YDCGPDGYEAFYCH

SEQ ID NO: 392 - protein, 14 aa, synthetic construct
  REGION 1..14: note = Description of Artificial Sequence: Synthetic peptide
NFCGPEIEFDYLCD

SEQ ID NO: 393 - protein, 14 aa, synthetic construct
  REGION 1..14: note = Description of Artificial Sequence: Synthetic peptide
DYCGPDGVEDFICN

SEQ ID NO: 394 - protein, 14 aa, synthetic construct
  REGION 1..14: note = Description of Artificial Sequence: Synthetic peptide
TDCWPDWEYIYSCS

SEQ ID NO: 395 - protein, 14 aa, synthetic construct
  REGION 1..14: note = Description of Artificial Sequence: Synthetic peptide
FYCGPEFEEITNCI

SEQ ID NO: 396 - protein, 14 aa, synthetic construct
  REGION 1..14: note = Description of Artificial Sequence: Synthetic peptide
FDCWPDWEESFFCH

SEQ ID NO: 397 - protein, 14 aa, synthetic construct
  REGION 1..14: note = Description of Artificial Sequence: Synthetic peptide
DFCGPDGDESVFCP

SEQ ID NO: 398 - protein, 14 aa, synthetic construct
  REGION 1..14: note = Description of Artificial Sequence: Synthetic peptide
HNCGPELDESLVCD

SEQ ID NO: 399 - protein, 14 aa, synthetic construct
  REGION 1..14: note = Description of Artificial Sequence: Synthetic peptide
IYCGPDGAEDYTCD

SEQ ID NO: 400 - protein, 14 aa, synthetic construct
  REGION 1..14: note = Description of Artificial Sequence: Synthetic peptide
FDCGPEFEFPVICF

SEQ ID NO: 401 - protein, 14 aa, synthetic construct
  REGION 1..14: note = Description of Artificial Sequence: Synthetic peptide
YNCGPELDESVTCD

SEQ ID NO: 402 - protein, 14 aa, synthetic construct
  REGION 1..14: note = Description of Artificial Sequence: Synthetic peptide
FDCGPELDESIHCA

SEQ ID NO: 403 - protein, 14 aa, synthetic construct
  REGION 1..14: note = Description of Artificial Sequence: Synthetic peptide
DICGPEVEEYFLCF

SEQ ID NO: 404 - protein, 14 aa, synthetic construct
  REGION 1..14: note = Description of Artificial Sequence: Synthetic peptide
FDCGPEVDESLTCF

SEQ ID NO: 405 - protein, 14 aa, synthetic construct
  REGION 1..14: note = Description of Artificial Sequence: Synthetic peptide
FDCGPEIEEFHLCF

SEQ ID NO: 406 - protein, 14 aa, synthetic construct
  REGION 1..14: note = Description of Artificial Sequence: Synthetic peptide
AYCGPELDESIICD

SEQ ID NO: 407 - protein, 14 aa, synthetic construct
  REGION 1..14: note = Description of Artificial Sequence: Synthetic peptide
YNCGPELDESITCN

SEQ ID NO: 408 - protein, 14 aa, synthetic construct
  REGION 1..14: note = Description of Artificial Sequence: Synthetic peptide
FDCWPDWEEPVDCL

SEQ ID NO: 409 - protein, 14 aa, synthetic construct
  REGION 1..14: note = Description of Artificial Sequence: Synthetic peptide
DVCGPELDESVLCP

SEQ ID NO: 410 - protein, 14 aa, synthetic construct
  REGION 1..14: note = Description of Artificial Sequence: Synthetic peptide
NYCGPELDESLPCP

SEQ ID NO: 411 - protein, 14 aa, synthetic construct
  REGION 1..14: note = Description of Artificial Sequence: Synthetic peptide
YYCWPDWEYDIFCS

SEQ ID NO: 412 - protein, 14 aa, synthetic construct
  REGION 1..14: note = Description of Artificial Sequence: Synthetic peptide
DDCGPEFDESTYCN

SEQ ID NO: 413 - protein, 14 aa, synthetic construct
  REGION 1..14: note = Description of Artificial Sequence: Synthetic peptide
FYCGPEFEEVFHCY

SEQ ID NO: 414 - protein, 14 aa, synthetic construct
  REGION 1..14: note = Description of Artificial Sequence: Synthetic peptide
TDCWPDWEEYFLCD

SEQ ID NO: 415 - protein, 14 aa, synthetic construct
  REGION 1..14: note = Description of Artificial Sequence: Synthetic peptide
VYCGPEWEESYLCP

SEQ ID NO: 416 - protein, 14 aa, synthetic construct
  REGION 1..14: note = Description of Artificial Sequence: Synthetic peptide
DDCGPNGYATFICY

SEQ ID NO: 417 - protein, 14 aa, synthetic construct
  REGION 1..14: note = Description of Artificial Sequence: Synthetic peptide
FLCGPEIEDDTHCY

SEQ ID NO: 418 - protein, 14 aa, synthetic construct
  REGION 1..14: note = Description of Artificial Sequence: Synthetic peptide
FACWPDWEETIPCH

SEQ ID NO: 419 - protein, 14 aa, synthetic construct
  REGION 1..14: note = Description of Artificial Sequence: Synthetic peptide
VLCGPEFDESYNCY

SEQ ID NO: 420 - protein, 14 aa, synthetic construct
  REGION 1..14: note = Description of Artificial Sequence: Synthetic peptide
YDCGPEFDESISCI

SEQ ID NO: 421 - protein, 14 aa, synthetic construct
  REGION 1..14: note = Description of Artificial Sequence: Synthetic peptide
YDCGPEFDESVNCY

SEQ ID NO: 422 - protein, 14 aa, synthetic construct
  REGION 1..14: note = Description of Artificial Sequence: Synthetic peptide
AICWPDWEEFVDCY

SEQ ID NO: 423 - protein, 14 aa, synthetic construct
  REGION 1..14: note = Description of Artificial Sequence: Synthetic peptide
LTCWPVREEIFACD

SEQ ID NO: 424 - protein, 14 aa, synthetic construct
  REGION 1..14: note = Description of Artificial Sequence: Synthetic peptide
VLCGPEFDESYYCN

SEQ ID NO: 425 - protein, 14 aa, synthetic construct
  REGION 1..14: note = Description of Artificial Sequence: Synthetic peptide
NVCGPEYDESAPCN

SEQ ID NO: 426 - protein, 14 aa, synthetic construct
  REGION 1..14: note = Description of Artificial Sequence: Synthetic peptide
HDCGPEFDESISCV

SEQ ID NO: 427 - protein, 14 aa, synthetic construct
  REGION 1..14: note = Description of Artificial Sequence: Synthetic peptide
FDCGPELDETVDCN

SEQ ID NO: 428 - protein, 14 aa, synthetic construct
  REGION 1..14: note = Description of Artificial Sequence: Synthetic peptide
YFCGPEVEEHFYCY

SEQ ID NO: 429 - protein, 14 aa, synthetic construct
  REGION 1..14: note = Description of Artificial Sequence: Synthetic peptide
DYCGPELDESIICH

SEQ ID NO: 430 - protein, 14 aa, synthetic construct
  REGION 1..14: note = Description of Artificial Sequence: Synthetic peptide
DDCGPEVPEDITCY

SEQ ID NO: 431 - protein, 14 aa, synthetic construct
  REGION 1..14: note = Description of Artificial Sequence: Synthetic peptide
DNCGPELDESVVCD

SEQ ID NO: 432 - protein, 14 aa, synthetic construct
  REGION 1..14: note = Description of Artificial Sequence: Synthetic peptide
VHCWPDWEPNYVCD

SEQ ID NO: 433 - protein, 14 aa, synthetic construct
  REGION 1..14: note = Description of Artificial Sequence: Synthetic peptide
DYCGPELDESLFCL

SEQ ID NO: 434 - protein, 14 aa, synthetic construct
  REGION 1..14: note = Description of Artificial Sequence: Synthetic peptide
DDCGPELDESVVCA

SEQ ID NO: 435 - protein, 14 aa, synthetic construct
  REGION 1..14: note = Description of Artificial Sequence: Synthetic peptide
AACGPELDESIVCD

SEQ ID NO: 436 - protein, 14 aa, synthetic construct
  REGION 1..14: note = Description of Artificial Sequence: Synthetic peptide
DFCGPEFEEINNCF

SEQ ID NO: 437 - protein, 14 aa, synthetic construct
  REGION 1..14: note = Description of Artificial Sequence: Synthetic peptide
YYCGPEFDESNACY

SEQ ID NO: 438 - protein, 14 aa, synthetic construct
  REGION 1..14: note = Description of Artificial Sequence: Synthetic peptide
VLCGPEFDESNSCY

SEQ ID NO: 439 - protein, 14 aa, synthetic construct
  REGION 1..14: note = Description of Artificial Sequence: Synthetic peptide
YDCGPEFDESIDCD

SEQ ID NO: 440 - protein, 14 aa, synthetic construct
  REGION 1..14: note = Description of Artificial Sequence: Synthetic peptide
DSCGPEFEFYYVCF

SEQ ID NO: 441 - protein, 14 aa, synthetic construct
  REGION 1..14: note = Description of Artificial Sequence: Synthetic peptide
YICGPELDESLICH

SEQ ID NO: 442 - protein, 14 aa, synthetic construct
  REGION 1..14: note = Description of Artificial Sequence: Synthetic peptide
FDCGPEVEEDYFCY

SEQ ID NO: 443 - protein, 14 aa, synthetic construct
  REGION 1..14: note = Description of Artificial Sequence: Synthetic peptide
FDCGPEYDESLYCF

SEQ ID NO: 444 - protein, 14 aa, synthetic construct
  REGION 1..14: note = Description of Artificial Sequence: Synthetic peptide
VLCGPDGDEYSFCH

SEQ ID NO: 445 - protein, 14 aa, synthetic construct
  REGION 1..14: note = Description of Artificial Sequence: Synthetic peptide
IPCGPEMDESVVCN

SEQ ID NO: 446 - protein, 14 aa, synthetic construct
  REGION 1..14: note = Description of Artificial Sequence: Synthetic peptide
IICGPDGYEDFTCD

SEQ ID NO: 447 - protein, 14 aa, synthetic construct
  REGION 1..14: note = Description of Artificial Sequence: Synthetic peptide
IFCGPELDESIICY

SEQ ID NO: 448 - protein, 14 aa, synthetic construct
  REGION 1..14: note = Description of Artificial Sequence: Synthetic peptide
VDCGPEFDESYDCY

SEQ ID NO: 449 - protein, 14 aa, synthetic construct
  REGION 1..14: note = Description of Artificial Sequence: Synthetic peptide
LSCGPEMDESLYCD

SEQ ID NO: 450 - protein, 14 aa, synthetic construct
  REGION 1..14: note = Description of Artificial Sequence: Synthetic peptide
YDCWPDWEYNIDCT

SEQ ID NO: 451 - protein, 14 aa, synthetic construct
  REGION 1..14: note = Description of Artificial Sequence: Synthetic peptide
NHCGPDGDETIVCF

SEQ ID NO: 452 - protein, 14 aa, synthetic construct
  REGION 1..14: note = Description of Artificial Sequence: Synthetic peptide
NFCGPELDESIPCH

SEQ ID NO: 453 - protein, 14 aa, synthetic construct
  REGION 1..14: note = Description of Artificial Sequence: Synthetic peptide
FHCGPEIEEYALCD

SEQ ID NO: 454 - protein, 14 aa, synthetic construct
  REGION 1..14: note = Description of Artificial Sequence: Synthetic peptide
VYCGPEVEDYNLCY

SEQ ID NO: 455 - protein, 14 aa, synthetic construct
  REGION 1..14: note = Description of Artificial Sequence: Synthetic peptide
VYCGPDGDELANCY

SEQ ID NO: 456 - protein, 14 aa, synthetic construct
  REGION 1..14: note = Description of Artificial Sequence: Synthetic peptide
LICGPVIAEDLPCN

SEQ ID NO: 457 - protein, 14 aa, synthetic construct
  REGION 1..14: note = Description of Artificial Sequence: Synthetic peptide
DICGPEIPEDVSCD

SEQ ID NO: 458 - protein, 14 aa, synthetic construct
  REGION 1..14: note = Description of Artificial Sequence: Synthetic peptide
IYCGPEWEEADYCD

SEQ ID NO: 459 - protein, 14 aa, synthetic construct
  REGION 1..14: note = Description of Artificial Sequence: Synthetic peptide
IDCWPDWEDDSICY

SEQ ID NO: 460 - protein, 14 aa, synthetic construct
  REGION 1..14: note = Description of Artificial Sequence: Synthetic peptide
VLCGPEVEDFTLCD

SEQ ID NO: 461 - protein, 14 aa, synthetic construct
  REGION 1..14: note = Description of Artificial Sequence: Synthetic peptide
LYCGPVIEEIYYCY

SEQ ID NO: 462 - protein, 14 aa, synthetic construct
  REGION 1..14: note = Description of Artificial Sequence: Synthetic peptide
FFCGPEFEVHSDCN

SEQ ID NO: 463 - protein, 14 aa, synthetic construct
  REGION 1..14: note = Description of Artificial Sequence: Synthetic peptide
NDCGPEVELVSDCN

SEQ ID NO: 464 - protein, 14 aa, synthetic construct
  REGION 1..14: note = Description of Artificial Sequence: Synthetic peptide
DLCGPELDESTVCD

SEQ ID NO: 465 - protein, 14 aa, synthetic construct
  REGION 1..14: note = Description of Artificial Sequence: Synthetic peptide
IPCGPEVEDYNLCN

SEQ ID NO: 466 - protein, 14 aa, synthetic construct
  REGION 1..14: note = Description of Artificial Sequence: Synthetic peptide
YYCGPELEWPVVCN

SEQ ID NO: 467 - protein, 14 aa, synthetic construct
  REGION 1..14: note = Description of Artificial Sequence: Synthetic peptide
YDCGPELDESVICN

SEQ ID NO: 468 - protein, 14 aa, synthetic construct
  REGION 1..14: note = Description of Artificial Sequence: Synthetic peptide
VYCGPDGDESFDCA

SEQ ID NO: 469 - protein, 14 aa, synthetic construct
  REGION 1..14: note = Description of Artificial Sequence: Synthetic peptide
NDCGPEWEDTYFCL

SEQ ID NO: 470 - protein, 14 aa, synthetic construct
  REGION 1..14: note = Description of Artificial Sequence: Synthetic peptide
PYCGPEMEELSNCS

SEQ ID NO: 471 - protein, 14 aa, synthetic construct
  REGION 1..14: note = Description of Artificial Sequence: Synthetic peptide
DDCGPEFEVISDCY

SEQ ID NO: 472 - protein, 14 aa, synthetic construct
  REGION 1..14: note = Description of Artificial Sequence: Synthetic peptide
DLCGPEFPEDVPCD

SEQ ID NO: 473 - protein, 14 aa, synthetic construct
  REGION 1..14: note = Description of Artificial Sequence: Synthetic peptide
IYCGPEFDESFVCY

SEQ ID NO: 474 - protein, 14 aa, synthetic construct
  REGION 1..14: note = Description of Artificial Sequence: Synthetic peptide
AYCGPEYEVFADCN

SEQ ID NO: 475 - protein, 14 aa, synthetic construct
  REGION 1..14: note = Description of Artificial Sequence: Synthetic peptide
IDCGPEYDESVDCL

SEQ ID NO: 476 - protein, 14 aa, synthetic construct
  REGION 1..14: note = Description of Artificial Sequence: Synthetic peptide
HICWPDWEEFHDCN

SEQ ID NO: 477 - protein, 14 aa, synthetic construct
  REGION 1..14: note = Description of Artificial Sequence: Synthetic peptide
YDCGPELDETITCL

SEQ ID NO: 478 - protein, 14 aa, synthetic construct
  REGION 1..14: note = Description of Artificial Sequence: Synthetic peptide
YLCGPELDETILCN

SEQ ID NO: 479 - protein, 14 aa, synthetic construct
  REGION 1..14: note = Description of Artificial Sequence: Synthetic peptide
FFCGPEFEEAFLCF

SEQ ID NO: 480 - protein, 14 aa, synthetic construct
  REGION 1..14: note = Description of Artificial Sequence: Synthetic peptide
ILCGPELDESFTCA

SEQ ID NO: 481 - protein, 14 aa, synthetic construct
  REGION 1..14: note = Description of Artificial Sequence: Synthetic peptide
FHCGPEVELYTDCN

SEQ ID NO: 482 - protein, 14 aa, synthetic construct
  REGION 1..14: note = Description of Artificial Sequence: Synthetic peptide
NLCGPEVEEYNFCY

SEQ ID NO: 483 - protein, 14 aa, synthetic construct
  REGION 1..14: note = Description of Artificial Sequence: Synthetic peptide
FDCGPEVEETYYCF

SEQ ID NO: 484 - protein, 14 aa, synthetic construct
  REGION 1..14: note = Description of Artificial Sequence: Synthetic peptide
VFCGPEFEEDHYCY

SEQ ID NO: 485 - protein, 14 aa, synthetic construct
  REGION 1..14: note = Description of Artificial Sequence: Synthetic peptide
AICGPEWEVVADCN

SEQ ID NO: 486 - protein, 14 aa, synthetic construct
  REGION 1..14: note = Description of Artificial Sequence: Synthetic peptide
FICWPDWEEDNYCN

SEQ ID NO: 487 - protein, 14 aa, synthetic construct
  REGION 1..14: note = Description of Artificial Sequence: Synthetic peptide
IYCGPEFDESFICD

SEQ ID NO: 488 - protein, 14 aa, synthetic construct
  REGION 1..14: note = Description of Artificial Sequence: Synthetic peptide
NLCGPEVEDVYDCH

SEQ ID NO: 489 - protein, 14 aa, synthetic construct
  REGION 1..14: note = Description of Artificial Sequence: Synthetic peptide
HYCGPEVEEYHNCN

SEQ ID NO: 490 - protein, 14 aa, synthetic construct
  REGION 1..14: note = Description of Artificial Sequence: Synthetic peptide
DICGPEYDESYSCT

SEQ ID NO: 491 - protein, 14 aa, synthetic construct
  REGION 1..14: note = Description of Artificial Sequence: Synthetic peptide
DYCGPELDETLICA

SEQ ID NO: 492 - protein, 14 aa, synthetic construct
  REGION 1..14: note = Description of Artificial Sequence: Synthetic peptide
IACGPEMPEDIDCY

SEQ ID NO: 493 - protein, 14 aa, synthetic construct
  REGION 1..14: note = Description of Artificial Sequence: Synthetic peptide
IDCGPELDESIFCD

SEQ ID NO: 494 - protein, 14 aa, synthetic construct
  REGION 1..14: note = Description of Artificial Sequence: Synthetic peptide
YFCGPDVEEDFACD

SEQ ID NO: 495 - protein, 14 aa, synthetic construct
  REGION 1..14: note = Description of Artificial Sequence: Synthetic peptide
YNCGPEWEYAILCD

SEQ ID NO: 496 - protein, 14 aa, synthetic construct
  REGION 1..14: note = Description of Artificial Sequence: Synthetic peptide
IYCGPEVEDYIVCN

SEQ ID NO: 497 - protein, 14 aa, synthetic construct
  REGION 1..14: note = Description of Artificial Sequence: Synthetic peptide
YDCGPEIDESTPCA

SEQ ID NO: 498 - protein, 14 aa, synthetic construct
  REGION 1..14: note = Description of Artificial Sequence: Synthetic peptide
DTCWPDWEHIYACD

SEQ ID NO: 499 - protein, 14 aa, synthetic construct
  REGION 1..14: note = Description of Artificial Sequence: Synthetic peptide
DICGPEMDESVTCN

SEQ ID NO: 500 - protein, 14 aa, synthetic construct
  REGION 1..14: note = Description of Artificial Sequence: Synthetic peptide
YDCWPDWERYFPCI

SEQ ID NO: 501 - protein, 14 aa, synthetic construct
  REGION 1..14: note = Description of Artificial Sequence: Synthetic peptide
HLCGPELDESVACS

SEQ ID NO: 502 - protein, 14 aa, synthetic construct
  REGION 1..14: note = Description of Artificial Sequence: Synthetic peptide
YDCGPDGDETTICA

SEQ ID NO: 503 - protein, 14 aa, synthetic construct
  REGION 1..14: note = Description of Artificial Sequence: Synthetic peptide
YYCGPEYEDVLDCF

SEQ ID NO: 504 - protein, 14 aa, synthetic construct
  REGION 1..14: note = Description of Artificial Sequence: Synthetic peptide
DFCGPEMDETISCD

SEQ ID NO: 505 - protein, 14 aa, synthetic construct
  REGION 1..14: note = Description of Artificial Sequence: Synthetic peptide
ILCGPELDESLVCD

SEQ ID NO: 506 - protein, 14 aa, synthetic construct
  REGION 1..14: note = Description of Artificial Sequence: Synthetic peptide
LICGPEWEVITNCD

SEQ ID NO: 507 - protein, 14 aa, synthetic construct
  REGION 1..14: note = Description of Artificial Sequence: Synthetic peptide
YDCGPEFDEYFGCP

SEQ ID NO: 508 - protein, 14 aa, synthetic construct
  REGION 1..14: note = Description of Artificial Sequence: Synthetic peptide
ALCGPEVEVYDVCV

SEQ ID NO: 509 - protein, 14 aa, synthetic construct
  REGION 1..14: note = Description of Artificial Sequence: Synthetic peptide
YHCWPDWEDVNFCY

SEQ ID NO: 510 - protein, 14 aa, synthetic construct
  REGION 1..14: note = Description of Artificial Sequence: Synthetic peptide
FLCGPMGGLTFYCY

SEQ ID NO: 511 - protein, 14 aa, synthetic construct
  REGION 1..14: note = Description of Artificial Sequence: Synthetic peptide
IICGPEFDEYVGCF

SEQ ID NO: 512 - protein, 14 aa, synthetic construct
  REGION 1..14: note = Description of Artificial Sequence: Synthetic peptide
FFCGPEMDESVHCF

SEQ ID NO: 513 - protein, 14 aa, synthetic construct
  REGION 1..14: note = Description of Artificial Sequence: Synthetic peptide
AFCGPEFDESLFCA

SEQ ID NO: 514 - protein, 14 aa, synthetic construct
  REGION 1..14: note = Description of Artificial Sequence: Synthetic peptide
NYCGPDGDETNICD

SEQ ID NO: 515 - protein, 14 aa, synthetic construct
  REGION 1..14: note = Description of Artificial Sequence: Synthetic peptide
YLCGPEWEWVHNCL

SEQ ID NO: 516 - protein, 14 aa, synthetic construct
  REGION 1..14: note = Description of Artificial Sequence: Synthetic peptide
ATCGPDGDESHICA

SEQ ID NO: 517 - protein, 14 aa, synthetic construct
  REGION 1..14: note = Description of Artificial Sequence: Synthetic peptide
VYCGPEVEVLDYCD

SEQ ID NO: 518 - protein, 14 aa, synthetic construct
  REGION 1..14: note = Description of Artificial Sequence: Synthetic peptide
IHCGPEWEFYTDCD

SEQ ID NO: 519 - protein, 14 aa, synthetic construct
  REGION 1..14: note = Description of Artificial Sequence: Synthetic peptide
DDCGPELDETVACD

SEQ ID NO: 520 - protein, 14 aa, synthetic construct
  REGION 1..14: note = Description of Artificial Sequence: Synthetic peptide
YLCGPEFDESIDCN

SEQ ID NO: 521 - protein, 14 aa, synthetic construct
  REGION 1..14: note = Description of Artificial Sequence: Synthetic peptide
LLCGPEVEDVFACY

SEQ ID NO: 522 - protein, 14 aa, synthetic construct
  REGION 1..14: note = Description of Artificial Sequence: Synthetic peptide
YDCGPELDESLTCD

SEQ ID NO: 523 - protein, 14 aa, synthetic construct
  REGION 1..14: note = Description of Artificial Sequence: Synthetic peptide
FDCGPELDETVNCY

SEQ ID NO: 524 - protein, 14 aa, synthetic construct
  REGION 1..14: note = Description of Artificial Sequence: Synthetic peptide
FACWPDWEEINDCH

SEQ ID NO: 525 - protein, 14 aa, synthetic construct
  REGION 1..14: note = Description of Artificial Sequence: Synthetic peptide
YYCGPEYEEDIYCN

SEQ ID NO: 526 - protein, 14 aa, synthetic construct
  REGION 1..14: note = Description of Artificial Sequence: Synthetic peptide
NVCGPEVEDYTFCY

SEQ ID NO: 527 - protein, 14 aa, synthetic construct
  REGION 1..14: note = Description of Artificial Sequence: Synthetic peptide
AYCGPELEEYDFCT

SEQ ID NO: 528 - protein, 14 aa, synthetic construct
  REGION 1..14: note = Description of Artificial Sequence: Synthetic peptide
FDCGPEIDESTICT

SEQ ID NO: 529 - protein, 14 aa, synthetic construct
  REGION 1..14: note = Description of Artificial Sequence: Synthetic peptide
NLCGPELDETLVCA

SEQ ID NO: 530 - protein, 14 aa, synthetic construct
  REGION 1..14: note = Description of Artificial Sequence: Synthetic peptide
YSCWPDWEEYLACN

SEQ ID NO: 531 - protein, 14 aa, synthetic construct
  REGION 1..14: note = Description of Artificial Sequence: Synthetic peptide
GICGPEVEDYNYCD

SEQ ID NO: 532 - protein, 14 aa, synthetic construct
  REGION 1..14: note = Description of Artificial Sequence: Synthetic peptide
FFCGPELDESVNCH

SEQ ID NO: 533 - protein, 14 aa, synthetic construct
  REGION 1..14: note = Description of Artificial Sequence: Synthetic peptide
YYCGPEYEEDFYCF

SEQ ID NO: 534 - protein, 14 aa, synthetic construct
  REGION 1..14: note = Description of Artificial Sequence: Synthetic peptide
LYCGPEYDESTDCY

SEQ ID NO: 535 - protein, 14 aa, synthetic construct
  REGION 1..14: note = Description of Artificial Sequence: Synthetic peptide
DYCGPEVEEDFLCY

SEQ ID NO: 536 - protein, 14 aa, synthetic construct
  REGION 1..14: note = Description of Artificial Sequence: Synthetic peptide
TLCGPEVELYIFCD

SEQ ID NO: 537 - protein, 14 aa, synthetic construct
  REGION 1..14: note = Description of Artificial Sequence: Synthetic peptide
FYCGPEFEQIADCY

SEQ ID NO: 538 - protein, 14 aa, synthetic construct
  REGION 1..14: note = Description of Artificial Sequence: Synthetic peptide
DDCGPEVEEYHLCD

SEQ ID NO: 539 - protein, 14 aa, synthetic construct
  REGION 1..14: note = Description of Artificial Sequence: Synthetic peptide
DYCGPELEDVTLCH

SEQ ID NO: 540 - protein, 14 aa, synthetic construct
  REGION 1..14: note = Description of Artificial Sequence: Synthetic peptide
VLCGPEVEDVNLCY

SEQ ID NO: 541 - protein, 14 aa, synthetic construct
  REGION 1..14: note = Description of Artificial Sequence: Synthetic peptide
DICGPELDETIDCY

SEQ ID NO: 542 - protein, 14 aa, synthetic construct
  REGION 1..14: note = Description of Artificial Sequence: Synthetic peptide
TYCGPEVEEDINCY

SEQ ID NO: 543 - protein, 14 aa, synthetic construct
  REGION 1..14: note = Description of Artificial Sequence: Synthetic peptide
IVCWPDWEEYPNCD

SEQ ID NO: 544 - protein, 14 aa, synthetic construct
  REGION 1..14: note = Description of Artificial Sequence: Synthetic peptide
SDCGPELDESIICT

SEQ ID NO: 545 - protein, 14 aa, synthetic construct
  REGION 1..14: note = Description of Artificial Sequence: Synthetic peptide
YDCGPELPEDYDCN

SEQ ID NO: 546 - protein, 14 aa, synthetic construct
  REGION 1..14: note = Description of Artificial Sequence: Synthetic peptide
NLCWPDWEEYYACD

SEQ ID NO: 547 - protein, 14 aa, synthetic construct
  REGION 1..14: note = Description of Artificial Sequence: Synthetic peptide
DDCGPELDESLPCH

SEQ ID NO: 548 - protein, 14 aa, synthetic construct
  REGION 1..14: note = Description of Artificial Sequence: Synthetic peptide
SLCGPDGQEDYTCF

SEQ ID NO: 549 - protein, 14 aa, synthetic construct
  REGION 1..14: note = Description of Artificial Sequence: Synthetic peptide
LICWPDWEEYNFCT

SEQ ID NO: 550 - protein, 14 aa, synthetic construct
  REGION 1..14: note = Description of Artificial Sequence: Synthetic peptide
FHCGPDGDETVPCI

SEQ ID NO: 551 - protein, 14 aa, synthetic construct
  REGION 1..14: note = Description of Artificial Sequence: Synthetic peptide
FDCGPEWEWIYDCF

SEQ ID NO: 552 - protein, 14 aa, synthetic construct
  REGION 1..14: note = Description of Artificial Sequence: Synthetic peptide
IYCGPEWDESLDCD

SEQ ID NO: 553 - protein, 14 aa, synthetic construct
  REGION 1..14: note = Description of Artificial Sequence: Synthetic peptide
LICGPEIDESASCN

SEQ ID NO: 554 - protein, 14 aa, synthetic construct
  REGION 1..14: note = Description of Artificial Sequence: Synthetic peptide
TSCWVDWEEFSDCI

SEQ ID NO: 555 - protein, 14 aa, synthetic construct
  REGION 1..14: note = Description of Artificial Sequence: Synthetic peptide
YYCGPEVEEDYVCD

SEQ ID NO: 556 - protein, 14 aa, synthetic construct
  REGION 1..14: note = Description of Artificial Sequence: Synthetic peptide
DDCGPEQEFIYACI

SEQ ID NO: 557 - protein, 14 aa, synthetic construct
  REGION 1..14: note = Description of Artificial Sequence: Synthetic peptide
ADCWPDWEEYADCY

SEQ ID NO: 558 - protein, 14 aa, synthetic construct
  REGION 1..14: note = Description of Artificial Sequence: Synthetic peptide
YYCGPEFDESIHCI

SEQ ID NO: 559 - protein, 14 aa, synthetic construct
  REGION 1..14: note = Description of Artificial Sequence: Synthetic peptide
SECWPDWEPFFDCN

SEQ ID NO: 560 - protein, 14 aa, synthetic construct
  REGION 1..14: note = Description of Artificial Sequence: Synthetic peptide
YHCGPEMDESLICT

SEQ ID NO: 561 - protein, 14 aa, synthetic construct
  REGION 1..14: note = Description of Artificial Sequence: Synthetic peptide
SDCWPDWEDAYFCI

SEQ ID NO: 562 - protein, 14 aa, synthetic construct
  REGION 1..14: note = Description of Artificial Sequence: Synthetic peptide
VDCGPEVEEYYHCD

SEQ ID NO: 563 - protein, 14 aa, synthetic construct
  REGION 1..14: note = Description of Artificial Sequence: Synthetic peptide
DNCGPEYDESIACN

SEQ ID NO: 564 - protein, 14 aa, synthetic construct
  REGION 1..14: note = Description of Artificial Sequence: Synthetic peptide
LYCGPVFEFYDYCY

SEQ ID NO: 565 - protein, 14 aa, synthetic construct
  REGION 1..14: note = Description of Artificial Sequence: Synthetic peptide
YTCGPEMDESVTCI

SEQ ID NO: 566 - protein, 14 aa, synthetic construct
  REGION 1..14: note = Description of Artificial Sequence: Synthetic peptide
NFCWPDWEVNSFCD

SEQ ID NO: 567 - protein, 14 aa, synthetic construct
  REGION 1..14: note = Description of Artificial Sequence: Synthetic peptide
NACWPDWEYIDFCN

SEQ ID NO: 568 - protein, 14 aa, synthetic construct
  REGION 1..14: note = Description of Artificial Sequence: Synthetic peptide
YNCGPEMDESIFCS

SEQ ID NO: 569 - protein, 14 aa, synthetic construct
  REGION 1..14: note = Description of Artificial Sequence: Synthetic peptide
LDCGPELDESITCY

SEQ ID NO: 570 - protein, 14 aa, synthetic construct
  REGION 1..14: note = Description of Artificial Sequence: Synthetic peptide
HYCGPEFDESINCD

SEQ ID NO: 571 - protein, 14 aa, synthetic construct
  REGION 1..14: note = Description of Artificial Sequence: Synthetic peptide
IICGPELPEDYVCT

SEQ ID NO: 572 - protein, 14 aa, synthetic construct
  REGION 1..14: note = Description of Artificial Sequence: Synthetic peptide
DICGPEFDESIDCS

SEQ ID NO: 573 - protein, 14 aa, synthetic construct
  REGION 1..14: note = Description of Artificial Sequence: Synthetic peptide
PYCGPEFDESVLCF

SEQ ID NO: 574 - protein, 14 aa, synthetic construct
  REGION 1..14: note = Description of Artificial Sequence: Synthetic peptide
VFCGPEFDESVDCY

SEQ ID NO: 575 - protein, 14 aa, synthetic construct
  REGION 1..14: note = Description of Artificial Sequence: Synthetic peptide
YDCWPDWEEALPCA

SEQ ID NO: 576 - protein, 14 aa, synthetic construct
  REGION 1..14: note = Description of Artificial Sequence: Synthetic peptide
DDCWPDWEDYVFCF

SEQ ID NO: 577 - protein, 14 aa, synthetic construct
  REGION 1..14: note = Description of Artificial Sequence: Synthetic peptide
HFCGPEWELFSDCY

SEQ ID NO: 578 - protein, 14 aa, synthetic construct
  REGION 1..14: note = Description of Artificial Sequence: Synthetic peptide
ITCWPDWEVNFPCY

SEQ ID NO: 579 - protein, 14 aa, synthetic construct
  REGION 1..14: note = Description of Artificial Sequence: Synthetic peptide
PDCGPELDESITCN

SEQ ID NO: 580 - protein, 14 aa, synthetic construct
  REGION 1..14: note = Description of Artificial Sequence: Synthetic peptide
NLCWPDWEAFFPCY

SEQ ID NO: 581 - protein, 14 aa, synthetic construct
  REGION 1..14: note = Description of Artificial Sequence: Synthetic peptide
FYCGPEFEYIRDCY

SEQ ID NO: 582 - protein, 14 aa, synthetic construct
  REGION 1..14: note = Description of Artificial Sequence: Synthetic peptide
FFCGPEFDESIICD

SEQ ID NO: 583 - protein, 14 aa, synthetic construct
  REGION 1..14: note = Description of Artificial Sequence: Synthetic peptide
VLCGPKGGPTYNCS

SEQ ID NO: 584 - protein, 14 aa, synthetic construct
  REGION 1..14: note = Description of Artificial Sequence: Synthetic peptide
LACWPVWEEPGHCD

SEQ ID NO: 585 - protein, 14 aa, synthetic construct
  REGION 1..14: note = Description of Artificial Sequence: Synthetic peptide
DYCGPEVEDVNDCY

SEQ ID NO: 586 - protein, 14 aa, synthetic construct
  REGION 1..14: note = Description of Artificial Sequence: Synthetic peptide
DYCGPEFEEAHYCN

SEQ ID NO: 587 - protein, 14 aa, synthetic construct
  REGION 1..14: note = Description of Artificial Sequence: Synthetic peptide
VLCGPELDETLTCI

SEQ ID NO: 588 - protein, 14 aa, synthetic construct
  REGION 1..14: note = Description of Artificial Sequence: Synthetic peptide
YYCGPEIEDYNLCN

SEQ ID NO: 589 - protein, 14 aa, synthetic construct
  REGION 1..14: note = Description of Artificial Sequence: Synthetic peptide
YICGPEVEEYYNCF

SEQ ID NO: 590 - protein, 14 aa, synthetic construct
  REGION 1..14: note = Description of Artificial Sequence: Synthetic peptide
DICGPELDESIFCF

SEQ ID NO: 591 - protein, 14 aa, synthetic construct
  REGION 1..14: note = Description of Artificial Sequence: Synthetic peptide
DICGPEVEEDYLCY

SEQ ID NO: 592 - protein, 14 aa, synthetic construct
  REGION 1..14: note = Description of Artificial Sequence: Synthetic peptide
TLCGPEFEEDAPCI

SEQ ID NO: 593 - protein, 14 aa, synthetic construct
  REGION 1..14: note = Description of Artificial Sequence: Synthetic peptide
NYCWPDWEYINSCV

SEQ ID NO: 594 - protein, 14 aa, synthetic construct
  REGION 1..14: note = Description of Artificial Sequence: Synthetic peptide
NDCGPEVEEYYYCT

SEQ ID NO: 595 - protein, 14 aa, synthetic construct
  REGION 1..14: note = Description of Artificial Sequence: Synthetic peptide
ITCGPEMDESIDCN

SEQ ID NO: 596 - protein, 14 aa, synthetic construct
  REGION 1..14: note = Description of Artificial Sequence: Synthetic peptide
YYCGPEMAEDLICD

SEQ ID NO: 597 - protein, 14 aa, synthetic construct
  REGION 1..14: note = Description of Artificial Sequence: Synthetic peptide
IDCGPELDESIVCT

SEQ ID NO: 598 - protein, 14 aa, synthetic construct
  REGION 1..14: note = Description of Artificial Sequence: Synthetic peptide
HTCWPDWEWDVYCD

SEQ ID NO: 599 - protein, 14 aa, synthetic construct
  REGION 1..14: note = Description of Artificial Sequence: Synthetic peptide
IHCGPEWELIDDCL

SEQ ID NO: 600 - protein, 14 aa, synthetic construct
  REGION 1..14: note = Description of Artificial Sequence: Synthetic peptide
YTCGPELDESITCT

SEQ ID NO: 601 - protein, 14 aa, synthetic construct
  REGION 1..14: note = Description of Artificial Sequence: Synthetic peptide
FHCGPEVEETVYCF

SEQ ID NO: 602 - protein, 14 aa, synthetic construct
  REGION 1..14: note = Description of Artificial Sequence: Synthetic peptide
DYCGPELDESLICN

SEQ ID NO: 603 - protein, 14 aa, synthetic construct
  REGION 1..14: note = Description of Artificial Sequence: Synthetic peptide
IYCGPEFDESDYCI

SEQ ID NO: 604 - protein, 14 aa, synthetic construct
  REGION 1..14: note = Description of Artificial Sequence: Synthetic peptide
DLCGPEIEEDLVCT

SEQ ID NO: 605 - protein, 14 aa, synthetic construct
  REGION 1..14: note = Description of Artificial Sequence: Synthetic peptide
NICGPELQEDIVCP

SEQ ID NO: 606 - protein, 14 aa, synthetic construct
  REGION 1..14: note = Description of Artificial Sequence: Synthetic peptide
NNCGPEMDESITCY

SEQ ID NO: 607 - protein, 14 aa, synthetic construct
  REGION 1..14: note = Description of Artificial Sequence: Synthetic peptide
HTCGPELDESIVCV

SEQ ID NO: 608 - protein, 14 aa, synthetic construct
  REGION 1..14: note = Description of Artificial Sequence: Synthetic peptide
YYCGPEIEDILVCT

SEQ ID NO: 609 - protein, 14 aa, synthetic construct
  REGION 1..14: note = Description of Artificial Sequence: Synthetic peptide
NTCGPEFEFVHLCP

SEQ ID NO: 610 - protein, 14 aa, synthetic construct
  REGION 1..14: note = Description of Artificial Sequence: Synthetic peptide
DICGPEMDESTVCD

SEQ ID NO: 611 - protein, 14 aa, synthetic construct
  REGION 1..14: note = Description of Artificial Sequence: Synthetic peptide
AICGPEVEIVNYCY

SEQ ID NO: 612 - protein, 14 aa, synthetic construct
  REGION 1..14: note = Description of Artificial Sequence: Synthetic peptide
HLCGPEVEDPTACV

SEQ ID NO: 613 - protein, 14 aa, synthetic construct
  REGION 1..14: note = Description of Artificial Sequence: Synthetic peptide
ADCGPELDESISCT

SEQ ID NO: 614 - protein, 14 aa, synthetic construct
  REGION 1..14: note = Description of Artificial Sequence: Synthetic peptide
FDCGPELDESVICD

SEQ ID NO: 615 - protein, 14 aa, synthetic construct
  REGION 1..14: note = Description of Artificial Sequence: Synthetic peptide
DVCGPEFDESIDCN

SEQ ID NO: 616 - protein, 14 aa, synthetic construct
  REGION 1..14: note = Description of Artificial Sequence: Synthetic peptide
YLCGPEVEEISICF

SEQ ID NO: 617 - protein, 14 aa, synthetic construct
  REGION 1..14: note = Description of Artificial Sequence: Synthetic peptide
SACGPEFDESLHCV

SEQ ID NO: 618 - protein, 14 aa, synthetic construct
  REGION 1..14: note = Description of Artificial Sequence: Synthetic peptide
HLCWPDWEEDSACN

SEQ ID NO: 619 - protein, 14 aa, synthetic construct
  REGION 1..14: note = Description of Artificial Sequence: Synthetic peptide
HTCWPDWEYDYDCF

SEQ ID NO: 620 - protein, 14 aa, synthetic construct
  REGION 1..14: note = Description of Artificial Sequence: Synthetic peptide
YDCGPEWEEVALCN

SEQ ID NO: 621 - protein, 14 aa, synthetic construct
  REGION 1..14: note = Description of Artificial Sequence: Synthetic peptide
TLCGPEIEEYIVCY

SEQ ID NO: 622 - protein, 14 aa, synthetic construct
  REGION 1..14: note = Description of Artificial Sequence: Synthetic peptide
SYCGPEFDESIFCT

SEQ ID NO: 623 - protein, 14 aa, synthetic construct
  REGION 1..14: note = Description of Artificial Sequence: Synthetic peptide
DICGPEFDESLHCY

SEQ ID NO: 624 - protein, 14 aa, synthetic construct
  REGION 1..14: note = Description of Artificial Sequence: Synthetic peptide
LFCGPEFEEAYLCI

SEQ ID NO: 625 - protein, 14 aa, synthetic construct
  REGION 1..14: note = Description of Artificial Sequence: Synthetic peptide
IFCGPEIEEDFVCT

SEQ ID NO: 626 - protein, 14 aa, synthetic construct
  REGION 1..14: note = Description of Artificial Sequence: Synthetic peptide
DDCGPEWEYYVACV

SEQ ID NO: 627 - protein, 14 aa, synthetic construct
  REGION 1..14: note = Description of Artificial Sequence: Synthetic peptide
DDCGPELDETTICY

SEQ ID NO: 628 - protein, 14 aa, synthetic construct
  REGION 1..14: note = Description of Artificial Sequence: Synthetic peptide
ASCGPELDESIACD

SEQ ID NO: 629 - protein, 14 aa, synthetic construct
  REGION 1..14: note = Description of Artificial Sequence: Synthetic peptide
YLCGPEVEDYDYCY

SEQ ID NO: 630 - protein, 14 aa, synthetic construct
  REGION 1..14: note = Description of Artificial Sequence: Synthetic peptide
DLCGPEWEETIFCA

SEQ ID NO: 631 - protein, 14 aa, synthetic construct
  REGION 1..14: note = Description of Artificial Sequence: Synthetic peptide
DFCGPDGEEFYICP

SEQ ID NO: 632 - protein, 14 aa, synthetic construct
  REGION 1..14: note = Description of Artificial Sequence: Synthetic peptide
VYCGPEVEENILCH

SEQ ID NO: 633 - protein, 14 aa, synthetic construct
  REGION 1..14: note = Description of Artificial Sequence: Synthetic peptide
DYCGPEVEENYFCF

SEQ ID NO: 634 - protein, 14 aa, synthetic construct
  REGION 1..14: note = Description of Artificial Sequence: Synthetic peptide
NDCWPDWYEFLSCD

SEQ ID NO: 635 - protein, 14 aa, synthetic construct
  REGION 1..14: note = Description of Artificial Sequence: Synthetic peptide
RDCWPDWEVPYFCD

SEQ ID NO: 636 - protein, 14 aa, synthetic construct
  REGION 1..14: note = Description of Artificial Sequence: Synthetic peptide
NLCGPEVEEAVYCY

SEQ ID NO: 637 - protein, 14 aa, synthetic construct
  REGION 1..14: note = Description of Artificial Sequence: Synthetic peptide
FDCWPDGELNYLCT

SEQ ID NO: 638 - protein, 14 aa, synthetic construct
  REGION 1..14: note = Description of Artificial Sequence: Synthetic peptide
YYCGPEVEDVNLCI

SEQ ID NO: 639 - protein, 14 aa, synthetic construct
  REGION 1..14: note = Description of Artificial Sequence: Synthetic peptide
DNCGPEYDESITCL

SEQ ID NO: 640 - protein, 14 aa, synthetic construct
  REGION 1..14: note = Description of Artificial Sequence: Synthetic peptide
LNCWPDWEEDYSCN

SEQ ID NO: 641 - protein, 14 aa, synthetic construct
  REGION 1..14: note = Description of Artificial Sequence: Synthetic peptide
PTCGPEVEELLSCN

SEQ ID NO: 642 - protein, 14 aa, synthetic construct
  REGION 1..14: note = Description of Artificial Sequence: Synthetic peptide
DHCGPEVELIFYCH

SEQ ID NO: 643 - protein, 14 aa, synthetic construct
  REGION 1..14: note = Description of Artificial Sequence: Synthetic peptide
FHCGPELDESIFCY

SEQ ID NO: 644 - protein, 14 aa, synthetic construct
  REGION 1..14: note = Description of Artificial Sequence: Synthetic peptide
FDCGPELEETVVCP

SEQ ID NO: 645 - protein, 14 aa, synthetic construct
  REGION 1..14: note = Description of Artificial Sequence: Synthetic peptide
DFCGPELDESLPCA

SEQ ID NO: 646 - protein, 14 aa, synthetic construct
  REGION 1..14: note = Description of Artificial Sequence: Synthetic peptide
DSCGPELDESIYCD

SEQ ID NO: 647 - protein, 14 aa, synthetic construct
  REGION 1..14: note = Description of Artificial Sequence: Synthetic peptide
ADCWPDWEEFLLCF

SEQ ID NO: 648 - protein, 14 aa, synthetic construct
  REGION 1..14: note = Description of Artificial Sequence: Synthetic peptide
ILCGPEVEELDFCN

SEQ ID NO: 649 - protein, 14 aa, synthetic construct
  REGION 1..14: note = Description of Artificial Sequence: Synthetic peptide
FFCGPEFEEIFLCY

SEQ ID NO: 650 - protein, 14 aa, synthetic construct
  REGION 1..14: note = Description of Artificial Sequence: Synthetic peptide
YNCGPDGDESYDCH

SEQ ID NO: 651 - protein, 14 aa, synthetic construct
  REGION 1..14: note = Description of Artificial Sequence: Synthetic peptide
DNCGPELDETITCF

SEQ ID NO: 652 - protein, 14 aa, synthetic construct
  REGION 1..14: note = Description of Artificial Sequence: Synthetic peptide
HNCGPDGDEAFICN

SEQ ID NO: 653 - protein, 14 aa, synthetic construct
  REGION 1..14: note = Description of Artificial Sequence: Synthetic peptide
DYCGPEVEEDLVCP

SEQ ID NO: 654 - protein, 14 aa, synthetic construct
  REGION 1..14: note = Description of Artificial Sequence: Synthetic peptide
DYCGPELDESLNCF

SEQ ID NO: 655 - protein, 14 aa, synthetic construct
  REGION 1..14: note = Description of Artificial Sequence: Synthetic peptide
DYCGPEFQEDFHCH

SEQ ID NO: 656 - protein, 14 aa, synthetic construct
  REGION 1..14: note = Description of Artificial Sequence: Synthetic peptide
YDCGPEWEFTDDCI

SEQ ID NO: 657 - protein, 14 aa, synthetic construct
  REGION 1..14: note = Description of Artificial Sequence: Synthetic peptide
DICGPEYEEDIICY

SEQ ID NO: 658 - protein, 14 aa, synthetic construct
  REGION 1..14: note = Description of Artificial Sequence: Synthetic peptide
FDCGPELDETVPCP

SEQ ID NO: 659 - protein, 14 aa, synthetic construct
  REGION 1..14: note = Description of Artificial Sequence: Synthetic peptide
NYCGPELDETSVCD

SEQ ID NO: 660 - protein, 14 aa, synthetic construct
  REGION 1..14: note = Description of Artificial Sequence: Synthetic peptide
YYCGPEWEFSFDCD

SEQ ID NO: 661 - protein, 14 aa, synthetic construct
  REGION 1..14: note = Description of Artificial Sequence: Synthetic peptide
YACGPELDESVTCD

SEQ ID NO: 662 - protein, 14 aa, synthetic construct
  REGION 1..14: note = Description of Artificial Sequence: Synthetic peptide
FLCGPEVEQDYFCV

SEQ ID NO: 663 - protein, 14 aa, synthetic construct
  REGION 1..14: note = Description of Artificial Sequence: Synthetic peptide
DNCGPEFDESVRCD

SEQ ID NO: 664 - protein, 14 aa, synthetic construct
  REGION 1..14: note = Description of Artificial Sequence: Synthetic peptide
NDCGPDGIETVDCY

SEQ ID NO: 665 - protein, 14 aa, synthetic construct
  REGION 1..14: note = Description of Artificial Sequence: Synthetic peptide
YFCGPEVEDYNDCF

SEQ ID NO: 666 - protein, 14 aa, synthetic construct
  REGION 1..14: note = Description of Artificial Sequence: Synthetic peptide
NLCGPEFDESIFCY

SEQ ID NO: 667 - protein, 14 aa, synthetic construct
  REGION 1..14: note = Description of Artificial Sequence: Synthetic peptide
IDCWPDWEEYIPCT

SEQ ID NO: 668 - protein, 14 aa, synthetic construct
  REGION 1..14: note = Description of Artificial Sequence: Synthetic peptide
IYCGPDGDESFICA

SEQ ID NO: 669 - protein, 14 aa, synthetic construct
  REGION 1..14: note = Description of Artificial Sequence: Synthetic peptide
DYCGPEFDESVVCY

SEQ ID NO: 670 - protein, 14 aa, synthetic construct
  REGION 1..14: note = Description of Artificial Sequence: Synthetic peptide
VFCGPEWEDITDCD

SEQ ID NO: 671 - protein, 14 aa, synthetic construct
  REGION 1..14: note = Description of Artificial Sequence: Synthetic peptide
LDCGPEVDETFTCH

SEQ ID NO: 672 - protein, 14 aa, synthetic construct
  REGION 1..14: note = Description of Artificial Sequence: Synthetic peptide
DDCGPEYDESFACH

SEQ ID NO: 673 - protein, 14 aa, synthetic construct
  REGION 1..14: note = Description of Artificial Sequence: Synthetic peptide
IYCGPEYQEDLPCN

SEQ ID NO: 674 - protein, 14 aa, synthetic construct
  REGION 1..14: note = Description of Artificial Sequence: Synthetic peptide
LDCGPEVEEYNYCV

SEQ ID NO: 675 - protein, 14 aa, synthetic construct
  REGION 1..14: note = Description of Artificial Sequence: Synthetic peptide
YVCGPEFDESSACN

SEQ ID NO: 676 - protein, 14 aa, synthetic construct
  REGION 1..14: note = Description of Artificial Sequence: Synthetic peptide
TPCGPELEEAIGCY

SEQ ID NO: 677 - protein, 14 aa, synthetic construct
  REGION 1..14: note = Description of Artificial Sequence: Synthetic peptide
YFCGPEFDESADCN

SEQ ID NO: 678 - protein, 14 aa, synthetic construct
  REGION 1..14: note = Description of Artificial Sequence: Synthetic peptide
DDCGPEFEEDIICD

SEQ ID NO: 679 - protein, 14 aa, synthetic construct
  REGION 1..14: note = Description of Artificial Sequence: Synthetic peptide
LYCGPVVEELNHCN

SEQ ID NO: 680 - protein, 14 aa, synthetic construct
  REGION 1..14: note = Description of Artificial Sequence: Synthetic peptide
VICGPDGEELIACA

SEQ ID NO: 681 - protein, 14 aa, synthetic construct
  REGION 1..14: note = Description of Artificial Sequence: Synthetic peptide
FACWPDWQETYVCN

SEQ ID NO: 682 - protein, 14 aa, synthetic construct
  REGION 1..14: note = Description of Artificial Sequence: Synthetic peptide
YICGPEVEFLFFCN

SEQ ID NO: 683 - protein, 14 aa, synthetic construct
  REGION 1..14: note = Description of Artificial Sequence: Synthetic peptide
TQCGPKGEPTYHCY

SEQ ID NO: 684 - protein, 14 aa, synthetic construct
  REGION 1..14: note = Description of Artificial Sequence: Synthetic peptide
NYCGPEVEEYHNCD

SEQ ID NO: 685 - protein, 14 aa, synthetic construct
  REGION 1..14: note = Description of Artificial Sequence: Synthetic peptide
VYCGPEWEFFSDCA

SEQ ID NO: 686 - protein, 14 aa, synthetic construct
  REGION 1..14: note = Description of Artificial Sequence: Synthetic peptide
FYCGPELEESFFCY

SEQ ID NO: 687 - protein, 14 aa, synthetic construct
  REGION 1..14: note = Description of Artificial Sequence: Synthetic peptide
DYCGPEIEENFYCY

SEQ ID NO: 688 - protein, 14 aa, synthetic construct
  REGION 1..14: note = Description of Artificial Sequence: Synthetic peptide
YACGPEVEEYVYCA

SEQ ID NO: 689 - protein, 14 aa, synthetic construct
  REGION 1..14: note = Description of Artificial Sequence: Synthetic peptide
VDCGPELDESIICD

SEQ ID NO: 690 - protein, 14 aa, synthetic construct
  REGION 1..14: note = Description of Artificial Sequence: Synthetic peptide
NDCGPEMDESIACY

SEQ ID NO: 691 - protein, 14 aa, synthetic construct
  REGION 1..14: note = Description of Artificial Sequence: Synthetic peptide
VYCGPEFDEYLACA

SEQ ID NO: 692 - protein, 14 aa, synthetic construct
  REGION 1..14: note = Description of Artificial Sequence: Synthetic peptide
VICGPEFDESANCD

SEQ ID NO: 693 - protein, 14 aa, synthetic construct
  REGION 1..14: note = Description of Artificial Sequence: Synthetic peptide
LTCWPDWEEDFFCN

SEQ ID NO: 694 - protein, 14 aa, synthetic construct
  REGION 1..14: note = Description of Artificial Sequence: Synthetic peptide
FYCGPEQEEINYCY

SEQ ID NO: 695 - protein, 14 aa, synthetic construct
  REGION 1..14: note = Description of Artificial Sequence: Synthetic peptide
TYCGPELEEFFLCY

SEQ ID NO: 696 - protein, 14 aa, synthetic construct
  REGION 1..14: note = Description of Artificial Sequence: Synthetic peptide
PDCGPELDESVACH

SEQ ID NO: 697 - protein, 14 aa, synthetic construct
  REGION 1..14: note = Description of Artificial Sequence: Synthetic peptide
NDCGPEFEEIIFCV

SEQ ID NO: 698 - protein, 14 aa, synthetic construct
  REGION 1..14: note = Description of Artificial Sequence: Synthetic peptide
YICGPEFDESFYCN

SEQ ID NO: 699 - protein, 14 aa, synthetic construct
  REGION 1..14: note = Description of Artificial Sequence: Synthetic peptide
HACGPELDESLLCN

SEQ ID NO: 700 - protein, 14 aa, synthetic construct
  REGION 1..14: note = Description of Artificial Sequence: Synthetic peptide
YYCGPEWEEAVLCA

SEQ ID NO: 701 - protein, 14 aa, synthetic construct
  REGION 1..14: note = Description of Artificial Sequence: Synthetic peptide
AFCGPEVEEYDLCN

SEQ ID NO: 702 - protein, 14 aa, synthetic construct
  REGION 1..14: note = Description of Artificial Sequence: Synthetic peptide
YNCGPEFDESVACS

SEQ ID NO: 703 - protein, 14 aa, synthetic construct
  REGION 1..14: note = Description of Artificial Sequence: Synthetic peptide
YLCGPEVEDDTLCA

SEQ ID NO: 704 - protein, 14 aa, synthetic construct
  REGION 1..14: note = Description of Artificial Sequence: Synthetic peptide
AVCGPEFDESVNCD

SEQ ID NO: 705 - protein, 14 aa, synthetic construct
  REGION 1..14: note = Description of Artificial Sequence: Synthetic peptide
NYCGPEWEVYSLCP

SEQ ID NO: 706 - protein, 14 aa, synthetic construct
  REGION 1..14: note = Description of Artificial Sequence: Synthetic peptide
DFCGPEVEEDTYCH

SEQ ID NO: 707 - protein, 14 aa, synthetic construct
  REGION 1..14: note = Description of Artificial Sequence: Synthetic peptide
ISCGPEYEWDYACN

SEQ ID NO: 708 - protein, 14 aa, synthetic construct
  REGION 1..14: note = Description of Artificial Sequence: Synthetic peptide
VNCGPELDESIICY

SEQ ID NO: 709 - protein, 14 aa, synthetic construct
  REGION 1..14: note = Description of Artificial Sequence: Synthetic peptide
YDCGPEFDETAPCY

SEQ ID NO: 710 - protein, 14 aa, synthetic construct
  REGION 1..14: note = Description of Artificial Sequence: Synthetic peptide
YLCGPEFEENFLCT

SEQ ID NO: 711 - protein, 14 aa, synthetic construct
  REGION 1..14: note = Description of Artificial Sequence: Synthetic peptide
FDCGPEVDESVDCA

SEQ ID NO: 712 - protein, 14 aa, synthetic construct
  REGION 1..14: note = Description of Artificial Sequence: Synthetic peptide
YDCGPEQEEISFCN

SEQ ID NO: 713 - protein, 14 aa, synthetic construct
  REGION 1..14: note = Description of Artificial Sequence: Synthetic peptide
IDCGPEIELYDDCF

SEQ ID NO: 714 - protein, 14 aa, synthetic construct
  REGION 1..14: note = Description of Artificial Sequence: Synthetic peptide
TFCGPELDESVYCY

SEQ ID NO: 715 - protein, 14 aa, synthetic construct
  REGION 1..14: note = Description of Artificial Sequence: Synthetic peptide
FFCGPEIDESNACV

SEQ ID NO: 716 - protein, 14 aa, synthetic construct
  REGION 1..14: note = Description of Artificial Sequence: Synthetic peptide
YHCWPDWEPIYICI

SEQ ID NO: 717 - protein, 14 aa, synthetic construct
  REGION 1..14: note = Description of Artificial Sequence: Synthetic peptide
AICGPEYEEDHYCY

SEQ ID NO: 718 - protein, 14 aa, synthetic construct
  REGION 1..14: note = Description of Artificial Sequence: Synthetic peptide
PLCGPDGFENYNCF

SEQ ID NO: 719 - protein, 14 aa, synthetic construct
  REGION 1..14: note = Description of Artificial Sequence: Synthetic peptide
FPCWPDWEWDNNCH

SEQ ID NO: 720 - protein, 14 aa, synthetic construct
  REGION 1..14: note = Description of Artificial Sequence: Synthetic peptide
VDCGPDGDELAACH

SEQ ID NO: 721 - protein, 14 aa, synthetic construct
  REGION 1..14: note = Description of Artificial Sequence: Synthetic peptide
VDCWPDWEEYYSCD

SEQ ID NO: 722 - protein, 14 aa, synthetic construct
  REGION 1..14: note = Description of Artificial Sequence: Synthetic peptide
VYCGPEYDESYDCT

SEQ ID NO: 723 - protein, 14 aa, synthetic construct
  REGION 1..14: note = Description of Artificial Sequence: Synthetic peptide
NLCGPEWENFADCF

SEQ ID NO: 724 - protein, 14 aa, synthetic construct
  REGION 1..14: note = Description of Artificial Sequence: Synthetic peptide
YLCGPELEVFFVCD

SEQ ID NO: 725 - protein, 14 aa, synthetic construct
  REGION 1..14: note = Description of Artificial Sequence: Synthetic peptide
IFCGPELEDYSICF

SEQ ID NO: 726 - protein, 14 aa, synthetic construct
  REGION 1..14: note = Description of Artificial Sequence: Synthetic peptide
DYCGPELEQYDLCF

SEQ ID NO: 727 - protein, 14 aa, synthetic construct
  REGION 1..14: note = Description of Artificial Sequence: Synthetic peptide
LLCGPVNEDPLDCY

SEQ ID NO: 728 - protein, 14 aa, synthetic construct
  REGION 1..14: note = Description of Artificial Sequence: Synthetic peptide
IDCGPEFDESVFCY

SEQ ID NO: 729 - protein, 14 aa, synthetic construct
  REGION 1..14: note = Description of Artificial Sequence: Synthetic peptide
IICGPEVEEIDICS

SEQ ID NO: 730 - protein, 14 aa, synthetic construct
  REGION 1..14: note = Description of Artificial Sequence: Synthetic peptide
IACWPDWEDYSSCP

SEQ ID NO: 731 - protein, 14 aa, synthetic construct
  REGION 1..14: note = Description of Artificial Sequence: Synthetic peptide
YYCGPEVEDINDCI

SEQ ID NO: 732 - protein, 14 aa, synthetic construct
  REGION 1..14: note = Description of Artificial Sequence: Synthetic peptide
NICGPEMDESIDCI

SEQ ID NO: 733 - protein, 14 aa, synthetic construct
  REGION 1..14: note = Description of Artificial Sequence: Synthetic peptide
FDCWPDWEELVSCY

SEQ ID NO: 734 - protein, 14 aa, synthetic construct
  REGION 1..14: note = Description of Artificial Sequence: Synthetic peptide
YFCGPEWEDHFFCD

SEQ ID NO: 735 - protein, 14 aa, synthetic construct
  REGION 1..14: note = Description of Artificial Sequence: Synthetic peptide
TYCGPEFEEDSYCD

SEQ ID NO: 736 - protein, 14 aa, synthetic construct
  REGION 1..14: note = Description of Artificial Sequence: Synthetic peptide
NLCGPEVELIDICS

SEQ ID NO: 737 - protein, 14 aa, synthetic construct
  REGION 1..14: note = Description of Artificial Sequence: Synthetic peptide
DNCGPEWEEVYLCN

SEQ ID NO: 738 - protein, 14 aa, synthetic construct
  REGION 1..14: note = Description of Artificial Sequence: Synthetic peptide
FLCGPEFDESDLCF

SEQ ID NO: 739 - protein, 14 aa, synthetic construct
  REGION 1..14: note = Description of Artificial Sequence: Synthetic peptide
HICGPEQDESIGCT

SEQ ID NO: 740 - protein, 14 aa, synthetic construct
  REGION 1..14: note = Description of Artificial Sequence: Synthetic peptide
FDCWPDWEDNSYCD

SEQ ID NO: 741 - protein, 14 aa, synthetic construct
  REGION 1..14: note = Description of Artificial Sequence: Synthetic peptide
TACGPEWEFDFNCD

SEQ ID NO: 742 - protein, 14 aa, synthetic construct
  REGION 1..14: note = Description of Artificial Sequence: Synthetic peptide
HHCWPDWEDYSTCP

SEQ ID NO: 743 - protein, 14 aa, synthetic construct
  REGION 1..14: note = Description of Artificial Sequence: Synthetic peptide
YYCGPEFDESVNCF

SEQ ID NO: 744 - protein, 14 aa, synthetic construct
  REGION 1..14: note = Description of Artificial Sequence: Synthetic peptide
LHCWPDWEEIDICD

SEQ ID NO: 745 - protein, 14 aa, synthetic construct
  REGION 1..14: note = Description of Artificial Sequence: Synthetic peptide
DICGPDGQEDFVCS

SEQ ID NO: 746 - protein, 14 aa, synthetic construct
  REGION 1..14: note = Description of Artificial Sequence: Synthetic peptide
DVCWPDWEVNYFCD

SEQ ID NO: 747 - protein, 14 aa, synthetic construct
  REGION 1..14: note = Description of Artificial Sequence: Synthetic peptide
VNCGPEMDESIDCA

SEQ ID NO: 748 - protein, 14 aa, synthetic construct
  REGION 1..14: note = Description of Artificial Sequence: Synthetic peptide
DNCGPEFDEATVCN

SEQ ID NO: 749 - protein, 14 aa, synthetic construct
  REGION 1..14: note = Description of Artificial Sequence: Synthetic peptide
DLCGPEFEEVHNCN

SEQ ID NO: 750 - protein, 14 aa, synthetic construct
  REGION 1..14: note = Description of Artificial Sequence: Synthetic peptide
VNCGPEFDESSYCF

SEQ ID NO: 751 - protein, 14 aa, synthetic construct
  REGION 1..14: note = Description of Artificial Sequence: Synthetic peptide
NICWPDWEEDNFCS

SEQ ID NO: 752 - protein, 14 aa, synthetic construct
  REGION 1..14: note = Description of Artificial Sequence: Synthetic peptide
FVCGPEWEVYDDCD

SEQ ID NO: 753 - protein, 14 aa, synthetic construct
  REGION 1..14: note = Description of Artificial Sequence: Synthetic peptide
AYCGPELEVVHLCV

SEQ ID NO: 754 - protein, 14 aa, synthetic construct
  REGION 1..14: note = Description of Artificial Sequence: Synthetic peptide
PFCGPEMDETIDCY

SEQ ID NO: 755 - protein, 14 aa, synthetic construct
  REGION 1..14: note = Description of Artificial Sequence: Synthetic peptide
LLCGPVMEDVFACY

SEQ ID NO: 756 - protein, 14 aa, synthetic construct
  REGION 1..14: note = Description of Artificial Sequence: Synthetic peptide
DLCGPEFDESTNCY

SEQ ID NO: 757 - protein, 14 aa, synthetic construct
  REGION 1..14: note = Description of Artificial Sequence: Synthetic peptide
HDCGPEMEEYYLCP

SEQ ID NO: 758 - protein, 14 aa, synthetic construct
  REGION 1..14: note = Description of Artificial Sequence: Synthetic peptide
TDCGPEYDESIICP

SEQ ID NO: 759 - protein, 14 aa, synthetic construct
  REGION 1..14: note = Description of Artificial Sequence: Synthetic peptide
VLCWPDWEDYADCN

SEQ ID NO: 760 - protein, 14 aa, synthetic construct
  REGION 1..14: note = Description of Artificial Sequence: Synthetic peptide
NDCGPELDESLTCD

SEQ ID NO: 761 - protein, 14 aa, synthetic construct
  REGION 1..14: note = Description of Artificial Sequence: Synthetic peptide
IYCGPELDESIACY

SEQ ID NO: 762 - protein, 14 aa, synthetic construct
  REGION 1..14: note = Description of Artificial Sequence: Synthetic peptide
NYCGPEVEEFNFCH

SEQ ID NO: 763 - protein, 14 aa, synthetic construct
  REGION 1..14: note = Description of Artificial Sequence: Synthetic peptide
DVCGPEIEEYSFCI

SEQ ID NO: 764 - protein, 14 aa, synthetic construct
  REGION 1..14: note = Description of Artificial Sequence: Synthetic peptide
DLCGPEVEEITDCA

SEQ ID NO: 765 - protein, 14 aa, synthetic construct
  REGION 1..14: note = Description of Artificial Sequence: Synthetic peptide
HDCGPEFDESVFCI

SEQ ID NO: 766 - protein, 14 aa, synthetic construct
  REGION 1..14: note = Description of Artificial Sequence: Synthetic peptide
PLCGPVLEEDIYCY

SEQ ID NO: 767 - protein, 14 aa, synthetic construct
  REGION 1..14: note = Description of Artificial Sequence: Synthetic peptide
DLCGPEFEDIIDCN

SEQ ID NO: 768 - protein, 14 aa, synthetic construct
  REGION 1..14: note = Description of Artificial Sequence: Synthetic peptide
DYCGPEVEVPSNCN

SEQ ID NO: 769 - protein, 14 aa, synthetic construct
  REGION 1..14: note = Description of Artificial Sequence: Synthetic peptide
IICGPELDESTACD

SEQ ID NO: 770 - protein, 14 aa, synthetic construct
  REGION 1..14: note = Description of Artificial Sequence: Synthetic peptide
DHCGPEFDESVNCD

SEQ ID NO: 771 - protein, 14 aa, synthetic construct
  REGION 1..14: note = Description of Artificial Sequence: Synthetic peptide
FDCGPEFDESLYCS

SEQ ID NO: 772 - protein, 14 aa, synthetic construct
  REGION 1..14: note = Description of Artificial Sequence: Synthetic peptide
FACWPDWEEVYICY

SEQ ID NO: 773 - protein, 14 aa, synthetic construct
  REGION 1..14: note = Description of Artificial Sequence: Synthetic peptide
LYCGPEFDESLDCS

SEQ ID NO: 774 - protein, 14 aa, synthetic construct
  REGION 1..14: note = Description of Artificial Sequence: Synthetic peptide
DLCGPELEEAFLCA

SEQ ID NO: 775 - protein, 14 aa, synthetic construct
  REGION 1..14: note = Description of Artificial Sequence: Synthetic peptide
FACGPELDETLTCL

SEQ ID NO: 776 - protein, 14 aa, synthetic construct
  REGION 1..14: note = Description of Artificial Sequence: Synthetic peptide
FDCGPEVEEISNCD

SEQ ID NO: 777 - protein, 14 aa, synthetic construct
  REGION 1..14: note = Description of Artificial Sequence: Synthetic peptide
VHCGPELEYPFDCN

SEQ ID NO: 778 - protein, 14 aa, synthetic construct
  REGION 1..14: note = Description of Artificial Sequence: Synthetic peptide
AYCGPEFEEHTTCN

SEQ ID NO: 779 - protein, 14 aa, synthetic construct
  REGION 1..14: note = Description of Artificial Sequence: Synthetic peptide
LYCGPVSEQFTFCI

SEQ ID NO: 780 - protein, 14 aa, synthetic construct
  REGION 1..14: note = Description of Artificial Sequence: Synthetic peptide
DYCGPELDESYDCN

SEQ ID NO: 781 - protein, 14 aa, synthetic construct
  REGION 1..14: note = Description of Artificial Sequence: Synthetic peptide
FYCGPEWEEFDVCI

SEQ ID NO: 782 - protein, 14 aa, synthetic construct
  REGION 1..14: note = Description of Artificial Sequence: Synthetic peptide
FTCGPEVEEYDHCI

SEQ ID NO: 783 - protein, 14 aa, synthetic construct
  REGION 1..14: note = Description of Artificial Sequence: Synthetic peptide
YNCGPEFDESVTCF

SEQ ID NO: 784 - protein, 14 aa, synthetic construct
  REGION 1..14: note = Description of Artificial Sequence: Synthetic peptide
AVCGPENDESNSCA

SEQ ID NO: 785 - protein, 14 aa, synthetic construct
  REGION 1..14: note = Description of Artificial Sequence: Synthetic peptide
IYCWPDWEVPNDCA

SEQ ID NO: 786 - protein, 14 aa, synthetic construct
  REGION 1..14: note = Description of Artificial Sequence: Synthetic peptide
YFCGPEFEEFFHCY

SEQ ID NO: 787 - protein, 14 aa, synthetic construct
  REGION 1..14: note = Description of Artificial Sequence: Synthetic peptide
YVCGPDGDESSFCD

SEQ ID NO: 788 - protein, 14 aa, synthetic construct
  REGION 1..14: note = Description of Artificial Sequence: Synthetic peptide
ISCGPEVEEFFYCY

SEQ ID NO: 789 - protein, 14 aa, synthetic construct
  REGION 1..14: note = Description of Artificial Sequence: Synthetic peptide
LICGPVFEEDVYCD

SEQ ID NO: 790 - protein, 14 aa, synthetic construct
  REGION 1..14: note = Description of Artificial Sequence: Synthetic peptide
VYCGPEVEDHNYCA

SEQ ID NO: 791 - protein, 14 aa, synthetic construct
  REGION 1..14: note = Description of Artificial Sequence: Synthetic peptide
LDCGPEFEFVYICA

SEQ ID NO: 792 - protein, 14 aa, synthetic construct
  REGION 1..14: note = Description of Artificial Sequence: Synthetic peptide
YDCGPEFEEDLPCI

SEQ ID NO: 793 - protein, 14 aa, synthetic construct
  REGION 1..14: note = Description of Artificial Sequence: Synthetic peptide
DVCGPEVEEDYYCD

SEQ ID NO: 794 - protein, 14 aa, synthetic construct
  REGION 1..14: note = Description of Artificial Sequence: Synthetic peptide
NDCWPDWEYDNVCV

SEQ ID NO: 795 - protein, 14 aa, synthetic construct
  REGION 1..14: note = Description of Artificial Sequence: Synthetic peptide
DLCGPEFEVANDCN

SEQ ID NO: 796 - protein, 14 aa, synthetic construct
  REGION 1..14: note = Description of Artificial Sequence: Synthetic peptide
HDCGPELDESISCN

SEQ ID NO: 797 - protein, 14 aa, synthetic construct
  REGION 1..14: note = Description of Artificial Sequence: Synthetic peptide
LDCWPDWEETTHCD

SEQ ID NO: 798 - protein, 14 aa, synthetic construct
  REGION 1..14: note = Description of Artificial Sequence: Synthetic peptide
IICGPEVEEDDYCL

SEQ ID NO: 799 - protein, 14 aa, synthetic construct
  REGION 1..14: note = Description of Artificial Sequence: Synthetic peptide
YYCWPDWEEVIICD

SEQ ID NO: 800 - protein, 14 aa, synthetic construct
  REGION 1..14: note = Description of Artificial Sequence: Synthetic peptide
FDCGPEIDEYTNCN

SEQ ID NO: 801 - protein, 14 aa, synthetic construct
  REGION 1..14: note = Description of Artificial Sequence: Synthetic peptide
ITCGPELDETINCD

SEQ ID NO: 802 - protein, 14 aa, synthetic construct
  REGION 1..14: note = Description of Artificial Sequence: Synthetic peptide
DSCGPEVEEDIYCI

SEQ ID NO: 803 - protein, 14 aa, synthetic construct
  REGION 1..14: note = Description of Artificial Sequence: Synthetic peptide
YLCGPEFDESGNCH

SEQ ID NO: 804 - protein, 14 aa, synthetic construct
  REGION 1..14: note = Description of Artificial Sequence: Synthetic peptide
DNCGPELPEDYFCD

SEQ ID NO: 805 - protein, 14 aa, synthetic construct
  REGION 1..14: note = Description of Artificial Sequence: Synthetic peptide
VLCGPEFEEVSNCN

SEQ ID NO: 806 - protein, 14 aa, synthetic construct
  REGION 1..14: note = Description of Artificial Sequence: Synthetic peptide
VDCWPDWEEDIVCD

SEQ ID NO: 807 - protein, 14 aa, synthetic construct
  REGION 1..14: note = Description of Artificial Sequence: Synthetic peptide
VYCWPDWEDNFPCY

SEQ ID NO: 808 - protein, 14 aa, synthetic construct
  REGION 1..14: note = Description of Artificial Sequence: Synthetic peptide
DYCGPEVEEHFNCH

SEQ ID NO: 809 - protein, 14 aa, synthetic construct
  REGION 1..14: note = Description of Artificial Sequence: Synthetic peptide
ADCGPEIEEDAYCY

SEQ ID NO: 810 - protein, 14 aa, synthetic construct
  REGION 1..14: note = Description of Artificial Sequence: Synthetic peptide
ILCWPDWEDATFCY

SEQ ID NO: 811 - protein, 14 aa, synthetic construct
  REGION 1..14: note = Description of Artificial Sequence: Synthetic peptide
IHCWPDWEDFNICP

SEQ ID NO: 812 - protein, 14 aa, synthetic construct
  REGION 1..14: note = Description of Artificial Sequence: Synthetic peptide
TICGPEVEDYNDCI

SEQ ID NO: 813 - protein, 14 aa, synthetic construct
  REGION 1..14: note = Description of Artificial Sequence: Synthetic peptide
DDCGPELDESVACI

SEQ ID NO: 814 - protein, 14 aa, synthetic construct
  REGION 1..14: note = Description of Artificial Sequence: Synthetic peptide
DDCWPDWEDHIFCF

SEQ ID NO: 815 - protein, 14 aa, synthetic construct
  REGION 1..14: note = Description of Artificial Sequence: Synthetic peptide
VNCGPEVEEIIFCD

SEQ ID NO: 816 - protein, 14 aa, synthetic construct
  REGION 1..14: note = Description of Artificial Sequence: Synthetic peptide
IFCWPDWEDDTVCI

SEQ ID NO: 817 - protein, 14 aa, synthetic construct
  REGION 1..14: note = Description of Artificial Sequence: Synthetic peptide
IICGPEFEEISDCL

SEQ ID NO: 818 - protein, 14 aa, synthetic construct
  REGION 1..14: note = Description of Artificial Sequence: Synthetic peptide
FDCGPEVEEYNDCD

SEQ ID NO: 819 - protein, 14 aa, synthetic construct
  REGION 1..14: note = Description of Artificial Sequence: Synthetic peptide
NDCGPELDETLYCI

SEQ ID NO: 820 - protein, 14 aa, synthetic construct
  REGION 1..14: note = Description of Artificial Sequence: Synthetic peptide
AICGPELEEDISCN

SEQ ID NO: 821 - protein, 14 aa, synthetic construct
  REGION 1..14: note = Description of Artificial Sequence: Synthetic peptide
HLCGPEFDESTNCY

SEQ ID NO: 822 - protein, 14 aa, synthetic construct
  REGION 1..14: note = Description of Artificial Sequence: Synthetic peptide
SYCGPELDESVACI

SEQ ID NO: 823 - protein, 14 aa, synthetic construct
  REGION 1..14: note = Description of Artificial Sequence: Synthetic peptide
LYCGPEFEQLADCT

SEQ ID NO: 824 - protein, 14 aa, synthetic construct
  REGION 1..14: note = Description of Artificial Sequence: Synthetic peptide
IDCGPELDESIACN

SEQ ID NO: 825 - protein, 14 aa, synthetic construct
  REGION 1..14: note = Description of Artificial Sequence: Synthetic peptide
FDCGPDGQEDLVCN

SEQ ID NO: 826 - protein, 14 aa, synthetic construct
  REGION 1..14: note = Description of Artificial Sequence: Synthetic peptide
NLCGPEFEEFFDCY

SEQ ID NO: 827 - protein, 14 aa, synthetic construct
  REGION 1..14: note = Description of Artificial Sequence: Synthetic peptide
SICGPELQEDIVCP

SEQ ID NO: 828 - protein, 14 aa, synthetic construct
  REGION 1..14: note = Description of Artificial Sequence: Synthetic peptide
NPCGPEYDESAHCD

SEQ ID NO: 829 - protein, 14 aa, synthetic construct
  REGION 1..14: note = Description of Artificial Sequence: Synthetic peptide
YDCGPELDEYYNCN

SEQ ID NO: 830 - protein, 14 aa, synthetic construct
  REGION 1..14: note = Description of Artificial Sequence: Synthetic peptide
VDCGPELDETIFCD

SEQ ID NO: 831 - protein, 14 aa, synthetic construct
  REGION 1..14: note = Description of Artificial Sequence: Synthetic peptide
DNCGPELDESVTCT

SEQ ID NO: 832 - protein, 14 aa, synthetic construct
  REGION 1..14: note = Description of Artificial Sequence: Synthetic peptide
VDCGPELDESSYCD

SEQ ID NO: 833 - protein, 14 aa, synthetic construct
  REGION 1..14: note = Description of Artificial Sequence: Synthetic peptide
YYCGPEFEFIDFCF

SEQ ID NO: 834 - protein, 14 aa, synthetic construct
  REGION 1..14: note = Description of Artificial Sequence: Synthetic peptide
YDCWPDWEVITYCN

SEQ ID NO: 835 - protein, 14 aa, synthetic construct
  REGION 1..14: note = Description of Artificial Sequence: Synthetic peptide
FDCGPEIEEDFFCV

SEQ ID NO: 836 - protein, 14 aa, synthetic construct
  REGION 1..14: note = Description of Artificial Sequence: Synthetic peptide
IFCWPDWDDINFCD

SEQ ID NO: 837 - protein, 14 aa, synthetic construct
  REGION 1..14: note = Description of Artificial Sequence: Synthetic peptide
NFCGPELPEDITCY

SEQ ID NO: 838 - protein, 14 aa, synthetic construct
  REGION 1..14: note = Description of Artificial Sequence: Synthetic peptide
SLCGPEFEEYYHCL

SEQ ID NO: 839 - protein, 14 aa, synthetic construct
  REGION 1..14: note = Description of Artificial Sequence: Synthetic peptide
ASCGPELDESLDCL

SEQ ID NO: 840 - protein, 14 aa, synthetic construct
  REGION 1..14: note = Description of Artificial Sequence: Synthetic peptide
SFCGPEREWDLACY

SEQ ID NO: 841 - protein, 14 aa, synthetic construct
  REGION 1..14: note = Description of Artificial Sequence: Synthetic peptide
HLCGPEFEDVLDCI

SEQ ID NO: 842 - protein, 14 aa, synthetic construct
  REGION 1..14: note = Description of Artificial Sequence: Synthetic peptide
LDCGPDGDEFYYCL

SEQ ID NO: 843 - protein, 14 aa, synthetic construct
  REGION 1..14: note = Description of Artificial Sequence: Synthetic peptide
DNCWPDWEEDIACT

SEQ ID NO: 844 - protein, 14 aa, synthetic construct
  REGION 1..14: note = Description of Artificial Sequence: Synthetic peptide
SDCGPELDETIHCI

SEQ ID NO: 845 - protein, 14 aa, synthetic construct
  REGION 1..14: note = Description of Artificial Sequence: Synthetic peptide
NSCGPDGDESVDCL

SEQ ID NO: 846 - protein, 14 aa, synthetic construct
  REGION 1..14: note = Description of Artificial Sequence: Synthetic peptide
DLCGPELDESTLCI

SEQ ID NO: 847 - protein, 14 aa, synthetic construct
  REGION 1..14: note = Description of Artificial Sequence: Synthetic peptide
FDCGPESDESFNCY

SEQ ID NO: 848 - protein, 14 aa, synthetic construct
  REGION 1..14: note = Description of Artificial Sequence: Synthetic peptide
DLCGPEFEEDDICY

SEQ ID NO: 849 - protein, 14 aa, synthetic construct
  REGION 1..14: note = Description of Artificial Sequence: Synthetic peptide
HACGPEFEEDTYCF

SEQ ID NO: 850 - protein, 14 aa, synthetic construct
  REGION 1..14: note = Description of Artificial Sequence: Synthetic peptide
SDCGPELDESVACI

SEQ ID NO: 851 - protein, 14 aa, synthetic construct
  REGION 1..14: note = Description of Artificial Sequence: Synthetic peptide
IVCGPELPEDYNCY

SEQ ID NO: 852 - protein, 14 aa, synthetic construct
  REGION 1..14: note = Description of Artificial Sequence: Synthetic peptide
DLCGPEFDESFICF

SEQ ID NO: 853 - protein, 14 aa, synthetic construct
  REGION 1..14: note = Description of Artificial Sequence: Synthetic peptide
YDCGPEFDETLTCN

SEQ ID NO: 854 - protein, 14 aa, synthetic construct
  REGION 1..14: note = Description of Artificial Sequence: Synthetic peptide
YDCGPEVEEIVNCD

SEQ ID NO: 855 - protein, 14 aa, synthetic construct
  REGION 1..14: note = Description of Artificial Sequence: Synthetic peptide
VFCGPEIEDDHVCI

SEQ ID NO: 856 - protein, 14 aa, synthetic construct
  REGION 1..14: note = Description of Artificial Sequence: Synthetic peptide
FICGPEWEDDYACS

SEQ ID NO: 857 - protein, 14 aa, synthetic construct
  REGION 1..14: note = Description of Artificial Sequence: Synthetic peptide
NFCGPEFDESVGCN

SEQ ID NO: 858 - protein, 14 aa, synthetic construct
  REGION 1..14: note = Description of Artificial Sequence: Synthetic peptide
NLCGPEVEEILICD

SEQ ID NO: 859 - protein, 14 aa, synthetic construct
  REGION 1..14: note = Description of Artificial Sequence: Synthetic peptide
DSCGPEVEEYDLCN

SEQ ID NO: 860 - protein, 14 aa, synthetic construct
  REGION 1..14: note = Description of Artificial Sequence: Synthetic peptide
TLCGPEFEEITDCN

SEQ ID NO: 861 - protein, 14 aa, synthetic construct
  REGION 1..14: note = Description of Artificial Sequence: Synthetic peptide
TVCGPKMEMNSTCD

SEQ ID NO: 862 - protein, 14 aa, synthetic construct
  REGION 1..14: note = Description of Artificial Sequence: Synthetic peptide
VHCWPDWEDAVSCN

SEQ ID NO: 863 - protein, 14 aa, synthetic construct
  REGION 1..14: note = Description of Artificial Sequence: Synthetic peptide
DLCGPDGNELDFCF

SEQ ID NO: 864 - protein, 14 aa, synthetic construct
  REGION 1..14: note = Description of Artificial Sequence: Synthetic peptide
DLCGPDGEEHYYCD

SEQ ID NO: 865 - protein, 14 aa, synthetic construct
  REGION 1..14: note = Description of Artificial Sequence: Synthetic peptide
FNCGPEVEEILLCT

SEQ ID NO: 866 - protein, 14 aa, synthetic construct
  REGION 1..14: note = Description of Artificial Sequence: Synthetic peptide
HYCGPEVENINDCI

SEQ ID NO: 867 - protein, 14 aa, synthetic construct
  REGION 1..14: note = Description of Artificial Sequence: Synthetic peptide
IDCGPELDESVICD

SEQ ID NO: 868 - protein, 14 aa, synthetic construct
  REGION 1..14: note = Description of Artificial Sequence: Synthetic peptide
IFCGPEIEQPALCY

SEQ ID NO: 869 - protein, 14 aa, synthetic construct
  REGION 1..14: note = Description of Artificial Sequence: Synthetic peptide
YDCGPEFQEDLVCP

SEQ ID NO: 870 - protein, 14 aa, synthetic construct
  REGION 1..14: note = Description of Artificial Sequence: Synthetic peptide
HACWPDWEEPNYCD

SEQ ID NO: 871 - protein, 14 aa, synthetic construct
  REGION 1..14: note = Description of Artificial Sequence: Synthetic peptide
NVCWPDWEEDYNCY

SEQ ID NO: 872 - protein, 14 aa, synthetic construct
  REGION 1..14: note = Description of Artificial Sequence: Synthetic peptide
SFCGQELGDNYDCI

SEQ ID NO: 873 - protein, 14 aa, synthetic construct
  REGION 1..14: note = Description of Artificial Sequence: Synthetic peptide
DDCGPELDETTVCY

SEQ ID NO: 874 - protein, 14 aa, synthetic construct
  REGION 1..14: note = Description of Artificial Sequence: Synthetic peptide
NFCGPEWEVATLCL

SEQ ID NO: 875 - protein, 14 aa, synthetic construct
  REGION 1..14: note = Description of Artificial Sequence: Synthetic peptide
DLCGPEVEEDTYCN

SEQ ID NO: 876 - protein, 14 aa, synthetic construct
  REGION 1..14: note = Description of Artificial Sequence: Synthetic peptide
ALCGPEVEQVDLCT

SEQ ID NO: 877 - protein, 14 aa, synthetic construct
  REGION 1..14: note = Description of Artificial Sequence: Synthetic peptide
DDCGPELDESVNCN

SEQ ID NO: 878 - protein, 14 aa, synthetic construct
  REGION 1..14: note = Description of Artificial Sequence: Synthetic peptide
IFCGPEFEQIIYCD

SEQ ID NO: 879 - protein, 14 aa, synthetic construct
  REGION 1..14: note = Description of Artificial Sequence: Synthetic peptide
SDCWPDWEEVYYCS

SEQ ID NO: 880 - protein, 14 aa, synthetic construct
  REGION 1..14: note = Description of Artificial Sequence: Synthetic peptide
FFCGPDGDEVAICD

SEQ ID NO: 881 - protein, 14 aa, synthetic construct
  REGION 1..14: note = Description of Artificial Sequence: Synthetic peptide
DDCWPDWEDDVYCY

SEQ ID NO: 882 - protein, 14 aa, synthetic construct
  REGION 1..14: note = Description of Artificial Sequence: Synthetic peptide
YDCGPEFDETVVCP

SEQ ID NO: 883 - protein, 14 aa, synthetic construct
  REGION 1..14: note = Description of Artificial Sequence: Synthetic peptide
SYCGPELDESVNCV

SEQ ID NO: 884 - protein, 14 aa, synthetic construct
  REGION 1..14: note = Description of Artificial Sequence: Synthetic peptide
AFCVSFQQSLPHCD

SEQ ID NO: 885 - protein, 14 aa, synthetic construct
  REGION 1..14: note = Description of Artificial Sequence: Synthetic peptide
VNCGPEVEEYFVCF

SEQ ID NO: 886 - protein, 14 aa, synthetic construct
  REGION 1..14: note = Description of Artificial Sequence: Synthetic peptide
TNCWPDWEEDFACV

SEQ ID NO: 887 - protein, 14 aa, synthetic construct
  REGION 1..14: note = Description of Artificial Sequence: Synthetic peptide
DDCGPEFEEIILCF

SEQ ID NO: 888 - protein, 14 aa, synthetic construct
  REGION 1..14: note = Description of Artificial Sequence: Synthetic peptide
NSCGPELEDYHLCP

SEQ ID NO: 889 - protein, 14 aa, synthetic construct
  REGION 1..14: note = Description of Artificial Sequence: Synthetic peptide
IHCGPDSDGFDFCD

SEQ ID NO: 890 - protein, 14 aa, synthetic construct
  REGION 1..14: note = Description of Artificial Sequence: Synthetic peptide
FDCGPELEEDHLCF

SEQ ID NO: 891 - protein, 14 aa, synthetic construct
  REGION 1..14: note = Description of Artificial Sequence: Synthetic peptide
TVCWPDFEEYADCD

SEQ ID NO: 892 - protein, 14 aa, synthetic construct
  REGION 1..14: note = Description of Artificial Sequence: Synthetic peptide
YFCWPDWEEAADCL

SEQ ID NO: 893 - protein, 14 aa, synthetic construct
  REGION 1..14: note = Description of Artificial Sequence: Synthetic peptide
VSCGPEFDESVDCI

SEQ ID NO: 894 - protein, 14 aa, synthetic construct
  REGION 1..14: note = Description of Artificial Sequence: Synthetic peptide
AVCGPELPEDIVCY

SEQ ID NO: 895 - protein, 14 aa, synthetic construct
  REGION 1..14: note = Description of Artificial Sequence: Synthetic peptide
VLCGPEVEEYHLCA

SEQ ID NO: 896 - protein, 14 aa, synthetic construct
  REGION 1..14: note = Description of Artificial Sequence: Synthetic peptide
SLCWPDWEEVDNCF

SEQ ID NO: 897 - protein, 14 aa, synthetic construct
  REGION 1..14: note = Description of Artificial Sequence: Synthetic peptide
TICGPDGQEDYNCH

SEQ ID NO: 898 - protein, 14 aa, synthetic construct
  REGION 1..14: note = Description of Artificial Sequence: Synthetic peptide
FLCGPEGEPTYLCT

SEQ ID NO: 899 - protein, 14 aa, synthetic construct
  REGION 1..14: note = Description of Artificial Sequence: Synthetic peptide
DDCWLEQHDIYVCA

SEQ ID NO: 900 - protein, 14 aa, synthetic construct
  REGION 1..14: note = Description of Artificial Sequence: Synthetic peptide
IFCGPEVEEVAFCF

SEQ ID NO: 901 - protein, 14 aa, synthetic construct
  REGION 1..14: note = Description of Artificial Sequence: Synthetic peptide
ALCGPEVEDDYDCL

SEQ ID NO: 902 - protein, 14 aa, synthetic construct
  REGION 1..14: note = Description of Artificial Sequence: Synthetic peptide
DDCGPEIELYLTCA

SEQ ID NO: 903 - protein, 14 aa, synthetic construct
  REGION 1..14: note = Description of Artificial Sequence: Synthetic peptide
TDCWPDWEDDSICD

SEQ ID NO: 904 - protein, 14 aa, synthetic construct
  REGION 1..14: note = Description of Artificial Sequence: Synthetic peptide
SICGPELEEIFLCN

SEQ ID NO: 905 - protein, 14 aa, synthetic construct
  REGION 1..14: note = Description of Artificial Sequence: Synthetic peptide
NDCGPELEEDILCF

SEQ ID NO: 906 - protein, 14 aa, synthetic construct
  REGION 1..14: note = Description of Artificial Sequence: Synthetic peptide
DDCGPELDETYSCY

SEQ ID NO: 907 - protein, 14 aa, synthetic construct
  REGION 1..14: note = Description of Artificial Sequence: Synthetic peptide
FVCGPEWEEIDLCI

SEQ ID NO: 908 - protein, 14 aa, synthetic construct
  REGION 1..14: note = Description of Artificial Sequence: Synthetic peptide
YDCGPELDESLSCP

SEQ ID NO: 909 - protein, 14 aa, synthetic construct
  REGION 1..14: note = Description of Artificial Sequence: Synthetic peptide
NLCGPELDESIICP

SEQ ID NO: 910 - protein, 14 aa, synthetic construct
  REGION 1..14: note = Description of Artificial Sequence: Synthetic peptide
IDCWPDWEEFNNCF

SEQ ID NO: 911 - protein, 14 aa, synthetic construct
  REGION 1..14: note = Description of Artificial Sequence: Synthetic peptide
AYCGPELEEHDYCY

SEQ ID NO: 912 - protein, 14 aa, synthetic construct
  REGION 1..14: note = Description of Artificial Sequence: Synthetic peptide
DDCSPQFDQIDLCD

SEQ ID NO: 913 - protein, 14 aa, synthetic construct
  REGION 1..14: note = Description of Artificial Sequence: Synthetic peptide
SYCGPESDESIYCY

SEQ ID NO: 914 - protein, 14 aa, synthetic construct
  REGION 1..14: note = Description of Artificial Sequence: Synthetic peptide
SLCGPEFQEDAPCN

SEQ ID NO: 915 - protein, 14 aa, synthetic construct
  REGION 1..14: note = Description of Artificial Sequence: Synthetic peptide
DACGPELDETTHCD

SEQ ID NO: 916 - protein, 14 aa, synthetic construct
  REGION 1..14: note = Description of Artificial Sequence: Synthetic peptide
NDCGPEVEEVADCF

SEQ ID NO: 917 - protein, 14 aa, synthetic construct
  REGION 1..14: note = Description of Artificial Sequence: Synthetic peptide
YICGPEFEQDYFCF

SEQ ID NO: 918 - protein, 14 aa, synthetic construct
  REGION 1..14: note = Description of Artificial Sequence: Synthetic peptide
TDCGPDGDETNYCF

SEQ ID NO: 919 - protein, 14 aa, synthetic construct
  REGION 1..14: note = Description of Artificial Sequence: Synthetic peptide
YDCGPEVDESVLCP

SEQ ID NO: 920 - protein, 14 aa, synthetic construct
  REGION 1..14: note = Description of Artificial Sequence: Synthetic peptide
IDCGPEFDESAYCT

SEQ ID NO: 921 - protein, 14 aa, synthetic construct
  REGION 1..14: note = Description of Artificial Sequence: Synthetic peptide
FDCGPEFEEFFHCY

SEQ ID NO: 922 - protein, 14 aa, synthetic construct
  REGION 1..14: note = Description of Artificial Sequence: Synthetic peptide
FPCGPEIEEYDYCV

SEQ ID NO: 923 - protein, 14 aa, synthetic construct
  REGION 1..14: note = Description of Artificial Sequence: Synthetic peptide
ADCGPIFESIDICV

SEQ ID NO: 924 - protein, 14 aa, synthetic construct
  REGION 1..14: note = Description of Artificial Sequence: Synthetic peptide
ISCGPDLWPTDICT

SEQ ID NO: 925 - protein, 14 aa, synthetic construct
  REGION 1..14: note = Description of Artificial Sequence: Synthetic peptide
DDCGPDGDEVHTCN

SEQ ID NO: 926 - protein, 14 aa, synthetic construct
  REGION 1..14: note = Description of Artificial Sequence: Synthetic peptide
IDCWPDWEGSFACN

SEQ ID NO: 927 - protein, 14 aa, synthetic construct
  REGION 1..14: note = Description of Artificial Sequence: Synthetic peptide
LNCGPDGDETFYCD

SEQ ID NO: 928 - protein, 14 aa, synthetic construct
  REGION 1..14: note = Description of Artificial Sequence: Synthetic peptide
IHCGPELGAYISCS

SEQ ID NO: 929 - protein, 14 aa, synthetic construct
  REGION 1..14: note = Description of Artificial Sequence: Synthetic peptide
TICWPDWEEDYFCY

SEQ ID NO: 930 - protein, 14 aa, synthetic construct
  REGION 1..14: note = Description of Artificial Sequence: Synthetic peptide
HSCLAQFDQDLVCI

SEQ ID NO: 931 - protein, 14 aa, synthetic construct
  REGION 1..14: note = Description of Artificial Sequence: Synthetic peptide
NNCASDLSEDNSCI

SEQ ID NO: 932 - protein, 14 aa, synthetic construct
  REGION 1..14: note = Description of Artificial Sequence: Synthetic peptide
VNCWPDWEEDVACN

SEQ ID NO: 933 - protein, 14 aa, synthetic construct
  REGION 1..14: note = Description of Artificial Sequence: Synthetic peptide
VYCGPELDESFYCF

SEQ ID NO: 934 - protein, 14 aa, synthetic construct
  REGION 1..14: note = Description of Artificial Sequence: Synthetic peptide
LFCGTVLDEFFDCD

SEQ ID NO: 935 - protein, 14 aa, synthetic construct
  REGION 1..14: note = Description of Artificial Sequence: Synthetic peptide
FDCGPELDESVSCA

SEQ ID NO: 936 - protein, 14 aa, synthetic construct
  REGION 1..14: note = Description of Artificial Sequence: Synthetic peptide
YYCGPEVEENVYCI

SEQ ID NO: 937 - protein, 14 aa, synthetic construct
  REGION 1..14: note = Description of Artificial Sequence: Synthetic peptide
DYCGPELDDSTICH

SEQ ID NO: 938 - protein, 14 aa, synthetic construct
  REGION 1..14: note = Description of Artificial Sequence: Synthetic peptide
VFCGPQWAEANACF

SEQ ID NO: 939 - protein, 14 aa, synthetic construct
  REGION 1..14: note = Description of Artificial Sequence: Synthetic peptide
FTCGPEYDESITCP

SEQ ID NO: 940 - protein, 14 aa, synthetic construct
  REGION 1..14: note = Description of Artificial Sequence: Synthetic peptide
LNCGPVNDESSVCI

SEQ ID NO: 941 - protein, 14 aa, synthetic construct
  REGION 1..14: note = Description of Artificial Sequence: Synthetic peptide
AICWPDWEEFSDCH

SEQ ID NO: 942 - protein, 14 aa, synthetic construct
  REGION 1..14: note = Description of Artificial Sequence: Synthetic peptide
YYCGVDLGANVYCY

SEQ ID NO: 943 - protein, 14 aa, synthetic construct
  REGION 1..14: note = Description of Artificial Sequence: Synthetic peptide
HYCGPEVEEDYHCD

SEQ ID NO: 944 - protein, 14 aa, synthetic construct
  REGION 1..14: note = Description of Artificial Sequence: Synthetic peptide
NDCGSLQYDIPTCV

SEQ ID NO: 945 - protein, 14 aa, synthetic construct
  REGION 1..14: note = Description of Artificial Sequence: Synthetic peptide
YVCRPQLDVYHYCN

SEQ ID NO: 946 - protein, 14 aa, synthetic construct
  REGION 1..14: note = Description of Artificial Sequence: Synthetic peptide
HDCGPDGDESIICS

SEQ ID NO: 947 - protein, 14 aa, synthetic construct
  REGION 1..14: note = Description of Artificial Sequence: Synthetic peptide
FDCGPELDETIICP

SEQ ID NO: 948 - protein, 14 aa, synthetic construct
  REGION 1..14: note = Description of Artificial Sequence: Synthetic peptide
VDCGSDRGENAACH

SEQ ID NO: 949 - protein, 14 aa, synthetic construct
  REGION 1..14: note = Description of Artificial Sequence: Synthetic peptide
NICLAQFNEDPTCN

SEQ ID NO: 950 - protein, 14 aa, synthetic construct
  REGION 1..14: note = Description of Artificial Sequence: Synthetic peptide
TNCGSKSQVSDHCI

SEQ ID NO: 951 - protein, 14 aa, synthetic construct
  REGION 1..14: note = Description of Artificial Sequence: Synthetic peptide
NHCHPQFWELTNCN

SEQ ID NO: 952 - protein, 14 aa, synthetic construct
  REGION 1..14: note = Description of Artificial Sequence: Synthetic peptide
LLCHPQGDLYHLCH

SEQ ID NO: 953 - protein, 14 aa, synthetic construct
  REGION 1..14: note = Description of Artificial Sequence: Synthetic peptide
TNCDSKLEGDDNCF

SEQ ID NO: 954 - protein, 14 aa, synthetic construct
  REGION 1..14: note = Description of Artificial Sequence: Synthetic peptide
NDCGPEMDESLLCD

SEQ ID NO: 955 - protein, 14 aa, synthetic construct
  REGION 1..14: note = Description of Artificial Sequence: Synthetic peptide
FHCGPEVDESINCN

SEQ ID NO: 956 - protein, 14 aa, synthetic construct
  REGION 1..14: note = Description of Artificial Sequence: Synthetic peptide
DFCGPDGDETYVCS

SEQ ID NO: 957 - protein, 14 aa, synthetic construct
  REGION 1..14: note = Description of Artificial Sequence: Synthetic peptide
VSCGPQFDENNTCN

SEQ ID NO: 958 - protein, 14 aa, synthetic construct
  REGION 1..14: note = Description of Artificial Sequence: Synthetic peptide
VYCHLESEQFDICI

SEQ ID NO: 959 - protein, 14 aa, synthetic construct
  REGION 1..14: note = Description of Artificial Sequence: Synthetic peptide
DDCGPEWEFVFFCD

SEQ ID NO: 960 - protein, 14 aa, synthetic construct
  REGION 1..14: note = Description of Artificial Sequence: Synthetic peptide
YNCEQQQDEDPSCI

SEQ ID NO: 961 - protein, 14 aa, synthetic construct
  REGION 1..14: note = Description of Artificial Sequence: Synthetic peptide
NTCGTEQHEFNGCL

SEQ ID NO: 962 - protein, 14 aa, synthetic construct
  REGION 1..14: note = Description of Artificial Sequence: Synthetic peptide
HPCQPGFEEVDYCV

SEQ ID NO: 963 - protein, 14 aa, synthetic construct
  REGION 1..14: note = Description of Artificial Sequence: Synthetic peptide
VACSRQLGEDAYCN

SEQ ID NO: 964 - protein, 14 aa, synthetic construct
  REGION 1..14: note = Description of Artificial Sequence: Synthetic peptide
DICGAQEVHVYTCP

SEQ ID NO: 965 - protein, 14 aa, synthetic construct
  REGION 1..14: note = Description of Artificial Sequence: Synthetic peptide
FFCEGNLDAYLLCL

SEQ ID NO: 966 - protein, 14 aa, synthetic construct
  REGION 1..14: note = Description of Artificial Sequence: Synthetic peptide
YYCGPDGEENIVCD